(19) United States
(12) Patent Application Publication
(10) Pub. No.: US 2025/0281556 A1
(43) Pub. Date: Sep. 11, 2025

(54) PRODUCTION OF RNA POLYNUCLEOTIDES ENCODING PICORNAVIRUS

(71) Applicant: ElevateBio Technologies, Inc., Waltham, MA (US)

(72) Inventors: Edward M. Kennedy, Andover, MA (US); Anton ALENKO, Medford, MA (US); Ana De Almeida, Stoneham, MA (US); Jeffrey David BRYANT, Cambridge, MA (US)

(21) Appl. No.: 18/859,955

(22) PCT Filed: Apr. 28, 2023
(86) PCT No.: PCT/US2023/066352

§ 371 Date: Oct. 24, 2024

Related U.S. Application Data
(60) Provisional application No. 63/336,601, filed on Apr. 29, 2022.

(51) Int. Cl. A61K 35/768; A61K 9/1271; A61K 9/51; C12N 7/00; C12N 15/86; C12N 15/88

(52) U.S. Cl. CPC A61K 35/768; A61K 9/1271; A61K 9/5123; C12N 7/00; C12N 15/86; C12N 15/88; C12N 2770/32032; C12N 2770/32043; C12N 2830/50

(57) ABSTRACT

The present disclosure relates to production of recombinant RNA molecules encoding an oncolytic virus genome, such as a picornavirus, with native 5′ end of the viral genome utilizing ribozymes. The present disclosure further relates to the design of corresponding DNA template and the use of the recombinant RNA molecules and/or corresponding particles for the treatment and prevention of cancer.

### CROSS-REFERENCE TO RELATED APPLICATIONS

This application claims the benefit of U.S. Provisional Application No. 63/336,601, filed Apr. 29, 2022, the content of which is herein incorporated by reference in its entirety.

### INCORPORATION OF THE TEXT FILE SUBMITTED ELECTRONICALLY

The contents of the text file submitted electronically herewith are incorporated herein by reference in their entirety. A computer readable format copy of the Sequence Listing (filename: ONCR_031_01WO_SeqList_ST26.xml, date created: Apr. 28, 2023, file size: 510,929 bytes).

### FIELD

The present disclosure generally relates to the fields of immunology, inflammation, and cancer therapeutics. More specifically, the present disclosure relates to production of RNA polynucleotides encoding the viral genome of an oncolytic virus such as a picornavirus (e.g., a Coxsackievirus) and the design of recombinant DNA molecules for such viral genome expression. The disclosure further relates to the treatment and prevention of proliferative disorders such as cancer.

### BACKGROUND

Oncolytic viruses are replication-competent viruses with lytic life-cycle able to infect and lyse tumor cells. Direct tumor cell lysis results not only in cell death, but also the generation of an innate and adaptive immune response against tumor antigens taken up and presented by local antigen presenting cells. Therefore, oncolytic viruses combat tumor cell growth through both direct cell lysis and by promoting antigen-specific adaptive responses capable of maintaining anti-tumor responses after viral clearance.

However, clinical use of replication-competent viruses poses several challenges. In general, viral exposure activates innate immune pathways, resulting in a broad, non-specific inflammatory response. If the patient has not been previously exposed to the virus, this initial innate immune response can lead to the development of an adaptive anti-viral response and the production of neutralizing antibodies. If a patient has been previously exposed to the virus, existing neutralizing anti-viral antibodies can prevent the desired lytic effects. In both instances, the presence of neutralizing antibodies not only prevents viral lysis of target cells, but also renders re-administration of the viral therapeutic ineffective. These factors limit the use of viral therapeutics in the treatment of metastatic cancers, as the efficacy of repeated systemic administration required for treatment of such cancers is hampered by naturally occurring anti-viral responses.

One strategy to overcome such challenges is to use artificial particles (e.g., lipid nanoparticles), rather than native viral particles, for delivering the polynucleotides that encode the viral genome to the target cells. Generating these artificial particles usually requires producing and packaging the recombinant polynucleotides encoding the viral genome in vitro, and such recombinant polynucleotides need to be able to efficiently generate the viral RNAs once introduced in cells. However, many viral genomes (e.g., those of various picornaviruses) require native 5′ and/or 3′ ends of the viral genome to efficiently replicate. And there remains a long-felt and unmet need in the art for compositions and methods related to producing recombinant polynucleotides that contain native 5′ and/or 3′ ends of the corresponding viral genome in vitro. The present disclosure provides such compositions and methods, and more.

### SUMMARY

In one aspect, the present disclosure provides a recombinant DNA molecule comprising, from 5′ to 3′, a promoter sequence, a 5′ junctional cleavage sequence, and a polynucleotide sequence encoding an RNA molecule comprising a synthetic RNA viral genome, wherein the 5′ junctional cleavage sequence comprises or consists of a ENV27 ribozyme encoding sequence.

In one aspect, the present disclosure provides a recombinant DNA molecule comprising, from 5′ to 3′, a promoter sequence, a 5′ junctional cleavage sequence, a polynucleotide sequence encoding an RNA molecule comprising a synthetic RNA viral genome, a poly-A tail, and a 3′ junctional cleavage sequence, wherein the 5′ junctional cleavage sequence comprises or consists of a ENV27 ribozyme encoding sequence.

In some embodiments, the ENV27 ribozyme encoding sequence comprises or consists of a polynucleotide sequence (excluding P3 stem insert) having at least 80% identity to SEQ ID NO: 132 (excluding its P3 stem insert corresponding to nucleotides 49-54 of SEQ ID NO: 132). In some embodiments, the polynucleotide sequence (excluding P3 stem insert) is 100% identical, or has at most 1, at most 2, at most 3, at most 4, at most 5, at most 6, at most 7, at most 8, at most 9, at most 10, or at most 11 mutations (insertions, deletions or substitutions) as compared to, SEQ ID NO: 132 (excluding its P3 stem insert corresponding to nucleotides 49-54 of SEQ ID NO: 132). In some embodiments, the mutation(s) are substitution(s). In some embodiments, the ENV27 ribozyme encoding sequence comprises the polynucleotides “TTTATT” or “TTTGTT” at the positions corresponding to nucleotides 25-30 of SEQ ID NO: 132. In some embodiments, the ENV27 ribozyme encoding sequence comprises the polynucleotides “TTTATT” at the positions corresponding to nucleotides 25-30 of SEQ ID NO: 132. In some embodiments, the ENV27 ribozyme encoding sequence comprises the P3 stem insert of about 1-30, about 1-20, about 6-20, or about 6-10 polynucleotides in length. In some embodiments, the P3 stem insert is at least 1, 2, 3, 4, 5, 6, 7, 8, 9, 10, 11, 12, 13, 14, 15, 16, 17, 18, 19, or 20 polynucleotides in length. In some embodiments, the P3 stem insert is 1, 2, 3, 4, 5, 6, 7, 8, 9, 10, 11, 12, 13, 14, 15, 16, 17, 18, 19, or 20 polynucleotides in length. In some embodiments, the P3 stem insert comprises or consists of the polynucleotides “AGATCT” at the region corresponding to nucleotides 49-54 of SEQ ID NO: 132. In some embodiments, the P3 stem insert comprises or consists of the polynucleotides “AGAGAAATCT” (SEQ ID NO: 137) at the region corresponding to nucleotides 49-54 of SEQ ID NO: 132. In some embodiments, the P3 stem insert comprises or consists of the polynucleotides “AGAACGAGAAATCGTTCT” (SEQ ID NO: 138) at the region corresponding to nucleotides 49-54 of SEQ ID NO: 132.

In some embodiments, the recombinant DNA molecule comprises, from 5′ to 3′, the promoter sequence, the 5′ junctional cleavage sequence, the polynucleotide sequence encoding the RNA molecule comprising the synthetic RNA viral genome, and a poly-A tail.

In some embodiments, the recombinant DNA molecule comprises, from 5′ to 3′, the promoter sequence, the 5′ junctional cleavage sequence, the polynucleotide sequence encoding the RNA molecule comprising the synthetic RNA viral genome, and a 3′ junctional cleavage sequence.

In some embodiments, the recombinant DNA molecule comprises, from 5′ to 3′, the promoter sequence, the 5′ junctional cleavage sequence, the polynucleotide sequence encoding the RNA molecule comprising the synthetic RNA viral genome, a poly-A tail, and a 3′ junctional cleavage sequence.

In some embodiments, the synthetic RNA viral genome encodes a picornavirus. In some embodiments, the picornavirus is a Coxsackievirus virus. In some embodiments, the 5′ end of the RNA viral genome starts with “UUAAA”. In some embodiments, the Coxsackievirus is a CVA21 strain. In some embodiments, the CVA21 strain is selected from the Kuykendall strain, the EF strain and the KY strain. In some embodiments, the 5′ end of the RNA viral genome comprises or consists of a polynucleotide sequence having at least 80%, at least 85%, at least 90%, at least 91%, at least 92%, at least 93%, at least 94%, at least 95%, at least 96%, at least 97%, at least 98%, at least 99%, or 100% identity to nucleotides 1-260 of any one of SEQ ID NO: 1, 5, or 9.

In some embodiments, the recombinant DNA molecule does not comprise additional nucleic acid between the 5′ junctional cleavage sequence and the polynucleotide sequence encoding the RNA molecule. In some embodiments, cleavage of the 5′ junctional cleavage sequence and/or the 3′ junctional cleavage sequence produces native 5′ and/or 3′ ends of the synthetic RNA viral genome after transcription.

In some embodiments, the recombinant DNA molecule of the disclosure further comprises a leader sequence between the promoter sequence and the 5′ junctional cleavage sequence. In some embodiments, the leader sequence is less than 100 bp, less than 90 bp, less than 80 bp, less than 70 bp, less than 60 bp, less than 50 bp, or less than 40 bp in length. In some embodiments, the leader sequence comprises or consists of a polynucleotide sequence having at least 70%, at least 80%, at least 90%, at least 95%, at least 96%, at least 97%, at least 98%, at least 99%, or 100% sequence identity according to SEQ ID NO: 135 or 136. In some embodiments, the leader sequence comprises or consists of a polynucleotide sequence having at least 70%, at least 80%, at least 90%, at least 95%, at least 96%, at least 97%, at least 98%, at least 99%, or 100% sequence identity according to SEQ ID NO: 135.

In some embodiments, the recombinant DNA molecule does not comprise additional nucleic acid between the promoter sequence and the leader sequence. In some embodiments, the recombinant DNA molecule does not comprise additional nucleic acid between the leader sequence and the 5′ junctional cleavage sequence.

In some embodiments, the promoter sequence is a T7 promoter sequence. In some embodiments, the T7 promoter sequence comprises or consists of SEQ ID NO: 91.

In some embodiments, the poly-A tail is about 50-90 bp in length or about 65-75 bp in length. In some embodiments, the poly-A tail is about 70 bp in length. In some embodiments, the poly-A tail is about 10-50 bp, or 25-35 bp in length.

In some embodiments, the 3′ junctional cleavage sequence comprises or consists of a ribozyme sequence. In some embodiments, the 3′ ribozyme sequence is a hepatitis delta virus ribozyme sequence.

In some embodiments, the 3′ junctional cleavage sequence comprises or consists of a restriction enzyme recognition sequence. In some embodiments, the 3′ junctional cleavage sequence comprises or consists of a Type IIS restriction enzyme recognition sequence. In some embodiments, the 3′ junctional cleavage sequence comprises or consists of a BsmBI recognition sequence. In some embodiments, the 3′ junctional cleavage sequence comprises or consists of a BsaI recognition sequence.

In some embodiments, the promoter sequence is a T7 promoter sequence, the leader sequence consists of a polynucleotide sequence according to SEQ ID NO: 135, the 5′ junctional cleavage sequence comprises or consists of a ENV27 ribozyme sequence according to any one of SEQ ID NO: 132-134, the poly-A tail is about 70 bp in length, and the 3′ junctional cleavage sequence comprises or consists of a BsmBI recognition sequence.

In some embodiments, the promoter sequence is a T7 promoter sequence, the leader sequence consists of a polynucleotide sequence according to SEQ ID NO: 135, the 5′ junctional cleavage sequence comprises or consists of a ENV27 ribozyme sequence according to any one of SEQ ID NO: 132-134, the poly-A tail is about 70 bp in length, and the 3′ junctional cleavage sequence comprises or consists of a BsaI recognition sequence.

In some embodiments, the recombinant DNA molecule does not comprise additional nucleic acid within the region spanning the promoter sequence and the 3′ junctional cleavage sequence.

In one aspect, the disclosure provides methods of producing a recombinant RNA molecule, comprising in vitro transcription of the recombinant DNA molecule of the disclosure and purification of the resulting recombinant RNA molecule. In some embodiments, the recombinant RNA molecule comprises 5′ and 3′ ends that are native to the viral genome encoded by the recombinant RNA molecule.

In one aspect, the disclosure provides recombinant RNA molecules transcribed from the recombinant DNA molecule of the disclosure. In some embodiments, at least 70%, at least 75%, at least 80%, at least 85%, at least 90%, at least 95%, at least 96%, at least 97%, at least 98%, at least 99%, at least 99.5%, or at least 99.9%, of the recombinant RNA molecules comprise 5′ and 3′ ends that are native to the viral genome encoded by the recombinant RNA molecule. In some embodiments, no more than 30%, no more than 25%, no more than 20%, no more than 15%, no more than 10%, no more than 5%, no more than 4%, no more than 3%, no more than 2%, no more than 1%, no more than 0.5%, or no more than 0.1%, of the recombinant RNA molecules comprise an RNA sequence encoded by the ENV27 ribozyme encoding sequence. In some embodiments, at least one of the recombinant RNA molecules comprises an RNA sequence encoded by the ENV27 ribozyme encoding sequence. In some embodiments, at least 0.0001%, at least 0.001%, at least 0.01%, at least 0.1%, or at least 1%, of the recombinant RNA molecules comprise an RNA sequence encoded by the ENV27 ribozyme encoding sequence.

In one aspect, the disclosure provides compositions comprising an effective amount of the recombinant RNA molecule of the disclosure, and a carrier suitable for administration to a mammalian subject.

In one aspect, the disclosure provides particles comprising the recombinant RNA molecule of the disclosure. In some embodiments, the particle is selected from the group consisting of a nanoparticle, an exosome, a liposome, and a lipoplex. In some embodiments, the particle is a lipid nanoparticle.

In one aspect, the disclosure provides pharmaceutical compositions comprising a plurality of particles of the disclosure. In some embodiments, delivery of the composition to a subject delivers the encapsulated recombinant RNA molecule to a target cell, and wherein the encapsulated recombinant RNA molecule produces an infectious virus capable of lysing the target cell.

In one aspect, the disclosure provides methods of killing a cancerous cell comprising exposing the cancerous cell to the particle of the disclosure, or compositions thereof, under conditions sufficient for the intracellular delivery of the particle to said cancerous cell, wherein the replication-competent virus produced by the encapsulated polynucleotide results in killing of the cancerous cell. In one aspect, the disclosure provides methods of treating a cancer in a subject comprising administering to a subject suffering from the cancer an effective amount of the particle of the disclosure, or compositions thereof. In some embodiments, the method is performed in vivo, in vitro, or ex vivo. In some embodiments, the cancer is lung cancer, breast cancer, colon cancer, or pancreatic cancer, and wherein the synthetic RNA viral genome comprises a polynucleotide sequence derived from the KY strain. In some embodiments, the cancer is bladder cancer, renal cell carcinoma, ovarian cancer, gastric cancer or liver cancer, and wherein the synthetic RNA viral genome comprises a polynucleotide sequence derived from the EF strain. In some embodiments, the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL). In some embodiments, the cancer is selected from the groups consisting of lung cancer, breast cancer, colon cancer, pancreatic cancer, bladder cancer, renal cell carcinoma, ovarian cancer, gastric cancer and liver cancer. In some embodiments, the cancer is renal cell carcinoma, lung cancer, or liver cancer. In some embodiments, the cancer is small cell lung cancer or non-small cell lung cancer (e.g., squamous cell lung cancer or lung adenocarcinoma). In some embodiments, the cancer is hepatocellular carcinoma (HCC) (e.g., Hepatitis B virus associated HCC). In some embodiments, the cancer is treatment-emergent neuroendocrine prostate cancer. In some embodiments, the cancer is lung cancer, liver cancer, prostate cancer (e.g., CRPC-NE), bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma, or melanoma. In some embodiments, the cancer is neuroblastoma.

### BRIEF DESCRIPTION OF THE DRAWINGS

FIG. 1 shows tumor volume in SK-MEL-28 tumor-bearing mice following intratumoral administration of PBS or CVA21-RNA molecules formulated with Lipofectamine or intravenous administration of LNPs comprising CVA21-Kuykendall strain RNA molecules (formulation ID: 70032-6C).

FIG. 2A shows an overview of an in vitro transcriptional approach to generate an authentic 3′ terminus for picornaviruses using 3′ Type IIS restriction enzyme recognition sites. FIG. 2B shows electrophoresis of DNA digestion by BsmBI or BsaI restriction enzyme.

FIG. 3 shows an RNaseH approach for generating an authentic 5′ terminus for picornaviruses using 5′ DNA primers and an RNaseH enzyme.

FIG. 4 shows a ribozyme approach for generating authentic 5′ termini for picornaviruses.

FIG. 5A-FIG. 5B show hammerhead ribozymes for generation of discrete 5′ termini. FIG. 5A shows a structural model of a minimal hammerhead ribozyme (HHR) that anneals and cleaves at the 5′ terminus at the arrow (SEQ ID NO: 75). FIG. 5B shows a structural model of a ribozyme with a stabilized stem I (STBL) for cleavage of 5′ terminus at the arrow (SEQ ID NO: 76).

FIG. 6A-FIG. 6B show pistol ribozymes for generation of discrete 5′ termini. FIG. 6A shows a schematic of wild type Pistol ribozyme characteristics (SEQ ID NO: 77). FIG. 6B shows Pistol ribozyme from P. polymyxa with a tetraloop added to fuse the P3 strands modeled by mFOLD. The dashed box is the area mutagenized to retain the fold of the ribozyme in the context of the viral sequence. The “GUC” sequence shown in the dashed box was mutated to “UCA” to generate Pistol 1 and the “GUC” sequence was mutated to “TTA” to generate Pistol 2. (SEQ ID NO: 78)

FIG. 7 shows the sequence alignment of multiple Pistol ribozyme variants and the location of the P2 motif.

FIG. 8 shows the in vitro transcription process for CVA21-RNA and Neg-RNA. Autocatalytic cleavage of CVA21-RNA by 5′ and 3′ ribozyme (Rib) generated CVA21-RNA with discrete 5′ and 3′ ends required for replication. In contrast, the Neg-RNA construct lacks ribozyme sequence and was not capable of replication and virion production.

FIG. 9A shows a general schematic of using junctional cleavage sequences to remove non-viral RNA polynucleotides from the genome transcripts in order to maintain the native 5′ and 3′ discrete ends of the virus. FIG. 9B shows a schematic of using junctional cleavage sequences to remove non-viral RNA polynucleotides from the genome transcripts in order to maintain the native 5′ and 3′ discrete ends of the virus wherein the 3′ junctional cleavage sequence comprises a restriction enzyme recognition site.

FIG. 10A shows the schematics of a non-limiting example of the CVA21 expression construct design and corresponding in vitro transcription process to generate synthetic RNA viral genomes with precise ends at 5′ and 3′. FIG. 10B shows the design of DNA constructs that test the cleavage efficiency of ribozymes as 5′ junctional cleavage sequence. FIG. 10C is a gel electrophoresis image showing the cleavage efficiency of the initial candidate ribozyme for either a shorter, ˜60 nt virus start or a longer, ˜260 nt virus start.

FIG. 11A shows the DNA sequences encoding various ENV27 ribozymes. FIG. 11B shows (left) a diagram of the ribozyme secondary structure and (right) the base pairings of the ribozyme P2 motif with the 5′ end of the viral genome.

FIG. 12A and FIG. 12B are gel electrophoresis images showing the cleavage efficiency of the indicated ribozyme constructs. FIG. 12C is a table summarizing the cleavage efficiency of each indicated ribozyme incorporated into viral genome expression constructs.

FIG. 13 is a chart showing the virus titer produced by cells transfected with the viral genome RNAs produced by the indicated DNA template.

FIG. 14A is a chart showing the growth of tumor in animals treated with LNPs comprising the viral genome RNA produced by the indicated DNA templates. FIG. 14B is a chart showing the body weight change of animals treated with LNPs comprising the viral genome RNA produced by the indicated DNA template. FIG. 14C shows tumor growth charts for NCI-H1299 animal tumor model treated with LNPs comprising the viral genome RNA produced by the indicated DNA templates.

FIG. 15A shows the UV A280 absorption profile of CVA21 viral genome with varying poly-A tail length using Oligo-dT chromatography. FIG. 15B shows the UV A280 absorption profile of SVV viral genome with varying poly-A tail length using Oligo-dT chromatography.

FIG. 16A is a diagram plotting the change of tumor sizes over time in animals treated with the indicated LNPs comprising RNA viral genomes obtained from in vitro transcription of DNA templates with different designs (e.g., varying poly-A tail length and 5′ ribozyme sequences), using a NCI-H1299 xenograft model. FIG. 16B shows diagrams plotting the change of tumor sizes over time in animals treated with the indicated LNPs.

### DETAILED DESCRIPTION

The present disclosure provides recombinant polynucleotides encoding viral genomes and design of template vectors for viral expression in vitro. Especially, the recombinant polynucleotides may be required to expose native 5′ and 3′ ends of the viral genome to allow efficient viral replication in cells. Producing such recombinant polynucleotides requires optimization of the design of vector template as well as the manufacturing process (e.g., expression, purification, encapsulation, and storage).

In some embodiments, the viral genomes are replication competent. The present disclosure also provides viral genomes that can be encapsulated in a non-immunogenic particle, such as a lipid nanoparticle, polymeric nanoparticle, or an exosome, which can be repeatedly administered to a subject. In some embodiments, the particle further encapsulates a polynucleotide encoding a payload molecule. Accordingly, the present disclosure enables the systemic delivery of a safe, efficacious recombinant polynucleotide vector, and provides methods for the treatment and prevention of a broad array of proliferative disorders (e.g., cancers).

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences). In this application, the use of “or” means “and/or” unless stated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 10%, in either direction (greater than or less than) of the stated reference value unless otherwise stated or otherwise evident from the context (except where such number would exceed 100% of a possible value). In some embodiments, the term “approximately” or “about” refers to a range of values that fall within 10% in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, 5, 6, 7, 8, 9, 10, 15, 20, 25, 30, 35, 40, 45, 50, 55, 60, 65, 70, 75, 80, 85, 90, 95, 99, or 100% as compared to a reference value. A decrease or reduction in a particular value may also be represented as a fold-change in the value compared to a reference value, for example, at least 1, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, 5, 6, 7, 8, 9, 10, 15, 20, 25, 30, 35, 40, 45, 50, 55, 60, 65, 70, 75, 80, 85, 90, 95, 99, 100, 200, 300, 400, 500% or more as compared to a reference value. An increase in a particular value may also be represented as a fold-change in the value compared to a reference value, for example, at least 1-fold, 2, 3, 4, 5, 6, 7, 8, 9, 10, 15, 20, 30, 40, 50, 60, 70, 80, 90, 100, 200, 500, 1000-fold or more, increase as compared to the level of a reference value.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. Unless noted otherwise, the term “sequence identity” in the claims refers to sequence identity as calculated by Clustal Omega® version 1.2.4 using default parameters.

The term “derived from” refers to a polypeptide or polynucleotide sequence that comprises all or a portion of a reference polypeptide or polynucleotide sequence. For example, an RNA polynucleotide encoding an SVV or CVA genome described herein may comprise a polynucleotide sequence derived from all or a portion of a reference SVV or CVA genome (e.g., a naturally occurring or modified SVV or CVA genome). A polypeptide or polynucleotide sequence “derived from” a reference polypeptide or polynucleotide sequence also includes polypeptide and/or polynucleotide sequences that comprise one more amino acid or nucleic acid mutations (e.g., substitutions, deletions, and/or insertions) relative to the reference polypeptide or polynucleotide sequence.

“Complementary” refers to the capacity for pairing, through base stacking and specific hydrogen bonding, between two sequences comprising naturally or non-naturally occurring (e.g., modified as described above) bases (nucleotides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

An “expression cassette” or “expression construct” refers to a polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject or contacting a composition with a cell and/or tissue.

“Treating” as used herein refers to delivering an agent or composition to a subject to affect a physiologic outcome. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state; and (c) curing the disease.

The term “effective amount” refers to the amount of an agent or composition required to result in a particular physiological effect (e.g., an amount required to increase, activate, and/or enhance a particular physiological effect). The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

“Effector function” refers to functions of an immune cell related to the generation, maintenance, and/or enhancement of an immune response against a target cell or target antigen.

The terms “microRNA,” “miRNA,” and “miR” are used interchangeably herein and refer to small non-coding endogenous RNAs of about 21-25 nucleotides in length that regulate gene expression by directing their target messenger RNAs (mRNA) for degradation or translational repression.

The term “composition” as used herein refers to a formulation of a recombinant RNA molecule or a particle-encapsulated recombinant RNA molecule described herein that is capable of being administered or delivered to a subject or cell.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals.

The term “replication-competent viral genome” refers to a viral genome encoding all of the viral genes necessary for viral replication and production of an infectious viral particle.

The term “oncolytic virus” refers to a virus that has been modified to, or naturally, preferentially infect cancer cells.

The term “vector” is used herein to refer to a nucleic acid molecule capable of transferring, encoding, or transporting another nucleic acid molecule.

The terms “corresponding to” or “correspond to”, as used herein in relation to the amino acid or nucleic acid position(s), refer to the position(s) in a first polypeptide/polynucleotide sequence that aligns with a given amino acid/nucleic acid in a reference polypeptide/polynucleotide sequence when the first and the reference polypeptide/polynucleotide sequences are aligned. Alignment is performed by one of skill in the art using software designed for this purpose, for example, Clustal Omega version 1.2.4 with the default parameters for that version.

The term “encapsulation efficiency” or “EE %” refers to the percentage of a target molecule (e.g., synthetic RNA viral genome) that is successfully entrapped into LNP. Encapsulation efficiency may be calculated using the formula:

(EE %)=(Wt/Wi)×100%
where Wt is the total amount of drug in the LNP suspension and Wi is the total quantity of drug added initially during preparation. As an illustrative example, if 97 mg of the target molecule are entrapped into LNPs out of a total 100 mg of the target molecule initially provided to the composition, the encapsulation efficiency may be given as 97%.

The term “lipid-nitrogen-to-phosphate ratio” or “(N:P)” refers to the ratio of positively-chargeable lipid amine groups to nucleic acid phosphate groups in a lipid nanoparticle.

The term “half-life” refers to a pharmacokinetic property of a molecule (e.g., a molecule encapsulated in a lipid nanoparticle). Half-life can be expressed as the time required to eliminate through biological processes (e.g., metabolism, excretion, accelerated blood clearance, etc.) fifty percent (50%) of a known quantity of a molecule in vivo, following its administration, from the subject's body (e.g., human patient or other mammal) or a specific compartment thereof, for example, as measured in serum, i.e., circulating half-life, or in other tissues. In general, an increase in half-life results in an increase in mean residence time (MRT) in circulation for the molecule administered.

The term “accelerated blood clearance” or “ABC” refers to a phenomenon in which certain pharmaceutical agents (e.g., PEG-containing LNPs) are rapidly cleared from the blood upon second and subsequent administrations. ABC has been observed for many lipid-delivery vehicles, including liposomes and LNPs.

As used herein, the term “ratio” when used in reference to lipid composition (e.g., as a percentage of total lipid content) refers to molar ratio, unless clearly indicated otherwise. The molar ratio as a percentage of total lipid content can also be represented by “mol %”. For example, “49:22:28.5:0.5 mol %” means a molar ratio of 49:22:28.5:0.5.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic,” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “aryl” used alone or as part of a larger moiety as in “aralkyl,” “aralkoxy,” or “aryloxyalkyl,” refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “haloaliphatic” refers to an aliphatic group that is substituted with one or more halogen atoms.

The term “haloalkyl” refers to a straight or branched alkyl group that is substituted with one or more halogen atoms.

The term “halogen” means F, Cl, Br, or I.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSIR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘}) R^{∘}; —C(O)C(O) R^{∘}; —C(O)CH₂C(O)R^{∘}; —C(NOR^{∘}) R^{∘}; —(CH₂)₀₋₄SSR^{∘}; —(CH₂)₀₋₄S(O)₂R^{∘}; (CH₂)₀₋₄S(O)₂OR^{∘}; —(CH₂)₀₋₄OS(O)₂R^{∘}; —S(O)₂NR^{∘}₂; (CH₂)₀₋₄S(O) R^{∘}; —N(R^{∘}S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘)}₂; SiR^{∘}₃; —(C₁₋₄ straight or branched)alkylene)O—N(R^{∘)}₂; or —(C₁₋₄ straight or branched alkylene)C(O)O—N(R^{∘)}₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}, —(CH₂)₀₋₂OH, —(CH₂)₀₋₂OR^{•}, —(CH₂)₀₋₂CH(OR)₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene) C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include-O and =S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, ═NOR*, —O(C(R*₂)₂₋₃O—, or —S(C(R*₂)₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The term “tertiary amine” is used to describe an amine (nitrogen atom) which is attached to three carbon-containing groups, each of the groups being covalently bonded to the amine group through a carbon atom within the group. A tertiary amine may be protonated or form a complex with a Lewis acid.

The recitation of a listing of chemical groups in any definition of a variable herein includes definitions of that variable as any single group or combination of listed groups.

The recitation of an embodiment for a variable herein includes that embodiment as any single embodiment or in combination with any other embodiments or portions thereof.

Unless otherwise stated, structures depicted herein are also meant to include all enantiomeric, diastereomeric, and geometric (or conformational) forms of the structure, for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Synthetic RNA Viral Genomes

In some embodiments, the present disclosure provides a recombinant RNA molecule encoding an oncolytic virus (e.g., an RNA genome). Such recombinant RNA molecules are referred to herein as “synthetic viral genomes” or “synthetic RNA viral genomes”. In such embodiments, the synthetic RNA viral genome is capable of producing an infectious, lytic virus when introduced into a cell by a non-viral delivery vehicle and does not require additional exogenous genes or proteins to be present in the cell in order to replicate and produce an infectious virus. Rather, the endogenous translational mechanisms in the host cell mediate expression of the viral proteins from the synthetic RNA viral genome. The expressed viral proteins then mediate viral replication and assembly into an infectious viral particle (which may comprise a capsid protein, an envelope protein, and/or a membrane protein) comprising the RNA viral genome. As such, the RNA polynucleotides described herein (i.e., the synthetic RNA viral genomes), when introduced into a host cell, produce a virus that can infect another host cell. In some embodiments, the oncolytic virus is a picornavirus. In some embodiments, the picornavirus is a CVA21. In some embodiments, the picornavirus is an SVV.

In some embodiments, the synthetic viral genome is provided as a recombinant ribonucleic acid (RNA) (i.e., a synthetic RNA viral genome). In some embodiments, the synthetic RNA viral genomes comprise one or more nucleic acid analogues. Examples of nucleic acid analogues include 2′-O-methyl-substituted RNA, 2′-O-methoxy-ethyl bases, 2′ Fluoro bases, locked nucleic acids (LNAs), unlocked nucleic acids (UNA), bridged nucleic acids (BNA), morpholinos, and peptide nucleic acids (PNA). In some embodiments, the synthetic RNA viral genome is a replicon, a RNA viral genome encoding a transgene, an mRNA molecule, or a circular RNA molecule (circRNA). In some embodiments, the synthetic RNA viral genome comprises a single stranded RNA (ssRNA) viral genome. In some embodiments, the single-stranded genome may be a positive sense or negative sense genome.

In some embodiments, the recombinant RNA molecule is a circular RNA molecule (circRNA). CircRNA molecules lack the free ends necessary for exonuclease mediated degradation, thus extending the half-life of the RNA molecule and enabling more stable protein production over time (See e.g., Wesselhoeft et al., Engineering circular RNA for potent and stable translation in eukaryotic cells. Nature Communications. (2018) 9:2629). In order to produce a functional RNA virus from a circRNA molecule, it is necessary to “break open” the circular construct once inside a cell so that the linear RNA genome with the appropriate 3′ and 5′ native ends can be produced. Therefore, in some embodiments, the recombinant RNA molecule encoding the oncolytic virus is provided as a circRNA molecule and further comprises one or more additional RNA sequences that facilitate the linearization of the circRNA molecule inside a cell. Examples of such additional RNA sequences include siRNA target sites, miRNA target sites, and guide RNA target sites. The corresponding siRNA, miRNA, or gRNA can be co-formulated with the circRNA molecule. Alternatively, the miRNA target site can be selected based on the expression of the cognate miRNA in a target cell, such that cleavage of the circRNA molecule and initial expression of the encoded oncolytic virus is limited to target cells expressing a particular miRNA.

The synthetic RNA viral genomes described herein encode an oncolytic virus. Examples of oncolytic viruses are known in the art including, but not limited to a picornavirus (e.g., a coxsackievirus), a polio virus, a measles virus, a vesicular stomatitis virus, an orthomyxovirus, and a maraba virus. In some embodiments, the oncolytic virus encoded by the synthetic RNA viral genome is a virus in the family Picornaviridae family such as a coxsackievirus, a polio virus (including a chimeric polio virus such as PVS—RIPO and other chimeric Picornaviruses), or a Seneca valley virus, or any virus of chimeric origin from any multitude of picornaviruses, a virus in the Arenaviridae family such a lassa virus, a virus in the Retroviridae family such as a murine leukemia virus, a virus in the family Orthomyxoviridae such as influenza A virus, a virus in the family Paramyxoviridae such as Newcastle disease virus or measles virus, a virus in the Reoviridae family such as mammalian orthoreovirus, a virus in the Togaviridae family such as sindbis virus, or a virus in the Rhabdoviridae family such as vesicular stomatitis virus (VSV) or a maraba virus.

### Positive-Sense, Single-Stranded RNA Viruses

In some embodiments, the synthetic RNA viral genomes described herein encode a single-stranded RNA (ssRNA) viral genome. In some embodiments, the ssRNA virus is a positive-sense, ssRNA (+ sense ssRNA) virus. Exemplary+sense ssRNA viruses include members of the Picornaviridae family (e.g. coxsackievirus, poliovirus, and Seneca Valley virus (SVV), including SVV-A), the Coronaviridae family (e.g., Alphacoronaviruses such as HCoV-229E and HCoV-NL63, Betacoronoaviruses such as HCoV—HKU1, HCoV-OC3, and MERS-CoV), the Retroviridae family (e.g., Murine leukemia virus), and the Togaviridae family (e.g., Alphaviruses such as the Semliki Forest virus, Sindbis virus, Ross River virus, or Chikungunya virus). Additional exemplary genera and species of positive-sense, ssRNA viruses are shown below in Table 1.

TABLE 1
Positive-sense ssRNA Viruses

| Family/Subfamily | Genus | Natural Host | Species |
|---|---|---|---|
| Picornaviridae | Cardiovirus | Human | |
| | Cosavirus | Human | |
| | Enterovirus | Human | Coxsackievirus |
| | | Human | Poliovirus |
| | Hepatovirus | Human | |
| | Kobuvirus | Human | |
| | Parechovirus | Human | |
| | Rosavirus | Human | |
| | Salivirus | Human | |
| | Pasivirus | Pigs | |
| | Senecavirus | Pigs | Seneca Valley |
| | | | Virus A |
| Caliciviridae | Sapovirus | Human | |
| | Norovirus | Human | |
| | Nebovirus | Bovine | |
| | Vesivirus | Felines/Swine | |
| Hepeviridae | Orthohepevirus | | |
| Astroviridae | Mamastrovirus | Human | |
| | Avastrovirus | Birds | |
| Flaviviridae | Hepacivirus | Human | |
| | Flavivirus | Arthropod | |
| | Pegivirus | | |
| | Pestivirus | Mammals | |
| Coronaviridae/Coronavirinae | Alphacoronavirus | | HCoV-229E |
| | | | HCoV-NL63 |
| | Betacoronavirus | | HCoV-HKU1 |
| | | | HCoV-OC3 |
| | | | MERS-CoV |
| | Deltacoronavirus | | |
| | Gammacoronavirus | | |
| Coronaviridae/Torovirinae | Bafinivirus | | |
| | Torovirus | | |
| Retroviridae | Gammaretrovirus | | Murine leukemia |
| | | | virus |
| Togaviridae | Alphavirus | | Sindbis virus |
| | | | Semliki Forest |
| | | | virus |
| | | | Ross River virus |
| | | | Chikungunya |
| | | | virus |
| | | | Venezuelan |
| | | | equine |
| | | | encephalitis virus |

In some embodiments, the recombinant RNA molecules described herein encode a Picornavirus selected from a coxsackievirus, poliovirus, and Seneca Valley virus (SVV). In some embodiments, the recombinant RNA molecules described herein encode a coxsackievirus. In some aspects of this embodiment, the recombinant RNA molecules a coxsackievirus and comprise the 5′ UTR sequence of SEQ ID NO: 2 (See e.g., Brown et al., Complete Genomic Sequencing Shows that Polioviruses and Members of Human Enterovirus Species C Are Closely Related in the Noncapsid Coding Region. Journal of Virology, (2003) 77:16, p. 8973-8984. GenBank Accession No. AF546702). In such embodiments, the 5′ UTR sequence of SEQ ID NO: 2 unexpectedly increases the production of a functional coxsackievirus compared to other previously described 5′ UTR sequences (See e.g., Newcombe et al., Cellular receptor interactions of C-cluster human group A coxsackieviruses Journal of General Virology (2003), 84, 3041-3050. GenBank Accession No. AF465515). In some aspects of this embodiment, the recombinant RNA molecules encode a coxsackievirus and comprise the sequence of SEQ ID NO: 1.

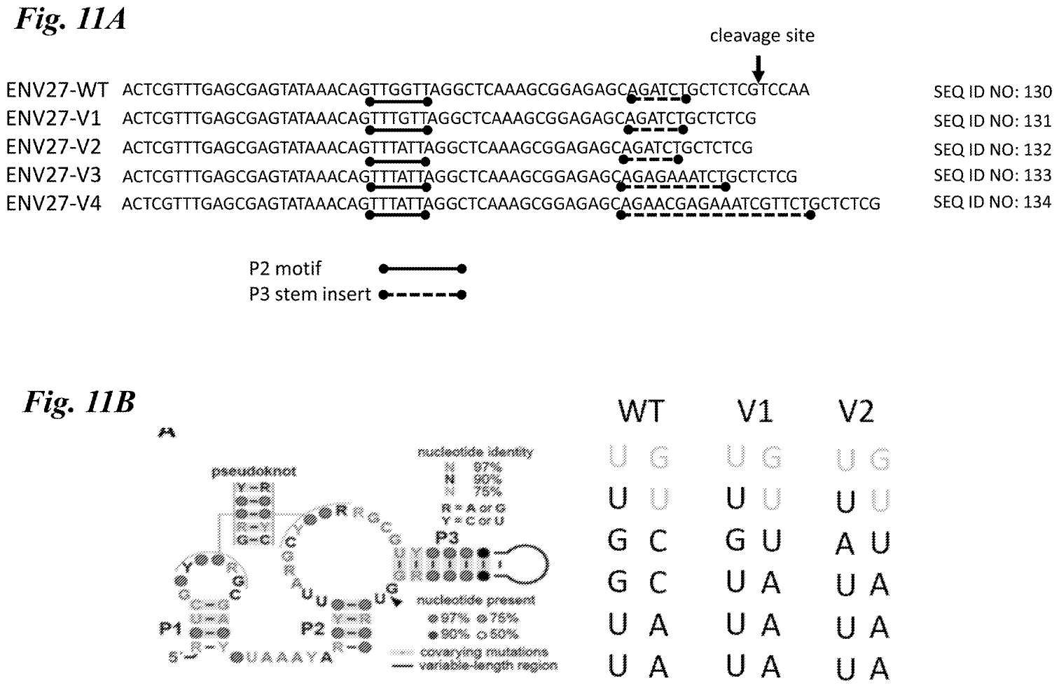

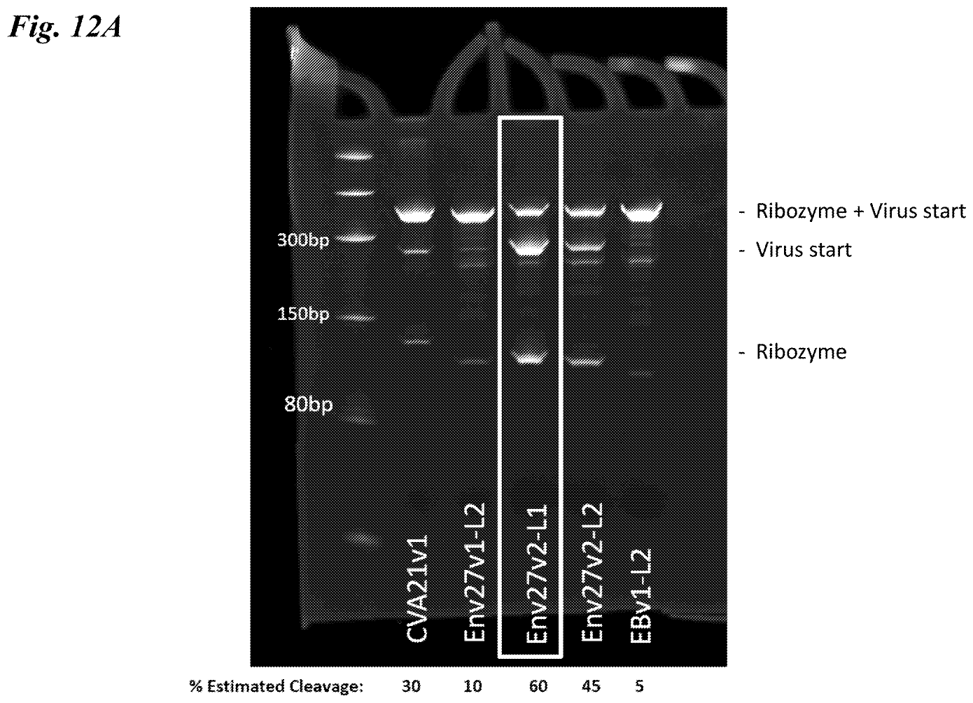

In some embodiments, the synthetic RNA viral genomes described herein encode a coxsackievirus. In some embodiments, the coxsackievirus is selected from CVB3, CVA21, and CVA9. The nucleic acid sequences of exemplary coxsackieviruses are provided GenBank Reference No. M33854.1 (CVB3), GenBank Reference No. KT161266.1 (CVA21), and GenBank Reference No. D00627.1 (CVA9). In some embodiments, the synthetic RNA viral genomes described herein encode a modified CVA21 virus comprising SEQ ID NO: 1, which is a Kuykendall (Kuyk) strain. In some embodiments, the sequence of the viral genome of the Kuykendall strain is according to GenBank Accession Number AF465515.1 or AF546702.1. In some embodiments, the synthetic RNA viral genomes described herein encode a chimeric coxsackievirus. In some embodiments, the synthetic RNA viral genomes described herein encode a CVA21 strain selected from the CVA21-EF strain and the CVA21-KY strain. In some embodiments, the synthetic RNA viral genomes described herein encode a CVA21-EF strain. An exemplary sequence of the viral genome of the EF strain is according to GenBank Accession Number EF015029.1. In some embodiments, the synthetic RNA viral genomes described herein encode a CVA21-KY strain. An exemplary sequence of the viral genome of the KY strain is according to GenBank Accession Number KY284011.1. As shown in FIGS. 11-26, the EF and KY strains provide therapeutic benefits over the Kuykendall lab strain and previously described synthetic picornavirus compositions.

The domain organization of the three CVA21 strains (EF, KY, and Kuykendall) are provided in FIG. 32 and the sequence identities between various regions of these three strains are provided below in Table 2.

TABLE 2
Sequence Identity between the Corresponding
Regions of Different CVA21 Strains

| | Strain 1 | Strain 2 | Sequence |
|---|---|---|---|
| Region | (Sequence) | (Sequence) | Identity |
| Whole Virus | CVA21-KY | CVA21-Kuykendall | 88.8% |
| | (SEQ ID NO: 5) | (SEQ ID NO: 1) | |
| | CVA21-EF | CVA21-Kuykendall | 79.6% |
| | (SEQ ID NO: 9) | (SEQ ID NO: 1) | |
| | CVA21-EF | CVA21-KY | 79.4% |
| | (SEQ ID NO: 9) | (SEQ ID NO: 5) | |
| 5′ UTR | CVA21-KY | CVA21-Kuykendall | 91.2% |
| (IRES) | (SEQ ID NO: 6) | (SEQ ID NO: 2) | |
| | CVA21-EF | CVA21-Kuykendall | 85.0% |
| | (SEQ ID NO: 10) | (SEQ ID NO: 2) | |
| | CVA21-EF | CVA21-KY | 86.1% |
| | (SEQ ID NO: 10) | (SEQ ID NO: 6) | |
| P1 | CVA21-KY | CVA21-Kuykendall | 87.5% |
| | (SEQ ID NO: 7) | (SEQ ID NO: 3) | |
| | CVA21-EF | CVA21-Kuykendall | 78.6% |
| | (SEQ ID NO: 11) | (SEQ ID NO: 3) | |
| | CVA21-EF | CVA21-KY | 78.3% |
| | (SEQ ID NO: 11) | (SEQ ID NO: 7) | |
| 3D | CVA21-KY | CVA21-Kuykendall | 89.9% |
| | (SEQ ID NO: 8) | (SEQ ID NO: 4) | |
| | CVA21-EF | CVA21-Kuykendall | 82.7% |
| | (SEQ ID NO: 12) | (SEQ ID NO: 4) | |
| | CVA21-EF | CVA21-KY | 82.5% |
| | (SEQ ID NO: 12) | (SEQ ID NO: 8) | |

One or more specific regions in the viral genome of the CVA21 EF or KY strain may contribute to the beneficial therapeutic effect observed for the EF and KY strains over the Kuykendall lab strain. In some embodiments, the one or more specific regions are selected from the group consisting of the 5′ UTR (IRES) region, the P1 region, and the 3D region. The nucleic acid positions of each of these specific regions for the virus strains described herein are as follows:
(a) The 5′ UTR (IRES) region of CVA21-Kuykendall encompasses nucleic acids 1-713 of SEQ ID NO: 1. The 5′ UTR (IRES) region of CVA21-KY encompasses nucleic acids 1-713 of SEQ ID NO: 5. The 5′ UTR (IRES) region of CVA21-EF encompasses nucleic acids 1-748 of SEQ ID NO: 9.
(b) The P1 region of CVA21-Kuykendall encompasses nucleic acids 714-3350 of SEQ ID NO: 1. The P1 region of CVA21-KY encompasses nucleic acids 714-3350 of SEQ ID NO: 5. The P1 region of CVA21-EF encompasses nucleic acids 749-3385 of SEQ ID NO: 9.
(c) The 3D region of CVA21-Kuykendall encompasses nucleic acids 5952-7340 of SEQ ID NO: 1. The 3D region of CVA21-KY encompasses nucleic acids 5952-7340 of SEQ ID NO: 5. The 3D region of CVA21-EF encompasses nucleic acids 5987-7375 of SEQ ID NO: 9.

In some embodiments, the synthetic RNA viral genome described herein encodes a CVA21-KY strain. In some embodiments, the synthetic RNA viral genome encoding the CVA21-KY strain comprises a polynucleotide sequence according to SEQ ID NO: 5. In some embodiments, the synthetic RNA viral genome encoding the CVA21-KY strain comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 5. In some embodiments, the synthetic RNA viral genome encoding the CVA21-KY strain comprises a polynucleotide sequences that is less than 95%, less than 90%, less than 85%, or less than 80% identical (including all ranges and subranges therebetween) to SEQ ID NO: 1.

In some embodiments, the synthetic RNA viral genome described herein encodes a CVA21-KY strain and comprises a 5′ UTR (IRES) sequence according to SEQ ID NO: 6 (corresponding to nucleic acids 1-713 of SEQ ID NO: 5). In some embodiments, the synthetic RNA viral genome described herein encodes a CVA21-KY strain and comprises a 5′ UTR (IRES) sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 6. In some embodiments, the synthetic RNA viral genome encoding the CVA21-KY strain comprises a 5′ UTR (IRES) sequence that is less than 95%, less than 90%, less than 85%, or less than 80% (including all ranges and subranges therebetween) identical to SEQ ID NO: 2.

In some embodiments, the synthetic RNA viral genome described herein encodes a CVA21-KY strain and comprises a P1 sequence according to SEQ ID NO: 7 (corresponding to nucleic acids 714-3350 of SEQ ID NO: 5). In some embodiments, the synthetic RNA viral genome described herein encodes a CVA21-KY strain and comprises a P1 sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 7. In some embodiments, the synthetic RNA viral genome encoding the CVA21-KY strain comprises a P1 sequence that is less than 95%, less than 90%, less than 85%, or less than 80% (including all ranges and subranges therebetween) identical to SEQ ID NO: 3.

In some embodiments, the synthetic RNA viral genome described herein encodes a CVA21-KY strain and comprises a 3D sequence according to SEQ ID NO: 8 (corresponding to nucleic acids 5952-7340 of SEQ ID NO: 5). In some embodiments, the synthetic RNA viral genome described herein encodes a CVA21-KY strain and comprises a 3D sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 8. In some embodiments, the synthetic RNA viral genome encoding the CVA21-KY strain comprises a 3D sequence that is less than 95%, less than 90%, less than 85%, or less than 80% (including all ranges and subranges therebetween) identical to SEQ ID NO: 4.

In some embodiments, the synthetic RNA viral genome described herein encodes a CVA21-EF strain. In some embodiments, the synthetic RNA viral genome encoding the CVA21-EF strain comprises a polynucleotide sequence according to SEQ ID NO: 9. In some embodiments, the synthetic RNA viral genome encoding the CVA21-EF strain comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 9. In some embodiments, the synthetic RNA viral genome encoding the CVA21-EF strain comprises a polynucleotide sequences that is less than 95%, less than 90%, less than 85%, or less than 80% (including all ranges and subranges therebetween) identical to SEQ ID NO: 1.

In some embodiments, the synthetic RNA viral genome described herein encodes a CVA21-EF strain and comprises a 5′ UTR (IRES) sequence according to SEQ ID NO: 10 (corresponding to nucleic acids 1-748 of SEQ ID NO: 9). In some embodiments, the synthetic RNA viral genome described herein encodes a CVA21-EF strain and comprises a 5′ UTR (IRES) sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 10. In some embodiments, the synthetic RNA viral genome encoding the CVA21-EF strain comprises a 5′ UTR (IRES) sequence that is less than 95%, less than 90%, less than 85%, or less than 80% (including all ranges and subranges therebetween) identical to SEQ ID NO: 2.

In some embodiments, the synthetic RNA viral genome described herein encodes a CVA21-EF strain and comprises a P1 sequence according to SEQ ID NO: 11 (corresponding to nucleic acids 749-3385 of SEQ ID NO: 9). In some embodiments, the synthetic RNA viral genome described herein encodes a CVA21-EF strain and comprises a P1 sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 11. In some embodiments, the synthetic RNA viral genome encoding the CVA21-EF strain comprises a P1 sequence that is less than 95%, less than 90%, less than 85%, or less than 80% (including all ranges and subranges therebetween) identical to SEQ ID NO: 3.

In some embodiments, the synthetic RNA viral genome described herein encodes a CVA21-EF strain and comprises a 3D sequence according to SEQ ID NO: 12 (corresponding to nucleic acids 5987-7375 of SEQ ID NO: 9). In some embodiments, the synthetic RNA viral genome described herein encodes a CVA21-EF strain and comprises a 3D sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 12. In some embodiments, the synthetic RNA viral genome encoding the CVA21-EF strain comprises a 3D sequence that is less than 95%, less than 90%, less than 85%, or less than 80% (including all ranges and subranges therebetween) identical to SEQ ID NO: 4.

In some embodiments, the CVA21 RNA viral genome described herein does not comprise the nucleotide sequence CGUCUC (SEQ ID NO: 83) or GAGACG (SEQ ID NO: 84). The corresponding, complementary DNA sequences, CGTCTC (SEQ ID NO: 85) and GAGACG (SEQ ID NO: 86), are BsmBI restriction enzyme recognition sites.

In some embodiments, the CVA21 RNA viral genome described herein does not comprise the nucleotide sequence GGUCUC (SEQ ID NO: 87) or GAGACC (SEQ ID NO: 88). The corresponding, complementary DNA sequences, GGTCTC (SEQ ID NO: 89) and GAGACC (SEQ ID NO: 90), are BsaI restriction enzyme recognition sites.

In some embodiments, the synthetic RNA viral genomes described herein encode a Seneca Valley virus (SVV). In some embodiments, the SVV is selected from a wild-type SVV (such as SVV-001, SEQ ID NO: 25) or a mutant SVV or a chimeric SVV (such as SVV-001-S177A encoded by SEQ ID NO: 26; or SVV-IRES-2-S177A encoded by SEQ ID NO: 68 or SEQ ID NO: 73).

In some embodiments, the SVV is a SVV-S177 mutant. In some embodiments, the SVV is an SVV-S177A mutant. As used herein in relation to the SVV viral genome, the term “S177 mutant” refers to a SVV viral genome encoding a VP2 protein comprising a mutation at amino acid S177 of the wildtype protein (amino acid numbering according to the VP2 protein encoded by SEQ ID NO: 25). Accordingly, the term “S177A mutant” refers to a SVV mutant having an amino acid substitution of S177A of the VP2 protein. In SEQ ID NO: 25, the VP2 S177 residue is encoded by the codon “UCU” at nucleic acid position 1645-1647. Accordingly, the SVV-S177 mutant comprises a nucleic acid mutation within the region corresponding to nucleic acid position 1645-1647 of SEQ ID NO: 25. In some embodiments, the SVV-S177A mutant comprises the codon sequence “GCU”, “GCC”, “GCA” or “GCG” at the region corresponding to nucleic acid position 1645-1647 of SEQ ID NO: 25. In some embodiments, the SVV-S177A mutant comprises the codon sequence “GCG” at the region corresponding to nucleic acid position 1645-1647 of SEQ ID NO: 25.

In some embodiments, the SVV RNA viral genome described herein does not comprise the nucleotide sequence GCUCUUC (SEQ ID NO: 79) or GAAGAGC (SEQ ID NO: 80). The corresponding, complementary DNA sequences, GCTCTTC (SEQ ID NO: 81) and GAAGAGC (SEQ ID NO: 82), are SapI restriction enzyme recognition sites. In some embodiments, a wildtype SVV RNA viral genome comprises SEQ ID NO: 79 at the position corresponding to nucleic acids 1504-1510 and/or nucleic acids 5293-5299 of SEQ ID NO: 25. In some embodiments, the SVV RNA viral genome of the disclosure comprises at least 1 nucleotide substitution as compared to SEQ ID NO: 79 within the region corresponding to nucleic acids 1504-1510 and/or nucleic acids 5293-5299 of SEQ ID NO: 25. In some embodiments, the at least 1 nucleotide substitution is a silent mutation that does not change the amino acids encoded by the corresponding region of the DNA. In some embodiments, the SVV RNA viral genome of the disclosure comprises a cytidine (“C”) at the position corresponding to nucleic acid 1509 and/or 5298 of SEQ ID NO: 25.

In some embodiments, the SVV RNA viral genome described herein does not comprise the nucleotide sequence GGUCUC (SEQ ID NO: 87) or GAGACC (SEQ ID NO: 88). The corresponding, complementary DNA sequences, GGTCTC (SEQ ID NO: 89) and GAGACC (SEQ ID NO: 90), are BsaI restriction enzyme recognition sites.

In some embodiments, the synthetic RNA viral genomes described herein encode a chimeric picornavirus (e.g., encode a virus comprising one portion, such as a capsid protein or an IRES, derived from a first picornavirus and another portion, such as a non-structural gene such as a protease or polymerase derived from a second picornavirus). In some embodiments, the synthetic RNA viral genomes described herein encode a chimeric SVV.

In some embodiments, the synthetic RNA viral genome described herein encodes a SVV comprising one or more specific regions derived from an SVV strain selected from the group consisting of SVV-001 (SEQ ID NO: 25 or SEQ ID NO: 72 (Genbank ID No.: DQ641257.1)), SVA/BRA/MG2/2015 (SEQ ID NO: 69; GenBank ID No.: KR063108.1), SVA/Canada/MB/NCFAD-104/2015 (SEQ ID NO: 70; GenBank ID No.: KY486156.1), and SVV-MN15-308 (SEQ ID NO: 71; GenBank ID No.: KU359214.1). In some embodiments, the one or more specific regions are selected from the group consisting of the 5′ UTR (IRES) region, the P1 region, and the P3 region. The nucleic acid positions of each of these specific regions for the virus strains described herein are as follows:
(a) The 5′ UTR (IRES) region of SVV-001 encompasses nucleic acids 1-668 of SEQ ID NO: 25. The 5′ UTR (IRES) region of SVA/BRA/MG2/2015 encompasses nucleic acids 1-656 of SEQ ID NO: 69. The 5′ UTR (IRES) region of SVA/Canada/MB/NCFAD-104/2015 encompasses nucleic acids 1-612 of SEQ ID NO: 70. The 5′ UTR (IRES) region of SVV-MN15-308 encompasses nucleic acids 1-610 of SEQ ID NO: 71.
(b) The P1 region of SVV-001 encompasses nucleic acids 669-3477 of SEQ ID NO: 25. The P1 region of SVA/BRA/MG2/2015 encompasses nucleic acids 657-3465 of SEQ ID NO: 69. The P1 region of SVA/Canada/MB/NCFAD-104/2015 encompasses nucleic acids 613-3421 of SEQ ID NO: 70. The P1 region of SVV-MN15-308 encompasses nucleic acids 611-3419 of SEQ ID NO: 71.
(c) The P3 region of SVV-001 encompasses nucleic acids 4855-7212 of SEQ ID NO: 25. The P3 region of SVA/BRA/MG2/2015 encompasses nucleic acids 4843-7200 of SEQ ID NO: 69. The P3 region of SVA/Canada/MB/NCFAD-104/2015 encompasses nucleic acids 4799-7156 of SEQ ID NO: 70. The P3 region of SVV-MN15-308 encompasses nucleic acids 4797-7154 of SEQ ID NO: 71.

In some embodiments, the synthetic RNA viral genome described herein encodes a SVV comprising a 5′ UTR (IRES) region derived from SVA/BRA/MG2/2015 (nucleic acids 1-656 of SEQ ID NO: 69). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 1-656 of SEQ ID NO: 69. In some embodiments, other than the one or more regions derived from SVA/BRA/MG2/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a 5′ UTR (IRES) region derived from SVA/Canada/MB/NCFAD-104/2015 (nucleic acids 1-612 of SEQ ID NO: 70). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 1-612 of SEQ ID NO: 70. In some embodiments, other than the one or more regions derived from SVA/Canada/MB/NCFAD-104/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a 5′ UTR (IRES) region derived from SVV-MN15-308 (nucleic acids 1-610 of SEQ ID NO: 71). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 1-610 of SEQ ID NO: 71. In some embodiments, other than the one or more regions derived from SVV-MN15-308, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P1 region derived from SVA/BRA/MG2/2015 (nucleic acids 657-3465 of SEQ ID NO: 69). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 657-3465 of SEQ ID NO: 69. In some embodiments, other than the one or more regions derived from SVA/BRA/MG2/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P1 region derived from SVA/Canada/MB/NCFAD-104/2015 (nucleic acids 613-3421 of SEQ ID NO: 70). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 613-3421 of SEQ ID NO: 70. In some embodiments, other than the one or more regions derived from SVA/Canada/MB/NCFAD-104/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P1 region derived from SVV-MN15-308 (nucleic acids 611-3419 of SEQ ID NO: 71). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 611-3419 of SEQ ID NO: 71. In some embodiments, other than the one or more regions derived from SVV-MN15-308, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P3 region derived from SVA/BRA/MG2/2015 (nucleic acids 4843-7200 of SEQ ID NO: 69). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 4843-7200 of SEQ ID NO: 69. In some embodiments, other than the one or more regions derived from SVA/BRA/MG2/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P3 region derived from SVA/Canada/MB/NCFAD-104/2015 (nucleic acids 4799-7156 of SEQ ID NO: 70). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 4799-7156 of SEQ ID NO: 70. In some embodiments, other than the one or more regions derived from SVA/Canada/MB/NCFAD-104/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P3 region derived from SVV-MN15-308 (nucleic acids 4797-7154 of SEQ ID NO: 71). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 4797-7154 of SEQ ID NO: 71. In some embodiments, other than the one or more regions derived from SVV-MN15-308, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a chimeric SVV comprising a 5′ UTR (IRES) region derived from SVA/Canada/MB/NCFAD-104/2015 (SEQ ID NO: 70) and the rest of the viral genome derived from SVV-001 (SEQ ID NO: 25). In some embodiments, the SVV is an SVV-S177 mutant (e.g., a S177A mutant). In some embodiments, the synthetic RNA viral genome has at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 68.

In some embodiments, the synthetic RNA viral genome has been engineered and comprises less than 100% sequence identity to that of a wildtype virus (e.g., a wildtype CVA21 or a wildtype SVV). In some embodiments, the synthetic RNA viral genome comprises less than 99.9%, less than 99.8%, less than 99.7%, less than 99.6%, less than 99.5%, less than 99%, less than 98%, less than 97%, less than 96%, less than 95%, less than 94%, less than 93%, less than 92%, less than 91%, or less than 90%, sequence identity to that of a corresponding wildtype virus.

In some embodiments, the synthetic RNA viral genome comprises a microRNA (miRNA) target sequence (miR-TS) cassette, wherein the miR-TS cassette comprises one or more miRNA target sequences, and wherein expression of one or more of the corresponding miRNAs in a cell inhibits replication of the encoded oncolytic virus in the cell. In some embodiments, the one or more miRNAs are selected from miR-124, miR-1, miR-143, miR-128, miR-219, miR-219a, miR-122, miR-204, miR-217, miR-137, miR-142, and miR-126. In some embodiments, the miR-TS cassette comprises one or more copies of a miR-124 target sequence, one or more copies of a miR-1 target sequence, and one or more copies of a miR-143 target sequence. In some embodiments, the miR-TS cassette comprises one or more copies of a miR-128 target sequence, one or more copies of a miR-219a target sequence, and one or more copies of a miR-122 target sequence. In some embodiments, the miR-TS cassette comprises one or more copies of a miR-128 target sequence, one or more copies of a miR-204 target sequence, and one or more copies of a miR-219 target sequence. In some embodiments, the miR-TS cassette comprises one or more copies of a miR-217 target sequence, one or more copies of a miR-137 target sequence, and one or more copies of a miR-126 target sequence.

In some embodiments, the synthetic RNA viral genome comprises one or more miR-TS cassettes is incorporated into the 5′ untranslated region (UTR) or 3′ UTR of one or more essential viral genes. In some embodiments, the synthetic RNA viral genome comprises one or more miR-TS cassettes is incorporated into the 5′ untranslated region (UTR) or 3′ UTR of one or more non-essential genes. In some embodiments, the synthetic RNA viral genome comprises one or more miR-TS cassettes is incorporated 5′ or 3′ of one or more essential viral genes.

In some embodiments, the synthetic RNA viral genome comprises a heterologous polynucleotide encoding a payload molecule. In such embodiments, the synthetic RNA viral genome drives production of an infectious oncolytic virus as well as expression of the payload molecule. In some embodiments, the expression of the payload molecule can increase the therapeutic efficacy of the oncolytic virus. In some embodiments, the payload molecule is selected from IL-12, GM-CSF, CXCL10, IL-36Y, CCL21, IL-18, IL-2, CCL4, CCL5, an anti-CD3-anti-FAP BiTE, an antigen binding molecule that binds DLL3, or an antigen binding molecule that binds EpCAM. In some embodiments, the payload molecule comprises or consists of MLKL 4HB domain. In some embodiments, the payload molecule comprises or consists of Gasdermin D N-terminal fragment. In some embodiments, the payload molecule comprises or consists of Gasdermin E N-terminal fragment. In some embodiments, the payload molecule comprises or consists of HMGB 1 Box B domain. In some embodiments, the payload molecule comprises or consists of SMAC/Diablo. In some embodiments, the payload molecule comprises or consists of Melittin. In some embodiments, the payload molecule comprises or consists of L-amino-acid oxidase (LAAO). In some embodiments, the payload molecule comprises or consists of disintegrin. In some embodiments, the payload molecule comprises or consists of TRAIL (TNFSF10). In some embodiments, the payload molecule comprises or consists of a nitroreductase (e.g., E. coli NfsB or NfsA). In some embodiments, the payload molecule comprises or consists of a reovirus FAST protein (e.g., ARV p14, BRV p15, or p14-p15 hybrid). In some embodiments, the payload molecule comprises or consists of a leptin/FOSL2. In some embodiments, the payload molecule comprises or consists of an α-1,3-galactosyltransferase. In some embodiments, the payload molecule comprises or consists of an adenosine deaminase 2 (ADA2). In some embodiments, the paylod molecule comprises or consists of a cytokine selected from IL-IL-36γ, IL-7, IL-12, IL-18, IL-21, IL2 or IFNγ. Further description of the types of payload molecules suitable for use in these embodiments is provided below.

### Methods of Producing Recombinant RNA Viral Genomes

In some embodiments, the disclosure provides recombinant DNA molecules encoding the synthetic RNA viral genomes described herein. Such recombinant DNA molecules are referred to herein as “DNA templates” or “recombinant DNA templates”. In some embodiments, the recombinant DNA molecules are used as templates for in vitro transcription of the encoded synthetic RNA viral genomes. In some embodiments, the recombinant DNA molecules (e.g., DNA templates) comprises, from 5′ to 3′, one or more of the following elements: (i) a promoter; (ii) a 5′ leader sequence; (iii) a 5′ junctional cleavage sequence; (iv) a DNA polynucleotide sequence encoding the synthetic RNA genome; (v) a polyA tail; and/or (vi) a 3′ junctional cleavage sequence. In some embodiments, the recombinant DNA molecules (e.g., DNA templates) encoding the recombinant RNA molecule comprises each of the following elements: (i) a promoter; (ii) a 5′ leader sequence; (iii) a 5′ junctional cleavage sequence; (iv) a DNA polynucleotide sequence encoding the synthetic RNA genome; (v) a polyA tail; and (vi) a 3′ junctional cleavage sequence. Each of these elements are described in detail below. The description provided for each individual element is such that the specific embodiments of each element can be combined into the final recombinant DNA molecules (e.g., DNA templates). For example, the disclosure of a specific leader sequence can be combined with the disclosure of a specific 5′ junctional cleavage sequence, etc.

In some embodiments, the recombinant DNA molecules (e.g., DNA templates) do not comprise additional nucleic acids between two adjacent elements but may comprise additional nucleic acids upstream to the promoter sequence or downstream to the 3′ junctional cleavage sequence. In some embodiments, the promoter sequence is a T7 promoter sequence. In some embodiments, the T7 promoter sequence comprises or consists of SEQ ID NO: 91.

In some embodiments, the promoter is suitable for in vitro transcription. In some embodiments, the promoter is a T7 promoter.

In some embodiments, the synthetic RNA viral genomes described herein are produced in vitro using one or more recombinant DNA templates comprising a polynucleotide encoding the synthetic RNA viral genomes. In other words, the recombinant DNA templates are vectors comprising the polynucleotide encoding the synthetic RNA viral genomes. The term “vector” is used herein to refer to a nucleic acid molecule capable of transferring, encoding, or transporting another nucleic acid molecule. The transferred nucleic acid is generally inserted into the vector nucleic acid molecule. A vector may include sequences that direct autonomous replication in a cell and/or may include sequences sufficient to allow integration into host cell DNA. In some embodiments, the recombinant RNA molecule encoding an oncolytic virus described herein is produced using one or more DNA vectors.

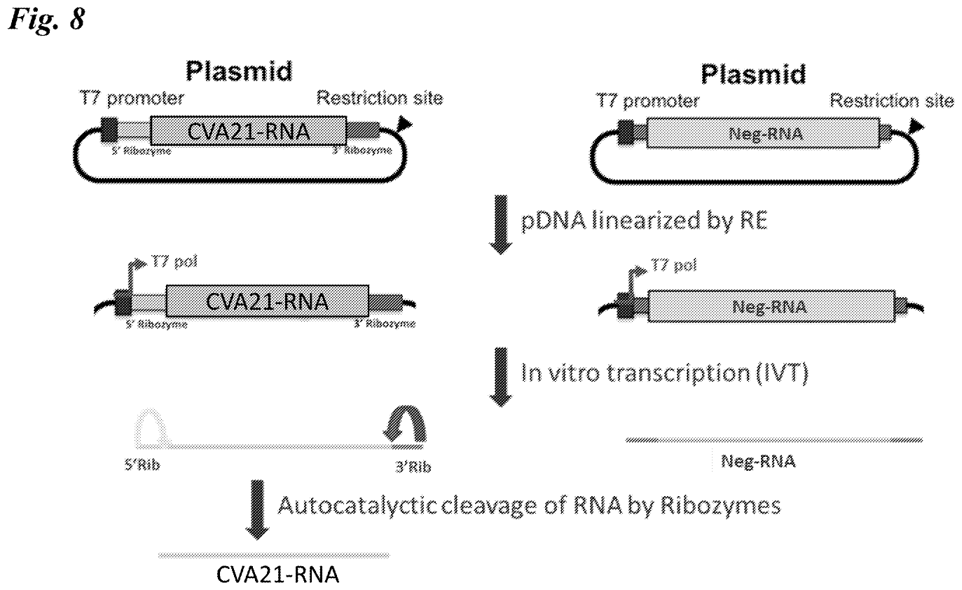

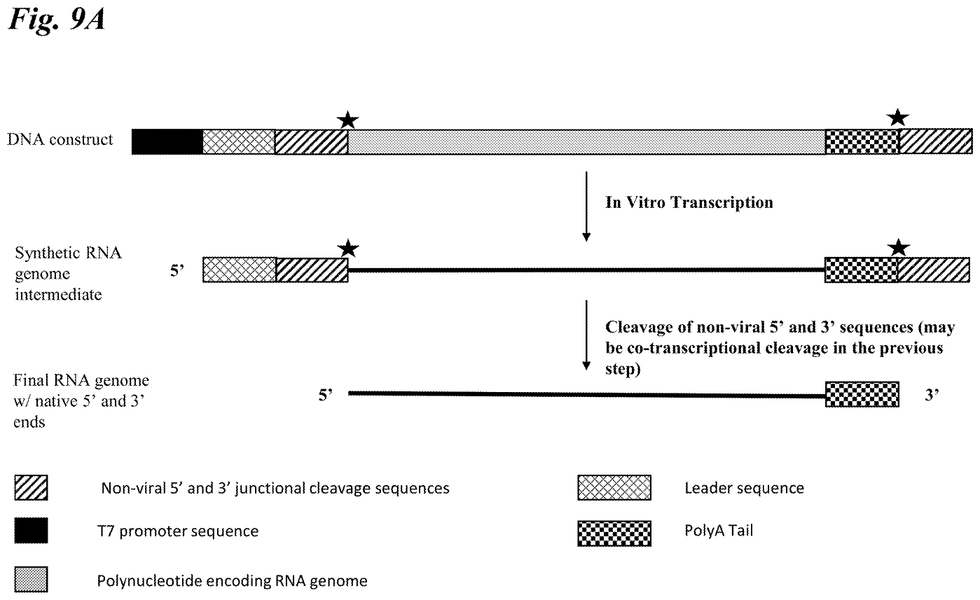

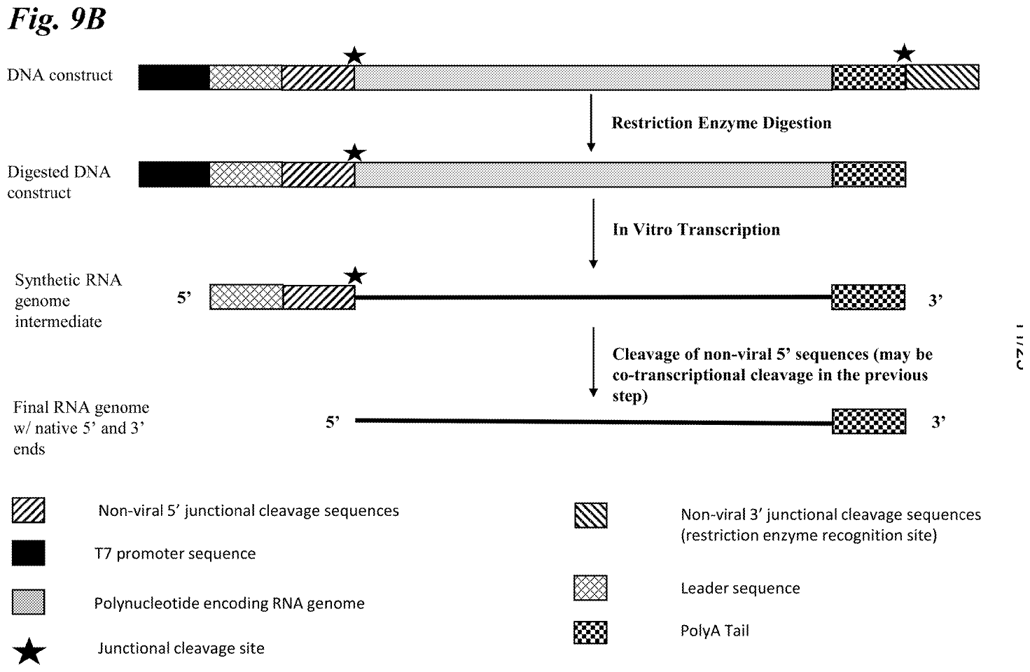

In some embodiments, the synthetic RNA viral genomes described herein are produced by introducing a recombinant DNA molecule (e.g., DNA template) comprising a polynucleotide encoding the recombinant RNA molecule (e.g., by means of transfection, transduction, electroporation, and the like) into a suitable host cell in vitro. Suitable host cells include insect and mammalian cell lines. The host cells are cultured for an appropriate amount of time to allow expression of the polynucleotides and production of the synthetic RNA viral genomes. The synthetic RNA viral genomes are then isolated from the host cell and formulated for therapeutic use (e.g., encapsulated in a particle). A schematic of the in vitro synthesis of the CVA21 RNA viral genomes with 3′ and 5′ ribozymes is shown in FIG. 8. The same schematic applies to the synthesis of RNA viral genomes (e.g., CVA21 or SVV viral genomes) using other combinations of junctional cleavage sequences (See e.g. FIG. 9A). When the 3′ junctional cleavage sequence comprises or consists of a restriction enzyme recognition site, the recombinant DNA molecule (e.g., DNA template) may be digested with the corresponding restriction enzyme before the in vitro transcription process, as shown in FIG. 9B.

### T7 Promoter

In some embodiments, the recombinant DNA molecule (e.g., DNA template) comprises a T7 promoter. In some embodiments, the T7 promoter comprises or consists of a polynucleotide sequence of SEQ ID NO: 91. In some embodiments, the T7 promoter comprises or consists of a polynucleotide sequence of SEQ ID NO: 91 with at most 1, 2, 3, or 4 mutations.

In some embodiments, the T7 promoter is placed immediately before the leader sequence, with no additional nucleotides in between. In some embodiments, the T7 promoter is placed immediately before the 5′ junctional cleavage sequence, with no additional nucleotides in between. In some embodiments, the viral genome encodes CVA21 or SVV.

### Junctional Cleavage Sequences

In some embodiments, the recombinant RNA molecules comprising the synthetic RNA viral genomes described herein require discrete 5′ and 3′ ends that are native to the virus. The RNA transcripts produced by T7 RNA polymerase in vitro or by mammalian RNA Pol II contain mammalian 5′ and 3′ UTRs do not contain the discrete, native ends required for production of an infectious RNA virus. For example, the T7 RNA polymerase requires a guanosine residue on the 5′ end of the template polynucleotide in order to initiate transcription. However, SVV begins with a uridine residue on its 5′ end. Thus, the T7 leader sequence, which is required for in vitro transcription of the SVV transcript must be removed to generate the native 5′ SVV terminus required for production of a functional infectious SVV. Therefore, in some embodiments, recombinant DNA molecules (e.g., DNA templates) suitable for use in the production of the synthetic RNA viral genomes described herein require additional non-viral 5′ and 3′ sequences that enable generation of the discrete 5′ and 3′ ends native to the virus. Such sequences are referred to herein as junctional cleavage sequences (JCS). In some embodiments, the junctional cleavage sequences act to cleave the T7 RNA polymerase or Pol II-encoded RNA transcript at the junction of the viral RNA and the mammalian mRNA sequence such that the non-viral RNA polynucleotides are removed from the transcript in order to maintain the native 5′ and 3′ discrete ends of the virus (See schematic shown in FIG. 9A). In some embodiments, the junctional cleavage sequences act to generate the appropriate ends during the linearization of the DNA plasmid encoding the synthetic viral genome (e.g., the use of 3′ restriction enzyme recognition sequences to produce the appropriate 3′ end upon linearization of the plasmid template and prior to in vitro transcription of the synthetic RNA genome).

In some embodiments, the recombinant DNA molecules (e.g., DNA templates) suitable for use in the production of the synthetic RNA viral genomes described herein comprise at least one 5′ junctional cleavage sequence and at least one 3′ junctional cleavage sequence. In some embodiments, the recombinant DNA molecules (e.g., DNA templates) suitable for use in the production of the synthetic RNA viral genomes described herein comprise one or more 5′ junctional cleavage sequences and at least one 3′ junctional cleavage sequence. In some embodiments, the recombinant DNA molecules (e.g., DNA templates) suitable for use in the production of the synthetic RNA viral genomes described herein comprise at least one 5′ junctional cleavage sequence and one or more 3′ junctional cleavage sequences. In some embodiments, the recombinant DNA molecules (e.g., DNA templates) suitable for use in the production of the synthetic RNA viral genomes described herein comprise one or more 5′ junctional cleavage sequences and one or more 3′ junctional cleavage sequences. In some embodiments, the recombinant DNA molecules (e.g., DNA templates) suitable for use in the production of the synthetic RNA viral genomes described herein comprise two 5′ junctional cleavage sequences and at least one 3′ junctional cleavage sequence. In some embodiments, the recombinant DNA molecules (e.g., DNA templates) suitable for use in the production of the synthetic RNA viral genomes described herein comprise at least one 5′ junctional cleavage sequence and two 3′ junctional cleavage sequences.

The nature of the junctional cleavage sequences and the removal of the non-viral RNA from the viral genome transcript can be accomplished by a variety of methods. For example, in some embodiments, the junctional cleavage sequences are targets for RNA interference (RNAi) molecules. “RNA interference molecule” as used herein refers to an RNA polynucleotide that mediates degradation of a target mRNA sequence through endogenous gene silencing pathways (e.g., Dicer and RNA-induced silencing complex (RISC)). Exemplary RNA interference agents include micro RNAs (miRNAs), artificial miRNA (amiRNAs), short hairpin RNAs (shRNAs), and small interfering RNAs (siRNAs). Further, any system for cleaving an RNA transcript at a specific site currently known the art or to be defined in the future can be used to generate the discrete ends native to the virus.

In some embodiments, the RNAi molecule is a miRNA. A miRNA refers to a naturally-occurring, small non-coding RNA molecule of about 18-25 nucleotides in length that is at least partially complementary to a target mRNA sequence. In animals, genes for miRNAs are transcribed to a primary miRNA (pri-miRNA), which is double stranded and forms a stem-loop structure. Pri-miRNAs are then cleaved in the nucleus by a microprocessor complex comprising the class 2 RNase III, Drosha, and the microprocessor subunit, DCGR8, to form a 70-100 nucleotide precursor miRNA (pre-miRNA). The pre-miRNA forms a hairpin structure and is transported to the cytoplasm where it is processed by the RNase III enzyme, Dicer, into a miRNA duplex of ˜ 18-25 nucleotides. Although either strand of the duplex may potentially act as a functional miRNA, typically one strand of the miRNA is degraded and only one strand is loaded onto the Argonaute (AGO) nuclease to produce the effector RNA-induced silencing complex (RISC) in which the miRNA and its mRNA target interact (Wahid et al., 1803:11, 2010, 1231-1243). In some embodiments, the 5′ and/or 3′ junctional cleavage sequences are miRNA target sequences.

In some embodiments, the RNAi molecule is an artificial miRNA (amiRNA) derived from a synthetic miRNA-embedded in a Pol II transcript. (See e.g., Liu et al., Nucleic Acids Res (2008) 36:9; 2811-2834; Zeng et al., Molecular Cell (2002), 9; 1327-1333; Fellman et al., Cell Reports (2013) 5; 1704-1713). In some embodiments, the 5′ and/or 3′ junctional cleavage sequences are amiRNA target sequences.

In some embodiments, the RNAi molecule is an siRNA molecule. siRNAs refer to double stranded RNA molecules typically about 21-23 nucleotides in length. The duplex siRNA molecule is processed in the cytoplasm by the associates with a multi protein complex called the RNA-induced silencing complex (RISC), during which the “passenger” sense strand is enzymatically cleaved from the duplex. The antisense “guide” strand contained in the activated RISC then guides the RISC to the corresponding mRNA by virtue of sequence complementarity and the AGO nuclease cuts the target mRNA, resulting in specific gene silencing. In some embodiments, the siRNA molecule is derived from an shRNA molecule. shRNAs are single stranded artificial RNA molecules ˜50-70 nucleotides in length that form stem-loop structures. Expression of shRNAs in cells is accomplished by introducing a DNA polynucleotide encoding the shRNA by plasmid or viral vector. The shRNA is then transcribed into a product that mimics the stem-loop structure of a pre-miRNA, and after nuclear export the hairpin is processed by Dicer to form a duplex siRNA molecule which is then further processed by the RISC to mediate target-gene silencing. In some embodiments, the 5′ and/or 3′ junctional cleavage sequences are siRNA target sequences.

In some embodiments, the junctional cleavage sequences are guide RNA (gRNA) target sequences. In such embodiments, gRNAs can be designed and introduced with a Cas endonuclease with RNase activity (e.g., Cas13) to mediate cleavage of the viral genome transcript at the precise junctional site. In some embodiments, the 5′ and/or 3′ junctional cleavage sequences are gRNA target sequences.

In some embodiments, the junctional cleavage sequences are pri-miRNA-encoding sequences. Upon transcription of the polynucleotide encoding the viral genome (e.g., the recombinant RNA molecule), these sequences form the pri-miRNA stem-loop structure which is then cleaved in the nucleus by Drosha to cleave the transcript at the precise junctional site. In some embodiments, the 5′ and/or 3′ junctional cleavage sequences are pri-mRNA target sequences.

In some embodiments, the junctional cleavage sequences are primer binding sequences that facilitate cleavage by the endoribonuclease, RNAseH. In such embodiments, a primer that anneals to the 5′ and/or 3′ junctional cleavage sequence is added to the in vitro reaction along with an RNAseH enzyme. RNAseH specifically hydrolyzes the phosphodiester bonds of RNA which is hybridized to DNA, therefore enabling cleavage of the synthetic RNA genome intermediates at the precise junctional cleavage sequence to produce the required 5′ and 3′ native ends.

In some embodiments, the junctional cleavage sequences comprise or consist of restriction enzyme recognition sites and result in the generation of discrete ends of viral transcripts during linearization of the plasmid template runoff RNA synthesis with T7 RNA Polymerase. In some embodiments, the junctional cleavage sequences are Type IIS restriction enzyme recognition sites. Type IIS restriction enzymes comprise a specific group of enzymes which recognize asymmetric DNA sequences and cleave at a defined distance outside of their recognition sequence, usually within 1 to 20 nucleotides. Exemplary Type IIS restriction enzymes include AcuI, AlwI, Bael, BbsI, BbvI, BccI, BceAI, BcgI, BciVI, BcoDI, BfuAI, BmrI, BpmI, BpuEI, BsaI, BsaXI, BseRI, BsgI, BsmAI, BsmBi, BsmFI, BsmI, BspCNI, BspMI, BspQI, BsrDI, BsrI, BtgZI, BtsCI, BstI, CaspCI, EarI, EciI, Esp3I, FauI, FokI, HgaI, HphI, HpyAV, MbolI, MlyI, MmeI, MnIL, NmeAIII, PleI, SapI, and SfaNI. The recognition sequences for these Type IIS restriction enzymes are known in the art. See the New England Biolabs website located at neb.com/tools-and-resources/selection-charts/type-iis-restriction-enzymes. In some embodiments, the junctional cleavage sequence comprises a SapI restriction enzyme recognition site. In some embodiments, the junctional cleavage sequence comprises a BsmBI restriction enzyme recognition site. In some embodiments, the junctional cleavage sequence comprises a BsaI restriction enzyme recognition site. A skilled person would understand that, because the cleavage site of the Type IIS restriction enzymes is typically outside the enzyme recognition site (e.g., offset by 1-5 nucleotides), the corresponding junctional cleavage sequence may also comprise the additional nucleotide(s) required by the corresponding restriction enzyme to create the discrete end of the viral transcript (e.g., the poly-A tail at 3′ end).

In some embodiments, the junctional cleavage sequences are sequences encoding ligand-inducible self-cleaving ribozymes, referred to as “aptazymes”. Aptazymes are ribozyme sequences that contain an integrated aptamer domain specific for a ligand. Ligand binding to the apatmer domain triggers activation of the enzymatic activity of the ribozyme, thereby resulting in cleavage of the RNA transcript. Exemplary aptazymes include theophylline-dependent aptazymes (e.g., hammerhead ribozyme linked to a theophylline-dependent apatmer, described in Auslander et al., Mol BioSyst. (2010) 6, 807-814), tetracycline-dependent aptazymes (e.g., hammerhead ribozyme linked to a Tet-dependent aptamer, described by Zhong et al., eLife 2016; 5: e18858 DOI: 10.7554/eLife.18858; Win and Smolke, PNAS (2007) 104; 14283-14288; Whittmann and Suess, Mol Biosyt (2011) 7; 2419-2427; Xiao et al., Chem & Biol (2008) 15; 125-1137; and Beilstein et al., ACS Syn Biol (2015) 4; 526-534), guanine-dependent aptazymes (e.g., hammerhead ribozyme linked to a guanine-dependent aptamer, described by Nomura et al., Chem Commun., (2012) 48 (57); 7215-7217). In some embodiments, the 5′ and/or 3′ junctional cleavage sequences are aptazyme-encoding sequences.

In some embodiments, the junctional cleavage sequences are target sequences for an RNAi molecule (e.g., an siRNA molecule, an shRNA molecule, an miRNA molecule, or an amiRNA molecule), a gRNA molecule, or an RNAseH primer. In such embodiments, the junctional cleavage sequence is at least partially complementary to the sequence of the RNAi molecule, gRNA molecule, or primer molecule. Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In some embodiments, the 5′ junctional cleavage sequence and 3′ junctional cleavage sequence are from the same group (e.g., are both RNAi target sequences, both ribozyme-encoding sequences, etc.). For example, in some embodiments, the junctional cleavage sequences are RNAi target sequences (e.g., siRNA, shRNA, amiRNA, or miRNA target sequences) and are incorporated into the 5′ and 3′ ends of the polynucleotide encoding the viral genome (e.g., the recombinant RNA molecule). In such embodiments, the 5′ and 3′ RNAi target sequence may be the same (i.e., targets for the same siRNA, amiRNA, or miRNA) or different (i.e., the 5′ sequence is a target for one siRNA, shmiRNA, or miRNA and the 3′ sequence is a target for another siRNA, amiRNA, or miRNA). In some embodiments, the junctional cleavage sequences are guide RNA target sequences and are incorporated into the 5′ and 3′ ends of the polynucleotide encoding the viral genome (e.g., the recombinant RNA molecule). In such embodiments, the 5′ and 3′ gRNA target sequences may be the same (i.e., targets for the same gRNA) or different (i.e., the 5′ sequence is a target for one gRNA and the 3′ sequence is a target for another gRNA). In some embodiments, the junctional cleavage sequences are pri-mRNA-encoding sequences and are incorporated into the 5′ and 3′ ends of the polynucleotide encoding the viral genome (e.g., the recombinant RNA molecule). In some embodiments, the junctional cleavage sequences are ribozyme-encoding sequences and are incorporated immediately 5′ and 3′ of the polynucleotide sequence encoding the viral genome (e.g., the recombinant RNA molecule).

In some embodiments, the 5′ junctional cleavage sequence and 3′ junctional cleavage sequence are from the same group but are different variants or types. For example, in some embodiments, the 5′ and 3′ junctional cleavage sequences may be target sequences for an RNAi molecule, wherein the 5′ junctional cleavage sequence is an siRNA target sequence and the 3′ junctional cleavage sequence is a miRNA target sequence (or vis versa). In some embodiments, the 5′ and 3′ junctional cleavage sequences may be ribozyme-encoding sequences, wherein the 5′ junctional cleavage sequence is a hammerhead ribozyme-encoding sequence and the 3′ junctional cleavage sequence is a hepatitis delta virus ribozyme-encoding sequence.

In some embodiments, the 5′ junctional cleavage sequence and 3′ junctional cleavage sequence are different types. For example, in some embodiments, the 5′ junctional cleavage sequence is an RNAi target sequence (e.g., an siRNA, an amiRNA, or a miRNA target sequence) and the 3′ junctional cleavage sequence is a ribozyme sequence, an aptazyme sequence, a pri-miRNA sequence, or a gRNA target sequence. In some embodiments, the 5′ junctional cleavage sequence is a ribozyme sequence and the 3′ junctional cleavage sequence is an RNAi target sequence (e.g., an siRNA, an amiRNA, or a miRNA target sequence), an aptazyme sequence, a pri-miRNA-encoding sequence, or a gRNA target sequence. In some embodiments, the 5′ junctional cleavage sequence is an aptazyme sequence and the 3′ junctional cleavage sequence is an RNAi target sequence (e.g., an siRNA, an amiRNA, or a miRNA target sequence), a ribozyme sequence, a pri-miRNA sequence, or a gRNA target sequence. In some embodiments, the 5′ junctional cleavage sequence is a pri-miRNA sequence and the 3′ junctional cleavage sequence is an RNAi target sequence (e.g., an siRNA, an amiRNA, or a miRNA target sequence), a ribozyme sequence, an aptazyme sequence, or a gRNA target sequence. In some embodiments, the 5′ junctional cleavage sequence is a gRNA target sequence and the 3′ junctional cleavage sequence is an RNAi target sequence (e.g., an siRNA, an amiRNA, or a miRNA target sequence), a ribozyme sequence, a pri-miRNA sequence, or an aptazyme sequence.

Exemplary arrangements of the junctional cleavage sequences relative to the polynucleotide encoding the synthetic viral genome are shown below in Tables 3 and 4.

TABLE 3
Symmetrical Junctional Cleavage Sequence (JSC) Arrangements

| | | | |
|---|---|---|---|
| 5′ | JCS | JCS | 3′ |

| | | |
|---|---|---|
| siRNA TS | synthetic genome | siRNA TS |
| miR TS | synthetic genome | miR TS |
| AmiR TS | synthetic genome | AmiR TS |
| gRNA TS | synthetic genome | gRNA TS |
| pri-miR | synthetic genome | pri-miR |
| ribozyme | synthetic genome | ribozyme |
| aptazyme | synthetic genome | aptazyme |
| RNAseH primer TS | synthetic genome | RNAseH primer TS |

TABLE 4
Asymmetrical JCS Arrangements

| | | | |
|---|---|---|---|
| 5′ | JCS | JCS | 3′ |

| | | |
|---|---|---|
| siRNA TS | synthetic genome | miR TS |
| siRNA TS | synthetic genome | AmiR TS |
| siRNA TS | synthetic genome | gRNA TS |
| siRNA TS | synthetic genome | pri-miR |
| siRNA TS | synthetic genome | ribozyme |
| siRNA TS | synthetic genome | aptazyme |
| siRNA TS | synthetic genome | RNAseH primer TS |
| siRNA TS | synthetic genome | Restr Enz RS |
| miR TS | synthetic genome | siRNA TS |
| miR TS | synthetic genome | AmiR TS |
| miR TS | synthetic genome | gRNA TS |
| miR TS | synthetic genome | pri-miR |
| miR TS | synthetic genome | ribozyme |
| miR TS | synthetic genome | aptazyme |
| miR TS | synthetic genome | RNAseH primer TS |
| miR TS | synthetic genome | Restr Enz RS |
| AmiR TS | synthetic genome | siRNA TS |
| AmiR TS | synthetic genome | miR TS |
| AmiR TS | synthetic genome | gRNA TS |
| AmiR TS | synthetic genome | pri-miR |
| AmiR TS | synthetic genome | ribozyme |
| AmiR TS | synthetic genome | aptazyme |
| AmiR TS | synthetic genome | RNAseH primer TS |
| AmiR TS | synthetic genome | Restr Enz RS |
| gRNA TS | synthetic genome | siRNA TS |
| gRNA TS | synthetic genome | miR TS |
| gRNA TS | synthetic genome | AmiR TS |
| gRNA TS | synthetic genome | pri-miR |
| gRNA TS | synthetic genome | ribozyme |
| gRNA TS | synthetic genome | aptazyme |
| gRNA TS | synthetic genome | RNAseH primer TS |
| gRNA TS | synthetic genome | Restr Enz RS |
| pri-miR | synthetic genome | siRNA TS |
| pri-miR | synthetic genome | miR TS |
| pri-miR | synthetic genome | AmiR TS |
| pri-miR | synthetic genome | gRNA TS |
| pri-miR | synthetic genome | ribozyme |
| pri-miR | synthetic genome | aptazyme |
| pri-miR | synthetic genome | RNAseH primer TS |
| pri-miR | synthetic genome | Restr Enz RS |
| ribozyme | synthetic genome | siRNA TS |
| ribozyme | synthetic genome | miR TS |
| ribozyme | synthetic genome | AmiR TS |
| ribozyme | synthetic genome | gRNA TS |
| ribozyme | synthetic genome | pri-miR |
| ribozyme | synthetic genome | aptazyme |
| ribozyme | synthetic genome | RNAseH primer TS |
| ribozyme | synthetic genome | Restr Enz RS |
| aptazyme | synthetic genome | siRNA TS |
| aptazyme | synthetic genome | miR TS |
| aptazyme | synthetic genome | AmiR TS |
| aptazyme | synthetic genome | gRNA TS |
| aptazyme | synthetic genome | pri-miR |
| aptazyme | synthetic genome | ribozyme |
| aptazyme | synthetic genome | RNAseH primer TS |
| aptazyme | synthetic genome | Restr Enz RS |
| RNAseH primer TS | synthetic genome | siRNA TS |
| RNAseH primer TS | synthetic genome | miR TS |
| RNAseH primer TS | synthetic genome | AmiR TS |
| RNAseH primer TS | synthetic genome | gRNA TS |
| RNAseH primer TS | synthetic genome | pri-miR |
| RNAseH primer TS | synthetic genome | ribozyme |
| RNAseH primer TS | synthetic genome | aptazyme |
| RNAseH primer TS | synthetic genome | Restr Enz RS |

*“Restr Enz RS” refers to restriction enzyme recognition site

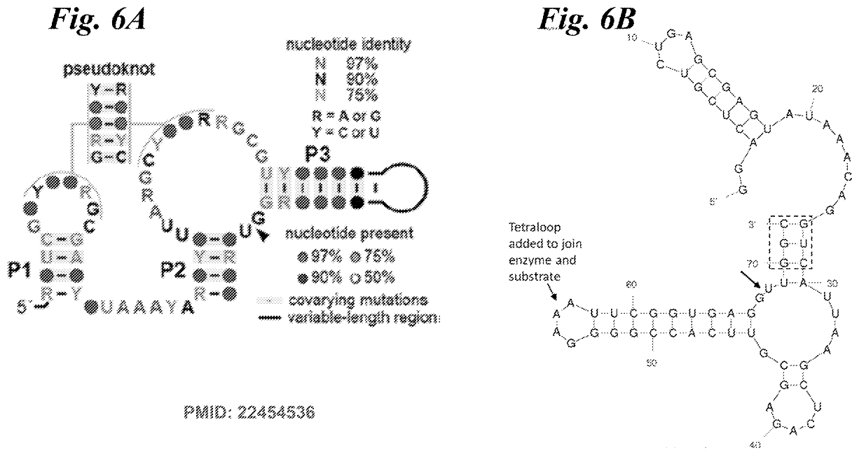

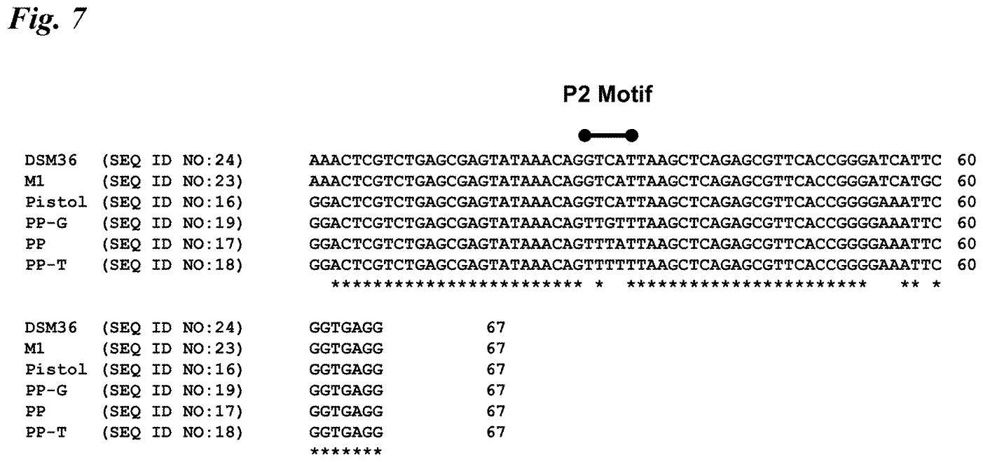

In some embodiments, the junctional cleavage sequences are ribozyme-encoding sequences and mediate self-cleavage of the synthetic RNA genome intermediates to produce the native discrete 5′ and/or 3′ ends of required for the final synthetic viral RNA genome and subsequent production of infectious RNA viruses. Exemplary ribozymes include the Hammerhead ribozyme (e.g., the Hammerhead ribozymes shown in FIG. 5A), the Varkud satellite (VS) ribozyme, the hairpin ribozyme, the GIRI branching ribozyme, the glmS ribozyme, the twister ribozyme, the twister sister ribozyme (e.g., twister sister 1 or twister sister 2), the pistol ribozyme (e.g., the pistol ribozymes shown in FIGS. 6A-6B and 7, the Env25 pistol ribozyme, or the Alistipes Putredinis Pistol Ribozyme), the hatchet ribozyme, and the Hepatitis delta virus ribozyme. In some embodiments, the 5′ and/or 3′ junctional cleavage sequences are ribozyme encoding sequences.

In some embodiments, the 5′ junctional cleavage sequence comprises or consists of a ribozyme sequence. In some embodiments, the 5′ ribozyme sequence are selected from a Hammerhead ribozyme sequence, a Pistol ribozyme sequence, or a Twister Sister ribozyme sequence.

In some embodiments, the 5′ junctional cleavage sequence comprises or consists of a 5′ Pistol ribozyme sequence. In some embodiments, the 5′ Pistol ribozyme sequence is derived from P. polymyxa. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to any one of SEQ ID NO: 16-19 and 23-24. In some embodiments, the 5′ Pistol ribozyme sequence comprises a P2 motif as indicated in FIGS. 6A and 6C, which is four nucleotides in length and locates at the region corresponding to nucleic acid positions 27-30 of SEQ ID NO: 16-19 and 23-24. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 17, wherein the polynucleotide sequence of its P2 motif is “TTTA”. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 18, wherein the polynucleotide sequence of its P2 motif is “TTTT”. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 19, wherein the polynucleotide sequence of its P2 motif is “TTGT”. In some embodiments, the 5′ Pistol ribozyme sequence is incorporated into the recombinant DNA molecule for in vitro transcription of a Coxsackievirus (e.g., CVA21) RNA viral genome.

In some embodiments, the 5′ junctional cleavage sequence comprises or consists of a 5′ Pistol ribozyme sequence. In some embodiments, the 5′ Pistol ribozyme sequence is derived from P. polymyxa. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 64 or 65. In some embodiments, the 5′ Pistol ribozyme sequence comprises a P2 motif, which is four nucleotides in length and locates at the region corresponding to nucleic acid positions 27-30 of SEQ ID NO: 64 or 65. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 64, wherein the polynucleotide sequence of its P2 motif is “TCAA”. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 65, wherein the polynucleotide sequence of its P2 motif is “TTAA”. In some embodiments, the 5′ Pistol ribozyme sequence is incorporated into the recombinant DNA molecule for in vitro transcription of an SVV (e.g., SVV-IRES-2) RNA viral genome.

In some embodiments, the 5′ junctional cleavage sequence comprises or consists of a ENV27 ribozyme encoding sequence. In some embodiments, the ENV27 ribozyme encoding sequence comprises or consists of a sequence having at least 80%, at least 85%, at least 90%, at least 95%, at least 96%, at least 97%, at least 98%, at least 99%, or 100% (including all ranges and subranges therebetween) sequence identity to any one of SEQ ID NO: 130-134. In some embodiments, the ENV27 ribozyme encoding sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 132.

In some embodiments, the ENV27 ribozyme encoding sequence contains modification (e.g., insertion) at the P3 stem insert region, which correspond to nucleotides 49-54 of SEQ ID NO: 132. Without wishing to be bound by any particular theory, it is hypothesized that extending the P3 stem insert region may facilitate the folding and/or cleavage efficiency of the ENV27 ribozyme. In some embodiments, the ENV27 ribozyme encoding sequence comprises a P3 stem insert of about 1-30, about 1-25, about 1-20, about 1-15, about 1-10, about 5-30, about 5-25, about 5-20, about 5-15, about 5-10, about 6-30, about 6-25, about 6-20, about 6-15, or about 6-10 polynucleotides in length. In some embodiments, the ENV27 ribozyme encoding sequence comprises the P3 stem insert of at least 1, 2, 3, 4, 5, 6, 7, 8, 9, 10, 11, 12, 13, 14, 15, 16, 17, 18, 19, or 20 polynucleotides in length. In some embodiments, the P3 stem insert comprises or consists of the polynucleotides “AGATCT” at the region corresponding to nucleotides 49-54 of SEQ ID NO: 132. In some embodiments, the P3 stem insert comprises or consists of the polynucleotides “AGAGAAATCT” (SEQ ID NO: 137) at the region corresponding to nucleotides 49-54 of SEQ ID NO: 132. In some embodiments, the P3 stem insert comprises or consists of the polynucleotides “AGAACGAGAAATCGTTCT” (SEQ ID NO: 138) at the region corresponding to nucleotides 49-54 of SEQ ID NO: 132.

In some embodiments, the ENV27 ribozyme encoding sequence comprises or consists of a sequence (excluding the P3 stem insert region) having at least 80%, at least 85%, at least 90%, at least 95%, at least 96%, at least 97%, at least 98%, at least 99%, or 100% (including all ranges and subranges therebetween) sequence identity to SEQ ID NO: 132 (excluding its P3 stem insert corresponding to nucleotides 49-54 of SEQ ID NO: 132). In some embodiments, the ENV27 ribozyme encoding sequence comprises or consists of a sequence (excluding the P3 stem insert region) that is 100% identical to SEQ ID NO: 132 (excluding its P3 stem insert corresponding to nucleotides 49-54 of SEQ ID NO: 132). In some embodiments, the ENV27 ribozyme encoding sequence comprises or consists of a sequence (excluding the P3 stem insert region) having at most 1, at most 2, at most 3, at most 5, at most 5, at most 6, at most 7, at most 8, at most 8, at most 10, or at most 11 mutations (insertions, deletions or substitutions) as compared to, SEQ ID NO: 132 (excluding its P3 stem insert corresponding to nucleotides 49-54 of SEQ ID NO: 132). In some embodiments, such mutation(s) are substitution(s).

In some embodiments, the ENV27 ribozyme encoding sequence comprises the polynucleotides “TTTATT” at the positions corresponding to nucleotides 25-30 of SEQ ID NO: 132.

In some embodiments, the ENV27 ribozyme encoding sequence comprises the polynucleotides “TTTGTT” at the positions corresponding to nucleotides 25-30 of SEQ ID NO: 132.

In some embodiments, the ENV27 sequence is incorporated into the recombinant DNA molecule for in vitro transcription of a Coxsackievirus (e.g., CVA21) RNA viral genome.

In some embodiments, the disclosure provides a plurality of recombinant RNA molecules, transcribed from the recombinant DNA molecule of the disclosure. In some embodiments, at least 70%, at least 75%, at least 80%, at least 85%, at least 90%, at least 95%, at least 96%, at least 97%, at least 98%, at least 99%, at least 99.5%, or at least 99.9%, of the recombinant RNA molecules comprise 5′ and 3′ ends that are native to the viral genome encoded by the recombinant RNA molecule. In some embodiments, no more than 30%, no more than 25%, no more than 20%, no more than 15%, no more than 10%, no more than 5%, no more than 4%, no more than 3%, no more than 2%, no more than 1%, no more than 0.5%, or no more than 0.1%, of the recombinant RNA molecules comprise an RNA sequence encoded by the ENV27 ribozyme encoding sequence. In some embodiments, at least one of the recombinant RNA molecules comprises an RNA sequence encoded by the ENV27 ribozyme encoding sequence. In some embodiments, at least 0.0001%, at least 0.001%, at least 0.01%, at least 0.1%, or at least 1%, of the recombinant RNA molecules comprise the ENV27 ribozyme (which is encoded by the ENV27 ribozyme encoding sequence).

In some embodiments, the 5′ junctional cleavage sequence comprises or consists of a Env25 Pistol Ribozyme. In some embodiments, the DNA sequence encoding the Env25 Pistol ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 96. In some embodiments, the Env25 Pistol ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 100.

In some embodiments, the 5′ junctional cleavage sequence comprises or consists of a Alistipes Putredinis Pistol Ribozyme. In some embodiments, the DNA sequence encoding the Alistipes Putredinis Pistol Ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 97. In some embodiments, the Alistipes Putredinis Pistol Ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 101.

In some embodiments, the 5′ junctional cleavage sequence comprises or consists of a Twister Sister 1 Ribozyme. In some embodiments, the DNA sequence encoding the Twister Sister 1 Ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 98. In some embodiments, the Twister Sister1 Ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 102.

In some embodiments, the 5′ junctional cleavage sequence comprises or consists of a Twister Sister 2 Ribozyme. In some embodiments, the DNA sequence encoding the Twister Sister 2 Ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 99. In some embodiments, the Twister Sister2 Ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 103.

### Leader Sequence

In some embodiments, the recombinant DNA molecule (e.g., DNA template) comprises a leader sequence in between the promoter sequence and the 5′ junctional cleavage sequence. In some embodiments, the presence of the leader sequence promotes or ensures the proper folding of the downstream 5′ junctional cleavage sequence (e.g., a 5′ ribozyme sequence).

In some embodiments, the leader sequence is about 5 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, or about 100 bp in length, including all ranges and subranges therebetween. In some embodiments, the leader sequence is at least 5 bp, at least 10 bp, at least 15 bp, at least 20 bp, at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 85 bp, at least 90 bp, at least 95 bp, or at least 100 bp in length, including all ranges and subranges therebetween. In some embodiments, the leader sequence is less than 5 bp, less than 10 bp, less than 15 bp, less than 20 bp, less than 25 bp, less than 30 bp, less than 35 bp, less than 40 bp, less than 45 bp, less than 50 bp, less than 55 bp, less than 60 bp, less than 65 bp, less than 70 bp, less than 75 bp, less than 80 bp, less than 85 bp, less than 90 bp, less than 95 bp, or less than 100 bp in length, including all ranges and subranges therebetween. In some embodiments, the leader sequence is about 50-70 bp, about 40-60 bp, about 60-80 bp, about 40-80 bp, about 30-70 bp, about 50-90 bp, about 30-90 bp, about 20-60 bp, or about 60-100 bp in length, including all ranges and subranges therebetween. In some embodiments, the leader sequence is about 57 bp or about 55-60 bp in length.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to any one of SEQ ID NO: 13-15. In some embodiments, the leader sequence comprises or consists of a polynucleotide sequence according to any one of SEQ ID NO: 13-15. In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to SEQ ID NO: 15. In some embodiments, the leader sequence comprises or consists of a polynucleotide sequence according to SEQ ID NO: 15. In some embodiment, the leader sequence is followed, or immediately followed, by a 5′ Pistol ribozyme sequence (e.g., a Pistol ribozyme from P. polymyxa or a variant thereof). In some embodiments, the leader sequence is incorporated into a recombinant DNA molecule (e.g., DNA template) for in vitro transcription of a CVA21 RNA viral genome.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to any one of SEQ ID NO: 135-136. In some embodiments, the leader sequence comprises or consists of a polynucleotide sequence according to any one of SEQ ID NO: 135-136. In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to SEQ ID NO: 135. In some embodiments, the leader sequence comprises or consists of a polynucleotide sequence according to SEQ ID NO: 135. In some embodiment, the leader sequence is followed, or immediately followed, by a ENV27 ribozyme sequence (e.g., any one of SEQ ID NO: 130-134 or a variant thereof). In some embodiments, the leader sequence is incorporated into a recombinant DNA molecule (e.g., DNA template) for in vitro transcription of a CVA21 RNA viral genome.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to any one of SEQ ID NO: 53-63. In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to any one of SEQ ID NO: 53-60 and 62-63. In some embodiments, the leader sequence comprises or consists of a polynucleotide sequence according to any one of SEQ ID NO: 53-60 and 62-63.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to SEQ ID NO: 53. In some embodiments, the leader sequence comprises or consists of a polynucleotide sequence according to SEQ ID NO: 53.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to SEQ ID NO: 58. In some embodiments, the leader sequence comprises or consists of a polynucleotide sequence according to SEQ ID NO: 58.

In some embodiment, the leader sequence is followed, or immediately followed, by a 5′ Pistol ribozyme sequence (e.g., a Pistol ribozyme according to SEQ ID NO: 64 or 65 or a variant thereof). In some embodiments, the leader sequence is incorporated into a recombinant DNA molecule (e.g., DNA template) for in vitro transcription of a SVV RNA viral genome.

### Poly-A Tail

In some embodiments, the recombinant DNA molecule (e.g., DNA template) comprises a sequence encoding a polyA tail. In some embodiments, a poly-A tail is attached to the 3′ end of the synthetic RNA viral genome. In some embodiments, the poly-A tail is 2-500 bp in length (i.e., 2-500 pA). In some embodiments, the poly-A tail is 2-100, 2-150, 2-200, 2-250, 2-300, 2-400, or 2-500 bp in length, including all ranges and subranges therebetween. In some embodiments, the poly-A tail is about 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 bp in length, including all ranges and subranges therebetween. In some embodiments, the poly-A tail is about 10-30, 20-40, 30-50, 40-60, 50-70, 60-80, 70-90, 80-100, 90-110, 100-120, 110-130, 120-140, 130-150, 140-160, 150-170, 160-180, 170-190, or 180-200 bp in length, including all ranges and subranges therebetween. In some embodiments, the poly-A tail is about 65-75, 60-80, 55-85, 50-90, 45-95, or 40-100 bp in length, including all ranges and subranges therebetween. In some embodiments, the poly-A tail is about 70 bp in length. In some embodiments, a longer poly-A tail (e.g., about 70 bp in length) improves the loading capacity of the synthetic RNA viral genome on an Oligo-dT chromatography as compared to a corresponding synthetic RNA viral genome with a shorter poly-A tail (e.g., about 30 bp in length). In some embodiments, the loading capacity is improved by at least 10%, at least 20%, at least 30%, at least 40%, at least 50%, at least 60%, at least 70%, at least 80%, at least 90%, at least 1-fold, at least 2-fold, at least 3-fold, at least 4-fold, at least 5-fold, at least 7-fold, or at least 10-fold, as compared to the synthetic RNA viral genome with a poly-A tail of about 30 bp in length.

### Non-Limiting Examples of Recombinant DNA Molecule Designs

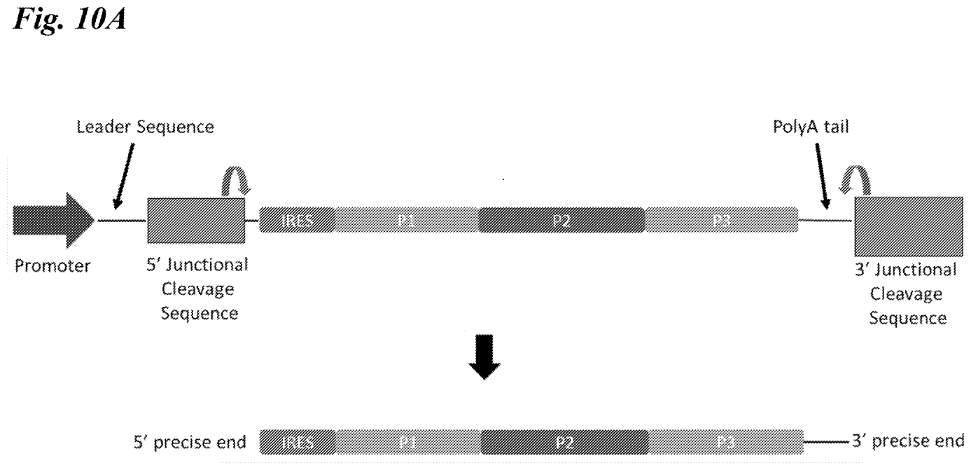

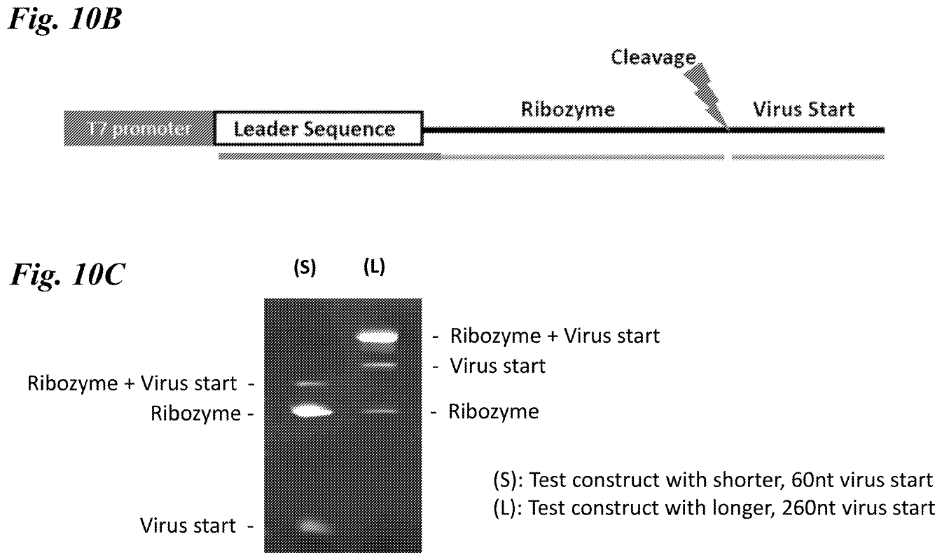

In some embodiments, the synthetic RNA viral genomes described herein are produced in vitro by in vitro RNA transcription (See, e.g., schematic in FIG. 8, FIG. 9A, FIG. 9B and FIG. 10A). The synthetic RNA viral genomes are then purified and formulated for therapeutic use (e.g., encapsulated into a lipid nanoparticle).

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a ribozyme sequence; (iii) a polynucleotide encoding the synthetic RNA viral genome; and (iv) a 3′ junctional cleavage sequence comprising or consisting of a ribozyme sequence. In some embodiments, the recombinant DNA molecule (e.g., DNA template) comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 5A and FIG. 5B); (iii) a polynucleotide encoding the synthetic RNA viral genome; and (iv) a 3′ hepatitis delta virus ribozyme sequence.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a ribozyme sequence; (iii) a polynucleotide encoding the synthetic RNA viral genome; and (iv) a 3′ junctional cleavage sequence comprising a restriction enzyme recognition site. In some embodiments, the recombinant DNA molecule (e.g., DNA template) comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 5A and FIG. 5B); (iii) a polynucleotide encoding the synthetic RNA viral genome; and (iv) a 3′ junctional cleavage sequence comprising or consisting of a SapI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a ribozyme sequence; (iii) a polynucleotide encoding the synthetic RNA viral genome; and (iv) a 3′ junctional cleavage sequence comprising or consisting of a restriction enzyme recognition site. In some embodiments, the DNA template comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 5A and FIG. 5B); (iii) a polynucleotide encoding the synthetic RNA viral genome; and (iv) a 3′ junctional cleavage sequence comprising or consisting of a BsaI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of an ENV27 ribozyme sequence; (iii) a polynucleotide encoding the synthetic RNA viral genome; and (iv) a 3′ junctional cleavage sequence comprising or consisting of a SapI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of an ENVY27 ribozyme sequence; (iii) a polynucleotide encoding the synthetic RNA viral genome; and (iv) a 3′ junctional cleavage sequence comprising or consisting of a BsaI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a 5′ RNAseHI primer binding site; (iii) a polynucleotide encoding the synthetic RNA viral genome; and (iv) a 3′ junctional cleavage sequence comprising a restriction enzyme recognition site. In some embodiments, the recombinant DNA molecule (e.g., DNA template) comprises a polynucleotide comprising, from 5′ to 3′: (i) a promoter sequence (e.g., a T7 polymerase promoter); (ii) a 5′ junctional cleavage sequence comprising or consisting of a 5′ RNAseH primer binding site; (iii) a polynucleotide encoding the synthetic RNA viral genome; and (iv) a 3′ junctional cleavage sequence comprising or consisting of a SapI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a 5′ RNAseHI primer binding site; (iii) a polynucleotide encoding the synthetic RNA viral genome; and (iv) a 3′ junctional cleavage sequence comprising a restriction enzyme recognition site. In some embodiments, the recombinant DNA molecule (e.g., DNA template) comprises a polynucleotide comprising, from 5′ to 3′: (i) a promoter sequence (e.g., a T7 polymerase promoter); (ii) a 5′ junctional cleavage sequence comprising or consisting of a 5′ RNAseH primer binding site; (iii) a polynucleotide encoding the synthetic RNA viral genome; and (iv) a 3′ junctional cleavage sequence comprising or consisting of a BsaI restriction enzyme recognition site.

In some embodiments, the synthetic RNA viral genome is a Coxsackievirus (CVA) genome. In some embodiments, the Coxsackievirus is a CVA21 strain. In some embodiments, the CVA21 strain is an EF strain. In some embodiments, the CVA21 strain is a KY strain.

In some embodiments, the recombinant DNA molecule (e.g., DNA template) comprises, from 5′ to 3′: (i) a promoter sequence (e.g., a T7 polymerase promoter); (ii) an optional leader sequence; (iii) a 5′ junctional cleavage sequence comprising or consisting of an ENV27 ribozyme sequence; (iv) a polynucleotide encoding the synthetic RNA viral genome; (v) a poly-A tail (e.g., about 20-80 bp in length, or about 30-70 bp in length), and (vi) a 3′ junctional cleavage sequence comprising or consisting of a restriction enzyme recognition site (e.g., for BsmBI or BsaI restriction enzyme).

In some embodiments, the recombinant DNA molecule (e.g., DNA template) comprises, from 5′ to 3′: (i) a promoter sequence (e.g., a T7 polymerase promoter); (ii) a leader sequence (e.g., SEQ ID NO: 135 or 136); (iii) a 5′ junctional cleavage sequence comprising or consisting of an ENV27 ribozyme sequence; (iv) a polynucleotide encoding a CVA21 synthetic RNA viral genome; (v) a poly-A tail (e.g., about 20-80 bp in length, or about 30-70 bp in length), and (vi) a 3′ junctional cleavage sequence comprising or consisting of a BsmBI restriction enzyme recognition site.

In some embodiments, the recombinant DNA molecule (e.g., DNA template) comprises, from 5′ to 3′: (i) a promoter sequence (e.g., a T7 polymerase promoter); (ii) a leader sequence (e.g., SEQ ID NO: 135 or 136); (iii) a 5′ junctional cleavage sequence comprising or consisting of an ENV27 ribozyme sequence; (iv) a polynucleotide encoding a CVA21 synthetic RNA viral genome; (v) a poly-A tail (e.g., about 20-80 bp in length, or about 30-70 bp in length), and (vi) a 3′ junctional cleavage sequence comprising or consisting of a BsaI restriction enzyme recognition site.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 135; (iii) a 5′ junctional cleavage sequence comprising or consisting of an ENV27 ribozyme sequence; (iv) a polynucleotide encoding a CVA21 synthetic RNA viral genome; (v) a poly-A tail, and (vi) a 3′ junctional cleavage sequence comprising or consisting of a BsmBI restriction enzyme recognition site, wherein the combination of the 5′ ENV27 ribozyme sequence and the poly-A tail is selected from one of Embodiments E1-E68 provided in Table 5 below.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 135; (iii) a 5′ junctional cleavage sequence comprising or consisting of an ENV27 ribozyme sequence; (iv) a polynucleotide encoding a CVA21 synthetic RNA viral genome; (v) a poly-A tail, and (vi) a 3′ junctional cleavage sequence comprising or consisting of a BsaI restriction enzyme recognition site, wherein the combination of the 5′ ENV27 ribozyme sequence and the poly-A tail is selected from one of Embodiments E1-E68 provided in Table 5 below.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 136; (iii) a 5′ junctional cleavage sequence comprising or consisting of an ENV27 ribozyme sequence; (iv) a polynucleotide encoding a CVA21 synthetic RNA viral genome; (v) a poly-A tail, and (vi) a 3′ junctional cleavage sequence comprising or consisting of a BsmBI restriction enzyme recognition site, wherein the combination of the 5′ ENV27 ribozyme sequence and the poly-A tail is selected from one of Embodiments E1-E68 provided in Table 5 below.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 136; (iii) a 5′ junctional cleavage sequence comprising or consisting of an ENV27 ribozyme sequence; (iv) a polynucleotide encoding a CVA21 synthetic RNA viral genome; (v) a poly-A tail, and (vi) a 3′ junctional cleavage sequence comprising or consisting of a BsaI restriction enzyme recognition site, wherein the combination of the 5′ ENV27 ribozyme sequence and the poly-A tail is selected from one of Embodiments E1-E68 provided in Table 5 below.

TABLE 5
Non-limiting Embodiments of Leader Sequence, 5′ Ribozyme Sequence,
and Poly-A Tail in the DNA template for Expressing CVA21 Viral Genome

| | 5′ ENV27 | 5′ ENV27 |
|---|---|---|
| | Ribozyme with | Ribozyme with |
| | “TTTATT” at the | “TTTGTT” at the |
| | positions | positions |
| | corresponding to | corresponding to |
| | nucleotides 25-30 of | nucleotides 25-30 of |
| | SEQ ID NO: 132 | SEQ ID NO: 132 |
| Leader sequence according to SEQ ID NO: | Embodiment E1 | Embodiment E2 |
| 135; | | |
| poly-A tail about 100 pA in length | | |
| Leader sequence according to SEQ ID NO: | Embodiment E3 | Embodiment E4 |
| 135; | | |
| poly-A tail about 90 pA in length | | |
| Leader sequence according to SEQ ID NO: | Embodiment E5 | Embodiment E6 |
| 135; | | |
| poly-A tail about 80 pA in length | | |
| Leader sequence according to SEQ ID NO: | Embodiment E7 | Embodiment E8 |
| 135; | | |
| poly-A tail about 70 pA in length | | |
| Leader sequence according to SEQ ID NO: | Embodiment E9 | Embodiment E10 |
| 135; | | |
| poly-A tail about 60 pA in length | | |
| Leader sequence according to SEQ ID NO: | Embodiment E11 | Embodiment E12 |
| 135; | | |
| poly-A tail about 50 pA in length | | |
| Leader sequence according to SEQ ID NO: | Embodiment E13 | Embodiment E14 |
| 135; | | |
| poly-A tail about 40 pA in length | | |
| Leader sequence according to SEQ ID NO: | Embodiment E15 | Embodiment E16 |
| 135; | | |
| poly-A tail about 30 pA in length | | |
| Leader sequence according to SEQ ID NO: | Embodiment E17 | Embodiment E18 |
| 135; | | |
| poly-A tail about 20 pA in length | | |
| Leader sequence according to SEQ ID NO: | Embodiment E19 | Embodiment E20 |
| 135; | | |
| poly-A tail about 80-100 pA in length | | |
| Leader sequence according to SEQ ID NO: | Embodiment E21 | Embodiment E22 |
| 135; | | |
| poly-A tail about 70-90 pA in length | | |
| Leader sequence according to SEQ ID NO: | Embodiment E23 | Embodiment E24 |
| 135; | | |
| poly-A tail about 60-80 pA in length | | |
| Leader sequence according to SEQ ID NO: | Embodiment E25 | Embodiment E26 |
| 135; | | |
| poly-A tail about 50-70 pA in length | | |
| Leader sequence according to SEQ ID NO: | Embodiment E27 | Embodiment E28 |
| 135; | | |
| poly-A tail about 40-60 pA in length | | |
| Leader sequence according to SEQ ID NO: | Embodiment E29 | Embodiment E30 |
| 135; | | |
| poly-A tail about 30-50 pA in length | | |
| Leader sequence according to SEQ ID NO: | Embodiment E31 | Embodiment E32 |
| 135; | | |
| poly-A tail about 20-40 pA in length | | |
| Leader sequence according to SEQ ID NO: | Embodiment E33 | Embodiment E34 |
| 135; | | |
| poly-A tail about 10-30 pA in length | | |
| Leader sequence according to SEQ ID NO: | Embodiment E35 | Embodiment E36 |
| 136; | | |
| poly-A tail about 100 pA in length | | |
| Leader sequence according to SEQ ID NO: | Embodiment E37 | Embodiment E38 |
| 136; | | |
| poly-A tail about 90 pA in length | | |
| Leader sequence according to SEQ ID NO: | Embodiment E39 | Embodiment E40 |
| 136; | | |
| poly-A tail about 80 pA in length | | |
| Leader sequence according to SEQ ID NO: | Embodiment E41 | Embodiment E42 |
| 136; | | |
| poly-A tail about 70 pA in length | | |
| Leader sequence according to SEQ ID NO: | Embodiment E43 | Embodiment E44 |
| 136; | | |
| poly-A tail about 60 pA in length | | |
| Leader sequence according to SEQ ID NO: | Embodiment E45 | Embodiment E46 |
| 136; | | |
| poly-A tail about 50 pA in length | | |
| Leader sequence according to SEQ ID NO: | Embodiment E47 | Embodiment E48 |
| 136; | | |
| poly-A tail about 40 pA in length | | |
| Leader sequence according to SEQ ID NO: | Embodiment E49 | Embodiment E50 |
| 136; | | |
| poly-A tail about 30 pA in length | | |
| Leader sequence according to SEQ ID NO: | Embodiment E51 | Embodiment E52 |
| 136; | | |
| poly-A tail about 20 pA in length | | |
| Leader sequence according to SEQ ID NO: | Embodiment E53 | Embodiment E54 |
| 136; | | |
| poly-A tail about 80-100 pA in length | | |
| Leader sequence according to SEQ ID NO: | Embodiment E55 | Embodiment E56 |
| 136; | | |
| poly-A tail about 70-90 pA in length | | |
| Leader sequence according to SEQ ID NO: | Embodiment E57 | Embodiment E58 |
| 136; | | |
| poly-A tail about 60-80 pA in length | | |
| Leader sequence according to SEQ ID NO: | Embodiment E59 | Embodiment E60 |
| 136; | | |
| poly-A tail about 50-70 pA in length | | |
| Leader sequence according to SEQ ID NO: | Embodiment E61 | Embodiment E62 |
| 136; | | |
| poly-A tail about 40-60 pA in length | | |
| Leader sequence according to SEQ ID NO: | Embodiment E63 | Embodiment E64 |
| 136; | | |
| poly-A tail about 30-50 pA in length | | |
| Leader sequence according to SEQ ID NO: | Embodiment E65 | Embodiment E66 |
| 136; | | |
| poly-A tail about 20-40 pA in length | | |
| Leader sequence according to SEQ ID NO: | Embodiment E67 | Embodiment E68 |
| 136; | | |
| poly-A tail about 10-30 pA in length | | |

In some embodiments, the recombinant DNA molecule (e.g., DNA template) comprises, from 5′ to 3′: (i) a T7 polymerase promoter sequence; (ii) a leader sequence according to SEQ ID NO: 135; (iii) a 5′ junctional cleavage sequence comprising or consisting of a 5′ ENV27 ribozyme sequence according to SEQ ID NO: 132; (iv) a polynucleotide encoding a CVA21 synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a BsmBI restriction enzyme recognition site.

In some embodiments, the recombinant DNA molecule (e.g., DNA template) comprises, from 5′ to 3′: (i) a T7 polymerase promoter sequence; (ii) a leader sequence according to SEQ ID NO: 135; (iii) a 5′ junctional cleavage sequence comprising or consisting of a 5′ ENV27 ribozyme sequence according to SEQ ID NO: 132; (iv) a polynucleotide encoding a CVA21 synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a BsaI restriction enzyme recognition site.

Exemplary embodiments of DNA templates encoding CVA viral genomes are provided below in Table 18.

TABLE 18
Exemplary DNA Template Structures

| | | | Encoded | PolyA | |
|---|---|---|---|---|---|
| Promoter | Leader | 5′ JCS | Genome | Tail | 3′ JCS |
| T7 | SEQ ID | SEQ ID | CVA21 KY | 70 AA | SEQ ID NO: 86 |
| Promoter | NO: 135 | NO: 132 | Strain | | (BsmBI) |
| T7 | SEQ ID | SEQ ID | CVA21 EF | 70 AA | SEQ ID NO: 86 |
| Promoter | NO: 135 | NO: 132 | Strain | | (BsmBI) |
| T7 | SEQ ID | SEQ ID | CVA21 | 70 AA | SEQ ID NO: 86 |
| Promoter | NO: 135 | NO: 132 | Kuykendall | | (BsmBI) |
| | | | Strain | | |
| T7 | SEQ ID | SEQ ID | CVA21 KY | 70 AA | SEQ ID NO: 90 |
| Promoter | NO: 135 | NO: 132 | Strain | | (BsaI) |
| T7 | SEQ ID | SEQ ID | CVA21 EF | 70 AA | SEQ ID NO: 90 |
| Promoter | NO: 135 | NO: 132 | Strain | | BsaI) |
| T7 | SEQ ID | SEQ ID | CVA21 | 70 AA | SEQ ID NO: 90 |
| Promoter | NO: 135 | NO: 132 | Kuykendall | | (BsaI) |
| | | | Strain | | |

### Particles Comprising Synthetic RNA Genomes

In some embodiments, the synthetic RNA genomes described herein are encapsulated in “particles.” As used herein, a particle refers to a non-tissue derived composition of matter such as liposomes, lipoplexes, nanoparticles, nanocapsules, microparticles, microspheres, lipid particles, exosomes, vesicles, and the like. In certain embodiments, the particles are non-proteinaceous and non-immunogenic. In such embodiments, encapsulation of the synthetic RNA genomes described herein allows for delivery of a viral genome without the induction of a systemic, anti-viral immune response and mitigates the effects of neutralizing anti-viral antibodies. Further, encapsulation of the synthetic RNA genomes described herein shields the genomes from degradation and facilitates the introduction into target host cells. In some embodiments, the present disclosure provides a nanoparticle comprising a synthetic RNA genome described herein. In some embodiments, the nanoparticle is a lipid nanoparticle. In some embodiments, the nanoparticle further comprises a second RNA molecule encoding a payload molecule.

In some embodiments, the particle is biodegradable in a subject. In such embodiments, multiple doses of the particles can be administered to a subject without an accumulation of particles in the subject. Examples of suitable particles include polystyrene particles, poly(lactic-co-glycolic acid) PLGA particles, polypeptide-based cationic polymer particles, cyclodextrin particles, chitosan, N,N,N-trimethyl chitosan particles, lipid based particles, poly(β-amino ester) particles, low-molecular-weight polyethylenimine particles, polyphosphoester particles, disulfide cross-linked polymer particles, polyamidoamine particles, polyethylenimine (PEI) particles, and PLURIONICS stabilized polypropylene sulfide particles.

In some embodiments, the polynucleotides described herein are encapsulated in inorganic particles. In some embodiments, the inorganic particles are gold nanoparticles (GNP), gold nanorods (GNR), magnetic nanoparticles (MNP), magnetic nanotubes (MNT), carbon nanohorns (CNH), carbon fullerenes, carbon nanotubes (CNT), calcium phosphate nanoparticles (CPNP), mesoporous silica nanoparticles (MSN), silica nanotubes (SNT), or a starlike hollow silica nanoparticles (SHNP).

Preferably, the particles described herein are nanoscopic in size, in order to enhance solubility, avoid clearance by phagocytic cells and possible complications caused by aggregation in vivo and to facilitate pinocytosis. In some embodiments, the particle has an average diameter of about less than about 1000 nm. In some embodiments, the particle has an average diameter of less than about 500 nm. In some embodiments, the particle has an average diameter of between about 30 and about 100 nm, between about 50 and about 100 nm, or between about 75 and about 100 nm. In some embodiments, the particle has an average diameter of between about 30 and about 75 nm or between about 30 and about 50 nm. In some embodiments, the particle has an average diameter between about 100 and about 500 nm. In some embodiments, the particle has an average diameter between about 200 and 400 nm. In some embodiments, the particle has an average size of about 350 nm.

### Exosomes

In some embodiments, the synthetic RNA genomes described herein are encapsulated in exosomes. Exosomes are small membrane vesicles of endocytic origin that are released into the extracellular environment following fusion of multivesicular bodies with the plasma membrane of the parental cell (e.g., the cell from which the exosome is released, also referred to herein as a donor cell). The surface of an exosome comprises a lipid bilayer derived from the parental cell's cell membrane and can further comprise membrane proteins expressed on the parental cell surface. In some embodiments, exosomes may also contain cytosol from the parental cell. Exosomes are produced by many different cell types including epithelial cells, B and T lymphocytes, mast cells (MC), and dendritic cells (DC) and have been identified in blood plasma, urine, bronchoalveolar lavage fluid, intestinal epithelial cells, and tumor tissues. Because the composition of an exosome is dependent on the parental cell type from which they are derived, there are no “exosome-specific” proteins. However, many exosomes comprise proteins associated with the intracellular vesicles from which the exosome originated in the parental cells (e.g., proteins associated with and/or expressed by endosomes and lysosomes). For example, exosomes can be enriched in antigen presentation molecules such as major histocompatibility complex I and II (MHC-I and MHC-II), tetraspanins (e.g., CD63), several heat shock proteins, cytoskeletal components such as actins and tubulins, proteins involved in intracellular membrane fusion, cell-cell interactions (e.g. CD54), signal transduction proteins, and cytosolic enzymes.

Exosomes may mediate transfer of cellular proteins from one cell (e.g., a parental cells) to a target or recipient cell by fusion of the exosomal membrane with the plasma membrane of the target cell. As such, modifying the material that is encapsulated by the exosome provides a mechanism by which exogenous agents, such as the polynucleotides described herein, may be introduced to a target cell. Exosomes that have been modified to contain one or more exogenous agents (e.g., a polynucleotide described herein) are referred to herein as “modified exosomes”. In some embodiments, modified exosomes are produced by introduction of the exogenous agent (e.g., a polynucleotide described herein) are introduced into a parental cell. In such embodiments, an exogenous nucleic acid is introduced into the parental, exosome-producing cells such that the exogenous nucleic acid itself, or a transcript of the exogenous nucleic acid is incorporated into the modified exosomes produced from the parental cell. The exogenous nucleic acids can be introduced to the parental cell by means known in the art, for example transduction, transfection, transformation, electroporation and/or microinjection of the exogenous nucleic acids.

In some embodiments, modified exosomes are produced by directly introducing a synthetic RNA genome described herein into an exosome. In some embodiments, a synthetic RNA genome described herein is introduced into an intact exosome. “Intact exosomes” refer to exosomes comprising proteins and/or genetic material derived from the parental cell from which they are produced. Methods for obtaining intact exosomes are known in the art (See e.g., Alvarez-Erviti L. et al., Nat Biotechnol. 2011 April; 29 (4): 34-5; Ohno S, et al., Mol Ther 2013 January; 21 (1): 185-91; and EP Patent Publication No. 2010663).

In particular embodiments, synthetic RNA genomes are introduced into empty exosomes. “Empty exosomes” refer to exosomes that lack proteins and/or genetic material (e.g., DNA or RNA) derived from the parental cell. Methods to produce empty exosomes (e.g., lacking parental cell-derived genetic material) are known in the art including UV-exposure, mutation/deletion of endogenous proteins that mediate loading of nucleic acids into exosomes, as well as electroporation and chemical treatments to open pores in the exosomal membranes such that endogenous genetic material passes out of the exosome through the open pores. In some embodiments, empty exosomes are produced by opening the exosomes by treatment with an aqueous solution having a pH from about 9 to about 14 to obtain exosomal membranes, removing intravesicular components (e.g., intravesicular proteins and/or nucleic acids), and reassembling the exosomal membranes to form empty exosomes. In some embodiments, intravesicular components (e.g., intravesicular proteins and/or nucleic acids) are removed by ultracentrifugation or density gradient ultracentrifugation. In some embodiments, the membranes are reassembled by sonication, mechanical vibration, extrusion through porous membranes, electric current, or combinations of one or more of these techniques. In particular embodiments, the membranes are reassembled by sonication.

In some embodiments, loading of intact or empty exosomes with a synthetic RNA genome described herein to produce a modified exosome can be achieved using conventional molecular biology techniques such as in vitro transformation, transfection, and/or microinjection. In some embodiments, the exogenous agents (e.g., the polynucleotides described herein) are introduced directly into intact or empty exosomes by electroporation. In some embodiments, the exogenous agents (e.g., the polynucleotides described herein) are introduced directly into intact or empty exosomes by lipofection (e.g., transfection). Lipofection kits suitable for use in the production of exosome according to the present disclosure are known in the art and are commercially available (e.g., FuGENE® HD Transfection Reagent from Roche, and LIPOFECTAMINE™ 2000 from Invitrogen). In some embodiments, the exogenous agents (e.g., the polynucleotides described herein) are introduced directly into intact or empty exosomes by transformation using heat shock. In such embodiments, exosomes isolated from parental cells are chilled in the presence of divalent cations such as Ca²⁺ (in CaCl₂)) in order to permeabilize the exosomal membrane. The exosomes can then be incubated with the exogenous nucleic acids and briefly heat shocked (e.g., incubated at 42° C. for 30-120 seconds). In particular embodiments, loading of empty exosomes with exogenous agents (e.g., the polynucleotides described herein) can be achieved by mixing or co-incubation of the agents with the exosomal membranes after the removal of intravesicular components. The modified exosomes reassembled from the exosomal membranes will, therefore, incorporate the exogenous agents into the intravesicular space. Additional methods for producing exosome encapsulated nucleic acids are known in the art (See e.g., U.S. Pat. Nos. 9,889,210; 9,629,929; and 9,085,778; International PCT Publication Nos. WO 2017/161010 and WO 2018/039119).

Exosomes can be obtained from numerous different parental cells, including cell lines, bone-marrow derived cells, and cells derived from primary patient samples. Exosomes released from parental cells can be isolated from supernatants of parental cell cultures by means known in the art. For example, physical properties of exosomes can be employed to separate them from a medium or other source material, including separation on the basis of electrical charge (e.g., electrophoretic separation), size (e.g., filtration, molecular sieving, etc.), density (e.g., regular or gradient centrifugation) and Svedberg constant (e.g., sedimentation with or without external force, etc). Alternatively, or additionally, isolation can be based on one or more biological properties, and include methods that can employ surface markers (e.g., for precipitation, reversible binding to solid phase, FACS separation, specific ligand binding, non-specific ligand binding, etc.). Analysis of exosomal surface proteins can be determined by flow cytometry using fluorescently labeled antibodies for exosome-associated proteins such as CD63. Additional markers for characterizing exosomes are described in International PCT Publication No. WO 2017/161010. In yet further contemplated methods, the exosomes can also be fused using chemical and/or physical methods, including PEG-induced fusion and/or ultrasonic fusion.

In some embodiments, size exclusion chromatography can be utilized to isolate the exosomes. In some embodiments, the exosomes can be further isolated after chromatographic separation by centrifugation techniques (of one or more chromatography fractions), as is generally known in the art. In some embodiments, the isolation of exosomes can involve combinations of methods that include, but are not limited to, differential centrifugation as previously described (See Raposo, G. et al., J. Exp. Med. 183, 1161-1172 (1996)), ultracentrifugation, size-based membrane filtration, concentration, and/or rate zonal centrifugation.

In some embodiments, the exosomal membrane comprises one or more of phospholipids, glycolipids, fatty acids, sphingolipids, phosphoglycerides, sterols, cholesterols, and phosphatidylserine. In addition, the membrane can comprise one or more polypeptides and one or more polysaccharides, such as glycans. Exemplary exosomal membrane compositions and methods for modifying the relative amount of one or more membrane component are described in International PCT Publication No. WO 2018/039119.

In some embodiments, the particles are exosomes and have a diameter between about 30 and about 100 nm, between about 30 and about 200 nm, or between about 30 and about 500 nm. In some embodiments, the particles are exosomes and have a diameter between about 10 nm and about 100 nm, between about 20 nm and about 100 nm, between about 30 nm and about 100 nm, between about 40 nm and about 100 nm, between about 50 nm and about 100 nm, between about 60 nm and about 100 nm, between about 70 nm and about 100 nm, between about 80 nm and about 100 nm, between about 90 nm and about 100 nm, between about 100 nm and about 200 nm, between about 100 nm and about 150 nm, between about 150 nm and about 200 nm, between about 100 nm and about 250 nm, between about 250 nm and about 500 nm, or between about 10 nm and about 1000 nm. In some embodiments, the particles are exosomes and have a diameter between about 20 nm and 300 nm, between about 40 nm and 200 nm, between about 20 nm and 250 nm, between about 30 nm and 150 nm, or between about 30 nm and 100 nm.

### Compounds

### Compounds of Formula (I)

In various embodiments, provided herein are compounds of Formula (I):

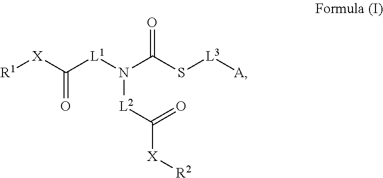

or a pharmaceutically acceptable salt or solvate thereof, wherein:
A is-N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or—N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain;
L³ is a bond, an optionally substituted C₁-C₆ alkylene chain, or a bivalent optionally substituted C₃-C₇ cycloalkylene.

In some embodiments, when A is —N(CH₃)(CH₃) and X is O, L³ is not a C₁-C₆ alkylene chain.

In some embodiments, the present disclosure includes a compound of Formula (I-a):

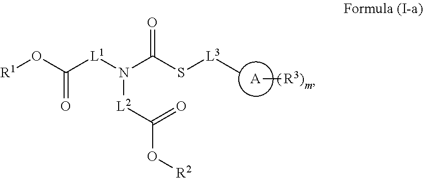

or a pharmaceutically acceptable salt or solvate thereof, wherein m is 0, 1, 2, 3, 4, 5, or 6.

In some embodiments, the present disclosure includes a compound of Formula (I-b):

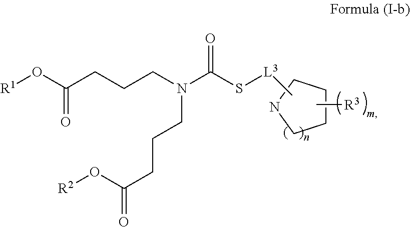

or a pharmaceutically acceptable salt or solvate thereof, wherein n is 0, 1, 2, or 3; and m is 0, 1, 2, 3, 4, 5, or 6.

In some embodiments, the present disclosure includes a compound of Formula (I-bi):

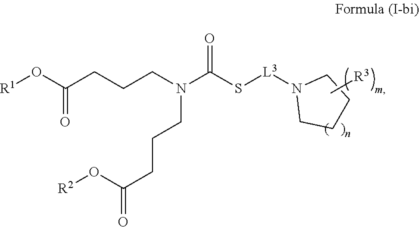

or a pharmaceutically acceptable salt or solvate thereof.

In some embodiments, the present disclosure includes a compound of Formula (I-bii):

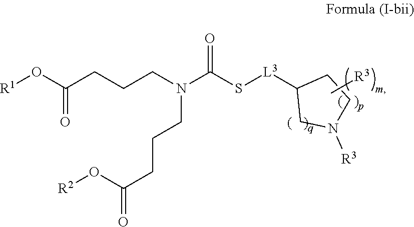

or a pharmaceutically acceptable salt or solvate thereof, wherein m is 0, 1, 2, or 3; and p and q are each 0, 1, 2, or 3, and wherein q+p is less than or equal to 3.

In some embodiments, the present disclosure includes a compound of Formula (I-biii):

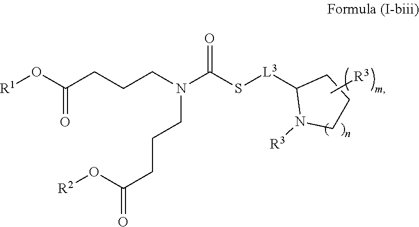

or a pharmaceutically acceptable salt or solvate thereof.

In some embodiments, the present disclosure includes a compound of Formula (I-c):

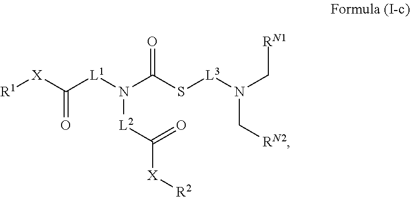

or a pharmaceutically acceptable salt or solvate thereof.

In some embodiments, A is-N(CH₂R^{N1}) (CH₂R^{N2}) or an optionally substituted 4-7-membered heterocyclyl ring containing at least one N.

In some embodiments, A is-N(CH₂R^{N1}) (CH₂R^{N2}). In some embodiments, R^{N1} and R^{N2} are each independently selected from hydrogen, hydroxy-C₁-C₃ alkylene, C₂-C₄ alkenyl, or C₃-C₄ cycloalkyl.).

In some embodiments, R^{N1} and R^{N2} are each independently selected from hydrogen, —CH₂CH—CH₂, CH₂CH₂OH,

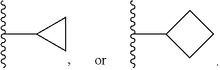

In some embodiments, R^{N1} and R^{N2} are the same. In some embodiments, R^{N1} and R^{N2} are each hydrogen. In some embodiments, R^{N1} and R^{N2} are each C₂-C₄ alkenyl, e.g., —CH₂CH═CH₂. In some embodiments, R^{N1} and R^{N2} are each hydroxy-C₁-C₃ alkylene, e.g., —CH₂CH₂OH. In some embodiments, R^{N1} and R^{N2} are different. In some embodiments, one of R^{N1} and R^{N2} is hydrogen and the other one is C₃-C₄ cycloalkyl. In some embodiments, one of R^{N1} and R^{N2} is hydrogen and the other one is

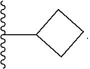 [SMILES: *C1CCC1]

In some embodiments, A is an optionally substituted 4-7-membered heterocyclyl ring containing at least one N. In some embodiments, A is an optionally substituted 4-7-membered heterocyclyl ring containing exactly one N. In some embodiments, A is an unsubstituted 4-7-membered heterocyclyl ring containing at least one N. In some embodiments, A is unsubstituted 4-7-membered heterocyclyl ring containing exactly one N. In some embodiments, A is an optionally substituted 5-6-membered heterocyclyl ring containing at least one N. In some embodiments, A is unsubstituted 5-6-membered heterocyclyl ring containing at least one N.

In some embodiments, A is an optionally substituted 4-7-membered heterocyclyl ring containing at least one N, and the N atom of A is a tertiary amine.

In some embodiments, A is an optionally substituted 4-7-membered heterocyclyl ring containing at least one N, further containing one or more S. In some embodiments, A is an optionally substituted 4-7-membered heterocyclyl ring containing at least one N, further containing exactly one S.

In some embodiments, A is selected from the group consisting of azetidine, pyrrolidine, piperidine, azepane, and thiomorpholine. In some embodiments, A is selected from the group consisting of pyrrolidine and piperidine.

In some embodiments, L¹ is selected from the group consisting of an optionally substituted C₁-C₂₀ alkylene chain and a bivalent optionally substituted C₁-C₂₀ alkenylene chain. In some embodiments, L² is selected from the group consisting of an optionally substituted C₁-C₂₀ alkylene chain and a bivalent optionally substituted C₁-C₂₀ alkenylene chain. In some embodiments, L¹ is an optionally substituted C₁-C₂₀ alkylene chain. In some embodiments, L² is an optionally substituted C₁-C₂₀ alkylene chain.

In some embodiments, L¹ and L² are the same. In some embodiments, L¹ and L² are different.

In some embodiments, L¹ is an optionally substituted C₁-C₁₀ alkylene chain. In some embodiments, L² is an optionally substituted C₁-C₁₀ alkylene chain. In some embodiments, L¹ is an optionally substituted C₁-C₅ alkylene chain. In some embodiments, L² is an optionally substituted C₁-C₅ alkylene chain.

In some embodiments, L¹ and L² are each—CH₂CH₂CH₂CH₂—. In some embodiments, L¹ and L² are each —CH₂CH₂CH₂—. In some embodiments, L¹ and L² are each—CH₂CH₂—.

In some embodiments, L³ is a bond, an optionally substituted C₁-C₆ alkylene chain, or a bivalent optionally substituted C₃-C₆ cycloalkylene. In some embodiments, L³ is a bond. In some embodiments, L³ is an optionally substituted C₁-C₆ alkylene chain. In some embodiments, L³ is an optionally substituted C₁-C₃ alkylene chain. In some embodiments, L³ is an unsubstituted C₁-C₃ alkylene chain. In some embodiments, L³ is —CH₂—. In some embodiments, L³ is —CH₂CH₂—. In some embodiments, L³ is —CH₂CH₂CH₂—. In some embodiments, L³ is a bivalent C₃-C₆ cyclcoalkylene. In some embodiments, L³ is

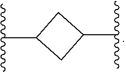 [SMILES: *C1CC(*)C1]

In some embodiments, the number of carbon atoms between the S of the thiolate of Formula (I) and the N of A is 2-10. In some embodiments, the number of carbon atoms between the S of the thiolate of Formula (I) and the N of A is 2-8. In some embodiments, the number of carbon atoms between the S of the thiolate of Formula (I) and the N of A is 2-5. In some embodiments, the number of carbon atoms between the S of the thiolate of Formula (I) and the N of A is 2-4. In some embodiments, the number of carbon atoms between the S of the thiolate of Formula (I) and the N of A is 2. In some embodiments, the number of carbon atoms between the S of the thiolate of Formula (I) and the N of A is 3. In some embodiments, the number of carbon atoms between the S of the thiolate of Formula (I) and the N of A is 4.

In some embodiments, R¹ is selected from the group consisting of optionally substituted C₁-C₃₁ aliphatic and optionally substituted steroidyl. In some embodiments, R² is selected from the group consisting of optionally substituted C₁-C₃₁ aliphatic and optionally substituted steroidyl. In some embodiments, R¹ is optionally substituted C₁-C₃₁ alkyl. In some embodiments, R² is optionally substituted C₁-C₃₁ alkyl. In some embodiments, R¹ is optionally substituted C₅-C₂₅ alkyl. In some embodiments, R² is optionally substituted C₅-C₂₅ alkyl. In some embodiments, R¹ is optionally substituted C₁₀-C₂₀ alkyl. In some embodiments, R² is optionally substituted C₁₀-C₂₀ alkyl. In some embodiments, R¹ is optionally substituted C₁₀-C₂₀ alkyl. In some embodiments, R² is optionally substituted C₁₀-C₂₀ alkyl. In some embodiments, R¹ is unsubstituted C₁₀-C₂₀ alkyl. In some embodiments, R² is unsubstituted C₁₀-C₂₀ alkyl.

In some embodiments, R¹ is optionally substituted C₁₄-C₁₆ alkyl. In some embodiments, R² is optionally substituted C₁₄-C₁₆ alkyl. In some embodiments, R¹ is unsubstituted C₁₄-C₁₆ alkyl. In some embodiments, R² is unsubstituted C₁₄-C₁₆ alkyl.

In some embodiments, R¹ is optionally substituted branched C₃-C₃₁ alkyl. In some embodiments, R² is optionally substituted branched C₃-C₃₁ alkyl. In some embodiments, R¹ is optionally substituted branched C₁₀-C₂₀ alkyl. In some embodiments, R² is optionally substituted branched C₁₀-C₂₀ alkyl. In some embodiments, R¹ is optionally substituted branched C₁₄-C₁₆ alkyl. In some embodiments, R² is optionally substituted branched C₁₄-C₁₆ alkyl. In some embodiments, R¹ is substituted branched C₃-C₃₁ alkyl. In some embodiments, R² is substituted branched C₃-C₃₁ alkyl. In some embodiments, R¹ is substituted branched C₁₀-C₂₀ alkyl. In some embodiments, R² is substituted branched C₁₀-C₂₀ alkyl. In some embodiments, R¹ is substituted branched C₁₄-C₁₆ alkyl. In some embodiments, R² is substituted branched C₁₄-C₁₆ alkyl.

In some embodiments, R¹ and R² are the same.

In some embodiments, R¹ and R² are different. In some embodiments, R¹ is optionally substituted C₆-C₂₀ alkenyl and R² is optionally substituted C₁₀-C₂₀ alkyl. In some embodiments, R¹ is C₆-C₂₀ alkenyl and R² is branched C₁₀-C₂₀ alkyl.

In some embodiments, A is 4-7-membered heterocyclyl ring containing at least one N and optionally substituted with 0-6 R³. In some embodiments, R³ is optionally substituted C₁-C₆ aliphatic. In some embodiments, R³ is optionally substituted C₁-C₃ aliphatic. In some embodiments, R³ is optionally substituted C₁-C₆ alkyl. In some embodiments, R³ is optionally substituted C₁-C₃ alkyl. In some embodiments, R³ is unsubstituted C₁-C₆ alkyl. In some embodiments, R³ is unsubstituted C₁-C₃ alkyl. In some embodiments, R³ is optionally substituted C₁-C₆ alkenyl. In some embodiments, R³ is optionally substituted C₁-C₃ alkenyl. In some embodiments, R³ is unsubstituted C₁-C₆ alkenyl. In some embodiments, R³ is unsubstituted C₁-C₃ alkenyl.

In some embodiments, R³ is substitute with 1-3 C₃-C₆ cycloalkyl. In some embodiments, R³ is substitute with 1 C₃-C₆ cycloalkyl. In some embodiments, R³ is substitute with a cyclopropanyl. In some embodiments, R³ is substitute with 1-3-OH. In some embodiments, R³ is substitute with 1-OH.

In some embodiments, m is 0, 1, 2, 3, 4, 5, or 6. In some embodiments m is 0 or 1. In some embodiments, m is 0. In some embodiments, m is 1. In some embodiments, m is 2. In some embodiments, m is 3. In some embodiments, m is 4. In some embodiments, m is 5. In some embodiments, m is 6.

In some embodiments, n is 0, 1, 2, or 3. In some embodiments n is 1 or 2. In some embodiments, n is 0. In some embodiments, n is 1. In some embodiments, n is 2. In some embodiments, n is 3.

In some embodiments, a compound of Formula (I) is a compound selected from 5 Table 21, or a pharmaceutically acceptable salt or solvate thereof.

TABLE 21

| Compound | |
|---|---|
| No. | Structure |
| CAT1 | 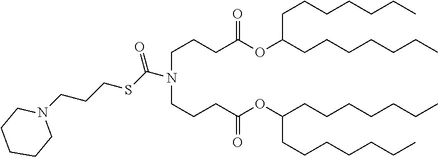 [SMILES: CCCCCCCC(CCCCCCC)OC(=O)CCCN(CCCC(=O)OC(CCCCCCC)CCCCCCC)C(=O)SCCCN1CCCCC1] |
| CAT2 | 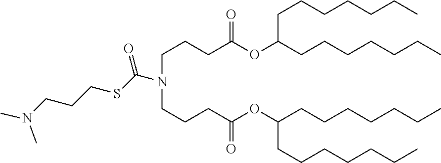 [SMILES: CCCCCCCC(CCCCCCC)OC(=O)CCCN(CCCC(=O)OC(CCCCCCC)CCCCCCC)C(=O)SCCCN(C)C] |
| CAT3 | 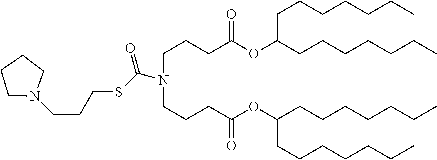 [SMILES: CCCCCCCC(CCCCCCC)OC(=O)CCCN(CCCC(=O)OC(CCCCCCC)CCCCCCC)C(=O)SCCCN1CCCC1] |
| CAT4 | 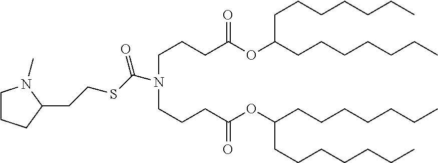 [SMILES: CCCCCCCC(CCCCCCC)OC(=O)CCCN(CCCC(=O)OC(CCCCCCC)CCCCCCC)C(=O)SCCC1CCCN1C] |
| CAT5 | 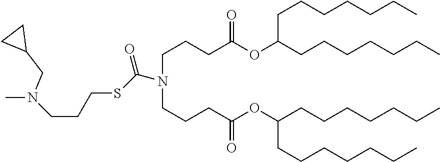 [SMILES: CCCCCCCC(CCCCCCC)OC(=O)CCCN(CCCC(=O)OC(CCCCCCC)CCCCCCC)C(=O)SCCCN(C)CC1CC1] |
| CAT6 | 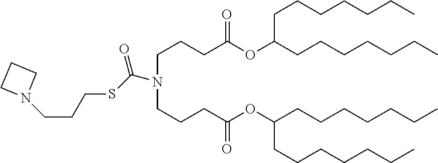 [SMILES: CCCCCCCC(CCCCCCC)OC(=O)CCCN(CCCC(=O)OC(CCCCCCC)CCCCCCC)C(=O)SCCCN1CCC1] |
| CAT7 | 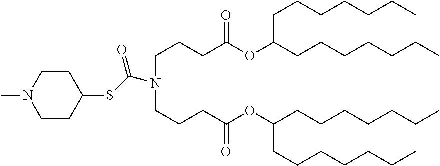 [SMILES: CCCCCCCC(CCCCCCC)OC(=O)CCCN(CCCC(=O)OC(CCCCCCC)CCCCCCC)C(=O)SC1CCN(C)CC1] |
| CAT8 | 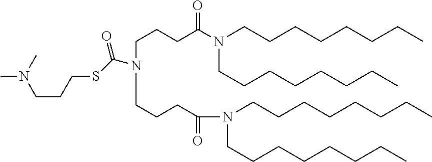 [SMILES: CCCCCCCCN(CCCCCCCC)C(=O)CCCN(CCCC(=O)N(CCCCCCCC)CCCCCCCC)C(=O)SCCCN(C)C] |
| CAT9 | 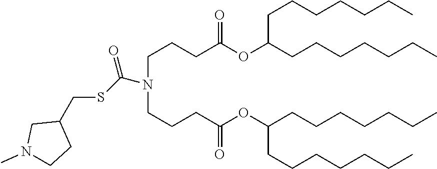 [SMILES: CCCCCCCC(CCCCCCC)OC(=O)CCCN(CCCC(=O)OC(CCCCCCC)CCCCCCC)C(=O)SCC1CCN(C)C1] |
| CAT10 | 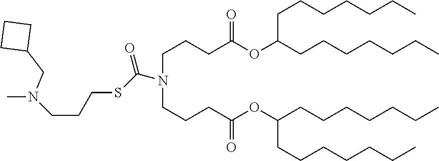 [SMILES: CCCCCCCC(CCCCCCC)OC(=O)CCCN(CCCC(=O)OC(CCCCCCC)CCCCCCC)C(=O)SCCCN(C)CC1CCC1] |
| CAT11 | 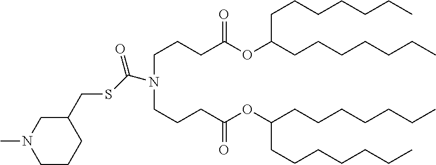 [SMILES: CCCCCCCC(CCCCCCC)OC(=O)CCCN(CCCC(=O)OC(CCCCCCC)CCCCCCC)C(=O)SCC1CCCN(C)C1] |
| CAT12 | 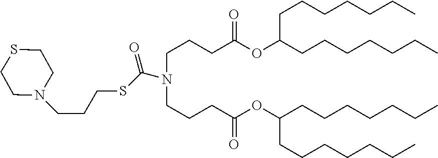 [SMILES: CCCCCCCC(CCCCCCC)OC(=O)CCCN(CCCC(=O)OC(CCCCCCC)CCCCCCC)C(=O)SCCCN1CCSCC1] |
| CAT13 | 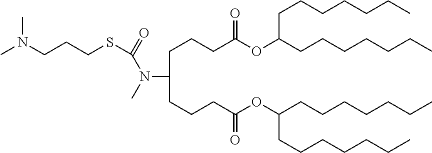 [SMILES: CCCCCCCC(CCCCCCC)OC(=O)CCCC(CCCC(=O)OC(CCCCCCC)CCCCCCC)N(C)C(=O)SCCCN(C)C] |
| CAT14 | 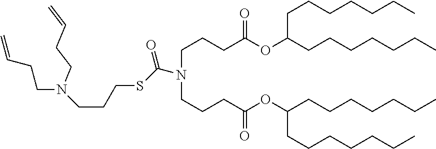 [SMILES: C=CCCN(CCC=C)CCCSC(=O)N(CCCC(=O)OC(CCCCCCC)CCCCCCC)CCCC(=O)OC(CCCCCCC)CCCCCCC] |
| CAT15 | 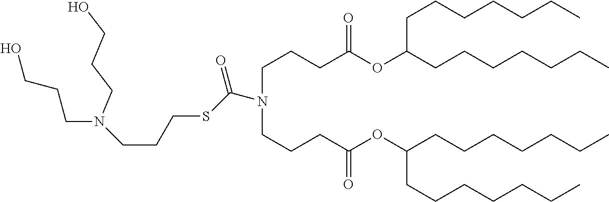 [SMILES: CCCCCCCC(CCCCCCC)OC(=O)CCCN(CCCC(=O)OC(CCCCCCC)CCCCCCC)C(=O)SCCCN(CCCO)CCCO] |
| CAT16 | 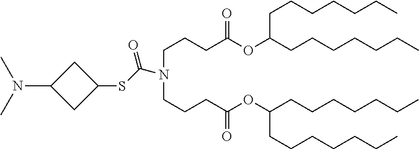 [SMILES: CCCCCCCC(CCCCCCC)OC(=O)CCCN(CCCC(=O)OC(CCCCCCC)CCCCCCC)C(=O)SC1CC(N(C)C)C1] |
| CAT17 | 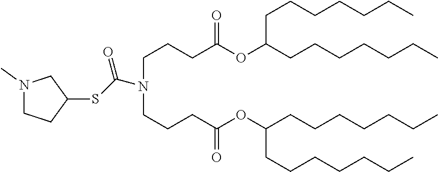 [SMILES: CCCCCCCC(CCCCCCC)OC(=O)CCCN(CCCC(=O)OC(CCCCCCC)CCCCCCC)C(=O)SC1CCN(C)C1] |
| CAT18 | 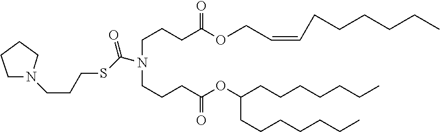 [SMILES: CCCCCC/C=C\COC(=O)CCCN(CCCC(=O)OC(CCCCCCC)CCCCCCC)C(=O)SCCCN1CCCC1] |
| CAT19 | 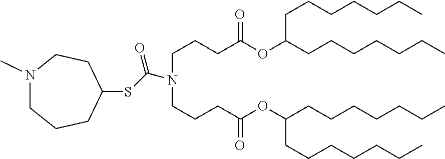 [SMILES: CCCCCCCC(CCCCCCC)OC(=O)CCCN(CCCC(=O)OC(CCCCCCC)CCCCCCC)C(=O)SC1CCCN(C)CC1] |
| CAT20 | 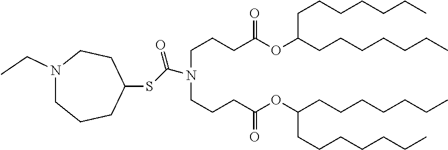 [SMILES: CCCCCCCC(CCCCCCC)OC(=O)CCCN(CCCC(=O)OC(CCCCCCC)CCCCCCC)C(=O)SC1CCCN(CC)CC1] |
| CAT21 | 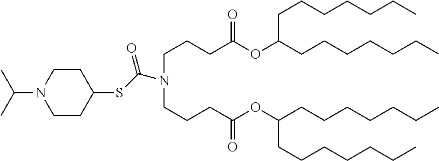 [SMILES: CCCCCCCC(CCCCCCC)OC(=O)CCCN(CCCC(=O)OC(CCCCCCC)CCCCCCC)C(=O)SC1CCN(C(C)C)CC1] |
| CAT22 | 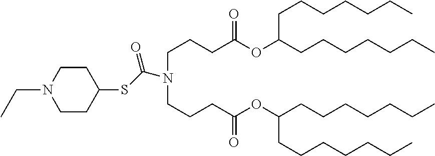 [SMILES: CCCCCCCC(CCCCCCC)OC(=O)CCCN(CCCC(=O)OC(CCCCCCC)CCCCCCC)C(=O)SC1CCN(CC)CC1] |
| CAT23 | 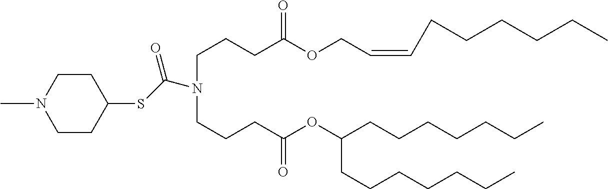 [SMILES: CCCCCC/C=C\COC(=O)CCCN(CCCC(=O)OC(CCCCCCC)CCCCCCC)C(=O)SC1CCN(C)CC1] |
| CAT24 | 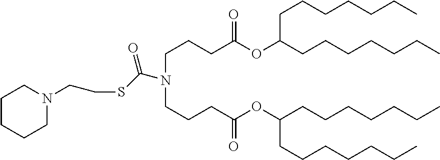 [SMILES: CCCCCCCC(CCCCCCC)OC(=O)CCCN(CCCC(=O)OC(CCCCCCC)CCCCCCC)C(=O)SCCN1CCCCC1] |
| CAT25 | 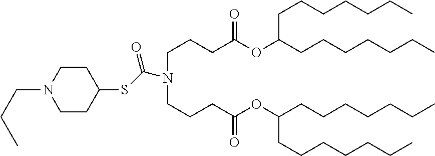 [SMILES: CCCCCCCC(CCCCCCC)OC(=O)CCCN(CCCC(=O)OC(CCCCCCC)CCCCCCC)C(=O)SC1CCN(CCC)CC1] |
| CAT26 | 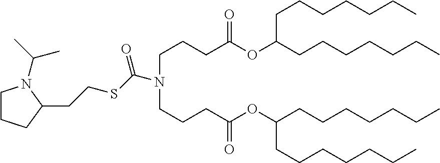 [SMILES: CCCCCCCC(CCCCCCC)OC(=O)CCCN(CCCC(=O)OC(CCCCCCC)CCCCCCC)C(=O)SCCC1CCCN1C(C)C] |
| CAT27 | 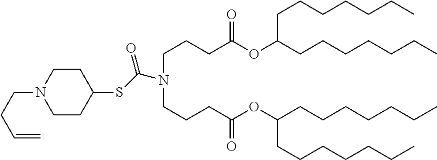 [SMILES: C=CCCN1CCC(SC(=O)N(CCCC(=O)OC(CCCCCCC)CCCCCCC)CCCC(=O)OC(CCCCCCC)CCCCCCC)CC1] |
| CAT28 | 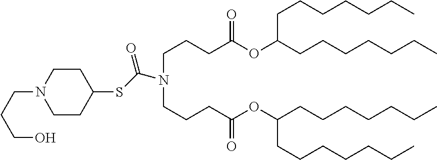 [SMILES: CCCCCCCC(CCCCCCC)OC(=O)CCCN(CCCC(=O)OC(CCCCCCC)CCCCCCC)C(=O)SC1CCN(CCCO)CC1] |
| CAT29 | 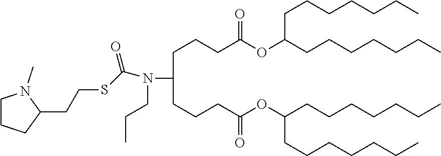 [SMILES: CCCCCCCC(CCCCCCC)OC(=O)CCCC(CCCC(=O)OC(CCCCCCC)CCCCCCC)N(CCC)C(=O)SCCC1CCCN1C] |
| CAT30 | 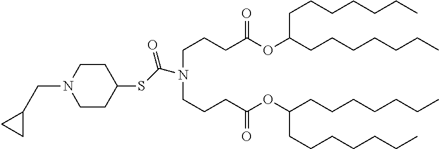 [SMILES: CCCCCCCC(CCCCCCC)OC(=O)CCCN(CCCC(=O)OC(CCCCCCC)CCCCCCC)C(=O)SC1CCN(CC2CC2)CC1] |
| CAT31 | 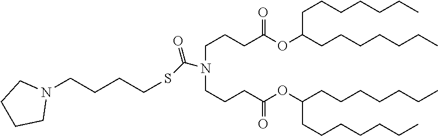 [SMILES: CCCCCCCC(CCCCCCC)OC(=O)CCCN(CCCC(=O)OC(CCCCCCC)CCCCCCC)C(=O)SCCCCN1CCCC1] |
| CAT32 | 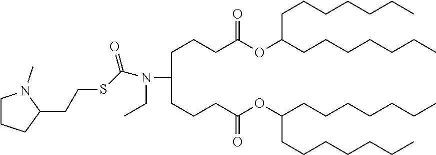 [SMILES: CCCCCCCC(CCCCCCC)OC(=O)CCCC(CCCC(=O)OC(CCCCCCC)CCCCCCC)N(CC)C(=O)SCCC1CCCN1C] |
| CAT33 | 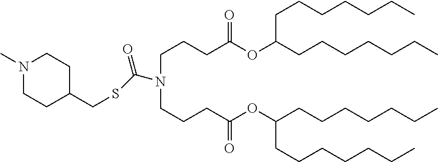 [SMILES: CCCCCCCC(CCCCCCC)OC(=O)CCCN(CCCC(=O)OC(CCCCCCC)CCCCCCC)C(=O)SCC1CCN(C)CC1] |
| CAT34 | 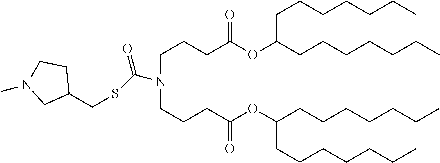 [SMILES: CCCCCCCC(CCCCCCC)OC(=O)CCCN(CCCC(=O)OC(CCCCCCC)CCCCCCC)C(=O)SCC1CCN(C)C1] |
| CAT35 | 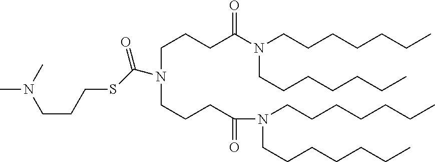 [SMILES: CCCCCCCN(CCCCCCC)C(=O)CCCN(CCCC(=O)N(CCCCCCC)CCCCCCC)C(=O)SCCCN(C)C] |

### Compounds of Formula (A)

In various embodiments, provided herein are compounds of Formula (A):

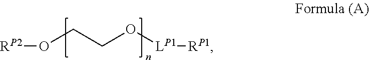

or a pharmaceutically acceptable salt thereof, wherein:
   n is an integer between 10 to 200, inclusive of all endpoints;
   L^{P1} is —[(CH₂)₀₋₃—C(O)O]₁₋₃—, —(CH₂)₀₋₃—C(O)O—(CH₂)₁₋₃—OC(O)—, or —C(O)N(H)—;
   R^{P1} is C₅-C₂₅ alkyl or C₅-C₂₅ alkenyl; and
   R^{P2} is hydrogen or —CH₃.

In some embodiments, Formula (A) is not HO—(CH₂CH₂O)ₙ—C(O)N(H)—(CH₂)₁₇CH₃.

In some embodiments, L^{P1} is —CH₂C(O)O—, —CH₂CH₂C(O)O—, —CH₂C(O)OCH₂C(O)O—, —CH₂C(O)OCH₂CH₂OC(O)—, or —C(O)N(H)—.

In some embodiments, the PEG-lipid is a compound of Formula (A-a), Formula (A-b), Formula (A-c), Formula (A-d), or Formula (A-e):

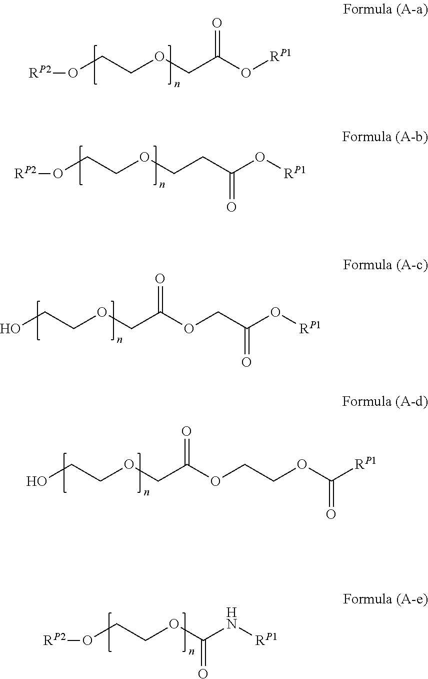

or a pharmaceutically acceptable salt thereof.

In some embodiments, R^{P1} is C₆-C₂₄, C₁₀-C₂₀, C₁₀-C₁₈, C₁₀-C₁₆, C₁₀-C₁₄, C₁₀-C₁₂, C₁₂-C₂₀, C₁₂-C₁₈, C₁₂-C₁₆, C₁₂-C₁₄, C₁₄-C₂₀, C₁₄-C₁₈, C₁₄-C₁₆, C₁₆-C₂₀, C₁₆-C₁₈, or C₁₈-C₂₀ alkyl. In some embodiments, R^{P1} is C₁₄-C₁₈ alkyl. In some embodiments, R^{P1} is C₁₄-C₁₆ alkyl. In some embodiments, R^{P1} is C₁₅-C₁₇ alkyl. In some embodiments, R^{P1} is C₁₆-C₁₈ alkyl. In some embodiments, R^{P1} is C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkyl. In some embodiments, R^{P1} is C₆-C₂₄, C₁₀-C₂₀, C₁₀-C₁₈, C₁₀-C₁₆, C₁₀-C₁₄, C₁₀-C₁₂, C₁₂-C₂₀, C₁₂-C₁₈, C₁₂-C₁₆, C₁₂-C₁₄, C₁₄-C₂₀, C₁₄-C₁₈, C₁₄-C₁₆, C₁₆-C₂₀, C₁₆-C₁₈, or C₁₈-C₂₀ alkenyl. In some embodiments, R^{P1} is C₁₄-C₁₈ alkenyl. In some embodiments, R^{P1} is C₁₄₋₁₆ alkenyl. In some embodiments, R^{P1} is C₁₅-C₁₇ alkenyl. In some embodiments, R^{P1} is C₁₆₋₁₈ alkenyl. In some embodiments, R^{P1} is C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkenyl.

In some embodiments, R^{P2} is hydrogen. In some embodiments, R^{P2} is —CH₃.

In some embodiments, n is, on average, 10 to 200, 10 to 180, 10 to 160, 10 to 140, 10 to 120, 10 to 100, 10 to 80, 10 to 60, 10 to 40, 10 to 20, 20 to 200, 20 to 180, 20 to 160, 20 to 140, 20 to 120, 20 to 100, 20 to 80, 20 to 60, 20 to 40, 40 to 200, 40 to 180, 40 to 160, 40 to 140, 40 to 120, 40 to 100, 40 to 80, 40 to 60, 60 to 200, 60 to 180, 60 to 160, 60 to 140, 60 to 120, 60 to 100, 60 to 80, 80 to 200, 80 to 180, 80 to 160, 80 to 140, 80 to 120, 80 to 100, 100 to 200, 100 to 180, 100 to 160, 100 to 140, 100 to 120, 120 to 200, 120 to 180, 120 to 160, 120 to 140, 140 to 200, 140 to 180, 140 to 160, 160 to 200, 160 to 180, or 180 to 200. In some embodiments, n is, on average, 10, 20, 30, 40, 50, 60, 70, 80, 90, 100, 110, 120, 130, 140, 150, 160, 170, 180, 190, or 200. In some embodiments, n is on average about 20. In some embodiments, n is on average about 40. In some embodiments, n is on average about 45. In some embodiments, n is on average about 50. In some embodiments, n is on average about 68. In some embodiments, n is on average about 75. In some embodiments, n is on average about 100.

In some embodiments, a compound of Formula (A) is a compound selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45;
   or a pharmaceutically acceptable salt thereof.

### ALTERNATIVE EMBODIMENTS

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Lipid Nanoparticles

In certain embodiments, the synthetic RNA viral genomes described herein are encapsulated in a lipid nanoparticle (LNP). In certain embodiments, the LNP comprises one or more lipids such as such as triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate). In some embodiments, the LNP comprises one or more cationic lipids, one or more structural lipids, and one or more helper lipids. In some embodiments, the LNP comprises one or more cationic lipids, a cholesterol, and one or more neutral lipids.

In some embodiments, compounds of the present disclosure are used to form a nanoparticle. In some embodiments, the nanoparticle is a lipid nanoparticle (LNP). In some embodiments, an LNP comprises a PEG-lipid, an ionizable lipid, a helper lipid, and a structural lipid. In some embodiments, LNPs described herein are formulated for delivery of therapeutic agents to a subject in need thereof. In some embodiments, LNPs described herein are formulated for delivery of nucleic acid molecules to a subject in need thereof.

The formulation of lipids in an LNP significantly impacts the therapeutic use and efficacy of a particular LNP. For example, LNP formulations such as SS—OC/Cholesterol/DSPC/PEG2k-DPG typically display increased clearance rate upon repeat intravenous (IV) administration, e.g., in mice, non-human primates (NHPs), and/or humans and a much shorter circulation time in vivo post-second dose than post-first dose. The shortened circulation time can negatively impact the delivery efficiency of the LNPs, likely due to less exposure of the LNPs to the target. Therefore, while such formulations may be useful in delivering agents that do not require multiple administrations, their use for delivery of agents that require subsequent administration may be constrained by this shortened circulation time.

There remains a need for LNP formulations that demonstrate tunable circulation and exposure to target cells, e.g., sustained circulation and consistent exposure, in vivo upon repeat dosing. The present disclosure provides such LNP formulations by incorporating ionizable lipid and/or PEG-lipid of the disclosure into the lipid formulation of the LNP. The sustained circulation of the LNP of the present disclosure upon repeat administration consequently allows for sustained therapeutic effect of the synthetic RNA viral genomes encapsulated therein.

In some embodiments, in the absence of the ionizable lipid and/or PEG-lipid of the disclosure, rapid clearance of the LNP and components thereof upon repeated dosing reduces the delivery efficiency of the encapsulated synthetic RNA viral genome in subsequent doses as the body may clear the LNP prior to the release of the synthetic RNA viral genome. In some embodiments, ionizable lipid and/or PEG-lipid of the disclosure, when incorporated into an LNP, delays clearance of the LNP upon repeated dosing, allowing for the sustained release and therapeutic effect of the encapsulated synthetic RNA viral genome.

### Polyethyleneglycol (PEG)-Lipid

In some embodiments, the PEG-lipid of the disclosure comprises a hydrophilic head group and a hydrophobic lipid tail. In some embodiments, the hydrophilic head group is a PEG moiety. In some embodiments, PEG-lipid of the disclosure comprises a mono lipid tail. In some embodiments, PEG-lipid of the disclosure comprises a mono alkyl lipid tail, a mono alkenyl lipid tail, a mono alkynyl lipid tail, or a mono acyl lipid tail. In some embodiments, the mono lipid tail comprises an ether group, a carbonyl group, or an ester group. In some embodiments, the PEG-lipid of the disclosure may contain a polyoxyethylene alkyl ether, a polyoxyethylene alkenyl ether, or a polyoxyethylene alkynyl ether (such molecules are also known as BRIJ™ or Brij molecules). In some embodiments, the PEG-lipid of the disclosure may contain a polyoxyethylene alkyl ester, a polyoxyethylene alkenyl ester, or a polyoxyethylene alkynyl ester (such molecules are also known as MYRJ™ molecules).

In some embodiments, the PEG-lipid may contain di-acyl lipid tails.

In some embodiments, the PEG-lipid is a compound of Formula (A)

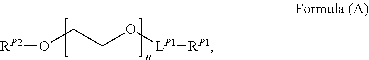

or a pharmaceutically acceptable salt or solvate thereof, wherein the variables are defined herein.

In some embodiments, the PEG-lipid is a compound of Formula (A′):

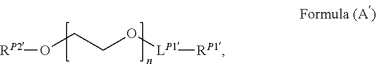

or a pharmaceutically acceptable salt thereof, wherein:
n is an integer between 10 to 200, inclusive of all endpoints;
L^{P1′} is a bond, —C(O)—, —[(CH₂)₀₋₃—C(O)O]₁₋₃—, —(CH₂)₀₋₃—C(O)O—(CH₂)₁₋₃—OC(O)—, or —C(O)N(H)—;
R^{P1′} is C₅-C₂₅ alkyl or C₅-C₂₅ alkenyl; and
R^{P2′} is hydrogen or —CH₃.

In some embodiments, L^{P1′} is a bond, —C(O)—, —CH₂C(O)O—, —CH₂CH₂C(O)O—, —CH₂C(O)OCH₂C(O)O—, —CH₂C(O)OCH₂CH₂OC(O)—, or —C(O)N(H)—. In some embodiments, R^{P1′} is R^{P1}. In some embodiments, R^{P2′} is R^{P2}.

In some embodiments, the PEG-lipid is a compound of Formula (A″):

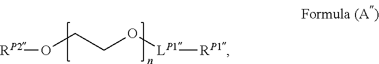

or a pharmaceutically acceptable salt thereof, wherein:
n is an integer between 10 to 200, inclusive of all endpoints;
L^{P1″} is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1″} is C₅-C₂₅ alkyl or C₅-C₂₅ alkenyl; and
R^{P2″} is hydrogen or —CH₃.

In some embodiments, L^{P1″} is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

In some embodiments, the PEG-lipid is a compound of Formula (A″-a), Formula (A″-b), Formula (A″-c), Formula (A″-cd), Formula (A″-e), or Formula (A″-f):

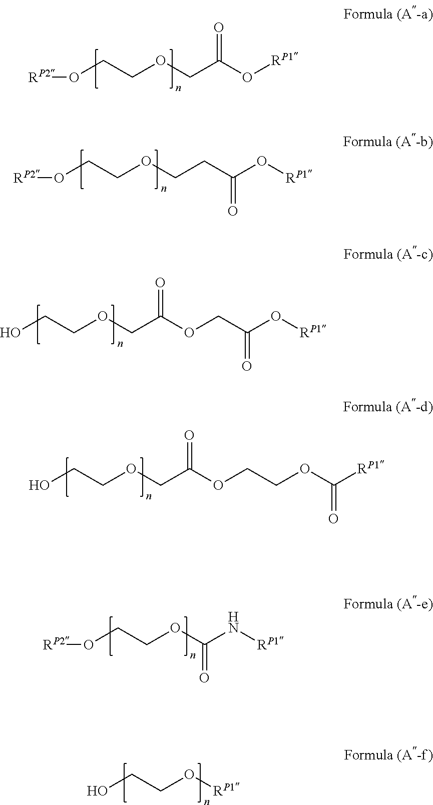

or a pharmaceutically acceptable salt thereof.

In some embodiments, R^{P1″} is R^{P1}. In some embodiments, R^{P2″} is R_{P2}.

In some embodiments, the PEG-lipid is a compound of Formula (A″-f1):

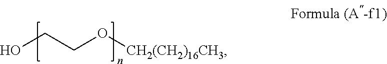

or a pharmaceutically acceptable salt thereof.

In some embodiments, the PEG-lipid is a compound of Formula (A″-f2):

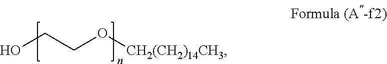

or a pharmaceutically acceptable salt thereof.

In some embodiments, the PEG-lipid is a compound of Formula (A″-f3):

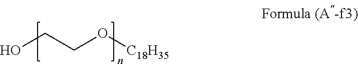

or a pharmaceutically acceptable salt thereof.

In some embodiments, a PEG-lipid of the disclosure is a compound of Formula (B):

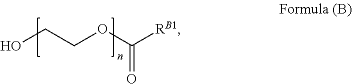

or a pharmaceutically acceptable salt thereof, wherein:
n is an integer between 10 to 200, inclusive of all endpoints; and
R^{B1} is C₅-C₂₅ alkyl or C₅-C₂₅ alkenyl.

In some embodiments, R^{B1} is R^{P1}.

In some embodiments, the PEG-lipid is a compound of Formula (B-a):

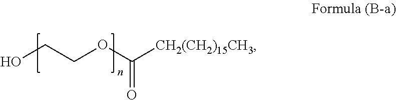

or a pharmaceutically acceptable salt thereof.

In some embodiments, the PEG-lipid is a compound of Formula (B-b):

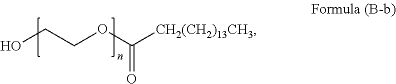

or a pharmaceutically acceptable salt thereof.

In some embodiments, n is, on average, 10 to 200, 10 to 180, 10 to 160, 10 to 140, 10 to 120, 10 to 100, 10 to 80, 10 to 60, 10 to 40, 10 to 20, 20 to 200, 20 to 180, 20 to 160, 20 to 140, 20 to 120, 20 to 100, 20 to 80, 20 to 60, 20 to 40, 40 to 200, 40 to 180, 40 to 160, 40 to 140, 40 to 120, 40 to 100, 40 to 80, 40 to 60, 60 to 200, 60 to 180, 60 to 160, 60 to 140, 60 to 120, 60 to 100, 60 to 80, 80 to 200, 80 to 180, 80 to 160, 80 to 140, 80 to 120, 80 to 100, 100 to 200, 100 to 180, 100 to 160, 100 to 140, 100 to 120, 120 to 200, 120 to 180, 120 to 160, 120 to 140, 140 to 200, 140 to 180, 140 to 160, 160 to 200, 160 to 180, or 180 to 200. In some embodiments, n is, on average, 10, 20, 30, 40, 50, 60, 70, 80, 90, 100, 110, 120, 130, 140, 150, 160, 170, 180, 190, or 200. In some embodiments, n is on average about 20. In some embodiments, n is on average about 40. In some embodiments, n is on average about 45. In some embodiments, n is on average about 50. In some embodiments, n is on average about 68. In some embodiments, n is on average about 75. In some embodiments, n is on average about 100.

In some embodiments, the PEG-lipid comprises a PEG moiety having an average molecular weight of about 500 to about 10,000 daltons. In some embodiments, the PEG-lipid comprises a PEG moiety having an average molecular weight of about 500 to about 5,000 daltons, about 500 to about 4,000 daltons, about 500 to about 3,000 daltons, about 500 to about 2,000 daltons, about 500 to about 1,000 daltons, about 500 to about 800 daltons, about 500 to about 600 daltons, about 600 to about 5,000 daltons, about 600 to about 4,000 daltons, about 600 to about 3,000 daltons, about 600 to about 2,000 daltons, about 600 to about 1,000 daltons, about 600 to about 800 daltons, about 800 to about 5,000 daltons, about 800 to about 4,000 daltons, about 800 to about 3,000 daltons, about 800 to about 2,000 daltons, about 800 to about 1,000 daltons, about 1,000 to about 5,000 daltons, about 1,000 to about 4,000 daltons, about 1,000 to about 3,000 daltons, about 1,000 to about 2,000 daltons, about 2,000 to about 5,000 daltons, about 2,000 to about 4,000 daltons, about 2,000 to about 3,000 daltons, about 3,000 to about 5,000 daltons, about 3,000 to about 4,000 daltons, about 5,000 to about 10,000 daltons, about 5,000 to about 7,500 daltons, or about 7,500 to about 10,000 daltons. In some embodiments, the PEG moiety of the PEG-lipid has an average molecular weight of about 1,500 to about 2,500 daltons. In some embodiments, the PEG moiety of the PEG-lipid has an average molecular weight of about 1,000 to about 5,000 daltons. In some embodiments, the PEG-lipid comprises a PEG moiety having an average molecular weight of about 500, about 600, about 800, about 1,000, about 1,500, about 2,000, about, 2500, about 3,000, about 3,500, about 4,000, about 4,500, about 5,000, about 6,000, about 7,000, about 8,000, about 9,000, or about 10,000 daltons. In some embodiments, the PEG-lipid comprises a PEG moiety having an average molecular weight of at least 500, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000, or at least 10,000 daltons. In some embodiments, the PEG-lipid comprises a PEG moiety having an average molecular weight of no more than 500, no more than 1,000, no more than 1,500, no more than 2,000, no more than 2,500, no more than 3,000, no more than 3,500, no more than 4,000, no more than 4,500, no more than 5,000, no more than 6,000, no more than 7,000, no more than 8,000, no more than 9,000, or no more than 10,000 daltons. All values are inclusive of all endpoints.

In some embodiments, the PEG-lipid is polyoxyethylene (100) stearyl ether, polyoxyethylene (20) cetyl ether, polyoxyethylene (20) oleyl ether, polyoxyethylene (20) stearyl ether, or a mixture thereof. In some embodiments, the PEG-lipid is polyoxyethylene (100) stearate, polyoxyethylene (50) stearate, polyoxyethylene (40) stearate, polyoxyethylene palmitate, or a mixture thereof.

In some embodiments of the disclosure, the PEG-lipid is

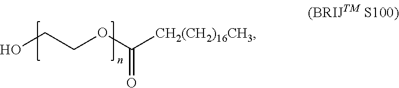

having a CAS number of 9005-00, a linear formula of C₁₈H₃₇ (OCH₂CH₂)ₙOH wherein n is 100. BRIJ™ S100 is also known, generically, as polyoxyethylene (100) stearyl ether. Accordingly, in some embodiments, the PEG-lipid is HO-PEG100-CH₂ (CH₂)₁₆CH₃.

In some embodiments of the disclosure, the PEG-lipid is

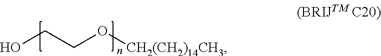

having a CAS number of 9004-95-9, a linear formula of C₁₆H₃₃ (OCH₂CH₂)ₙOH wherein n is 20. BRIJ™ C20 is also known as BRIJ™ 58, and, generically, as polyethylene glycol hexadecyl ether, polyoxyethylene (20) cetyl ether. Accordingly, in some embodiments, the PEG-lipid is HO-PEG20-CH₂ (CH₂)₁₄CH₃.

In some embodiments of the disclosure, the PEG-lipid is

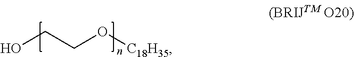

having a CAS number of 9004-98-2, a linear formula of C₁₈H₃₅ (OCH₂CH₂)ₙOH wherein n is 20. BRIJ™ O20 is also known, generically, as polyoxyethylene (20) oleyl ether. Accordingly, in some embodiments, the PEG-lipid is HO-PEG20-C₁₈H₃₅.

In some embodiments of the disclosure, the PEG-lipid is

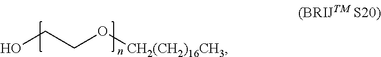

having a CAS number of 9005-00-9, a linear formula of C₁₈H₃₇ (OCH₂CH₂)ₙOH wherein n is 20. BRIJ™ S20 is also known, generically, as polyethylene glycol octadecyl ether or polyoxyethylene (20) stearyl ether. Accordingly, in some embodiments, the PEG-lipid is HO-PEG20-CH₂(CH₂)₁₆CH₃.

In some embodiments of the disclosure, the PEG-lipid is

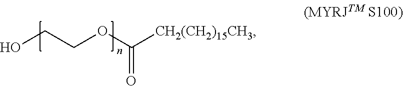

having a CAS number of 9004-99-3, a linear formula of C₁₇H₃₅C(O)(OCH₂CH₂)ₙOH wherein n is 100. MYRJ™ S100 is also known, generically, as polyoxyethylene (100) stearate. Accordingly, in some embodiments, the PEG-lipid is HO-PEG100-CH₂ (CH₂)₁₅CH₃.

In some embodiments of the disclosure, the PEG-lipid is

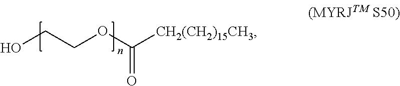

having a CAS number of 9004-99-3, a linear formula of C₁₇H₃₅C(O) (OCH₂CH₂)ₙOH wherein n is 50. MYRJ™ S50 is also known, generically, as polyoxyethylene (50) stearate. Accordingly, in some embodiments, the PEG-lipid is HO-PEG50-CH₂ (CH₂)₁₅CH₃.

In some embodiments of the disclosure, PEG-lipid is

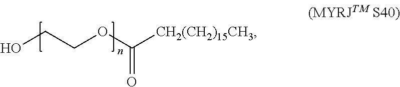

having a CAS number of 9004-99-3, a linear formula of C₁₇H₃₅C(O)(OCH₂CH₂)OH wherein n is 40. MYRJ™ S40 is also known, generically, as polyoxyethylene (40) stearate. Accordingly, in some embodiments, the PEG-lipid is HO-PEG40-CH₂ (CH₂)₁₅CH₃.

In some embodiments of the disclosure, the PEG-lipid is

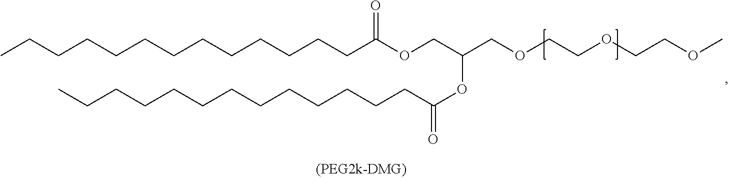

having a CAS number of 1607430-62-04, a linear formula of C122H242O50. PEG2k-DMG is also known as 1,2-dimyristoyl-rac-glycero-3-methoxypolyethylene glycol-2000.

In some embodiments of the disclosure, the PEG-lipid is:

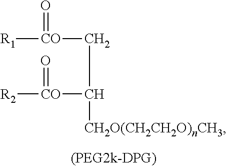

having an alkyl composition of R₁COO═C16:0, R₂COO═C16:0. PEG2k-DPG is also known, generically, as 1,2-Dipalmitoyl-rac-glycero-3-methylpolyoxyethylene.

In some embodiments of the disclosure, the PEG-lipid may be PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, PEG-distearoylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol) ether), 1,2-dimyristoyl-sn-glycero-3-phosphoethanolamine-N-[methoxy (polyethylene glycol)-2000] (PEG2k-DMG), 1,2-distearoyl-sn-glycero-3-phosphoethanolamine-N-[methoxy (polyethylene glycol)-2000] (PEG2k-DSPE), 1,2-distearoyl-snglycerol, methoxypolyethylene glycol (PEG2k-DSG), poly(ethylene glycol)-2000-dimethacrylate (PEG2k-DMA), or 1,2-distearyloxypropyl-3-amine-N-[methoxy (polyethylene glycol)-2000] (PEG2k-DSA). In some embodiments, the PEG-lipid may be PEG2k-DMG. In some embodiments, the PEG-lipid may be PEG2k-DSG. In other embodiments, the PEG-lipid may be PEG2k-DSPE. In some embodiments, the PEG-lipid may be PEG2k-DMA. In yet other embodiments, the PEG-lipid may be PEG2k-C-DMA. In some embodiments, the PEG-lipid may be PEG2k-DSA. In other embodiments, the PEG-lipid may be PEG2k-C11. In some embodiments, the PEG-lipid may be PEG2k-C14. In some embodiments, the PEG-lipid may be PEG2k-C16. In some embodiments, the PEG-lipid may be PEG2k-C18.

In some embodiments, a PEG-lipid having single lipid tail of the disclosure (e.g., PEG-lipid of Formula (A), (A′), (A″), or (B)) may reduce accelerated blood clearance (ABC) upon administration and/or repeat administration of an LNP composition of the disclosure. In some embodiments, a PEG-lipid having single lipid tail of the disclosure may reduce or deplete PEG-specific antibodies (e.g., anti-PEG IgM) generated by a subject's immune system upon administration and/or repeat administration of an LNP composition of the disclosure.

In some embodiments, the PEG-lipid comprises a poly(ethylene) glycol chain of up to 5 kDa in length covalently attached to a lipid comprising one or more C6-C20 alkyls. In some embodiments, the PEG-lipid is 1,2-Distearoyl-sn-glycero-3-phosphoethanolamine-Poly(ethylene glycol) (DSPE-PEG), or 1,2-distearoyl-sn-glycero-3-phosphoethanolamine-N-[amino (polyethylene glycol)] (DSPE-PEG-amine). In some embodiments, the PEG-lipid is selected from 1,2-distearoyl-sn-glycero-3-phosphoethanolamine-N-[amino (polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K). In some embodiments, the PEG-lipid is DSPE-PEG5K. In some embodiments, the PEG-lipid is DPG-PEG2K. In some embodiments, the PEG-lipid is DSG-PEG2K. In some embodiments, the PEG-lipid is DMG-PEG2K. In some embodiments, the PEG-lipid is DSG-PEG5K. In some embodiments, the PEG-lipid is DMG-PEG5K.

In some embodiments, the PEG lipid is a cleavable PEG lipid. Examples of PEG derivatives with cleavable bonds include those modified with peptide bonds (Kulkarni et al. (2014). Mmp-9 responsive PEG cleavable nanovesicles for efficient delivery of chemotherapeutics to pancreatic cancer. Mol Pharmaceutics 11:2390-9; Lin et al. (2015). Drug/dye-loaded, multifunctional peg-chitosan-iron oxide nanocomposites for methotrexate synergistically self-targeted cancer therapy and dual model imaging. ACS Appl Mater Interfaces 7:11908-20.), disulfide keys (Yan et al (2014). A method to accelerate the gelation of disulfide-crosslinked hydrogels. Chin J Polym Sci 33:118-27; Wu & Yan (2015). Copper nanopowder catalyzed cross-coupling of diaryl disulfides with aryl iodides in PEG-400. Synlett 26:537-42), vinyl ether bonds, hydrazone bonds (Kelly et al. (2016). Polymeric prodrug combination to exploit the therapeutic potential of antimicrobial peptides against cancer cells. Org Biomol Chem 14:9278-86.), and ester bonds (Xu et al. (2008). Esterase-catalyzed dePEGylation of pH-sensitive vesicles modified with cleavable PEG-lipid derivatives. J Control Release 130:238-45). See also, Fang et al., (2017) Cleaveable PEGylation: a strategy for overcoming the “PEG dilemma” in efficient drug delivery. Drug Delivery 24:2, 22-32.

In some embodiments, the PEG lipid is an activated PEG lipid. Exemplary activated PEG lipids include PEG-NH2, PEG-MAL, PEG-NHS, and PEG-ALD. Such functionalized PEG lipids are useful in the conjugation of targeting moieties to lipid nanoparticles to direct the particles to a particular target cell or tissue (e.g., by the attachment of antigen-binding molecules, peptides, glycans, etc.). In some embodiments, the functionalized moiety (e.g., —NH2, _MAL, —NHS, -ALD) is added to the free end of the PEG moiety of the PEG-lipid of the disclosure (e.g., BRIJ™ or MYRJ™ family PEG lipid)

### Cationic Lipid

In some embodiments, the LNP provided herein comprises one or more cationic lipids. “Cationic lipid” and “ionizable lipid” are used interchangeably herein.

Cationic lipids refer to any of a number of lipid species that carry a net positive charge at a selected pH, such as physiological pH. Such lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3-dioleoyloxy) propyl)-N,N,N-trimethylammonium chloride (DOTAP); 3-(N—(N′,N′-dimethylaminoethane)-carbamoyl) cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C₁₈ alkyl chains, ether linkages between the head group and alkyl chains, and 0 to 3 double bonds. Such lipids include, e.g., DSDMA, DLinDMA, DLenDMA, and DODMA.

In some embodiments, the cationic lipids comprise a protonatable tertiary amine head group. Such lipids are referred to herein as ionizable lipids. Ionizable lipids refer to lipid species comprising an ionizable amine head group and typically comprising a pKa of less than about 7. Therefore, in environments with an acidic pH, the ionizable amine head group is protonated such that the ionizable lipid preferentially interacts with negatively charged molecules (e.g., nucleic acids such as the recombinant polynucleotides described herein) thus facilitating nanoparticle assembly and encapsulation. Therefore, in some embodiments, ionizable lipids can increase the loading of nucleic acids into lipid nanoparticles. In environments where the pH is greater than about 7 (e.g., physiologic pH of ≈7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle. In some embodiments, the LNP comprises an ionizable lipid, e.g., a 7.SS-cleavable and pH-responsive Lipid Like Material (such as the COATSOME® SS-Series).

In some embodiments, the cationic lipid of the LNP is DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME® SS—OP, Di((Z)-non-2-en-1-yl) 9-((4-dimethylamino) butanoyl)oxy) heptadecanedioate (L-319), N-(2,3-dioleoyloxy) propyl)-N,N,N-trimethylammonium chloride (DOTAP), or a mixture thereof.

In some embodiments the cationic lipid of the LNP is a compound of Formula (I):

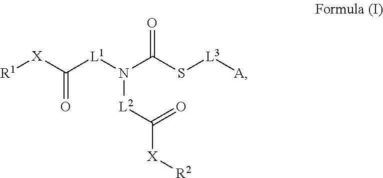

or a pharmaceutically acceptable salt or solvate thereof, wherein the variables are defined herein.

In some embodiments, cationic lipid of the disclosure is a compound selected from Table 21 or a pharmaceutically acceptable salt thereof.

In some embodiments, the cationic lipid of the LNP is a compound of Formula (II-1):

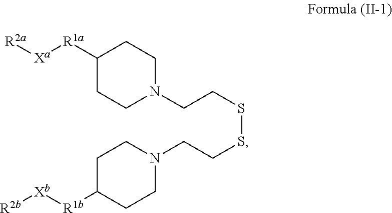

or a pharmaceutically acceptable salt or solvate thereof, wherein:
   R^{1a} and R^{1b} are each independently C₁-C₈ aliphatic or—O(C₁-C₈ aliphatic)-, wherein the O atom, when present, is bonded to the piperidine ring;
   X^{a} and X^{b} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)O—*, or —O—, wherein —* indicates the attachment point to R^{2a} or R^{2b}, respectively and wherein each occurrence of Rₓ¹ is independently selected from hydrogen and optionally substituted C₁-C₄ alkyl; and
   R^{2a} and R^{2b} are each independently a sterol residue, a liposoluble vitamin residue, or an C₁₃-C₂₃ aliphatic.

In some embodiments, the cationic lipid of the LNP is a compound of Formula (II-2):

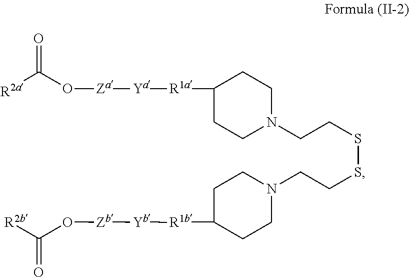

or a pharmaceutically acceptable salt or solvate thereof, wherein:
   R^{1a′} and R^{1b′} are each independently C₁-C₈ alkylene or —O(C₁-C₈alkylene), wherein the O atom, when present, is bonded to the piperidine ring;
   Y^{a′} and Y^{b′} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C—O)O—*, —N(Rₓ¹)C(O)N(Rₓ¹)—, or —O—, wherein —* indicates the attachment point to R^{2a} or R^{2b}, and wherein each occurrence of Rₓ¹ is independently selected from hydrogen and optionally substituted C₁-C₄ alkyl;
Z^{a′} and Z^{b′} are each independently optionally substituted arylene-C₀-C₈ alkylene or optionally substituted arylene-C₀-C₈ heteroalkylene, wherein the alkylene or heteroalkylene group is bonded to Y^{a′} and Y^{b′}, respectively;
R^{2a′} and R^{2b′} are each independently a sterol residue, a liposoluble vitamin residue, or an C₁₂-C₂₂ aliphatic.

In some embodiments, the cationic lipid of the LNP is a compound of Formula (II-1a) (COATSOME® SS—OC) or Formula (II-2a) (COATSOME® SS—OP):

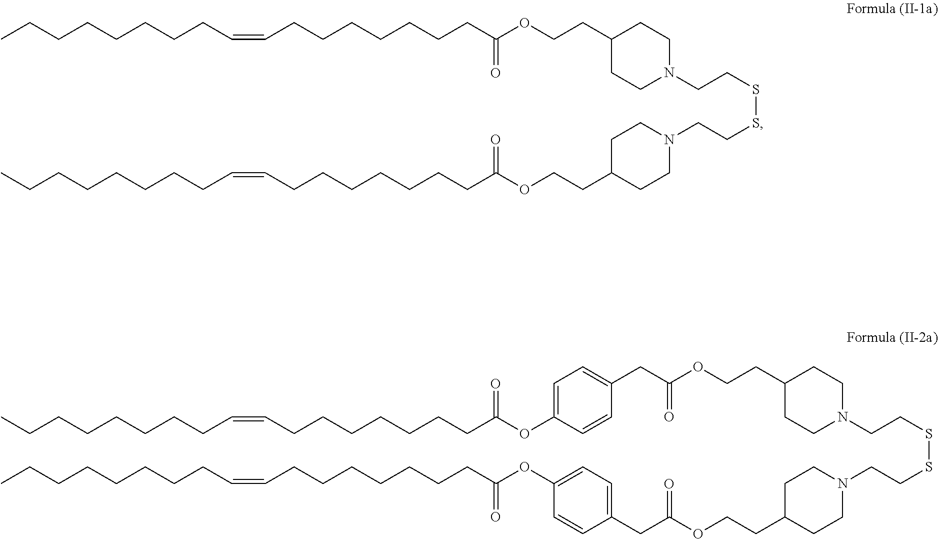 [SMILES: CCCCCCCC/C=C\CCCCCCCC(=O)OCCC1CCN(CCSSCCN2CCC(CCOC(=O)CCCCCCC/C=C\CCCCCCCC)CC2)CC1 ; CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In some embodiments, the cationic lipid of the LNP is a compound of Formula (II-1a) (COATSOME® SS—OC). COATSOME® SS—OC is also known as SS-18/4PE-16.

In some embodiments, the cationic lipid of the LNP is a compound of Formula (II-2a) (COATSOME® SS—OP).

In some embodiments, the cationic lipid of the LNP is 1,2-dioleoyl-3-trimethylammonium-propane (DOTAP).

### Helper Lipid

In some embodiments, the LNP described herein comprises one or more helper lipids. The term “helper lipid” refers to a lipid capable of increasing the delivery of the LNP to a target, e.g., into a cell. Without wishing to be bound by any particular theory, it is contemplated that a helper lipid may enhance the stability and/or membrane fusogenicity of the lipid nanoparticle. In some embodiments, the helper lipid is a phospholipid. In some embodiments, the helper lipid is a phospholipid substitute or replacement. In some embodiments the helper lipid is an alkyl resorcinol.

In some embodiments, the helper lipid is a phosphatidyl choline (PC). In some embodiments, the helper lipid is not a phosphatidyl choline (PC). In some embodiments the helper lipid is a phospholipid or a phospholipid substitute. In some embodiments, the phospholipid or phospholipid substitute can be, for example, one or more saturated or (poly) unsaturated phospholipids, or phospholipid substitutes, or a combination thereof. In general, phospholipids comprise a phosphate head group and one or more fatty acid tails. In some embodiments, a phospholipid may include one or more multiple (e.g., double or triple) bonds (i.e. one or more unsaturations). In some embodiments, the helper lipid is non-cationic.

A phosphate head group can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid tail can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-sn-glycero-3-phosphocholine (DSPC) substitute.

In some embodiments, the phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

In some embodiments, a phosphate head group can be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid tail can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

In some embodiments, the phospholipid is a compound according to Formula (III):

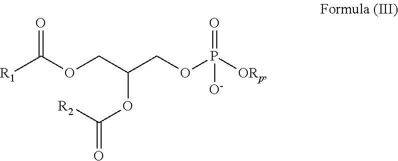

wherein: Rₚ represents a phosphate head group and R¹ and R² represent fatty acid tails with or without unsaturation that may be the same or different. A phosphate head group may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid tail may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of an LNP to facilitate membrane permeation or cellular recognition or in conjugating an LNP to a useful component such as a targeting or imaging moiety (e.g., a dye).

In some embodiments, the LNPs comprise one or more non-cationic helper lipids (e.g., neutral lipids). Exemplary neutral helper lipids include (1,2-dilauroyl-sn-glycero-3-phosphoethanolamine) (DLPE), 1,2-diphytanoyl-sn-glycero-3-phosphoethanolamine (DiPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-dioleoyl-sn-glycero-3-phosphocholine (DOPC), 1,2-distearoyl-sn-glycero-3-phosphoethanolamine (DSPE), ceramides, and sphingomyelins. In some embodiments, the one or more helper lipids are selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); and 1,2-dioleoyl-sn-glycero-3-phosphoethanolamine (DOPE). In some embodiments, the helper lipid of the LNPs comprises, consists essentially of, or consist of 1,2-Dilauroyl-sn-glycero-3-phosphoethanolamine (DLPE) or 1,2-Dioleoyl-sn-glycero-3-phosphoethanolamine (DOPE). In some embodiments, the LNP comprises DSPC. In some embodiments, the LNP comprises DOPC. In some embodiments, the LNP comprises DLPE. In some embodiments, the LNP comprises DOPE.

In some embodiments, the phospholipid is selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE (18:2/18:2), 1,2-dilinolenoyl-sn-glycero-3-phosphoethanol amine (PE 18:3 (9Z,12Z,15Z), 1,2-diarachidonoyl-sn-glycero-3-phosphoethanolamine (DAPE 18:3 (9Z,12Z,15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin.

In some embodiments, a helper lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine, dimethylphosphatidylethanolamine, 18-1-trans PE, 1-stearoyl-2-oleoylphosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidyl serine, phosphatidylinositol, sphingomyelin, egg sphingomyelin (ESM), cephalin, cardiolipin, phosphatidicacid, cerebrosides, dicetylphosphate, lysophosphatidylcholine, and dilinoleoylphosphatidylcholine.

In some embodiments, the helper lipid of the disclosure is DSPC.

In some embodiments, an LNP includes DSPC. In some embodiments, an LNP includes DOPE. In some embodiments, an LNP includes DMPE. In some embodiments, an LNP includes both DSPC and DOPE.

In some embodiments, a helper lipid is selected from the group consisting of DSPC, DMPE, and DOPC or combinations thereof.

In some embodiments of the disclosure, the helper lipid is

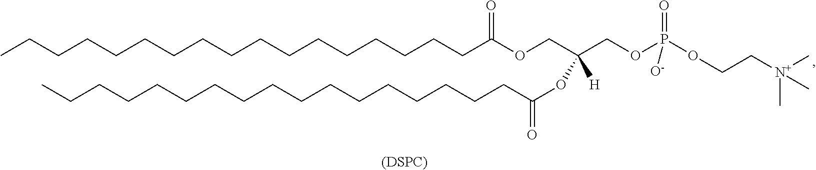

having a CAS number of 816-94-4, a linear formula of C44H88NO8P. DSPC is also known as 1,2-distearoyl-sn-glycero-3-phosphocholine.

In some embodiments, a phospholipid of the disclosure comprises a modified tail. In some embodiments, the phospholipid is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof.

In some embodiments, the helper lipid of the disclosure is an alternative lipid that is not a phospholipid.

In some embodiments, a phospholipid useful in the present disclosure comprises a modified tail. In some embodiments, a phospholipid useful in the present disclosure is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof.

In some embodiments, a phospholipid useful in the present disclosure comprises a modified phosphocholine moiety, wherein the alkyl chain linking the quaternary amine to the phosphoryl group is not ethylene (e.g., n is not 2).

In some embodiments, the LNP of the disclosure comprises an oleic acid or an oleic acid analog as the helper lipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the LNP of the disclosure comprises a different zwitterionic group in place of a phospholipid as the helper lipid.

In some embodiments, the helper lipid of the disclosure is a naturally occurring membrane lipid. In some embodiments, the helper lipid of the disclosure is 1,2-Dipalmitoyl-sn-glycero-3-O-4′-(N,N,N-trimethyl)-homoserine (DGTS), Monogalactosyldiacylglycerol (MGDG), Digalactosyldiacylglycerol (DGDG), Sulfoquinovosyldiacylglycerol (SQDG), 1-Palmitoyl-2-cis-9,10-methylenehexadecanoyl-sn-glycero-3-phosphocholine (Cyclo PC), or a combination thereof. In some embodiments, the LNP of the disclosure comprises a combination of helper lipids. In some embodiments, the combinatoin of helper lipids does not comprise DSPC. In some embodiments, the combination of helper lipid comprises DSPC. In some embodiments, the LNP comprising one or more naturally occurring membrane lipids (e.g., DGTS) has improved liver transfection/delivery of the target molecule encapsulated in the LNP as compared to the LNP comprising DSPC as the only helper lipid.

In some embodiments, the helper lipid of disclosure is 5-heptadecylresorcinol or a derivative thereof.

### Structural Lipid

In some embodiments, the LNP of the disclosure comprises one or more structural lipids. Incorporation of structural lipids in the lipid nanoparticle may help mitigate aggregation of other lipids in the particle. Structural lipids may be, but are not limited to, sterols or lipids containing sterol moieties.

In some embodiments, the structural lipid of the LNP is a sterol (e.g., phytosterols or zoosterols). In some embodiments, the sterol is cholesterol, or an analog or a derivative thereof. In some embodiments, the sterol is cholesterol. In some embodiments, the sterol is cholesterol, β-sitosterol, fecosterol, ergosterol, sitosterol, campesterol, stigmasterol, brassicasterol, ergosterol, tomatidine, tomatine, ursolic acid, alpha-tocopherol, including analogs, salts or esters thereof, alone or in combination.

In some embodiments, the structural lipid of the LNP is a cholesterol, a corticosteroid (such as, for example, prednisolone, dexamethasone, prednisone, and hydrocortisone), or a combination thereof.

In some embodiments, the structural lipid of the LNP is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, A5-avenaserol, A7-avenaserol or a A7-stigmasterol, including analogs, salts or esters thereof, alone or in combination.

In some embodiments, the LNP comprises one or more phytosterols. In some embodiments, the phytosterol component of the LNP is a single phytosterol. In some embodiments, the phytosterol component of the LNP of the disclosure is a mixture of different phytosterols (e.g. 2, 3, 4, 5 or 6 different phytosterols). In some embodiments, the phytosterol component of the LNP of the disclosure is a blend of one or more phytosterols and one or more zoosterols, such as a blend of a phytosterol (e.g., a sitosterol, such as beta-sitosterol) and cholesterol.

In some embodiments of the disclosure, the structural lipid of the LNP is cholesterol:

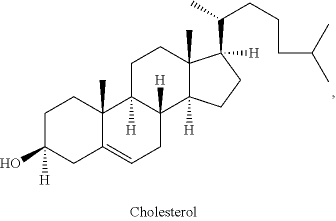

having a CAS number of 57-88-5, a linear formula of C₂₇H₄₆O.

In some embodiments, the LNP comprises a cationic lipid and one or more helper lipids, wherein the cationic lipid is DOTAP. In some embodiments, the LNP comprises a cationic lipid and one or more helper lipids, wherein the cationic lipid is DLin-MC3-DMA (MC3). In some embodiments, the LNP comprises a cationic lipid and one or more helper lipids, wherein the cationic lipid is COATSOME® SS-EC. In some embodiments, the LNP comprises a cationic lipid and one or more helper lipids, wherein the cationic lipid is COATSOME® SS-LC. In some embodiments, the LNP comprises a cationic lipid and one or more helper lipids, wherein the cationic lipid is COATSOME® SS—OC. In some embodiments, the LNP comprises a cationic lipid and one or more helper lipids, wherein the cationic lipid is COATSOME® SS—OP. In some embodiments, the LNP comprises a cationic lipid and one or more helper lipids, wherein the cationic lipid is L-319. In some embodiments, the LNP further comprises a structural lipid. In some embodiments, the structural lipid is cholesterol.

In some embodiments, the LNP comprises a cationic lipid and one or more helper lipids. In some embodiments, the LNP comprises a cationic lipid and one or more helper lipids, wherein the one or more helper lipids comprises DLPE. In some embodiments, the LNP comprises a cationic lipid and one or more helper lipids, wherein the one or more helper lipids comprises DSPC. In some embodiments, the LNP comprises a cationic lipid and one or more helper lipids, wherein the one or more helper lipids comprises DOPE. In some embodiments, the LNP comprises a cationic lipid and one or more helper lipids, wherein the one or more helper lipids comprises DOPC. In some embodiments, the LNP further comprises a structural lipid. In some embodiments, the structural lipid is cholesterol.

In some embodiments, the LNP comprises a cationic lipid, a helper lipid, and a structural lipid. In some embodiments, the structural lipid is cholesterol. In some embodiments, the cationic lipid is DOTAP, and the helper lipid is DLPE. In some embodiments, the cationic lipid is MC3, and the helper lipid is DSPC. In some embodiments, the helper lipid is DOPE. In some embodiments, the helper lipid is DSPC. In some embodiments, the LNP comprises a cationic lipid, a structural lipid, and at least two helper lipids, wherein the cationic lipid is DOTAP, and the at least two helper lipids comprise DLPE and DSPE. In some embodiments, the LNP comprises a cationic lipid, a structural lipid, and at least two helper lipids, wherein the cationic lipid is MC3, and the at least two helper lipids comprise DSPC and DMG. In some embodiments, the at least two helper lipids comprise DOPE and DSPE. In some embodiments, the at least two helper lipids comprise DSPC, and DMG. In some embodiments, the structural lipid is cholesterol. In some embodiments, the LNP comprises DOTAP, cholesterol, and DLPE. In some embodiments, the LNP comprises MC3, cholesterol, and DSPC. In some embodiments, the LNP comprises DOTAP, cholesterol, and DOPE. In some embodiments, the LNP comprises DOTAP, cholesterol, DLPE, and DSPE. In some embodiments, the LNP comprises MC3, cholesterol, DSPC, and DMG. In some embodiments, the LNP comprises DOTAP, cholesterol, DLPE, and DSPE-PEG. In some embodiments, the LNP comprises MC3, cholesterol, DSPC, and DMG-PEG. In some embodiments, the LNP comprises DOTAP, cholesterol, DOPE, and DSPE. In some embodiments, the LNP comprises DOTAP, cholesterol, DOPE, and DSPE-PEG. In some embodiments, the LNP comprises SS—OC, DSPC, cholesterol, and DPG-PEG (e.g., DPG-PEG2K). In some embodiments, the LNP comprises SS—OC, DSPC, cholesterol, and a PEG-lipid of formula (I) (e.g., BRIJ™ S100).

### Lipid Molar Ratio in the LNP Composition

In some embodiments, the LNP of the disclosure comprises between 40 mol % and 70 mol % of the cationic lipid, up to 50 mol % of the helper lipid, between 10 mol % and 50 mol % of the structural lipid, and between 0.001 mol % and 5 mol % of the PEG-lipid, inclusive of all endpoints. In some embodiments, the total mol % of the cationic lipid, the helper lipid, the structural lipid and the PEG-lipid is 100%.

In some embodiments, the mol % of the cationic lipid in the LNP is 40-70 mol %, 40-55 mol %, 40-50 mol %, 40-45 mol %, 44-54 mol %, 45-60 mol %, 45-55 mol %, 45-50 mol %, 50-60 mol %, 49-64 mol %, 50-55 mol %, or 55-60 mol %. In some embodiments, the mol % of the cationic lipid in the LNP is 44-54 mol %. In some embodiments, the mol % of the cationic lipid in the LNP is 40, 41, 42, 43, 44, 45, 46, 47, 48, 49, 50, 51, 52, 53, 54, 55, 56, 57, 58, 59, or 60 mol %. In some embodiments, the mol % of the cationic lipid in the LNP is about 40, about 41, about 42, about 43, about 44, about 45, about 46, about 47, about 48, about 49, about 50, about 51, about 52, about 53, about 54, about 55, about 56, about 57, about 58, about 59, or about 60 mol %. All values are inclusive of all endpoints.

In some embodiments, the mol % of the structural lipid in the LNP is 10-60 mol %, 10-30 mol %, 15-35 mol %, 20-40 mol %, 20-45 mol %, 25-33 mol %, 24-32 mol %, 25-45 mol %, 30-50 mol %, 35-43 mol %, 35-55 mol %, or 40-60 mol %. In some embodiments, the mol % of the structural lipid in the LNP is 20-45 mol %. In some embodiments, the mol % of the structural lipid in the LNP is 24-32 mol %. In some embodiments, the mol % of the structural lipid in the LNP is 25-33 mol %. In some embodiments, the mol % of the structural lipid in the LNP is 22-28 mol %. In some embodiments, the mol % of the structural lipid in the LNP is 35-45 mol %. In some embodiments, the mol % of the structural lipid in the LNP is 35-43 mol %. In some embodiments, the mol % of the structural lipid in the LNP is 10-60 mol %. In some embodiments, the mol % of the structural lipid in the LNP is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %. In some embodiments, the mol % of the structural lipid in the LNP is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 mol %. In some embodiments, the structural lipid is cholesterol. All values are inclusive of all endpoints.

In some embodiments, the mol % of the helper lipid in the LNP is 1-50 mol %. In some embodiments, the mol % of the helper lipid in the LNP is up to 29 mol %. In some embodiments, the mol % of the helper lipid in the LNP is 1-10 mol %, 5-9 mol %, 5-15 mol %, 8-14 mol %, 18-22%, 19-25 mol %, 10-20 mol %, 10-25 mol %, 15-25 mol %, 20-30 mol %, 25-35 mol %, 30-40 mol %, or 35-50 mol %. In some embodiments, the mol % of the helper lipid in the LNP is 10-25 mol %. In some embodiments, the mol % of the helper lipid in the LNP is 5-9 mol %. In some embodiments, the mol % of the helper lipid in the LNP is 8-14 mol %. In some embodiments, the mol % of the helper lipid in the LNP is 18-22 mol %. In some embodiments, the mol % of the helper lipid in the LNP is 19-25 mol %. In some embodiments, the mol % of the helper lipid in the LNP is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mol %. In some embodiments, the mol % of the helper lipid in the LNP is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 mol %. In some embodiments, the helper lipid is DSPC. All values are inclusive of all endpoints.

In some embodiments, the mol % of the PEG-lipid in the LNP is greater than 0 mol % and up to 5 mol % of the total lipid present in the LNP. In some embodiments, the mol % of the PEG-lipid is 0.1 mol %, 0.2 mol %, 0.25 mol %, 0.3 mol %, 0.4 mol %, 0.5 mol %, 0.6 mol %, 0.7 mol %, 0.8 mol %, 0.9 mol %, 1.0 mol %, 1.1 mol %, 1.2 mol %, 1.3 mol %, 1.4 mol %, 1.5 mol %, 1.6 mol %, 1.7 mol %, 1.8 mol %, 1.9 mol %, 2.0 mol %, 2.1 mol %, 2.2 mol %, 2.3 mol %, 2.4 mol %, 2.5 mol %, 2.6 mol %, 2.7 mol %, 2.8 mol %, 2.9 mol %, 3.0 mol %, 3.1 mol %, 3.2 mol %, 3.3 mol %, 3.4 mol %, 3.5 mol %, 4.0 mol %, 4.5 mol %, or 5 mol % of the total lipid present in the LNP. In some embodiments, the mol % of the PEG-lipid is about 0.1 mol %, about 0.2 mol %, about 0.25 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 4.0 mol %, about 4.5 mol %, or about 5 mol % of the total lipid present in the LNP. In some embodiments, the mol % of the PEG-lipid is at least 0.1 mol %, at least 0.2 mol %, at least 0.25 mol %, at least 0.3 mol %, at least 0.4 mol %, at least 0.5 mol %, at least 0.6 mol %, at least 0.7 mol %, at least 0.8 mol %, at least 0.9 mol %, at least 1.0 mol %, at least 1.1 mol %, at least 1.2 mol %, at least 1.3 mol %, at least 1.4 mol %, at least 1.5 mol %, at least 1.6 mol %, at least 1.7 mol %, at least 1.8 mol %, at least 1.9 mol %, at least 2.0 mol %, at least 2.1 mol %, at least 2.2 mol %, at least 2.3 mol %, at least 2.4 mol %, at least 2.5 mol %, at least 2.6 mol %, at least 2.7 mol %, at least 2.8 mol %, at least 2.9 mol %, at least 3.0 mol %, at least 3.1 mol %, at least 3.2 mol %, at least 3.3 mol %, at least 3.4 mol %, at least 3.5 mol %, at least 4.0 mol %, at least 4.5 mol %, or at least 5 mol % of the total lipid present in the LNP. In some embodiments, the mol % of the PEG-lipid is at most 0.1 mol %, at most 0.2 mol %, at most 0.25 mol %, at most 0.3 mol %, at most 0.4 mol %, at most 0.5 mol %, at most 0.6 mol %, at most 0.7 mol %, at most 0.8 mol %, at most 0.9 mol %, at most 1.0 mol %, at most 1.1 mol %, at most 1.2 mol %, at most 1.3 mol %, at most 1.4 mol %, at most 1.5 mol %, at most 1.6 mol %, at most 1.7 mol %, at most 1.8 mol %, at most 1.9 mol %, at most 2.0 mol %, at most 2.1 mol %, at most 2.2 mol %, at most 2.3 mol %, at most 2.4 mol %, at most 2.5 mol %, at most 2.6 mol %, at most 2.7 mol %, at most 2.8 mol %, at most 2.9 mol %, at most 3.0 mol %, at most 3.1 mol %, at most 3.2 mol %, at most 3.3 mol %, at most 3.4 mol %, at most 3.5 mol %, at most 4.0 mol %, at most 4.5 mol %, or at most 5 mol % of the total lipid present in the LNP. In some embodiments, the mol % of the PEG-lipid is between 0.1-4 mol % of the total lipid present in the LNP. In some embodiments, the mol % of the PEG-lipid is between 0.1-2 mol % of the total lipid present in the LNP. In some embodiments, the mol % of the PEG-lipid is between 0.2-0.8 mol %, 0.4-0.6 mol %, 0.7-1.3 mol %, 1.2-1.8 mol %, or 1-3.5 mol % of the total lipid present in the LNP. In some embodiments, the mol % of the PEG-lipid is 0.1-0.7 mol %, 0.2-0.8 mol %, 0.3-0.9 mol %, 0.4-0.8 mol %, 0.4-0.6 mol %, 0.4-1 mol %, 0.5-1.1 mol %, 0.6-1.2 mol %, 0.7-1.3 mol %, 0.8-1.4 mol %, 0.9-1.5 mol %, 1-3.5 mol % 1-1.6 mol %, 1.1-1.7 mol %, 1.2-1.8 mol %, 1.3-1.9 mol %, 1.4-2 mol %, 1.5-2.1 mol %, 1.6-2.2 mol %, 1.7-2.3 mol %, 1.8-2.4 mol %, 1.9-2.5 mol %, 2-2.6 mol %, 2.4-3.8 mol %, or 2.6-3.4 mol % of the total lipid present in the LNP. All values are inclusive of all endpoints.

In some embodiments, the LNP of the disclosure comprises 44-60 mol % of the cationic lipid, 19-25 mol % of the helper lipid, 25-33 mol % of the structural lipid, and 0.2-0.8 mol % of the PEG-lipid, inclusive of the endpoints. In some embodiments, the LNP of the disclosure comprises 44-54 mol % of the cationic lipid, 19-25 mol % of the helper lipid, 24-32 mol % of the structural lipid, and 1.2-1.8 mol % of the PEG-lipid, inclusive of the endpoints. In some embodiments, the LNP of the disclosure comprises 44-54 mol % of the cationic lipid, 8-14 mol % of the helper lipid, 35-43 mol % of the structural lipid, and 1.2-1.8 mol % of the PEG-lipid, inclusive of the endpoints. In some embodiments, the LNP of the disclosure comprises 45-55 mol % of the cationic lipid, 5-9 mol % of the helper lipid, 36-44 mol % of the structural lipid, and 2.5-3.5 mol % of the PEG-lipid, inclusive of the endpoints.

In some embodiments, the LNP of the disclosure comprises one or more of the cationic lipids of the disclosure, one or more helper lipids of the disclosure, one or more structural lipids of the disclosure, and one or more PEG-lipid of the disclosure at a mol % of total lipid (or the mol % range of total lipid) in the LNP according to Table 6 below. In some embodiments, the total mol % of these four lipid components equals 100%. In some embodiments, the total mol % of these four lipid components is less than 100%. In some embodiments, the cationic lipid is a compound of Formula (I) or a compound selected from Table 21. In some embodiments, the structural lipid is cholesterol. In some embodiments, the helper lipid is DSPC. In some embodiments, the PEG-lipid is of Formula (A), Formula (A′), or Formula (A″).

TABLE 6
Mol % of the Lipid Components in the LNP

| Cationic Lipid | Structural Lipid | Helper Lipid | PEG-lipid |
|---|---|---|---|
| (mol %) | (mol %) | (mol %) | (mol %) |
| 49 | 28.5 | 22 | 0.5 |
| 47-52 | 27-30 | 21-23 | 0.4-0.6 |
| 44-54 | 25-32 | 19-25 | 0.2-0.8 |
| 44-54 | 25-33 | 19-25 | 0.2-0.8 |
| 49 | 27.5 | 22 | 1.5 |
| 47-52 | 26-29 | 21-23 | 1.3-1.7 |
| 44-54 | 24-31 | 19-25 | 1.1-1.9 |
| 44-54 | 24-32 | 19-25 | 1.2-1.8 |
| 49 | 39.5 | 11 | 0.5 |
| 47-52 | 38-41 | 11-13 | 0.4-0.6 |
| 44-54 | 36-43 | 9-15 | 0.2-0.8 |
| 49 | 38.5 | 11 | 1.5 |
| 47-52 | 37-40 | 11-13 | 1.3-1.7 |
| 44-54 | 35-42 | 9-15 | 1.1-1.9 |
| 44-54 | 35-43 | 8-14 | 1.2-1.8 |
| 20-60 | 10-60 | ≥20 | 0.5 |
| 20-60 | 10-60 | ≥20 | 0.3-0.7 |
| 20-60 | 10-60 | ≥20 | 0.1-0.9 |
| 20-60 | 10-60 | 10 | 1.5 |
| 20-60 | 10-60 | 8-12 | 1.3-1.7 |
| 20-60 | 10-60 | 6-14 | 1.1-1.9 |
| 50 | 40 | 7 | 3 |
| 45-55 | 35-45 | 5-9 | 2.5-3.5 |
| 54.5 | 25 | 20 | 0.5 |
| 50-60 | 22-28 | 18-22 | 0.3-0.7 |
| 54.6 | 25.1 | 20.1 | 0.25 |
| 44.5 | 50 | 5 | 0.5 |
| 40 | 50 | 8.75 | 1.25 |
| 60 | 25 | 14.5 | 0.5 |
| 60 | 34.3 | 5 | 0.7 |
| 50 | 42.5 | 7 | 0.5 |
| 58 | 33.5 | 7 | 1.5 |
| 58 | 34.5 | 7 | 0.5 |
| 35-65 | 25-55 | 5-25 | 0.3-3 |
| 49 | 28.5 | 22 | 0.5 |
| 40 | 39.5 | 20 | 0.5 |
| 40 | 50 | 8.75 | 1.25 |
| 60 | 34.3 | 5 | 0.7 |
| 54.6 | 25.1 | 20.1 | 0.25 |
| 50.1 | 42.6 | 7 | 0.24 |

In some embodiments, the LNP of the disclosure comprises 44-54 mol % of the cationic lipid, 19-25 mol % of the helper lipid, 25-33 mol % of the structural lipid, and 0.2-0.8 mol % of the PEG-lipid, inclusive of the endpoints. In some embodiments, the LNP of the disclosure comprises 44-54 mol % of the compound of Formula (II-1a), 19-25 mol % of the DSPC, 25-33 mol % of the cholesterol, and 0.2-0.8 mol % of the PEG-lipid selected from HO-PEG100-CH₂ (CH₂)₁₆CH₃, HO-PEG20-CH₂ (CH₂)₁₆CH₃, HO-PEG20-CH₂ (CH₂)₁₄CH₃, HO-PEG20-C₁₈H₃₅, HO-PEG100-C(O)—CH₂ (CH₂)₁₃CH₃, HO-PEG50-C(O)—CH₂ (CH₂)₁₃CH₃, HO-PEG40-C(O)—CH₂ (CH₂)₁₃CH₃, HO-PEG100-C(O)—CH₂ (CH₂)₁₅CH₃, HO-PEG50-C(O)—CH₂ (CH₂)₁₅CH₃, and HO-PEG40-C(O)—CH₂ (CH₂)₁₅CH₃, inclusive of the endpoints.

In some embodiments, the LNP of the disclosure comprises 44-54 mol % of the cationic lipid, 19-25 mol % of the helper lipid, 24-32 mol % of the structural lipid, and 1.2-1.8 mol % of the PEG-lipid, inclusive of the endpoints. In some embodiments, the LNP of the disclosure comprises 44-54 mol % of the compound of Formula (II-la), 19-25 mol % of the DSPC, 24-32 mol % of the cholesterol, and 1.2-1.8 mol % of the PEG-lipid selected from HO-PEG100-CH₂ (CH₂)₁₆CH₃, HO-PEG20-CH₂ (CH₂)₁₆CH₃, HO-PEG20-CH₂ (CH₂)₁₄CH₃, HO-PEG20-C₁₈H₃₅, HO-PEG100-C(O)—CH₂ (CH₂)₁₃CH₃, HO-PEG50-C(O)—CH₂ (CH₂)₁₃CH₃, HO-PEG40—C(O)—CH₂ (CH₂)₁₃CH₃, HO-PEG100-C(O)—CH₂ (CH₂)₁₅CH₃, HO-PEG50-C(O)—CH₂ (CH₂)₁₅CH₃, and HO-PEG40-C(O)—CH₂ (CH₂)₁₅CH₃, inclusive of the endpoints.

In some embodiments, the LNP of the disclosure comprises 44-54 mol % of the cationic lipid, 8-14 mol % of the helper lipid, 35-43 mol % of the structural lipid, and 1.2-1.8 mol % of the PEG-lipid, inclusive of the endpoints. In some embodiments, the LNP of the disclosure comprises 44-54 mol % of the compound of Formula (II-1a), 8-14 mol % of the DSPC, 35-43 mol % of the cholesterol, and 1.2-1.8 mol % of the PEG-lipid selected from HO-PEG100-CH₂ (CH₂)₁₆CH₃, HO-PEG20-CH₂ (CH₂)₁₆CH₃, HO-PEG20-CH₂ (CH₂)₁₄CH₃, HO-PEG20-C₁₈H₃₅, HO-PEG100-C(O)—CH₂ (CH₂)₁₃CH₃, HO-PEG50-C(O)—CH₂ (CH₂)₁₃CH₃, HO-PEG40-C(O)—CH₂ (CH₂)₁₃CH₃, HO-PEG100-C(O)—CH₂ (CH₂)₁₅CH₃, HO-PEG50-C(O)—CH₂ (CH₂)₁₅CH₃, and HO-PEG40-C(O)—CH₂ (CH₂)₁₅CH₃, inclusive of the endpoints.

In some embodiments, the LNP comprises SS—OC, DSPC, cholesterol (Chol), and a PEG-lipid, wherein the ratio of SS—OC:DSPC:Chol:PEG-lipid (as a percentage of total lipid content) is about A:B:C:D, wherein A=40 mol %-60 mol %, B=10 mol %-25 mol %, C=20 mol %-30 mol %, and D=0 mol %-3 mol % and wherein A+B+C+D=100 mol %. In some embodiments, the LNP comprises SS—OC, DSPC, cholesterol (Chol), and a PEG-lipid, wherein the ratio of SS—OC:DSPC:Chol:PEG-lipid (as a percentage of total lipid content) is about A:B:C:D, wherein A=45 mol %-50 mol %, B=20 mol %-25 mol %, C=25 mol %-30 mol %, and D=0 mol %-1 mol % and wherein A+B+C+D=100 mol %. In some embodiments, the LNP comprises SS—OC, DSPC, cholesterol (Chol), and a PEG-lipid, wherein the ratio of SS—OC:DSPC:Chol:PEG-lipid (as a percentage of total lipid content) is about 49:22:28.5:0.5. In some embodiments, the PEG-lipid is a compound of Formula (A), Formula (A′), or Formula (A″). In some embodiments, the PEG-lipid is selected from the group consisting of BRIJ™ S100, BRIJ™ S20, BRIJ™ O20 and BRIJ™ C20. In some embodiments, the PEG-lipid is BRIJ™ S100.

In some embodiments, the LNP comprises DOTAP, cholesterol (Chol), and DLPE, wherein the ratio of DOTAP:Chol:DLPE (as a percentage of total lipid content) is about 50:35:15. In some embodiments, the LNP comprises DOTAP, cholesterol (Chol), and DLPE, wherein the ratio of DOTAP:Chol:DOPE (as a percentage of total lipid content) is about 50:35:15. In some embodiments, the LNP comprises DOTAP, cholesterol (Chol), DLPE, DSPE-PEG, wherein the ratio of DOTP:Chol:DLPE (as a percentage of total lipid content) is about 50:35:15 and wherein the particle comprises about 0.2 mol % DSPE-PEG. In some embodiments, the LNP comprises MC3, cholesterol (Chol), DSPC, and DMG-PEG, wherein the ratio of MC3:Chol:DSPC:DMG-PEG (as a percentage of total lipid content) is about 49:38.5:11:1.5.

In some embodiments, the LNP comprises SS—OC, DSPC, cholesterol (Chol), and DPG-PEG2K), wherein the ratio of SS—OC:DSPC:Chol:DPG-PEG2K (as a percentage of total lipid content) is about A:B:C:D, wherein A=40 mol %-60 mol %, B=10 mol %-25 mol %, C=20 mol %-30 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-50 mol %, B=20 mol %-25 mol %, C=25 mol %-30 mol %, and D=0 mol %-1 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about 49:22:28.5:0.5.

In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=40 mol %-60 mol %, B=10 mol %-30 mol %, C=20 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=40 mol %-60 mol %, B=10 mol %-30 mol %, C=25 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-55 mol %, B=10 mol %-20 mol %, C=30 mol %-40 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-50 mol %, B=10 mol %-15 mol %, C=35 mol %-40 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is 49:11:38.5:1.5.

In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-65 mol %, B=5 mol %-20 mol %, C=20 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=50 mol %-60 mol %, B=5 mol %-15 mol %, C=30 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=55 mol %-60 mol %, B=5 mol %-15 mol %, C=30 mol %-40 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=55 mol %-60 mol %, B=5 mol %-10 mol %, C=30 mol %-35 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is 58:7:33.5:1.5.

In some embodiments, the nanoparticle is coated with a glycosaminoglycan (GAG) in order to modulate or facilitate uptake of the nanoparticle by target cells. The GAG may be heparin/heparin sulfate, chondroitin sulfate/dermatan sulfate, keratin sulfate, or hyaluronic acid (HA). In a particular embodiment, the surface of the nanoparticle is coated with HA and targets the particles for uptake by tumor cells. In some embodiments, the lipid nanoparticle is coated with an arginine-glycine-aspartate tri-peptide (RGD peptides) (See Ruoslahti, Advanced Materials, 24, 2012, 3747-3756; and Bellis et al., Biomaterials, 32 (18), 2011, 4205-4210).

### Properties of LNP Composition

The disclosure provides compositions (e.g., pharmaceutical compositions) comprising a plurality of LNPs as described herein. Also provided herein are compositions comprising LNPs as described herein and encapsulated molecules.

In some embodiments, the LNP of the present disclosure may reduce immune response in vivo as compared to a control LNP. In some embodiments, the control LNP is an LNP comprising a PEG-lipid that is not of Formula (A), Formula (A′), or Formula (A″). In some embodiments, the PEG-lipid of the control LNP is PEG2k-DPG. In some embodiments, the PEG-lipid of the control LNP is PEG2k-DMG. In some embodiments, the control LNP has the same molar ratio of the PEG-lipid as the LNP of the present disclosure. In some embodiments, the control LNP is identical to an LNP of the present disclosure except that the control LNP comprises a PEG-lipid that is not of Formula (A), Formula (A′), or Formula (A″) (e.g., the control LNP may comprise PEG2k-DPG or PEG2k-DMG as PEG-lipid).

In some embodiments, the control LNP is an LNP comprising a cationic lipid that is not of Formula (I). In some embodiments, the cationic lipid of the control LNP is SS—OC. In some embodiments, the control LNP has the same molar ratio of the cationic lipid as the LNP of the present disclosure. In some embodiments, the control LNP is identical to an LNP of the present disclosure except that the control LNP comprises a cationic lipid that is not of Formula (I) (e.g., the control LNP may comprise SS—OC as cationic lipid).

In some embodiments, the reduced immune response may be a reduction in accelerated blood clearance (ABC). In some embodiments, the ABC is associated with the secretion of natural IgM and/or anti-PEG IgM. The term “natural IgM,” as used herein, refers to circulating IgM in the serum that exists independent of known immune exposure (e.g., the exposure to a LNP of the disclosure). The term “reduction of ABC” refers to any reduction in ABC in comparison to a control LNP. In some embodiments, a reduction in ABC may be a reduced clearance of the LNP upon a second or subsequent dose, relative to a control LNP. In some embodiments, the reduction may be at least 10%, 15%, 20%, 25%, 30%, 35%, 40%, 45%, 50%, 55%, 60%, 65%, 70%, 75%, 80%, 85%, 90%, 95%, 98%, or 100%. In some embodiments, the reduction is about 10% to about 100%, about 10 to about 50%, about 20 to about 100%, about 20 to about 50%, about 30 to about 100%, about 30 to about 50%, about 40% to about 100%, about 40 to about 80%, about 50 to about 90%, or about 50 to about 100%. In some embodiments, a reduction in ABC may be measured by an increase in or a sustained detectable level of an encapsulated synthetic RNA viral genome following a second or subsequent administration. In some embodiments, a reduction in ABC may result in an increase (e.g., a 2-fold, a 3-fold, a 4-fold, a 5-fold, or higher fold increase) in the level of the encapsulated synthetic RNA viral genome relative to the level of encapsulated synthetic RNA viral genome following administration of a control LNP. In some embodiments, the reduced ABC is associated with a lower serum level of anti-PEG IgM.

In some embodiments, the LNP of the present disclosure may delay clearance of the LNP and components thereof upon repeat dosing compared to a control LNP, which may be cleared prior to release of encapsulated molecule. Accordingly, the LNP of the present disclosure may increase the delivery efficiency of the encapsulated molecule (e.g., synthetic RNA viral genome) in subsequent doses.

In some embodiments, the LNPs have an average size (i.e., average outer diameter) of about 50 nm to about 500 nm. In some embodiments, the LNPs have an average size of about 50 nm to about 200 nm, about 100 nm to about 200 nm, about 150 nm to about 200 nm, about 50 nm to about 100 nm, about 50 nm to about 150 nm, about 100 nm to about 150 nm, about 200 nm to about 250 nm, about 250 nm to about 300 nm, about 300 nm to about 400 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the LNPs have an average size of about 50 nm, 60 nm, 70 nm, 80 nm, 90 nm, 100 nm, 110 nm, about 120, or about 125 nm. In some embodiments, the LNPs have an average size of about 100 nm. In some embodiments, the LNPs have an average size of 50 nm to 150 nm. In some embodiments, the LNPs have an average size (average outer diameter) of 50 nm to 150 nm, 50 nm to 125 nm, 50 nm to 100 nm, 50 nm to 75 nm, 75 nm to 150 nm, 75 nm to 125 nm, 75 nm to 100 nm, 100 nm to 150 nm, 100 nm to 125 nm, or 125 nm to 150 nm. In some embodiments, the LNPs have an average size of 70 nm to 90 nm, 80 nm to 100 nm, 90 nm to 110 nm, 100 nm to 120 nm, 110 nm to 130 nm, 120 nm to 140 nm, or 130 nm to 150 nm. In some embodiments, the LNPs have an average size of 90 nm to 110 nm. All values are inclusive of end points.

In some embodiments, the LNPs have an average size (i.e., average outer diameter) of about 50 nm to about 150 nm. In some embodiments, the disclosure provides a therapeutic composition comprising a plurality of lipid nanoparticles, wherein the plurality of LNPs have an average size of about 60 nm to about 130 nm. In some embodiments, the disclosure provides a therapeutic composition comprising a plurality of lipid nanoparticles, wherein the plurality of LNPs have an average size of about 70 nm to about 120 nm. In some embodiments, the disclosure provides a therapeutic composition comprising a plurality of lipid nanoparticles, wherein the plurality of LNPs have an average size of about 70 nm. In some embodiments, the disclosure provides a therapeutic composition comprising a plurality of lipid nanoparticles, wherein the plurality of LNPs have an average size of about 80 nm. In some embodiments, the disclosure provides a therapeutic composition comprising a plurality of lipid nanoparticles, wherein the plurality of LNPs have an average size of about 90 nm. In some embodiments, the disclosure provides a therapeutic composition comprising a plurality of lipid nanoparticles, wherein the plurality of LNPs have an average size of about 100 nm. In some embodiments, the disclosure provides a therapeutic composition comprising a plurality of lipid nanoparticles, wherein the plurality of LNPs have an average size of about 110 nm. All values are inclusive of end points.

In some embodiments, the encapsulation efficiency of the synthetic RNA viral genome by the LNP is about 70%, about 75%, about 80%, about 85%, about 90%, about 91%, about 92%, about 93%, about 94%, about 95%, about 96%, about 97%, about 98%, about 99%, or 100%. In some embodiments, about 70%, about 75%, about 80%, about 90%, about 95%, about 97%, about 98%, or about 99% of the plurality of LNPs comprises an encapsulated synthetic RNA viral genome. In some embodiments, the encapsulation efficiency of the synthetic RNA viral genome by the LNP is at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, at least 70%, at least 75%, at least 80%, at least 90%, at least 95%, at least 97%, at least 98%, or at least 99% of the plurality of LNPs comprises an encapsulated synthetic RNA viral genome. In some embodiments, about 70% to 100%, about 75% to 100%, about 80% to 100%, about 85% to 100%, about 90% to 100%, about 91% to 100%, about 92% to 100%, about 93% to 100%, about 94% to 100%, about 95% to 100%, about 96% to 100%, about 97% to 100%, about 98% to 100%, about 99% to 100% of the plurality of LNPs comprises an encapsulated synthetic RNA viral genome.

In some embodiments, the LNPs have a neutral charge (e.g., an average zeta-potential of between about 0 mV and 1 mV). In some embodiments, the LNPs have an average zeta-potential of between about 40 mV and about −40 mV. In some embodiments, the LNPs have an average zeta-potential of between about 40 mV and about 0 mV. In some embodiments, the LNPs have an average zeta-potential of between about 35 mV and about 0 mV, about 30 mV and about 0 mV, about 25 mV to about 0 mV, about 20 mV to about 0 mV, about 15 mV to about 0 mV, about 10 mV to about 0 mV, or about 5 mV to about 0 mV. In some embodiments, the LNPs have an average zeta-potential of between about 20 mV and about −40 mV. In some embodiments, the LNPs have an average zeta-potential of between about 20 mV and about −20 mV. In some embodiments, the LNPs have an average zeta-potential of between about 10 mV and about −20 mV. In some embodiments, the LNPs have an average zeta-potential of between about 10 mV and about −10 mV. In some embodiments, the LNPs have an average zeta-potential of about 10 mV, about 9 mV, about 8 mV, about 7 mV, about 6 mV, about 5 mV, about 4 mV, about 3 mV, about 2 mV, about 1 mV, about 0 mV, about −1 mV, about −2 mV, about −3 mV, about −4 mV, about −5 mV, about −6 mV, about −7 mV, about −8 mV, about −9 mV, about −9 mV or about −10 mV.

In some embodiments, the LNPs have an average zeta-potential of between about 0 mV and −20 mV. In some embodiments, the LNPs have an average zeta-potential of less than about −20 mV. For example in some embodiments, the LNPs have an average zeta-potential of less than about less than about −30 mV, less than about 35 mV, or less than about −40 mV. In some embodiments, the LNPs have an average zeta-potential of between about −50 mV to about −20 mV, about −40 mV to about −20 mV, or about −30 mV to about −20 mV. In some embodiments, the LNPs have an average zeta-potential of about 0 mV, about −1 mV, about −2 mV, about −3 mV, about −4 mV, about −5 mV, about −6 mV, about −7 mV, about −8 mV, about −9 mV, about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, about −30 mV, about −31 mV, about −32 mV, about −33 mV, about −34 mV, about −35 mV, about −36 mV, about −37 mV, about −38 mV, about −39 mV, or about −40 mV. In some embodiments, the LNPs have an average zeta-potential of less than about −20 mV, less than about −30 mV, less than about 35 mV, or less than about −40 mV.

In some embodiments, the LNP comprises a recombinant nucleic acid molecule described herein and has a mass ratio of lipid (L) to nucleic acid (N) of about 10:1 to about 60:1. In some embodiments, the LNP comprises a recombinant nucleic acid molecule described herein and has a mass ratio of lipid (L) to nucleic acid (N) of about 20:1. In some embodiments, the LNP comprises a recombinant nucleic acid molecule described herein and has a mass ratio of lipid (L) to nucleic acid (N) of about 30:1. In some embodiments, the LNP comprises a recombinant nucleic acid molecule described herein and has a mass ratio of lipid (L) to nucleic acid (N) of about 40:1. In some embodiments, the LNP comprises a recombinant nucleic acid molecule described herein and has an L:N mass ratio of about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 237:1, about 28:1, about 39:1, about 40:1, about 41:1, about 42:1, about 43:1, about 44:1, or about 45:1.

In some embodiments, the LNP has a lipid (L) to nucleic acid molecule (N) mass ratio of between 10:1 and 60:1, between 20:1 and 60:1, between 30:1 and 60:1, between 40:1 and 60:1, between 50:1 and 60:1, between 10:1 and 50:1, between 20:1 and 50:1, between 30:1 and 50:1, between 40:1 and 50:1, between 10:1 and 40:1, between 20:1 and 40:1, between 30:1 and 40:1, between 10:1 and 30:1, between 20:1 and 30:1, or between 10:1 and 20:1, inclusive of all endpoints. In some embodiments, the LNP has a lipid:nucleic acid molecule mass ratio of between 30:1 and 40:1. In some embodiments, the LNP has a lipid:nucleic acid molecule mass ratio of between 30:1 and 36:1.

In some embodiments, the LNP comprises a recombinant nucleic acid molecule described herein and has a mass ratio of lipid (L) to nucleic acid (N) of about 10:1 to about 60:1. In some embodiments, the LNP comprises a recombinant nucleic acid molecule described herein and has a mass ratio of lipid (L) to nucleic acid (N) of about 20:1. In some embodiments, the LNP comprises a recombinant nucleic acid molecule described herein and has a mass ratio of lipid (L) to nucleic acid (N) of about 30:1 (L:N). In some embodiments, the LNP comprises a recombinant nucleic acid molecule described herein and has a mass ratio of lipid (L) to nucleic acid (N) of about 40:1 (L:N). In some embodiments, the LNP comprises a recombinant nucleic acid molecule described herein and has an L:N mass ratio of about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 237:1, about 28:1, about 39:1, about 40:1, about 41:1, about 42:1, about 43:1, about 44:1, or about 45:1.

In some embodiments, the LNP comprises a nucleic acid molecule and has a lipid-nitrogen-to-phosphate ratio (N:P) of between 1 to 25. In some embodiments, the N:P is 1, 2, 3, 4, 5, 6, 7, 8, 9, 10, 11, 12, 13, 14, 15, 16, 17, 18, 19, 20, 21, 22, 23, 24, or 25. In some embodiments, the N:P is between 1 to 25, between 1 to 20, between 1 to 15, between 1 to 10, between 1 to 5, between 5 to 25, between 5 to 20, between 5 to 15, between 5 to 10, between 10 to 25, between 10 to 20, between 10 to 15, between 15 to 25, between 15 to 20, or between 20 to 25. In some embodiments, the LNP comprises a nucleic acid molecule and has a lipid-nitrogen-to-phosphate ratio (N:P) of 14.

In some embodiments, the LNP comprises a synthetic RNA viral genome encoding an oncolytic virus, wherein the encoded oncolytic virus is capable of reducing the size of a tumor that is remote from the site of LNP administration to a subject. For example, as demonstrated in the examples provided herein, intravenous administration of the LNPs described herein results in viral replication in tumor tissue and reduction of tumor size. These data indicate that the LNPs of the present disclosure are capable of localizing to tumors or cancerous tissues that are remote from the site of LNP administration. Such effects enable the use of the LNP-encapsulated oncolytic viruses described herein in the treatment of tumors that are not easily accessible and therefore not suitable for intratumoral delivery of treatment.

### Method of LNP Preparation

In some embodiments, the disclosure provides methods for preparing a composition of lipid nanoparticles (LNPs) containing a nucleic acid molecule, comprising the steps of:
(a) diluting the nucleic acid molecule to a desired concentration in an aqueous solution;
(b) mixing organic lipid phase comprising all lipid components of the LNPs with the aqueous phase containing the nucleic acid molecule using microfluidic flow to form the LNPs;
(c) dialyzing the LNPs against a buffer to remove the organic solvent;
(d) concentrating the LNPs to a target volume; and
(e) optionally, filtered through a sterile filter.

In some embodiments, the organic lipid phase and the aqueous phase are mixed at a ratio of between 1:1 (v:v) and 1:10 (v:v). In some embodiments, the organic lipid phase and the aqueous phase are mixed at a ratio of 1:1 (v:v), 1:2 (v:v), 1:3 (v:v), 1:4 (v:v), 1:5 (v:v), 1:6 (v:v), 1:7 (v:v), 1:8 (v:v), 1:9 (v:v), or 1:10 (v:v). In some embodiments, the organic lipid phase and the aqueous phase are mixed at a ratio of between 1:1 (v:v) and 1:3 (v:v), between 1:2 (v:v) and 1:4 (v:v), between 1:3 (v:v) and 1:5 (v:v), between 1:4 (v:v) and 1:6 (v:v), between 1:5 (v:v) and 1:7 (v:v), between 1:6 (v:v) and 1:8 (v:v), between 1:7 (v:v) and 1:9 (v:v), or between 1:8 (v:v) and 1:10 (v:v). In some embodiments, the organic lipid phase and the aqueous phase are mixed at a ratio of between 1:3 (v:v) and 1:5 (v:v). In some embodiments, the organic lipid phase and the aqueous phase are mixed at a ratio of 1:3 (v:v). In some embodiments, the organic lipid phase and the aqueous phase are mixed at a ratio of 1:5 (v:v).

In some embodiments, the total flow rate of the microfluidic flow is 5-20 mL/min. In some embodiments, the total flow rate of the microfluidic flow is 5, 6, 7, 8, 9, 10, 11, 12, 13, 14, 15, 16, 17, 18, 19, or 20 mL/min. In some embodiments, the total flow rate of the microfluidic flow is 9-20 mL/min. In some embodiments, the total flow rate of the microfluidic flow is 11-13 mL/min.

In some embodiments, the solvent in the organic lipid phase in step (b) is ethanol. In some embodiments, heat is applied to the organic lipid phase in step (b). In some embodiments, about 40, 45, 50, 55, 60, 65, 70, 75, or 80° C. is applied to the organic lipid phase in step (b). In some embodiments, 60° C. heat is applied to the organic lipid phase in step (b). In some embodiments, no heat is applied to the organic lipid phase in step (b).

In some embodiments, the aqueous solution in step (a) has a pH of between 1 and 7. In some embodiments, the aqueous solution in step (a) has a pH of between 1 and 3, between 2 and 4, between 3 and 5, between 4 and 6, or between 5 and 7. In some embodiments, the aqueous solution in step (a) has a pH of 1, 1.5, 2, 2.5, 3, 3.5, 4, 4.5, 5, 5.5, 6, 6.5, or 7. In some embodiments, the aqueous solution in step (a) has a pH of 3. In some embodiments, the aqueous solution in step (a) has a pH of 5.

In some embodiments, the total lipid concentration is between 5 mM and 80 mM. In some embodiments, the total lipid concentration is about 5, 10, 15, 20, 25, 30, 35, 40, 45, 50, 55, 60, 65, 70, 75, or 80 mM. In some embodiments, the total lipid concentration is about 20 mM. In some embodiments, the total lipid concentration is about 40 mM.

In some embodiments, the LNP generated by the method has a lipid-nitrogen-to-phosphate ratio (N:P) of between 1 to 25. In some embodiments, the N:P is 1, 2, 3, 4, 5, 6, 7, 8, 9, 10, 11, 12, 13, 14, 15, 16, 17, 18, 19, 20, 21, 22, 23, 24, or 25. In some embodiments, the N:P is between 1 to 25, between 1 to 20, between 1 to 15, between 1 to 10, between 1 to 5, between 5 to 25, between 5 to 20, between 5 to 15, between 5 to 10, between 10 to 25, between 10 to 20, between 10 to 15, between 15 to 25, between 15 to 20, or between 20 to 25. In some embodiments, the LNP comprises a nucleic acid molecule and has a lipid-nitrogen-to-phosphate ratio (N:P) of 14.

In some embodiments, the buffer in step (c) has a neutral pH (e.g., 1×PBS, pH 7.2). In some embodiments, step (d) uses centrifugal filtration for concentrating.

In some embodiments, the encapsulation efficiency of the method of the disclosure is at least 70%, at least 75%, at least 75%, at least 80%, at least 90%, at least 95%, at least 97%, at least 98%, or at least 99%. In some embodiments, the encapsulation efficiency of the method of the disclosure is at least 90%. In some embodiments, the encapsulation efficiency of the method of the disclosure is at least 95%. In some embodiments, the encapsulation efficiency is determined by RiboGreen.

In some embodiments, the LNPs produced by the method of the disclosure have an average size (i.e., average outer diameter) of about 50 nm to about 500 nm. In some embodiments, the LNPs have an average size of about 50 nm to about 200 nm, about 100 nm to about 200 nm, about 150 nm to about 200 nm, about 50 nm to about 100 nm, about 50 nm to about 150 nm, about 100 nm to about 150 nm, about 200 nm to about 250 nm, about 250 nm to about 300 nm, about 300 nm to about 400 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average size of about 50 nm, 60 nm, 70 nm, 80 nm, 90 nm, 100 nm, 110 nm, about 120, or about 125 nm. In some embodiments, the plurality of LNPs have an average size of about 100 nm. In some embodiments, the plurality of LNPs have an average size of 50 nm to 150 nm. In some embodiments, the plurality of LNPs have an average size (average outer diameter) of 50 nm to 150 nm, 50 nm to 125 nm, 50 nm to 100 nm, 50 nm to 75 nm, 75 nm to 150 nm, 75 nm to 125 nm, 75 nm to 100 nm, 100 nm to 150 nm, 100 nm to 125 nm, or 125 nm to 150 nm. In some embodiments, the plurality of LNPs have an average size of 70 nm to 90 nm, 80 nm to 100 nm, 90 nm to 110 nm, 100 nm to 120 nm, 110 nm to 130 nm, 120 nm to 140 nm, or 130 nm to 150 nm. In some embodiments, the plurality of LNPs have an average size of 90 nm to 110 nm.

In some embodiments, the polydispersity index of the plurality of LNPs is between 0.01 and 0.3. In some embodiments, the polydispersity index of the plurality of LNPs is between 0.1 and 0.15. In some embodiments, the polydispersity index of the plurality of LNPs is about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 016, about 0.17, about 0.18, about 0.19, about 0.20, about 0.21, about 0.22, about 0.23, about 0.24, about 0.25, about 0.26, about 0.27, about 0.28, about 0.29, or about 0.30. In some embodiments, the polydispersity index of the plurality of LNPs is about 0.10, about 0.11, about 0.12, about 0.13, about 0.14, or about 0.15. In some embodiments, the average diameter and/or the polydispersity is determined via dynamic light scattering.

### Payload Molecules

In some embodiments, the particles comprise a synthetic RNA viral genome and further comprise a recombinant RNA polynucleotide encoding a payload molecule. In some embodiments, the particles are lipid nanoparticles and comprise a synthetic RNA viral genome and further comprise a recombinant RNA polynucleotide encoding a payload molecule. In some embodiments, the synthetic RNA viral genome in the particle (e.g., LNP) comprises the recombinant RNA polynucleotide encoding the payload molecule. In some embodiments, the particle (e.g., LNP) comprises 1) the synthetic RNA viral genome (which may or may not encode a payload molecule) and 2) a second recombinant RNA polynucleotide encoding a payload molecule. In some embodiments, the synthetic RNA viral genome and the second recombinant RNA polynucleotide encoding the payload molecule are not linked in the particle (e.g., LNP). In some embodiments, the synthetic RNA viral genome and the second recombinant RNA polynucleotide encoding the payload molecule are non-covalently linked. In some embodiments, the synthetic RNA viral genome and the second recombinant RNA polynucleotide encoding the payload molecule are covalently linked via a covalent bond other than a regular 3′, 5′ phosphodiester linkage. In some embodiments, one or more miRNA target sequences are incorporated into the 3′ or 5′ UTR of the RNA polynucleotide encoding the payload molecule. In some embodiments, one or more miRNA target sequences are inserted into the polynucleotide encoding the payload molecule. In such embodiments, translation and subsequent expression of the payload does not occur, or is substantially reduced, in cells where the corresponding miRNA is expressed. In some embodiments, the recombinant RNA polynucleotide encoding a payload molecule is a replicon.

In some embodiments, the payload is a cytotoxic peptide. As used herein, a “cytotoxic peptide” refers to a protein capable of inducing cell death when expressed in a host cell and/or cell death of a neighboring cell when secreted by the host cell. In some embodiments, the cytotoxic peptide is a caspase, p53, diphtheria toxin (DT), Pseudomonas Exotoxin A (PEA), Type I ribozyme inactivating proteins (RIPs) (e.g., saporin and gelonin), Type II RIPs (e.g., ricin), Shiga-like toxin 1 (Slt1), photosensitive reactive oxygen species (e.g. killer-red). In certain embodiments, the cytotoxic peptide is encoded by a suicide gene resulting in cell death through apoptosis, such as a caspase gene.

In some embodiments, the payload is an immune modulatory peptide. As used herein, an “immune modulatory peptide” is a peptide capable of modulating (e.g., activating or inhibiting) a particular immune receptor and/or pathway. In some embodiments, the immune modulatory peptides can act on any mammalian cell including immune cells, tissue cells, and stromal cells. In a preferred embodiment, the immune modulatory peptide acts on an immune cell such as a T cell, an NK cell, an NKT T cell, a B cell, a dendritic cell, a macrophage, a basophil, a mast cell, or an eosinophil. Exemplary immune modulatory peptides include antigen-binding molecules such as antibodies or antigen binding fragments thereof, cytokines, chemokines, soluble receptors, cell-surface receptor ligands, bipartite peptides, and enzymes.

In some embodiments, the payload is a cytokine such as IL-1, IL-12, IL-15, IL-18, IL-36γ, TNFα, IFNα, IFNβ, IFNγ, or TNFSF14. In some embodiments, the payload is a chemokine such as CXCL10, CXCL9, CCL21, CCL4, or CCL5. In some embodiments, the payload is a ligand for a cell-surface receptor such as an NKG2D ligand, a neuropilin ligand, Flt3 ligand, a CD47 ligand (e.g., SIRP1α). In some embodiments, the payload is a soluble receptor, such as a soluble cytokine receptor (e.g., IL-13R, TGFβR1, TGFβR2, IL-35R, IL-15R, IL-2R, IL-12R, and interferon receptors) or a soluble innate immune receptor (e.g., Toll-like receptors, complement receptors, etc.). In some embodiments, the payload is a dominant agonist mutant of a protein involved in intracellular RNA and/or DNA sensing (e.g. a dominant agonist mutant of STING, RIG-1, or MDA-5).

In some embodiments, the payload is an antigen-binding molecule such as an antibody or antigen-binding fragments thereof (e.g., a single chain variable fragment (scFv), an F(ab), etc.). In some embodiments, the antigen-binding molecule specifically binds to a cell surface receptor, such as an immune checkpoint receptor (e.g., PD-1, PD-L1, and CTLA4) or additional cell surface receptors involved in cell growth and activation (e.g., OX40, CD200R, CD47, CSFIR, TREM2, 4-1BB, CD40, and NKG2D).

In some embodiments, the payload molecule is a scorpion polypeptide such as chlorotoxin, BmKn-2, neopladine 1, neopladine 2, and mauriporin. In some embodiments, the payload molecule is a snake polypeptide such as contortrostatin, apoxin-I, bothropstoxin-I, BJcuL, OHAP-1, rhodostomin, drCT-I, CTX-III, BIL, and ACTX-6. In some embodiments, the payload molecule is a spider polypeptide such as a latarcin and hyaluronidase. In some embodiments, the payload molecule is a bee polypeptide such as melittin and apamin. In some embodiments, the payload molecule is a frog polypeptide such as PsT-1, PdT-1, and PdT-2.

In some embodiments, the payload molecule is an enzyme. In some embodiments, the enzyme is capable of modulating the tumor microenvironment by way of altering the extracellular matrix. In such embodiments, the enzyme may include, but is not limited to, a matrix metalloprotease (e.g., MMP9), a collagenase, a hyaluronidase, a gelatinase, or an elastase. In some embodiments, the enzyme is part of a gene directed enzyme prodrug therapy (GDEPT) system, such as herpes simplex virus thymidine kinase, cytosine deaminase, nitroreductase, carboxypeptidase G2, purine nucleoside phosphorylase, or cytochrome P450. In some embodiments, the enzyme is capable of inducing or activating cell death pathways in the target cell (e.g., a caspase). In some embodiments, the enzyme is capable of degrading an extracellular metabolite or message (e.g. adenosine deaminase or arginase or 15-Hydroxyprostaglandin Dehydrogenase).

In some embodiments, the payload molecule is MLKL. In some embodiments, the MLKL polypeptide comprises or consists of an amino acid sequence having at least 70%, at least 75%, at least 80%, at least 85%, at least 90%, at least 95%, at least 97%, at least 98%, at least 99%, or 100% identity to SEQ ID NO: 104. In some embodiments, the payload molecule comprises or consists of a MLKL 4HB domain. In some embodiments, the MLKL 4HB domain comprises or consists of an amino acid sequence having at least 70%, at least 75%, at least 80%, at least 85%, at least 90%, at least 95%, at least 97%, at least 98%, at least 99%, or 100% identity to amino acids 1-120 of SEQ ID NO: 104.

In some embodiments, the payload molecule is a Gasdermin D (GSDMD). In some embodiments, the Gasdermin D (GSDMD) comprises or consists of an amino acid sequence having at least 70%, at least 75%, at least 80%, at least 85%, at least 90%, at least 95%, at least 97%, at least 98%, at least 99%, or 100% identity to SEQ ID NO: 105. In some embodiments, the payload molecule comprises or consists of a Gasdermin D N-terminal fragment. In some embodiments, the Gasdermin D N-terminal fragment comprises or consists of an amino acid sequence having at least 70%, at least 75%, at least 80%, at least 85%, at least 90%, at least 95%, at least 97%, at least 98%, at least 99%, or 100% identity to amino acids 1-233 of SEQ ID NO: 105. In some embodiments, the payload molecule comprises a mutation corresponding to L192A of SEQ ID NO: 105.

In some embodiments, the payload molecule is a Gasdermin E (GSDME). In some embodiments, the Gasdermin E (GSDME) comprises or consists of an amino acid sequence having at least 70%, at least 75%, at least 80%, at least 85%, at least 90%, at least 95%, at least 97%, at least 98%, at least 99%, or 100% identity to SEQ ID NO: 106. In some embodiments, the payload molecule comprises or consists of a Gasdermin E N-terminal fragment. In some embodiments, the Gasdermin E N-terminal fragment comprises or consists of an amino acid sequence having at least 70%, at least 75%, at least 80%, at least 85%, at least 90%, at least 95%, at least 97%, at least 98%, at least 99%, or 100% identity to amino acids 1-237 of SEQ ID NO: 106.

In some embodiments, the payload molecule is a HMGB1. In some embodiments, the HMGB1 polypeptide comprises or consists of an amino acid sequence having at least 70%, at least 75%, at least 80%, at least 85%, at least 90%, at least 95%, at least 97%, at least 98%, at least 99%, or 100% identity to SEQ ID NO: 107. In some embodiments, the payload molecule comprises or consists of a HMGB1 Box B domain. In some embodiments, the HMGB1 Box B domain comprises or consists of an amino acid sequence having at least 70%, at least 75%, at least 80%, at least 85%, at least 90%, at least 95%, at least 97%, at least 98%, at least 99%, or 100% identity to amino acids 96-162 of SEQ ID NO: 107.

In some embodiments, the payload molecule is a SMAC/Diablo. In some embodiments, the SMAC/Diablo comprises or consists of an amino acid sequence having at least 70%, at least 75%, at least 80%, at least 85%, at least 90%, at least 95%, at least 97%, at least 98%, at least 99%, or 100% identity to SEQ ID NO: 108. In some embodiments, the payload molecule comprises or consists of an amino acid sequence having at least 70%, at least 75%, at least 80%, at least 85%, at least 90%, at least 95%, at least 97%, at least 98%, at least 99%, or 100% identity to amino acids 56-239 of SEQ ID NO: 108.

In some embodiments, the payload molecule is a Melittin. In some embodiments, the Melittin comprises or consists of an amino acid sequence having at least 70%, at least 75%, at least 80%, at least 85%, at least 90%, at least 95%, at least 97%, at least 98%, at least 99%, or 100% identity to SEQ ID NO: 109.

In some embodiments, the payload molecule is a L-amino-acid oxidase (LAAO). In some embodiments, the L-amino-acid oxidase (LAAO) comprises or consists of an amino acid sequence having at least 70%, at least 75%, at least 80%, at least 85%, at least 90%, at least 95%, at least 97%, at least 98%, at least 99%, or 100% identity to SEQ ID NO: 110.

In some embodiments, the payload molecule is a disintegrin. In some embodiments, the disintegrin comprises or consists of an amino acid sequence having at least 70%, at least 75%, at least 80%, at least 85%, at least 90%, at least 95%, at least 97%, at least 98%, at least 99%, or 100% identity to SEQ ID NO: 111.

In some embodiments, the payload molecule is a TRAIL (TNFSF10). In some embodiments, the TRAIL (TNFSF10) comprises or consists of an amino acid sequence having at least 70%, at least 75%, at least 80%, at least 85%, at least 90%, at least 95%, at least 97%, at least 98%, at least 99%, or 100% identity to SEQ ID NO: 112.

In some embodiments, the payload molecule is a nitroreductase. In some embodiments, the nitroreductase is NfsB (e.g., from E. coli). In some embodiments, the NfsB comprises or consists of an amino acid sequence having at least 70%, at least 75%, at least 80%, at least 85%, at least 90%, at least 95%, at least 97%, at least 98%, at least 99%, or 100% identity to SEQ ID NO: 113. In some embodiments, the nitroreductase is NfsA (e.g., from E. coli). In some embodiments, the NfsA comprises or consists of an amino acid sequence having at least 70%, at least 75%, at least 80%, at least 85%, at least 90%, at least 95%, at least 97%, at least 98%, at least 99%, or 100% identity to SEQ ID NO: 114.

In some embodiments, the payload molecule is a reovirus FAST protein. In some embodiments, the reovirus FAST protein is an ARV p14, a BRV p15, or a p14-p15 hybrid. In some embodiments, the payload molecule is an ARV p14. In some embodiments, the ARV p14 comprises or consists of an amino acid sequence having at least 70%, at least 75%, at least 80%, at least 85%, at least 90%, at least 95%, at least 97%, at least 98%, at least 99%, or 100% identity to SEQ ID NO: 115. In some embodiments, the payload molecule is a BRV p15. In some embodiments, the BRV p15 comprises or consists of an amino acid sequence having at least 70%, at least 75%, at least 80%, at least 85%, at least 90%, at least 95%, at least 97%, at least 98%, at least 99%, or 100% identity to SEQ ID NO: 116. In some embodiments, the payload molecule is a p14-p15 hybrid. In some embodiments, the p14-p15 hybrid comprises or consists of an amino acid sequence having at least 70%, at least 75%, at least 80%, at least 85%, at least 90%, at least 95%, at least 97%, at least 98%, at least 99%, or 100% identity to SEQ ID NO: 117.

In some embodiments, the payload molecule is a Leptin/FOSL2. In some embodiments, the Leptin/FOSL2 comprises or consists of an amino acid sequence having at least 70%, at least 75%, at least 80%, at least 85%, at least 90%, at least 95%, at least 97%, at least 98%, at least 99%, or 100% identity to SEQ ID NO: 118.

In some embodiments, the payload molecule is an adenosine deaminase 2 (ADA2). In some embodiments, the adenosine deaminase (ADA2) comprises or consists of an amino acid sequence having at least 70%, at least 75%, at least 80%, at least 85%, at least 90%, at least 95%, at least 97%, at least 98%, at least 99%, or 100% identity to SEQ ID NO: 119.

In some embodiments, the payload molecule is an α-1,3-galactosyltransferase. In some embodiments, the α-1,3-galactosyltransferase comprises or consists of an amino acid sequence having at least 70%, at least 75%, at least 80%, at least 85%, at least 90%, at least 95%, at least 97%, at least 98%, at least 99%, or 100% identity to SEQ ID NO: 120.

In some embodiments, the payload molecule is IL-2. In some embodiments, the IL-2 comprises or consists of an amino acid sequence having at least 70%, at least 75%, at least 80%, at least 85%, at least 90%, at least 95%, at least 97%, at least 98%, at least 99%, or 100% identity to SEQ ID NO: 121.

In some embodiments, the payload molecule is IL-7. In some embodiments, the IL-7 comprises or consists of an amino acid sequence having at least 70%, at least 75%, at least 80%, at least 85%, at least 90%, at least 95%, at least 97%, at least 98%, at least 99%, or 100% identity to SEQ ID NO: 122.

In some embodiments, the payload molecule is IL12. In some embodiments, the payload molecule comprises an IL-12 beta subunit comprising or consisting of an amino acid sequence having at least 70%, at least 75%, at least 80%, at least 85%, at least 90%, at least 95%, at least 97%, at least 98%, at least 99%, or 100% identity to SEQ ID NO: 123. In some embodiments, the payload molecule comprises an IL-12 alpha subunit comprising or consisting of an amino acid sequence having at least 70%, at least 75%, at least 80%, at least 85%, at least 90%, at least 95%, at least 97%, at least 98%, at least 99%, or 100% identity to SEQ ID NO: 124.

In some embodiments, the payload molecule is IL18. In some embodiments, the IL18 comprises or consists of an amino acid sequence having at least 70%, at least 75%, at least 80%, at least 85%, at least 90%, at least 95%, at least 97%, at least 98%, at least 99%, or 100% identity to SEQ ID NO: 125.

In some embodiments, the payload molecule is IL-21. In some embodiments, the IL-21 comprises or consists of an amino acid sequence having at least 70%, at least 75%, at least 80%, at least 85%, at least 90%, at least 95%, at least 97%, at least 98%, at least 99%, or 100% identity to SEQ ID NO: 126.

In some embodiments, the payload molecule is IL-36γ. In some embodiments, the IL-36γ comprises or consists of an amino acid sequence having at least 70%, at least 75%, at least 80%, at least 85%, at least 90%, at least 95%, at least 97%, at least 98%, at least 99%, or 100% identity to SEQ ID NO: 127.

In some embodiments, the payload molecule is IFNγ. In some embodiments, the IFNγ comprises or consists of an amino acid sequence having at least 70%, at least 75%, at least 80%, at least 85%, at least 90%, at least 95%, at least 97%, at least 98%, at least 99%, or 100% identity to SEQ ID NO: 128.

In some embodiments, the payload molecule is CCL21. In some embodiments, the CCL21 comprises or consists of an amino acid sequence having at least 70%, at least 75%, at least 80%, at least 85%, at least 90%, at least 95%, at least 97%, at least 98%, at least 99%, or 100% identity to SEQ ID NO: 129.

In some embodiments, the payload molecule is encoded by a polynucleotide molecule according to one of the embodiments provided in Table 20 below.

TABLE 20
Non-limiting Embodiments of Payload Configurations

| | | LNP comprising | | |
|---|---|---|---|---|
| | | a CVA (e.g., | | LNP comprising |
| | | CVA21) viral | | a SVV viral |
| | CVA (e.g., | genome and a | | genome and a |
| | CVA21) viral | second RNA | SVV viral | second RNA |
| | genome | molecule | genome | molecule |
| | encoding the | encoding the | encoding the | encoding the |
| Payload | payload | payload | payload | payload |
| MLKL | x | x | x | x |
| MLKL 4HB domain | x | x | x | x |
| Gasdermin D | x | x | x | x |
| Gasdermin D with | x | x | x | x |
| L192A | | | | |
| Gasdermin D N- | x | x | x | x |
| terminal fragment | | | | |
| Gasdermin D N- | x | x | x | x |
| terminal fragment | | | | |
| with L192A | | | | |
| Gasdermin E | x | x | x | x |
| Gasdermin E N- | x | x | x | x |
| terminal fragment | | | | |
| HMGB1 | x | x | x | x |
| HMGB1 Box B | x | x | x | x |
| domain | | | | |
| SMAC/Diablo | x | x | x | x |
| Melittin | x | x | x | x |
| L-amino-acid | x | x | x | x |
| oxidase (LAAO) | | | | |
| disintegrin | x | x | x | x |
| TRAIL (TNFSF10) | x | x | x | x |
| nitroreductase | x | x | x | x |
| NfsB | x | x | x | x |
| NfsA | x | x | x | x |
| reovirus FAST | x | x | x | x |
| protein | | | | |
| ARV p14 | x | x | x | x |
| BRV p15 | x | x | x | x |
| p14-p15 hybrid | x | x | x | x |
| Leptin/FOSL2 | x | x | x | x |
| Adenosine | x | x | x | x |
| deaminase 2 | | | | |
| (ADA2) | | | | |
| alpha-1,3- | x | x | x | x |
| galactosyltransferase | | | | |
| IL-2 | x | x | x | x |
| IL-7 | x | x | x | x |
| IL-12 | x | x | x | x |
| IL-12 beta subunit | x | x | x | x |
| IL-12 alpha subunit | x | x | x | x |
| IL-18 | x | x | x | x |
| IL-21 | x | x | x | x |
| IL-36 gamma | x | x | x | x |
| IFN gamma | x | x | x | x |
| CCL21 | x | x | x | x |

*Each ‘x’ indicates an embodiment

In some embodiments, the payload molecule is a bipartite peptide. As used herein, a “bipartite peptide” refers to a multimeric protein comprised of a first domain capable of binding a cell surface antigen expressed on a non-cancerous effector cell and a second domain capable of binding a cell-surface antigen expressed by a target cell (e.g., a cancerous cell, a tumor cell, or an effector cell of a different type). In some embodiments, the individual polypeptide domains of a bipartite polypeptide may comprise an antibody or binding fragment thereof (e.g, a single chain variable fragment (scFv) or an F(ab)), a nanobody, a diabody, a flexibody, a DOCK-AND-LOCK™ antibody, or a monoclonal anti-idiotypic antibody (mAb2). In some embodiments, the structure of the bipartite polypeptides may be a dual-variable domain antibody (DVD-Ig™), a Tandab®, a bi-specific T cell engager (BiTE™), a DuoBody®, or a dual affinity retargeting (DART) polypeptide. In some embodiments, the bipartite polypeptide is a BiTE and comprises a domain that specifically binds to an antigen shown in Table 8 and/or 9. Exemplary BiTEs are shown below in Table 7.

TABLE 7
Validated BiTEs used in preclinical and clinical studies

| Target | Name | Target Disease | Clinical Status | References |
|---|---|---|---|---|
| CD19 | Blinatumomab/MT- | NHL, ALL | Phase I/II/III | 1, 2, 3, 4, 5, 6 |
| | 103/MEDI-538 | | | |
| EpCAM | MT110 | Solid tumors | Phase I | 7, 8, 9, 10 |
| CEA | MT111/MEDI-565 | GI adenocarcinoma | Phase I | 11, 12 |
| PSMA | BAY2010112/AMG112 | Prostate | Phase I | 13 |
| CD33 | AMG330 | AML | Preclinical | 14, 15 |
| EGFR | C-BiTE and P-BiTE | Colorectal cancer | Preclinical | 16 |
| | antibodies | | | |
| Her2 | FynomAb, | Breast and gastric | Preclinical | 17, 18 |
| | COVA420, HER2- | carcinoma | | |
| | BsAb | | | |
| EphA2 | bscEphA2xCD3 | Multiple solid | Preclinical | 19 |
| | | tumors | | |
| MCSP | MCSP-BITE | Melanoma | Preclinical | 20 |
| ADAM17 | A300E | Prostate cancer | Preclinical | 21 |
| PSCA | CD3-PSCA(MB1) | Prostate cancer | Preclinical | 22 |
| 17-A1 | CD3/17-1A-bispecific | Colorectal cancer | Preclinical | 23 |
| NKG2D | scFv-NKG2D, | Multiple solid and | Preclinical | 24, 25 |
| ligands | huNKG2D-OKT3 | liquid tumors | | |
| DLL3 | AMG757 | Small Cell Lung | Clinical | 26 |
| | | Cancer | | |

In some embodiments, the cell-surface antigen expressed on an effector cell is selected from Table 8 below. In some embodiments, the cell-surface antigen expressed on a tumor cell or effector cell is selected from Table 9 below. In some embodiments, the cell-surface antigen expressed on a tumor cell is a tumor antigen. In some embodiments, the tumor antigen is selected from CD19, EpCAM, CEA, PSMA, CD33, EGFR, Her2, EphA2, MCSP, ADAM17, PSCA, 17-A1, an NKGD2 ligand, CSFIR, FAP, GD2, DLL3, TROP2, Nectin 4, or neuropilin. In other embodiments, the antigen is a viral antigen associated with the development of cancer. In some embodiments, the viral antigen associated with the development of cancer is HBV-core (Hepatitis B core antigen), HBV-pol, HbS-Ag, HPV E6, HPV E7, Merkel cell polyoma large T antigen, or Epstein Barr virus antigen EBNA2 or BZLF1. In some embodiments, the tumor antigen is selected from those listed in Table 9.

TABLE 8
Exemplary effector cell target antigens

| T cell | | NKT cell | NK Cell | Other |
|---|---|---|---|---|
| CD3 | CD30 | CD3 | CD16 | CD48 |
| CD3γ | CD38 | CD3γ | CD94/NKG2 | LIGHT |
| | | | (e.g., NKG2D) | |
| CD3δ | CD40 | CD3δ | NKp30 | CD44 |
| CD3ε | CD57 | CD3ε | NKp44 | CD45 |
| CD3ξ | CD69 | CD3ξ | NKp46 | IL-1R2 |
| CD2 | CD70 | invariant TCR | KARs | IL-1Rα |
| CD4 | CD73 | | | IL-1Rα2 |
| CD5 | CD81 | | | IL-13Rα2 |
| CD6 | CD82 | | | IL-15Ra |
| CD7 | CD96 | | | CCR5 |
| CD8 | CD134 | | | CCR8 |
| CD16 | CD137 | | | |
| CD25 | CD152 | | | |
| CD27 | CD278 | | | |
| CD28 | | | | |

TABLE 9
Exemplary target cell antigens
Target Cell Antigens

| | | | |
|---|---|---|---|
| 8H9 | CRISP3 | Lewis-Y | Fas |
| GnT-V, β1,6-N | DC-SIGN | LIV-1 (SLC39A6) | SOX2 |
| AFP | DHFR | Livin | STEAP1 |
| ART1 | EGP40 | LAMP1 | SLITRK6 |
| ART4 | EZH2 | MAGEA3 | NaPi2a |
| ABCG2 | EpCAM | MAGEA4 | SOX1 |
| B7-H3 | EphA2 | MAGEB6 | SOX11 |
| B7-H4 | EphA2/Eck | MAGEA1 | SPANXA1 |
| B7-H6 | EGFRvIII | MART-1 | SART-1 |
| BCMA | E-cadherin | MCSP | SSX4 |
| B-cyclin | EGP2 | MME | SSX5 |
| BMI1 | ETA | mesothelin (MSLN) | Survivin |
| CA-125 | ERBB3 | MAPK1 | SSX2 |
| cadherin | ERBB3/4 | MUC16 | TAG72 |
| CABYR | ERBB4 | MUC1 | TEM1 |
| CTAG2 | EPO | MRP-3 | TEM8 |
| CA6 | F3 | MyoD-1 | TSGA10 |
| CAIX | FAR | NCAM | TSSK6 |
| CEA | FBP | nectin 4 | thyroglobulin |
| CEACAM5 | FTHL17 | Nestin | transferrin receptor |
| CEACAM6 | fetal AchR | NEP | TACSTD2 (TROP2) |
| Cav-1 | FAP | NY-ESO-1 | TMEM97 |
| CD10 | FGFR3 | hHLA-A | TRP-2 |
| CD117 | FR-a | H60 | TULP2 |
| CD123 | Fra-l/Fosl 1 | OLIG2 | TROP2 |
| CD133 | GAGE1 | 5T4 | tyrosinase |
| CD138 | GD2 | p53 | TRP1 |
| CD15 | GD3 | P-Cadherin | UPAR |
| CD171 | Glil | PB | VEGF |
| CD19 | GP100 | P-glycoprotein | VEGF receptors |
| CD20 | GPA33 | PMCT (SLC13A5) | VEGRR2 |
| CD21 | | PRAME | BRAF |
| CD22 | Glypican-3 | PROXl | WT-1 |
| CD30 | HIV gp120 | PSA | XAGE2 |
| CD33 | HLA-A | PSCA | ZNF165 |
| CD37 | HLA-A2 | PSMA | α_{ν}β₆ integrin |
| CD38 | HLA-AI | PSC1 | β-catenin |
| CD44v6 | HLA-B | PVRL4 | cathepsin B |
| CD44v7/8 | HLA-C | Ras | CSAG2 |
| CD74 | HMW-MAA | ROR1 | CTAG2 |
| Cd79b | Her2/Neu | SART2 | EGFR |
| CD124 (IL-4R) | Her3 | SART3 | EGP40 |
| CDH3 | u70/80 | oncofetal variants | EZH2 |
| | | of fibronectin | |
| Ki-67 | LICAM | tenascin | HIV sp120 |
| CSPG4 | ULBP1 | LICAM | kappa light chain |
| CALLA | ULBP2 | Rae-1α | LDHC |
| CSAG2 | ULBP3 | Rae-1β | TRP-1 |
| COX-2 | ULBP6 | Rae-1δ | Fas-L |
| Lambda | MICA | Rae-1γ | DLL3 |
| LAYN | MICB | PDGF | |
| LeuM-1 | Her3 | TROP2 | |
| KDR | EGF | | |
| CD47 | SIRP1α | | |

### Pharmaceutical Compositions and Methods of Use

One aspect of the disclosure relates to pharmaceutical compositions comprising the recombinant RNA molecules described herein, or particles comprising a recombinant RNA molecule described herein, and methods for the treatment of cancer. In some embodiments, the present disclosure provides methods of treating cancer in a subject in need thereof comprising administering an effective amount of a CVA21-EF, a CVA21-KY, or an SVV virus or the corresponding RNA viral genome to the subject. Compositions described herein can be formulated in any manner suitable for a desired delivery route. Typically, formulations include all physiologically acceptable compositions including derivatives or prodrugs, solvates, stereoisomers, racemates, or tautomers thereof with any pharmaceutically acceptable carriers, diluents, and/or excipients.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, and caffeine.

The present disclosure provides methods of killing a cancerous cell or a target cell comprising exposing the cell to an RNA polynucleotide or particle described herein, or composition thereof, under conditions sufficient for the intracellular delivery of the composition to the cancerous cell. As used herein, a “cancerous cell” or a “target cell” refers to a mammalian cell selected for treatment or administration with a polynucleotide or particle described herein, or composition thereof described herein. As used herein “killing a cancerous cell” refer specifically to the death of a cancerous cell by means of apoptosis or necrosis. Killing of a cancerous cell may be determined by methods known in the art including but not limited to, tumor size measurements, cell counts, and flow cytometry for the detection of cell death markers such as Annexin V and incorporation of propidium iodide.

The present disclosure further provides for a method of treating or preventing cancer in a subject in need thereof wherein an effective amount of the pharmaceutical compositions described herein is administered to the subject. The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration. The encapsulated polynucleotide compositions described herein are particularly useful in the treatment of metastatic cancers, wherein systemic administration may be necessary to deliver the compositions to multiple organs and/or cell types. Therefore, in a particular embodiment, the compositions described herein are administered systemically.

An “effective amount” or an “effective dose,” used interchangeably herein, refers to an amount and or dose of the compositions described herein that results in an improvement or remediation of the symptoms of the disease or condition. The improvement is any improvement or remediation of the disease or condition, or symptom of the disease or condition. The improvement is an observable or measurable improvement or may be an improvement in the general feeling of well-being of the subject. Thus, one of skill in the art realizes that a treatment may improve the disease condition but may not be a complete cure for the disease. Improvements in subjects may include, but are not limited to, decreased tumor burden, decreased tumor cell proliferation, increased tumor cell death, activation of immune pathways, increased time to tumor progression, decreased cancer pain, increased survival, or improvements in the quality of life.

In some embodiments, administration of an effective dose may be achieved with administration a single dose of a composition described herein. As used herein, “dose” refers to the amount of a composition delivered at one time. In some embodiments, the dose of the recombinant RNA molecules is measured as the 50% Tissue culture Infective Dose (TCID₅₀). In some embodiments, the TCID₅₀ is at least about 10³-10⁹ TCID₅₀/mL, for example, at least about 10³ TCID₅₀/mL, about 10⁴ TCID₅₀/mL, about 10⁵ TCID₅₀/mL, about 10⁶ TCID₅₀/mL, about 10⁷ TCID₅₀/mL, about 10⁸ TCID₅₀/mL, or about 10⁹ TCID₅₀/mL. In some embodiments, a dose may be measured by the number of particles in a given volume (e.g., particles/mL). In some embodiments, a dose may be further refined by the genome copy number of the RNA polynucleotides described herein present in each particle (e.g., # of particles/mL, wherein each particle comprises at least one genome copy of the polynucleotide). In some embodiments, delivery of an effective dose may require administration of multiple doses of a composition described herein. As such, administration of an effective dose may require the administration of at least 2, 3, 4, 5, 6, 7, 8, 9, 10, 20, 30, 40, 50, or more doses of a composition described herein.

In embodiments wherein multiple doses of a composition described herein are administered, each dose need not be administered by the same actor and/or in the same geographical location. Further, the dosing may be administered according to a predetermined schedule. For example, the predetermined dosing schedule may comprise administering a dose of a composition described herein daily, every other day, weekly, bi-weekly, monthly, bi-monthly, annually, semi-annually, or the like. The predetermined dosing schedule may be adjusted as necessary for a given patient (e.g., the amount of the composition administered may be increased or decreased and/or the frequency of doses may be increased or decreased, and/or the total number of doses to be administered may be increased or decreased).

As used herein “prevention” or “prophylaxis” can mean complete prevention of the symptoms of a disease, a delay in onset of the symptoms of a disease, or a lessening in the severity of subsequently developed disease symptoms.

The term “subject” or “patient” as used herein, is taken to mean any mammalian subject to which a composition described herein is administered according to the methods described herein. In a specific embodiment, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish, and reptiles.

In some embodiments, the present disclosure provides a method of treating a cancer in a subject in need thereof, comprising administering a therapeutically effective amount of an oncolytic Coxsackievirus, wherein the Coxsackievirus is a CVA21 strain, or a polynucleotide encoding the CVA21 to the subject, wherein the cancer is classified as sensitive to CVA21 infection based on the expression level of ICAM-1 and/or the percentage of ICAM-1 positive cancer cells in the cancer. In some embodiments, the CVA21 strain is CVA21-KY.

Intracellular adhesion molecule 1 (ICAM-1, also known as BB2, CD54, P3.58) is a protein (UniProt Ref: P03562) encoded by the ICAMI gene (NCBI Gene ID: 3383) and is important in stabilizing cell-cell interactions and facilitating leukocyte endothelial transmigration. In some embodiments, treatment decisions for a particular cancer are made based on ICAM-1 expression, wherein the expression of ICAM-1 is determined in the cancer and the cancer is identified as sensitive or resistant to CVA21 expression based on the level of ICAM-1 expression. In general, higher (% of positive tumor cells or intensity or both) expression of ICAM-1 indicates greater sensitivity to CVA21 infection (See Example 8). ICAM-1 expression can be determined by means known in the art for mRNA and/or protein expression. mRNA expression can be determined by northern blots, ribonuclease protection assays, PCR-based methods, sequencing methods, and the like. Protein expression can be determined by immunoblotting (e.g., western blot), immunohistochemistry, immunofluorescence, enzyme-linked immunosorbent assay (ELISA), flow cytometry, cytometric bead array, mass spectroscopy, proteomics-based methods, and the like.

In some embodiments, the present disclosure provides a method of treating a cancer in a subject in need thereof, comprising: (a) determining the expression level of ICAM-1 and/or the percentage of ICAM-1 positive cancer cells in the cancer; (b) classifying the cancer as sensitive to Coxsackievirus 21 (CVA21) infection based on the expression level of ICAM-1 and/or the percentage of ICAM-1 positive cancer cells determined in (a); and (c) administering a therapeutically effective amount of CVA21 or a polynucleotide encoding the CVA21 to the subject if the cancer is classified as sensitive to CVA21 infection in step (b). In some embodiments, the CVA21 strain is CVA21-KY.

In some embodiments, the present disclosure provides a method of selecting a subject suffering from a cancer for treatment with a Coxsackievirus 21 (CVA21) or a polynucleotide encoding the CVA21, comprising: (a) determining the expression level of ICAM-1 and/or the percentage of ICAM-1 positive cancer cells in the cancer; (b) classifying the cancer as sensitive to CVA21 infection based on the expression level of ICAM-1 and/or the percentage of ICAM-1 positive cancer cells as determined in (a); (c) selecting the subject for treatment with the CVA21 or the polynucleotide encoding the CVA21 if the cancer is classified as sensitive to CVA21 infection in (b); and (d) administering the CVA21 or the polynucleotide encoding the CVA21 to the selected subject. In some embodiments, the CVA21 strain is CVA21-KY.

### Lipid Nanoparticle Composition and Methods of Use

In some embodiments, the disclosure provides methods of treating a disease or disorder in a subject in need thereof, comprising administering to the subject a therapeutically effective amount of a composition (e.g., pharmaceutical composition) of the disclosure. In some embodiments, the disease or disorder comprises a cancer. In some embodiments, the composition comprises a PEG-lipid of the disclosure. In some embodiments, the composition comprises an LNP of the disclosure comprising a PEG-lipid. In some embodiments, the composition comprises an LNP of the disclosure comprising a PEG-lipid and an encapsulated molecule of the disclosure (e.g., synthetic RNA viral genome).

The method may be a method of treating a subject having or at risk of having a condition that benefits from the encapsulated molecule, particularly if the encapsulated molecule is a therapeutic agent. Alternatively, the method may be a method of diagnosing a subject, in which case the encapsulated molecule may be is a diagnostic agent.

In prophylactic applications, pharmaceutical compositions comprising an LNP of the disclosure are administered to a subject susceptible to, or otherwise at risk of, a particular disorder in an amount sufficient to eliminate or reduce the risk or delay the onset of the disorder. In therapeutic applications, compositions comprising an LNP of the disclosure are administered to a subject suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, the pharmaceutical composition can be administered in several dosages until a sufficient response has been achieved. Typically, the response is monitored and repeated dosages are given if the desired response starts to fade.

For administration, the LNP of the disclosure may be formulated as a pharmaceutical composition. In some embodiments, the LNP comprises an encapsulated molecule. A pharmaceutical composition may comprise: (i) an LNP of the disclosure; and (ii) a pharmaceutically acceptable carrier, diluent or excipient. A pharmaceutical composition can be formulated according to known methods to prepare pharmaceutically useful compositions, whereby the therapeutic molecule is combined in a mixture with a pharmaceutically acceptable carrier, diluent, or excipient. A carrier is said to be a “pharmaceutically acceptable carrier” if its administration can be tolerated by a recipient subject. Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising LNPs of the disclosure may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition may be administered to a subject in a therapeutically effective amount. According to the methods of the disclosure, a composition can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, intratumoral, and oral routes of administration. For prevention and treatment purposes, a composition can be administered to a subject in a single bolus delivery, via continuous delivery (e.g., continuous transdermal delivery) over an extended time period, or in a repeated administration protocol (e.g., on an hourly, daily, weekly, or monthly basis).

Administration can occur by injection, irrigation, inhalation, consumption, electro-osmosis, hemodialysis, iontophoresis, and other methods known in the art. The route of administration will vary, naturally, with the location and nature of the disease being treated, and may include, for example auricular, buccal, conjunctival, cutaneous, dental, endocervical, endosinusial, endotracheal, enteral, epidural, interstitial, intra-articular, intra-arterial, intra-abdominal, intraauricular, intrabiliary, intrabronchial, intrabursal, intracavernous, intracerebral, intracisternal, intracorneal, intracronal, intracoronary, intracranial, intradermal, intradiscal, intraductal, intraduodenal, intraduodenal, intradural, intraepicardial, intraepidermal, intraesophageal, intragastric, intragingival, intrahepatic, ir intraileal, intralesional, intralingual, intraluminal, intralymphatic, intramammary, intramedulleray, intrameningeal, instramuscular, intranasal, intranodal, intraocular, intraomentum, intraovarian, intraperitoneal, intrapericardial, intrapleural, intraprostatic, intrapulmonary, intraruminal, intrasinal, intraspinal, intrasynovial, intratendinous, intratesticular, intratracheal, intrathecal, intrathoracic, intratubular, intratumoral, intratympanic, intrauterine, intraperitoneal, intravascular, intraventricular, intravesical, intravestibular, intravenous, intravitreal, larangeal, nasal, nasogastric, oral, ophthalmic, oropharyngeal, parenteral, percutaneous, periarticular, peridural, perineural, periodontal, respiratory, retrotubular, rectal, spinal, subarachnoid, subconjunctival, subcutaneous, subdermal, subgingival, sublingual, submucosal, subretinal, topical, transdermal, transendocardial, transmucosal, transplacental, trantracheal, transtympanic, ureteral, urethral, and/or vaginal perfusion, lavage, direct injection, and oral administration.

In some embodiments, the pharmaceutical composition is formulated for systemic administration. In some embodiments, the systemic administration comprises intravenous administration, intra-arterial administration, intraperitoneal administration, intramuscular administration, intradermal administration, subcutaneous administration, intranasal administration, oral administration, or a combination thereof. In some embodiments, the pharmaceutical composition is formulated for intravenous administration. In some embodiments, the pharmaceutical composition is formulated for local administration. In some embodiments, the pharmaceutical composition is formulated for intratumoral administration.

Effective doses of the compositions of the disclosure vary depending upon many different factors, including means of administration, target site, physiological state of the subject, whether the subject is human or an animal, other medications administered, whether treatment is prophylactic or therapeutic, as well as the specific activity of the composition itself and its ability to elicit the desired response in the individual. In some embodiments, the subject is a human. In some embodiments, the subject can be a nonhuman mammal. Typically, dosage regimens are adjusted to provide an optimum therapeutic response, i.e., to optimize safety and efficacy.

Determination of effective dosages in this context is typically based on animal model studies followed up by human clinical trials and is guided by determining effective dosages and administration protocols that significantly reduce the occurrence or severity of the subject disorder in model subjects. Compositions of the disclosure may be suitably administered to the subject at one time or over a series of treatments and may be administered to the subject at any time from diagnosis onwards. Compositions of the disclosure may be administered as the sole treatment, as a monotherapy, or in conjunction with other drugs or therapies, as a combinatorial therapy, useful in treating the condition in question.

In some embodiments, the therapeutically effective amount of a composition of the disclosure is between about 1 ng/kg body weight to about 100 mg/kg body weight. In some embodiments, the range of a composition of the disclosure administered is from about 1 ng/kg body weight to about 1 μg/kg body weight, about 1 ng/kg body weight to about 100 ng/kg body weight, about 1 ng/kg body weight to about 10 ng/kg body weight, about 10 ng/kg body weight to about 1 μg/kg body weight, about 10 ng/kg body weight to about 100 ng/kg body weight, about 100 ng/kg body weight to about 1 μg/kg body weight, about 100 ng/kg body weight to about 10 pg/kg body weight, about 1 μg/kg body weight to about 10 pg/kg body weight, about 1 μg/kg body weight to about 100 pg/kg body weight, about 10 pg/kg body weight to about 100 pg/kg body weight, about 10 pg/kg body weight to about 1 mg/kg body weight, about 100 μg/kg body weight to about 10 mg/kg body weight, about 1 mg/kg body weight to about 100 mg/kg body weight, or about 10 mg/kg body weight to about 100 mg/kg body weight. Dosages within this range can be achieved by single or multiple administrations, including, e.g., multiple administrations per day or daily, weekly, bi-weekly, or monthly administrations. Compositions of the disclosure may be administered, as appropriate or indicated, as a single dose by bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. In some embodiments, a composition of the disclosure is administered weekly. In some embodiments, a composition of the disclosure is administered biweekly. In some embodiments, a composition of the disclosure is administered every three weeks. However, other dosage regimens may be useful. The progress of this therapy is easily monitored by conventional techniques.

For administration to a human adult subject, the therapeutically effective amount may be administered in doses in the range of 0.0006 mg to 1000 mg per dose, including but not limited to 0.0006 mg per dose, 0.001 mg per dose, 0.003 mg per dose, 0.006 mg per dose, 0.01 mg per dose, 0.03 mg per dose, 0.06 mg per dose, 0.1 mg per dose, 0.3 mg per dose, 0.6 mg per dose, 1 mg per dose, 3 mg per dose, 6 mg per dose, 10 mg per dose, 30 mg per dose, 60 mg per dose, 100 mg per dose, 300 mg per dose, 600 mg per dose and 1000 mg per dose, and multiple, usually consecutive daily doses may be administered in a course of treatment. In some embodiments, a composition of the disclosure is administered at a dose level of about 0.001 mg/kg/dose to about 10 mg/kg/dose, about 0.001 mg/kg/dose to about 6 mg/kg/dose, about 0.001 mg/kg/dose to about 3 mg/kg/dose, about 0.001 mg/kg/dose to about 1 mg/kg/dose, about 0.001 mg/kg/dose to about 0.6 mg/kg/dose, about 0.001 mg/kg/dose to about 0.3 mg/kg/dose, about 0.001 mg/kg/dose to about 0.1 mg/kg/dose, about 0.001 mg/kg/dose to about 0.06 mg/kg/dose, about 0.001 mg/kg/dose to about 0.03 mg/kg/dose, about 0.001 mg/kg/dose to about 0.01 mg/kg/dose, about 0.001 mg/kg/dose to about 0.006 mg/kg/dose, about 0.001 mg/kg/dose to about 0.003 mg/kg/dose, about 0.003 mg/kg/dose to about 10 mg/kg/dose, about 0.003 mg/kg/dose to about 6 mg/kg/dose, about 0.003 mg/kg/dose to about 3 mg/kg/dose, about 0.003 mg/kg/dose to about 1 mg/kg/dose, about 0.003 mg/kg/dose to about 0.6 mg/kg/dose, about 0.003 mg/kg/dose to about 0.3 mg/kg/dose, about 0.003 mg/kg/dose to about 0.1 mg/kg/dose, about 0.003 mg/kg/dose to about 0.06 mg/kg/dose, about 0.003 mg/kg/dose to about 0.03 mg/kg/dose, about 0.003 mg/kg/dose to about 0.01 mg/kg/dose, about 0.003 mg/kg/dose to about 0.006 mg/kg/dose, about 0.006 mg/kg/dose to about 10 mg/kg/dose, about 0.006 mg/kg/dose to about 6 mg/kg/dose, about 0.006 mg/kg/dose to about 3 mg/kg/dose, about 0.006 mg/kg/dose to about 1 mg/kg/dose, about 0.006 mg/kg/dose to about 0.6 mg/kg/dose, about 0.006 mg/kg/dose to about 0.3 mg/kg/dose, about 0.006 mg/kg/dose to about 0.1 mg/kg/dose, about 0.006 mg/kg/dose to about 0.06 mg/kg/dose, about 0.006 mg/kg/dose to about 0.03 mg/kg/dose, about 0.006 mg/kg/dose to about 0.01 mg/kg/dose, about 0.01 mg/kg/dose to about 10 mg/kg/dose, about 0.01 mg/kg/dose to about 6 mg/kg/dose, about 0.01 mg/kg/dose to about 3 mg/kg/dose, about 0.01 mg/kg/dose to about 1 mg/kg/dose, about 0.01 mg/kg/dose to about 0.6 mg/kg/dose, about 0.01 mg/kg/dose to about 0.3 mg/kg/dose, about 0.01 mg/kg/dose to about 0.1 mg/kg/dose, about 0.01 mg/kg/dose to about 0.06 mg/kg/dose, about 0.01 mg/kg/dose to about 0.03 mg/kg/dose, about 0.03 mg/kg/dose to about 10 mg/kg/dose, about 0.03 mg/kg/dose to about 6 mg/kg/dose, about 0.03 mg/kg/dose to about 3 mg/kg/dose, about 0.03 mg/kg/dose to about 1 mg/kg/dose, about 0.03 mg/kg/dose to about 0.6 mg/kg/dose, about 0.03 mg/kg/dose to about 0.3 mg/kg/dose, about 0.03 mg/kg/dose to about 0.1 mg/kg/dose, about 0.03 mg/kg/dose to about 0.06 mg/kg/dose, about 0.06 mg/kg/dose to about 10 mg/kg/dose, about 0.06 mg/kg/dose to about 6 mg/kg/dose, about 0.06 mg/kg/dose to about 3 mg/kg/dose, about 0.06 mg/kg/dose to about 1 mg/kg/dose, about 0.06 mg/kg/dose to about 0.6 mg/kg/dose, about 0.06 mg/kg/dose to about 0.3 mg/kg/dose, about 0.06 mg/kg/dose to about 0.1 mg/kg/dose, about 0.1 mg/kg/dose to about 10 mg/kg/dose, about 0.1 mg/kg/dose to about 6 mg/kg/dose, about 0.1 mg/kg/dose to about 3 mg/kg/dose, about 0.1 mg/kg/dose to about 1 mg/kg/dose, about 0.1 mg/kg/dose to about 0.6 mg/kg/dose, about 0.1 mg/kg/dose to about 0.3 mg/kg/dose, about 0.3 mg/kg/dose to about 10 mg/kg/dose, about 0.3 mg/kg/dose to about 6 mg/kg/dose, about 0.3 mg/kg/dose to about 3 mg/kg/dose, about 0.3 mg/kg/dose to about 1 mg/kg/dose, about 0.3 mg/kg/dose to about 0.6 mg/kg/dose, about 0.6 mg/kg/dose to about 10 mg/kg/dose, about 0.6 mg/kg/dose to about 6 mg/kg/dose, about 0.6 mg/kg/dose to about 3 mg/kg/dose, about 0.6 mg/kg/dose to about 1 mg/kg/dose, about 1 mg/kg/dose to about 10 mg/kg/dose, about 1 mg/kg/dose to about 6 mg/kg/dose, about 1 mg/kg/dose to about 3 mg/kg/dose, about 3 mg/kg/dose to about 10 mg/kg/dose, about 3 mg/kg/dose to about 6 mg/kg/dose, or about 6 mg/kg/dose to about 10 mg/kg/dose. In some embodiments, a composition of the disclosure is administered at a dose level of about 0.001 mg/kg/dose, about 0.003 mg/kg/dose, about 0.006 mg/kg/dose, about 0.01 mg/kg/dose, about 0.03 mg/kg/dose, about 0.06 mg/kg/dose, about 0.1 mg/kg/dose, about 0.3 mg/kg/dose, about 0.6 mg/kg/dose, about 1 mg/kg/dose, about 3 mg/kg/dose, about 6 mg/kg/dose, or about 10 mg/kg/dose. Compositions of the disclosure can be administered at different times of the day. In one embodiment the optimal therapeutic dose can be administered in the evening. In another embodiment the optimal therapeutic dose can be administered in the morning. As expected, the dosage will be dependent on the condition, size, age, and condition of the subject.

Dosage of the pharmaceutical composition can be varied by the attending clinician to maintain a desired concentration at a target site. Higher or lower concentrations can be selected based on the mode of delivery. Dosage should also be adjusted based on the release rate of the administered formulation.

In some embodiments, the pharmaceutical composition of the disclosure is administered to a subject for multiple times (e.g., multiple doses). In some embodiments, the pharmaceutical composition is administered two or more times, three or more times, four or more times, etc. In some embodiments, administration of the pharmaceutical composition may be repeated once, twice, 3, 4, 5, 6, 7, 8, 9, 10, or more times. The pharmaceutical composition may be administered chronically or acutely, depending on its intended purpose.

In some embodiments, the interval between two consecutive doses of the pharmaceutical composition is less than 4, less than 3, less than 2, or less than 1 weeks. In some embodiments, the interval between two consecutive doses is less than 3 weeks. In some embodiments, the interval between two consecutive doses is less than 2 weeks. In some embodiments, the interval between two consecutive doses is less than 1 week. In some embodiments, the interval between two consecutive doses is less than 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the interval between two consecutive doses of the pharmaceutical composition is at least 4, at least 3, at least 2, or at least 1 weeks. In some embodiments, the interval between two consecutive doses of the pharmaceutical composition of the disclosure is at least 3 weeks. In some embodiments, the interval between two consecutive doses of the pharmaceutical composition of the disclosure is at least 2 weeks. In some embodiments, the interval between two consecutive doses of the pharmaceutical composition of the disclosure is at least 1 week. In some embodiments, the interval between two consecutive doses of the pharmaceutical composition of the disclosure is at least 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the subject is administered a dose of the pharmaceutical composition of the disclosure once daily, every 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the subject is administered a dose of the pharmaceutical composition of the disclosure once every 4, 5, 6, 7, 8, 9, 10, 11, or 12 weeks. In some embodiments, the subject is administered a dose of the pharmaceutical composition of the disclosure once every 1, 2, 3, 4, 5, 6, 7, 8, 9, 10, 11, or 12 months.

In some embodiments, the pharmaceutical composition of the disclosure is administered multiple times, wherein the serum half-life of the LNP in the subject following the second and/or subsequent administration is at least 40%, 50%, 60%, 70%, 80%, 85%, 90%, or 95% of the serum half-life of the LNP following the first administration.

In some embodiments, the second and subsequent doses of the pharmaceutical composition comprising an encapsulated molecule (e.g., encapsulated in an LNP) may maintain an activity of the encapsulated molecule of at least 50% of the activity of the first dose, or at least 60% of the first dose, or at least 70% of the first dose, or at least 75% of the first dose, or at least 80% of the first dose, or at least 85% of the first dose, or at least 90% of the first dose, or at least 95% of the first dose, or more, for at least 1 day, 2 days, 3 days, 4 days, 5 days, 6 days, or 7 days after second administration or subsequent administration.

In some embodiments, the pharmaceutical composition of the disclosure has an duration of therapeutic effect in vivo of about 1 hour or longer, about 2 hours or longer, about 3 hours or longer, about 4 hours or longer, about 5 hours or longer, about 6 hours or longer, about 7 hours or longer, about 8 hours or longer, about 9 hours or longer, about 10 hours or longer, about 12 hours or longer, about 14 hours or longer, about 16 hours or longer, about 18 hours or longer, about 20 hours or longer, about 25 hours or longer, about 30 hours or longer, about 35 hours or longer, about 40 hours or longer, about 45 hours or longer, or about 50 hours or longer. In some embodiments, the pharmaceutical composition of the disclosure has an duration of therapeutic effect in vivo of at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1.5 days, 2 days, 3 days, 4 days, 5 days, 6 days, 7 days, 8 days, 9 days, or 10 days.

In some embodiments, the pharmaceutical composition of the disclosure has a half-life in vivo comparable to that of a pre-determined threshold value. In some embodiments, the pharmaceutical composition of the disclosure has a half-life in vivo greater than that of a pre-determined threshold value. In some embodiments, the pharmaceutical composition of the disclosure has a half-life in vivo shorter than that of a pre-determined threshold value. In some embodiments, the pre-determined threshold value is the half-life of a control composition comprising the same payload molecule and LNP except that the LNP comprises (i) a PEG-lipid that is not of Formula (A), (A′), or (A″) (for example, the PEG-lipid of the LNP in the control composition may be PEG2k-DPG); or (ii) a cationic lipid that is not of Formula (I).

In some embodiments, the pharmaceutical composition of the disclosure has an AUC (area under the blood concentration-time curve) following a repeat dose that is at least 30%, at least 40%, at least 50%, at least 60%, at least 70%, at least 80%, at least 90%, or at least 100% of the AUC following the previous dose. In some embodiments, the pharmaceutical composition has an AUC that is at least 60% of the AUC following the previous dose. In some embodiments, following a repeat dose, AUC of the pharmaceutical composition decreases less than 70%, less than 60%, less than 60%, less than 40%, less than 30%, less than 20%, less than 10%, or less than 5% compared to the AUC following the previous dose. In some embodiments, following a repeat dose, AUC of the pharmaceutical composition decreases less than 40% compared to the AUC following the previous dose.

In some embodiments, the pharmaceutical composition of the disclosure comprises a nucleic acid molecule encoding viral genome of an oncolytic virus, and wherein administration of the pharmaceutical composition to a subject bearing a tumor delivers the nucleic acid molecule into tumor cells. In some embodiments, the nucleic acid molecule is a RNA molecule. In some embodiments, administration of the pharmaceutical composition results in replication of the oncolytic virus in tumor cells. In some embodiments, administration of the pharmaceutical composition to a subject bearing a tumor results in selective replication of the oncolytic virus in tumor cells as compared to normal cells.

In some embodiments, administration of the pharmaceutical composition of the disclosure to a subject bearing a tumor inhibits growth of the tumor. In some embodiments, administration of the pharmaceutical composition inhibits growth of the tumor for at least 1 week, at least 1 month, at least 2 months, at least 3 months, at least 4 months, at least 6 months, at least 9 months, at least 12 months, at least 2 years, or longer. In some embodiments, inhibiting growth of the tumor means controlling the size of the tumor within 100% of the size of the tumor just before administration of the pharmaceutical composition for a specified time period. In some embodiments, inhibiting growth of the tumor means controlling the size of the tumor within 110%, within 120%, within 130%, within 140%, or within 150%, of the size of the tumor just before administration of the pharmaceutical composition.

In some embodiments, administration of the pharmaceutical composition to a subject bearing a tumor leads to tumor shrinkage or elimination. In some embodiments, administration of the pharmaceutical composition leads to tumor shrinkage or elimination for at least 1 week, at least 1 month, at least 2 months, at least 3 months, at least 4 months, at least 6 months, at least 9 months, at least 12 months, at least 2 years, or longer. In some embodiments, administration of the pharmaceutical composition leads to tumor shrinkage or elimination within 1 week, within 2 weeks, within 3 weeks, within 4 weeks, within 1 month, within 2 months, within 3 months, within 4 months, within 6 months, within 9 months, within 12 months, or within 2 years. In some embodiments, tumor shrinkage means reducing the size of the tumor by at least 10%, at least 20%, at least 30%, at least 40%, at least 50%, at least 60%, at least 70%, at least 80%, at least 90%, or at least 95%, compared to the size of the tumor just before administration of the pharmaceutical composition. In some embodiments, tumor shrinkage means reducing the size of the tumor at least 30% compared to the size of the tumor just before administration of the pharmaceutical composition.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition

The disclosure relates to a method of treating cancer in a subject in need thereof, comprising administering a therapeutically effective amount of a composition as described herein to the subject.

In some embodiments, the disclosure provides methods of delivering a encapsulated molecule to a cell, the method comprising contacting the cell with the LNP or pharmaceutical composition thereof, wherein the LNP comprises the encapsulated molecule. In some embodiments, the encapsulated molecule is a nucleic acid molecule encoding a virus, and wherein contacting the cell with the LNP results in production of viral particles by the cell, and wherein the viral particles are infectious and lytic.

In some embodiments, the disclosure provides methods of delivering an LNP to a subject, comprising administering the LNP or the pharmaceutical composition thereof of the disclosure to the subject. In some embodiments, the method comprises multiple administrations. In some embodiments, the interval between two consecutive administrations of the pharmaceutical composition is less than 4, less than 3, less than 2, or less than 1 weeks. In some embodiments, the interval between two consecutive administrations is less than 2 weeks. In some embodiments, the interval between two consecutive administrations is less than 1 week. In some embodiments, the interval between two consecutive administrations is less than 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the interval between two consecutive administrations of the pharmaceutical composition is at least 4, at least 3, at least 2, or at least 1 weeks. In some embodiments, the interval between two consecutive administrations of the pharmaceutical composition of the disclosure is at least 2 weeks. In some embodiments, the interval between two consecutive administrations of the pharmaceutical composition of the disclosure is at least 1 week. In some embodiments, the interval between two consecutive administrations of the pharmaceutical composition of the disclosure is at least 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the method comprises administering to a subject the pharmaceutical composition of the disclosure every 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the method comprises administering to a subject the pharmaceutical composition of the disclosure once every 4, 5, 6, 7, 8, 9, 10, 11, or 12 weeks. In some embodiments, the method comprises administering to a subject the pharmaceutical composition of the disclosure once every 1, 2, 3, 4, 5, 6, 7, 8, 9, 10, 11, or 12 months.

In some embodiments, the disclosure provides methods of delivering an LNP to a subject, comprising administering the LNP or the pharmaceutical composition thereof of the disclosure to the subject, wherein the method comprises multiple administrations. In some embodiments, serum half-life of the LNP in the subject following the second and/or subsequent administration of the method is at least 40%, 50%, 60%, 70%, 80%, 85%, 90%, or 95% of the serum half-life of the LNP following the first administration.

In some embodiments, the LNP has an AUC following a repeat dose that is at least 30%, at least 40%, at least 50%, at least 60%, at least 70%, at least 80%, at least 90%, or at least 100% of the AUC following the previous dose. In some embodiments, the LNP has an AUC that is at least 60% of the AUC following the previous dose. In some embodiments, following a repeat dose, AUC of the LNP decreases less than 70%, less than 60%, less than 60%, less than 40%, less than 30%, less than 20%, less than 10%, or less than 5% compared to the AUC following the previous dose. In some embodiments, following a repeat dose, AUC of the LNP decreases less than 40% compared to the AUC following the previous dose.

In some embodiments, the disclosure provides methods of delivering an LNP to a subject, comprising administering the LNP or the pharmaceutical composition thereof of the disclosure to the subject, wherein the LNP comprises a nucleic acid molecule encoding a viral genome of an oncolytic virus, wherein the subject has a tumor, and wherein administration of the LNP delivers the nucleic acid molecule into tumor cells. In some embodiments, administration of the LNP results in replication of the oncolytic virus in tumor cells. In some embodiments, administration of the LNP results in selective replication of the oncolytic virus in tumor cells as compared to normal cells.

In some embodiments, the disclosure provides methods of delivering an LNP to a subject, comprising administering the LNP or the pharmaceutical composition thereof of the disclosure to the subject, wherein administration of the LNP to a subject bearing a tumor inhibits growth of the tumor. In some embodiments, the method inhibits growth of the tumor for at least 1 week, at least 1 month, at least 2 months, at least 3 months, at least 4 months, at least 6 months, at least 9 months, at least 12 months, at least 2 years, or longer. In some embodiments, inhibiting growth of the tumor means controlling the size of the tumor within 100% of the size of the tumor just before administration of the pharmaceutical composition for a specified time period. In some embodiments, inhibiting growth of the tumor means controlling the size of the tumor within 110%, within 120%, within 130%, within 140%, or within 150%, of the size of the tumor just before administration of the pharmaceutical composition.

In some embodiments, the disclosure provides methods of delivering an LNP to a subject, comprising administering the LNP or the pharmaceutical composition thereof of the disclosure to the subject, wherein administration of the LNP to a subject bearing a tumor leads to tumor shrinkage or elimination. In some embodiments, the method results in tumor shrinkage or elimination for at least 1 week, at least 1 month, at least 2 months, at least 3 months, at least 4 months, at least 6 months, at least 9 months, at least 12 months, at least 2 years, or longer. In some embodiments, the method results in tumor shrinkage or elimination within 1 week, within 2 weeks, within 3 weeks, within 4 weeks, within 1 month, within 2 months, within 3 months, within 4 months, within 6 months, within 9 months, within 12 months, or within 2 years. In some embodiments, tumor shrinkage means reducing the size of the tumor by at least 10%, at least 20%, at least 30%, at least 40%, at least 50%, at least 60%, at least 70%, at least 80%, at least 90%, or at least 95%, compared to the size of the tumor just before administration of the pharmaceutical composition. In some embodiments, tumor shrinkage means reducing the size of the tumor at least 30% compared to the size of the tumor just before administration of the pharmaceutical composition.

In some embodiments, the disclosure provides methods of delivering an LNP to a subject, comprising administering the LNP or the pharmaceutical composition thereof of the disclosure to the subject, wherein administration of the LNP to a subject bearing a tumor inhibits the metastasis of the cancer.

In some embodiments, the subject is a mammal. In some embodiments, the subject is a human. In some embodiments, the subject has a cancer, and wherein the method inhibits or slows the growth and/or metastasis of the cancer.

In some embodiments, the disclosure provides methods of delivering an LNP to a subject, comprising systemically administering the LNP or pharmaceutical composition thereof. In some embodiments, the administration is intravenous, intra-arterial, intraperitoneal, intramuscular, intradermal, subcutaneous, intranasal, oral, or a combination thereof.

In some embodiments, the disclosure provides methods of delivering an LNP to a subject, comprising locally administering the LNP or pharmaceutical composition thereof. In some embodiments, the administration is intratumoral.

### Cancer

In some embodiments, the disclosure provides methods of killing a cancerous cell comprising exposing the cancerous cell to the lipid nanoparticles, the recombinant RNA molecules, or compositions thereof of the disclosure. In some embodiments, the cancerous cells are exposed under conditions sufficient for the intracellular delivery of the particles/recombinant RNA molecules/compositions to said cancerous cell, wherein the replication-competent virus produced by the encapsulated polynucleotide results in killing of the cancerous cell.

In some embodiments, the disclosure provides methods of treating a cancer in a subject comprising administering to a subject suffering from the cancer an effective amount of the particles, the recombinant RNA molecules, or compositions thereof of the disclosure.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, and chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. In some embodiments, the cancer is a neuroendocrine cancer. Furthermore, benign (i.e., noncancerous) hyperproliferative diseases, disorders and conditions, including benign prostatic hypertrophy (BPH), meningioma, schwannoma, neurofibromatosis, keloids, myoma and uterine fibroids and others may also be treated using the disclosure disclosed herein. In some embodiments, the cancer is selected from small cell lung cancer (SCLC), small cell bladder cancer, large cell neuroendocrine carcinoma (LCNEC), castration-resistant small cell neuroendocrine prostate cancer (CRPC-NE), carcinoid (e.g., pulmonary carcinoid), and glioblastoma multiforme-IDH mutant (GBM-IDH mutant).

In some embodiments, the cancer is a metastatic cancer. In some embodiments, the cancer has metastasized. In some embodiments, the cancer is a non-metastatic cancer.

In some embodiments, the cancer is selected from the group consisting of lung cancer, breast cancer, colon cancer, pancreatic cancer, bladder cancer, renal cell carcinoma, ovarian cancer, gastric cancer and liver cancer. In some embodiments, the cancer is renal cell carcinoma, lung cancer, or liver cancer. In some embodiments, the lung cancer is NSCLC (non-small cell lung cancer). In some embodiments, the liver cancer is HCC (hepatocellular carcinoma). In some embodiments, the liver cancer is metastatic. In some embodiments, the breast cancer is TNBC (triple-negative breast cancer). In some embodiments, the bladder cancer is urothelial carcinoma. In some embodiments, the cancer is selected from the group consisting of breast cancer, esophageal cancer, stomach cancer, lung cancer, kidney cancer and skin cancer, and wherein the cancer has metastasized into liver. In some embodiments, the cancer is a metastasized cancer in the liver, wherein the cancer is originated from the group consisting of breast cancer, esophageal cancer, stomach cancer, lung cancer, kidney cancer and skin cancer. In some embodiments, the cancer is a hematologic cancer. In some embodiments, the hematologic cancer is multiple myeloma (see, e.g., Bradley, et al., Oncolytic Virotherapy, 2014:3 47-55, the content of which is incorporated by reference in its entirety). In some embodiments, the hematologic cancer is a leukemia or a lymphoma.

In some embodiments, the particles, the recombinant RNA molecules, or compositions thereof comprises a polynucleotide sequence derived from a CVA21-KY strain for treating cancer or killing cancer cells of lung cancer (e.g., NSCLC), breast cancer, colon cancer, or pancreatic cancer. In some embodiments, the cancer is lung cancer (e.g., NSCLC).

In some embodiments, the particles, the recombinant RNA molecules, or compositions thereof comprises a polynucleotide sequence derived from a CVA21-EF strain for treating cancer or killing cancer cells of bladder cancer, renal cell carcinoma, ovarian cancer, gastric cancer, or liver cancer (e.g., HCC). In some embodiments, the cancer is renal cell carcinoma. In some embodiments, the cancer is liver cancer (e.g., HCC). In some embodiments, the liver cancer is metastatic.

In some embodiments, the particles, the recombinant RNA molecules, or compositions thereof comprises a polynucleotide sequence derived from an SVV (e.g., a SVV-IRES-2 chimeric virus) for treating cancer or killing cancer cells of lung cancer, liver cancer, prostate cancer, bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma (MCC), or melanoma. In some embodiments, the cancer is small cell lung cancer (SCLC). In some embodiments, the cancer is neuroblastoma. In some embodiments, the cancer is neuroendocrine cancer. In some embodiments, the cancer is rhabdomyosarcoma. In some embodiments, the cancer is castration-resistant prostate cancer with neuroendocrine phenotype (CRPC-NE). In some embodiments, the cancer is Merkel cell carcinoma (MCC).

In some embodiments, the disclosure provides methods of treating a cancer in a subject comprising administering to a subject suffering from the cancer (i) an effective amount of a particle (e.g., LNPs), a recombinant RNA molecule, or compositions thereof of the disclosure, and (ii) an effective amount of an immune checkpoint inhibitor. In some embodiments, the immune checkpoint inhibitor is an antibody or an antigen binding fragment thereof. In some embodiments, the immune checkpoint inhibitor binds to PD-1 (e.g., the inhibitor is an anti-PD-1 antibody). Anti-PD1 antibodies are known in the art, for example, Nivolumab, Pembrolizumab, Lambrolizumab, Pidilzumab, Cemiplimab, and AMP-224 (AstraZeneca/MedImmune and GlaxoSmithKline), JTX-4014 by Jounce Therapeutics, Spartalizumab (PDR001, Novartis), Camrelizumab (SHR1210, Jiangsu HengRui Medicine Co., Ltd), Sintilimab (IBI308, Innovent and Eli Lilly), Tislelizumab (BGB-A317), Toripalimab (JS 001), Dostarlimab (TSR-042, WBP-285, GlaxoSmithKline), INCMGA00012 (MGA012, Incyte and MacroGenics), and AMP-514 (MEDI0680, AstraZeneca). In some embodiments, the immune checkpoint inhibitor binds to PD-L¹ (e.g., the inhibitor is an anti-PD-L¹ antibody). Anti-PDL1 antibodies are known in the art, for example, MEDI-4736, MPDL3280A, Atezolizumab (Tecentriq, Roche Genentech), Avelumab (Bavencio, Merck Serono and Pfizer), and Durvalumab (Imfinzi, AstraZeneca). In some embodiments, the immune checkpoint inhibitor binds to CTLA4 (e.g., the inhibitor is an anti-CTLA4 antibody). Anti-CTLA4 antibodies are known in the art, for example, ipilumumab, tremelimumab, or any of the antibodies disclosed in WO2014/207063. In some embodiments, the immune checkpoint inhibitor is an anti-TIGIT antibody or fragment thereof. Anti-TIGIT antibodies are known in the art, for example tiragolumab (Roche), EOS-448 (iTeos Therapeutics), Vibostolimab (Merck), Domvanalimab (Arcus, Gilead), BMS-986207 (BMS), Etigilimab (Mereo), COM902 (Compugen), ASP8374 (Astellas), SEA-TGT (Seattle Genetics) BGB-A1217 (BeiGene), IBI-939 (Innovent), and M6223 (EMD Serono).

In some embodiments, both of 1) the particles, the recombinant RNA molecules, or compositions thereof and 2) the immune checkpoint inhibitor are concurrently administered. In some embodiments, these two therapeutic components are administered sequentially. In some embodiments, one or both therapeutic components are administered multiple times. In some embodiments, the particles, the recombinant RNA molecules, or compositions thereof comprises a polynucleotide sequence derived from an SVV (e.g., a SVV-IRES-2 chimeric virus), and the immune checkpoint inhibitor binds to PD-1.

In some embodiments, the disclosure provides methods of treating a cancer in a subject comprising administering to a subject suffering from the cancer (i) an effective amount of a particle (e.g., LNPs), a recombinant RNA molecule, or compositions thereof of the disclosure, and (ii) an effective amount of an engineered immune cell comprising an “engineered antigen receptor.” Engineered antigen receptors refer to non-naturally occurring antigen-specific receptors such as a chimeric antigen receptors (CARs) or a recombinant T cell receptor (TCRs). In some embodiments, the engineered antigen receptor is a CAR comprising an extracellular antigen binding domain fused via hinge and transmembrane domains to a cytoplasmic domain comprising a signaling domain. In some embodiments, the CAR extracellular domain binds to an antigen expressed by a target cell in an MHC-independent manner leading to activation and proliferation of the engineered immune cell cell. In some embodiments, the extracellular domain of a CAR recognizes a tag fused to an antibody or antigen-binding fragment thereof. In such embodiments, the antigen-specificity of the CAR is dependent on the antigen-specificity of the labeled antibody, such that a single CAR construct can be used to target multiple different antigens by substituting one antibody for another (See e.g., U.S. Pat. Nos. 9,233,125 and 9,624,279; US Patent Application Publication Nos. 20150238631 and 20180104354). In some embodiments, the extracellular domain of a CAR may comprise an antigen binding fragment derived from an antibody. Antigen binding domains that are useful in the present disclosure include, for example, scFvs; antibodies; antigen binding regions of antibodies; variable regions of the heavy/light chains; and single chain antibodies.

In some embodiments, the intracellular signaling domain of a CAR may be derived from the TCR complex zeta chain (such as CD3ξ signaling domains), FcγRIII, FcεRI, or the T-lymphocyte activation domain. In some embodiments, the intracellular signaling domain of a CAR further comprises a costimulatory domain, for example a 4-1BB, CD28, CD40, MyD88, or CD70 domain. In some embodiments, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US20130287748; WO 2015/142675; WO 2014/055657; and WO 2015/090229, incorporated herein by reference).

CARs specific for a variety of tumor antigens are known in the art, for example CD171-specific CARs (Park et al., Mol Ther (2007) 15 (4): 825-833), EGFRvIII-specific CARs (Morgan et al., Hum Gene Ther (2012) 23 (10): 1043-1053), EGF-R-specific CARs (Kobold et al., J Natl Cancer Inst (2014) 107 (1): 364), carbonic anhydrase K-specific CARs (Lamers et al., Biochem Soc Trans (2016) 44 (3): 951-959), FR-α-specific CARs (Kershaw et al., Clin Cancer Res (2006) 12 (20): 6106-6015), HER2-specific CARs (Ahmed et al., J Clin Oncol (2015) 33 (15) 1688-1696; Nakazawa et al., Mol Ther (2011) 19 (12): 2133-2143; Ahmed et al., Mol Ther (2009) 17 (10): 1779-1787; Luo et al., Cell Res (2016) 26 (7): 850-853; Morgan et al., Mol Ther (2010) 18 (4): 843-851; Grada et al., Mol Ther Nucleic Acids (2013) 9 (2): 32), CEA-specific CARs (Katz et al., Clin Cancer Res (2015) 21 (14): 3149-3159), IL13Ra2-specific CARs (Brown et al., Clin Cacner Res (2015) 21 (18): 4062-4072), GD2-specific CARs (Louis et al., Blood (2011) 118 (23): 6050-6056; Caruana et al., Nat Med (2015) 21 (5): 524-529), ErbB2-specific CARs (Wilkie et al., J Clin Immunol (2012) 32 (5): 1059-1070), VEGF-R-specific CARs (Chinnasamy et al., Cancer Res (2016) 22 (2): 436-447), FAP-specific CARs (Wang et al., Cancer Immunol Res (2014) 2 (2): 154-166), MSLN-specific CARs (Moon et al, Clin Cancer Res (2011) 17 (14): 4719-30), NKG2D-specific CARs (VanSeggelen et al., Mol Ther (2015) 23 (10): 1600-1610), CD19-specific CARs (Axicabtagene ciloleucel (Yescarta®) and Tisagenlecleucel (Kymriah®). See also, Li et al., J Hematol and Oncol (2018) 11 (22), reviewing clinical trials of tumor-specific CARs.

In some embodiments, the engineered antigen receptor is an engineered TCR. Engineered TCRs comprise TCRα and/or TCRβ chains that have been isolated and cloned from T cell populations recognizing a particular target antigen. For example, TCRα and/or TCRβ genes (i.e., TRAC and TRBC) can be cloned from T cell populations isolated from individuals with particular malignancies or T cell populations that have been isolated from humanized mice immunized with specific tumor antigens or tumor cells. Engineered TCRs recognize antigen through the same mechanisms as their endogenous counterparts (e.g., by recognition of their cognate antigen presented in the context of major histocompatibility complex (MHC) proteins expressed on the surface of a target cell). This antigen engagement stimulates endogenous signal transduction pathways leading to activation and proliferation of the TCR-engineered cells.

Engineered TCRs specific for tumor antigens are known in the art, for example WT1-specific TCRs (JTCR016, Juno Therapeutics; WT1-TCRc4, described in US Patent Application Publication No. 20160083449), MART-1 specific TCRs (including the DMF4T clone, described in Morgan et al., Science 314 (2006) 126-129); the DMF5T clone, described in Johnson et al., Blood 114 (2009) 535-546); and the ID3T clone, described in van den Berg et al., Mol. Ther. 23 (2015) 1541-1550), gp100-specific TCRs (Johnson et al., Blood 114 (2009) 535-546), CEA-specific TCRs (Parkhurst et al., Mol Ther. 19 (2011) 620-626), NY-ESO and LAGE-1 specific TCRs (1G4T clone, described in Robbins et al., J Clin Oncol 26 (2011) 917-924; Robbins et al., Clin Cancer Res 21 (2015) 1019-1027; and Rapoport et al., Nature Medicine 21 (2015) 914-921), and MAGE-A3-specific TCRs (Morgan et al., J Immunother 36 (2013) 133-151) and Linette et al., Blood 122 (2013) 227-242). (See also, Debets et al., Seminars in Immunology 23 (2016) 10-21).

In some embodiments, the engineered antigen receptor is directed against a target antigen selected from a cluster of differentiation molecule, such as CD3, CD4, CD8, CD16, CD24, CD25, CD33, CD34, CD45, CD64, CD71, CD78, CD80 (also known as B7-1), CD86 (also known as B7-2), CD96, CD116, CD117, CD123, CD133, and CD138, CD371 (also known as CLL1); a tumor-associated surface antigen, such as 5T4, BCMA (also known as CD269 and TNFRSF17, UniProt #Q02223), carcinoembryonic antigen (CEA), carbonic anhydrase 9 (CAIX or MN/CAIX), CD19, CD20, CD22, CD30, CD40, disialogangliosides such as GD2, ELF2M, ductal-epithelial mucin, ephrin B2, epithelial cell adhesion molecule (EpCAM), ErbB2 (HER2/neu), FCRL5 (UniProt #Q68SN8), FKBP11 (UniProt #Q9NYL4), glioma-associated antigen, glycosphingolipids, gp36, GPRC5D (UniProt #Q9NZD1), mut hsp70-2, intestinal carboxyl esterase, IGF-I receptor, ITGA8 (UniProt #P53708), KAMP3, LAGE-la, MAGE, mesothelin, neutrophil elastase, NKG2D, Nkp30, NY-ESO-1, PAP, prostase, prostate-carcinoma tumor antigen-1 (PCTA-1), prostate specific antigen (PSA), PSMA, prostein, RAGE-1, ROR1, RU1 (SFMBT1), RU2 (DCDC2), SLAMF7 (UniProt #Q9NQ25), survivin, TAG-72, and telomerase; a major histocompatibility complex (MHC) molecule presenting a tumor-specific peptide epitope; tumor stromal antigens, such as the extra domain A (EDA) and extra domain B (EDB) of fibronectin; the A1 domain of tenascin-C(TnC A1) and fibroblast associated protein (FAP); cytokine receptors, such as epidermal growth factor receptor (EGFR), EGFR variant III (EGFRVIII), TFGB-R or components thereof such as endoglin; a major histocompatibility complex (MHC) molecule; a virus-specific surface antigen such as an HIV-specific antigen (such as HIV gp120); an EBV-specific antigen, a CMV-specific antigen, a HPV-specific antigen, a Lassa virus-specific antigen, an Influenza virus-specific antigen as well as any derivate or variant of these surface antigens.

SELECTED SEQUENCES OF THE DISCLOSURE

| SEQ | | | |
|---|---|---|---|
| ID | | | |
| NO | Name | Sequence | Type |
| 130 | ENV27-WT | ACTCGTTTGAGCGAGTATAAACAGT | DNA |
| | | TGGTTAGGCTCAAAGCGGAGAGCAG | |
| | | ATCTGCTCTCGTCCAA | |
| 131 | ENV27-V1 | ACTCGTTTGAGCGAGTATAAACAGT | DNA |
| | | TTGTTAGGCTCAAAGCGGAGAGCAG | |
| | | ATCTGCTCTCG | |
| 132 | ENV27-V2 | ACTCGTTTGAGCGAGTATAAACAGT | DNA |
| | | TTATTAGGCTCAAAGCGGAGAGCAG | |
| | | ATCTGCTCTCG | |
| 133 | ENV27-V3 | ACTCGTTTGAGCGAGTATAAACAGT | DNA |
| | | TTATTAGGCTCAAAGCGGAGAGCAG | |
| | | AGAAATCTGCTCTCG | |
| 134 | ENV27-V4 | ACTCGTTTGAGCGAGTATAAACAGT | DNA |
| | | TTATTAGGCTCAAAGCGGAGAGCAG | |
| | | AACGAGAAATCGTTCTGCTCTCG | |
| 135 | CVA21-L1 | GGATGTTAATCAGGATATACATACA | DNA |
| | | GAAGGAAAAGTGAACTAGGGCATGG | |
| 136 | CVA21-L2 | ACATAATTCTGGTGTAAACGACTTA | DNA |
| | | ACAATACGAAAGCCTGCGATCACAA | |

### FURTHER NUMBER EMBODIMENTS

Further numbered embodiments of the invention are provided as follows:

Embodiment 1. A recombinant DNA molecule comprising, from 5′ to 3′, a promoter sequence, a 5′ junctional cleavage sequence, and a polynucleotide sequence encoding an RNA molecule comprising a synthetic RNA viral genome, wherein the 5′ junctional cleavage sequence comprises or consists of a ENV27 ribozyme encoding sequence.

Embodiment 2. The recombinant DNA molecule of Embodiment 1, wherein the ENV27 ribozyme encoding sequence comprises or consists of a polynucleotide sequence (excluding P3 stem insert) having at least 80% identity to SEQ ID NO: 132 (excluding its P3 stem insert corresponding to nucleotides 49-54 of SEQ ID NO: 132).

Embodiment 3. The recombinant DNA molecule of Embodiment 2, wherein the polynucleotide sequence (excluding P3 stem insert) is 100% identical, or has at most 1, at most 2, at most 3, at most 4, at most 5, at most 6, at most 7, at most 8, at most 9, at most 10, or at most 11 mutations (insertions, deletions or substitutions), as compared to SEQ ID NO: 132 (excluding its P3 stem insert corresponding to nucleotides 49-54 of SEQ ID NO: 132).

Embodiment 4. The recombinant DNA molecule of any one of Embodiments 1-3, wherein the polynucleotide sequence is 100% identical, or has at most 1, at most 2, at most 3, at most 4, at most 5, at most 6, at most 7, at most 8, at most 9, at most 10, or at most 11 mutations (insertions, deletions or substitutions), as compared to any one of SEQ ID NO: 130-134.

Embodiment 5. The recombinant DNA molecule of Embodiment 3 or 4, wherein the mutation(s) are substitution(s).

Embodiment 6. The recombinant DNA molecule of any one of Embodiments 1-5, wherein the ENV27 ribozyme encoding sequence comprises the polynucleotides “TTTATT” or “TTTGTT” at the positions corresponding to nucleotides 25-30 of SEQ ID NO: 132.

Embodiment 7. The recombinant DNA molecule of Embodiment 6, wherein the ENV27 ribozyme encoding sequence comprises the polynucleotides “TTTATT” at the positions corresponding to nucleotides 25-30 of SEQ ID NO: 132.

Embodiment 8. The recombinant DNA molecule of any one of Embodiments 2-7, wherein the ENV27 ribozyme encoding sequence comprises the P3 stem insert of about 1-30, about 1-20, about 6-20, or about 6-10 polynucleotides in length.

Embodiment 9. The recombinant DNA molecule of any one of Embodiments 2-8, wherein the P3 stem insert is at least 1, 2, 3, 4, 5, 6, 7, 8, 9, 10, 11, 12, 13, 14, 15, 16, 17, 18, 19, or 20 polynucleotides in length.

Embodiment 10. The recombinant DNA molecule of any one of Embodiments 2-9, wherein the P3 stem insert is 1, 2, 3, 4, 5, 6, 7, 8, 9, 10, 11, 12, 13, 14, 15, 16, 17, 18, 19, or 20 polynucleotides in length.

Embodiment 11. The recombinant DNA molecule of any one of Embodiments 8-10, wherein the P3 stem insert comprises or consists of the polynucleotides “AGATCT” at the region corresponding to nucleotides 49-54 of SEQ ID NO: 132.

Embodiment 12. The recombinant DNA molecule of any one of Embodiments 8-10, wherein the P3 stem insert comprises or consists of the polynucleotides “AGAGAAATCT” (SEQ ID NO: 137) at the region corresponding to nucleotides 49-54 of SEQ ID NO: 132.

Embodiment 13. The recombinant DNA molecule of any one of Embodiments 8-10, wherein the P3 stem insert comprises or consists of the polynucleotides “AGAACGAGAAATCGTTCT” (SEQ ID NO: 138) at the region corresponding to nucleotides 49-54 of SEQ ID NO: 132.

Embodiment 14. The recombinant DNA molecule of any one of Embodiments 1-13, comprising, from 5′ to 3′, the promoter sequence, the 5′ junctional cleavage sequence, the polynucleotide sequence encoding the RNA molecule comprising the synthetic RNA viral genome, and a poly-A tail.

Embodiment 15. The recombinant DNA molecule of Embodiment 14, comprising, from 5′ to 3′, the promoter sequence, the 5′ junctional cleavage sequence, the polynucleotide sequence encoding the RNA molecule comprising the synthetic RNA viral genome, the poly-A tail, and a 3′ junctional cleavage sequence.

Embodiment 16. The recombinant DNA molecule of any one of Embodiments 1-15, wherein the synthetic RNA viral genome encodes a picornavirus.

Embodiment 17. The recombinant DNA molecule of Embodiment 16, wherein the picornavirus is a coxsackievirus virus.

Embodiment 18. The recombinant DNA molecule of any one of Embodiments 1-17, wherein the 5′ end of the RNA viral genome starts with “UUAAA”.

Embodiment 19. The recombinant DNA molecule of any one of Embodiments 17-18, wherein the Coxsackievirus is a CVA21 strain.

Embodiment 20. The recombinant DNA molecule of Embodiment 19, wherein the CVA21 strain is selected from the Kuykendall strain, the EF strain and the KY strain.

Embodiment 21. The recombinant DNA molecule of any one of Embodiments 1-20, wherein the 5′ end of the RNA viral genome comprises a polynucleotide sequence having at least 80%, at least 85%, at least 90%, at least 91%, at least 92%, at least 93%, at least 94%, at least 95%, at least 96%, at least 97%, at least 98%, at least 99%, or 100% identity to nucleotides 1-260 of any one of SEQ ID NO: 1, 5, or 9.

Embodiment 22. The recombinant DNA molecule of any one of Embodiments 1-21, wherein the recombinant DNA molecule does not comprise additional nucleic acid between the 5′ junctional cleavage sequence and the polynucleotide sequence encoding the RNA molecule.

Embodiment 23. The recombinant DNA molecule of any one of Embodiments 1-22, wherein cleavage of the 5′ junctional cleavage sequence and/or the 3′ junctional cleavage sequence produces native 5′ and/or 3′ ends of the synthetic RNA viral genome after transcription.

Embodiment 24. The recombinant DNA molecule of any one of Embodiments 1-23, further comprising a leader sequence between the promoter sequence and the 5′ junctional cleavage sequence.

Embodiment 25. The recombinant DNA molecule of Embodiment 24, wherein the leader sequence is less than 100 bp, less than 90 bp, less than 80 bp, less than 70 bp, less than 60 bp, less than 50 bp, or less than 40 bp in length.

Embodiment 26. The recombinant DNA molecule of any one of Embodiments 24-25, wherein the leader sequence comprises or consists of a polynucleotide sequence having at least 70%, at least 80%, at least 90%, at least 95%, at least 96%, at least 97%, at least 98%, at least 99%, or 100% sequence identity according to SEQ ID NO: 135 or 136.

Embodiment 27. The recombinant DNA molecule of any one of Embodiments 24-25, wherein the leader sequence comprises or consists of a polynucleotide sequence having at least 70%, at least 80%, at least 90%, at least 95%, at least 96%, at least 97%, at least 98%, at least 99%, or 100% sequence identity according to SEQ ID NO: 135.

Embodiment 28. The recombinant DNA molecule of any one of Embodiments 24-27, wherein the recombinant DNA molecule does not comprise additional nucleic acid between the promoter sequence and the leader sequence.

Embodiment 29. The recombinant DNA molecule of any one of Embodiments 24-28, wherein the recombinant DNA molecule does not comprise additional nucleic acid between the leader sequence and the 5′ junctional cleavage sequence.

Embodiment 30. The recombinant DNA molecule of any one of Embodiments 1-27, wherein the promoter sequence is a T7 promoter sequence.

Embodiment 31. The recombinant DNA molecule of Embodiment 30, wherein the T7 promoter sequence comprises or consists of SEQ ID NO: 91.

Embodiment 32. The recombinant DNA molecule of any one of Embodiments 1-31, wherein the poly-A tail is about 50-90 bp in length or about 65-75 bp in length.

Embodiment 33. The recombinant DNA molecule of Embodiment 32, wherein the poly-A tail is about 70 bp in length.

Embodiment 34. The recombinant DNA molecule of any one of Embodiments 1-31, wherein the poly-A tail is about 10-50 bp, or 25-35 bp in length.

Embodiment 35. The recombinant DNA molecule of any one of Embodiments 1-34, wherein the 3′ junctional cleavage sequence comprises or consists of a ribozyme sequence.

Embodiment 36. The recombinant DNA molecule of Embodiment 35, wherein the 3′ ribozyme sequence is a hepatitis delta virus ribozyme sequence.

Embodiment 37. The recombinant DNA molecule of any one of Embodiments 1-34, wherein the 3′ junctional cleavage sequence comprises or consists of a restriction enzyme recognition sequence.

Embodiment 38. The recombinant DNA molecule of any one of Embodiments 1-34, wherein the 3′ junctional cleavage sequence comprises or consists of a Type IIS restriction enzyme recognition sequence.

Embodiment 39. The recombinant DNA molecule of any one of Embodiments 1-38, wherein the 3′ junctional cleavage sequence comprises or consists of a BsmBI recognition sequence.

Embodiment 40. The recombinant DNA molecule of any one of Embodiments 1-38, wherein the 3′ junctional cleavage sequence comprises or consists of a BsaI recognition sequence.

Embodiment 41. The recombinant DNA molecule of any one of Embodiments 1-40, wherein the promoter sequence is a T7 promoter sequence, wherein the leader sequence consists of a polynucleotide sequence according to SEQ ID NO: 135, wherein the 5′ junctional cleavage sequence comprises or consists of a ENV27 ribozyme sequence according to any one of SEQ ID NO: 132-134, wherein the poly-A tail is about 70 bp in length, and wherein the 3′ junctional cleavage sequence comprises or consists of a BsmBI recognition sequence.

Embodiment 42. The recombinant DNA molecule of any one of Embodiments 1-40, wherein the promoter sequence is a T7 promoter sequence, wherein the leader sequence consists of a polynucleotide sequence according to SEQ ID NO: 135, wherein the 5′ junctional cleavage sequence comprises or consists of a ENV27 ribozyme sequence according to any one of SEQ ID NO: 132-134, wherein the poly-A tail is about 70 bp in length, and wherein the 3′ junctional cleavage sequence comprises or consists of a BsaI recognition sequence.

Embodiment 43. The recombinant DNA molecule of any one of Embodiments 1-42, wherein the recombinant DNA molecule does not comprise additional nucleic acid within the region spanning the promoter sequence and the 3′ junctional cleavage sequence.

Embodiment 44. A method of producing a recombinant RNA molecule, comprising in vitro transcription of the recombinant DNA molecule of any one of Embodiments 1-43 and purification of the resulting recombinant RNA molecule.

Embodiment 45. The method of Embodiment 44, wherein the recombinant RNA molecule comprises 5′ and 3′ ends that are native to the viral genome encoded by the recombinant RNA molecule.

Embodiment 46. A recombinant RNA molecule, or a plurality of recombinant RNA molecules, transcribed from the recombinant DNA molecule of any one of Embodiments 1-43.

Embodiment 47. The recombinant RNA molecules of Embodiment 46, wherein at least 70%, at least 75%, at least 80%, at least 85%, at least 90%, at least 95%, at least 96%, at least 97%, at least 98%, at least 99%, at least 99.5%, or at least 99.9%, of the recombinant RNA molecules comprise 5′ and 3′ ends that are native to the viral genome encoded by the recombinant RNA molecule.

Embodiment 48. The recombinant RNA molecules of Embodiment 46 or 47, wherein no more than 30%, no more than 25%, no more than 20%, no more than 15%, no more than 10%, no more than 5%, no more than 4%, no more than 3%, no more than 2%, no more than 1%, no more than 0.5%, or no more than 0.1%, of the recombinant RNA molecules comprise an RNA sequence encoded by the ENV27 ribozyme encoding sequence.

Embodiment 48.1 The recombinant RNA molecules of any one of Embodiments 46-48, wherein at least one of the recombinant RNA molecules comprises an RNA sequence encoded by the ENV27 ribozyme encoding sequence.

Embodiment 48.2 The recombinant RNA molecules of any one of Embodiments 46-48, wherein at least 0.0001%, at least 0.001%, at least 0.01%, at least 0.1%, or at least 1%, of the recombinant RNA molecules comprise an RNA sequence encoded by the ENV27 ribozyme encoding sequence.

Embodiment 49. A composition comprising an effective amount of the recombinant RNA molecules of any one of Embodiments 46-48.2, and a carrier suitable for administration to a mammalian subject.

Embodiment 50. A particle comprising the recombinant RNA molecules of any one of Embodiments 46-48.2.

Embodiment 51. The particle of Embodiment 50, wherein the particle is selected from the group consisting of a nanoparticle, an exosome, a liposome, and a lipoplex.

Embodiment 52. The particle of Embodiment 51, wherein the particle is a lipid nanoparticle.

Embodiment 53. A pharmaceutical composition comprising a plurality of particles according to any one of Embodiments 50-52.

Embodiment 54. The pharmaceutical composition of Embodiment 53, wherein delivery of the composition to a subject delivers the encapsulated recombinant RNA molecule to a target cell, and wherein the encapsulated recombinant RNA molecule produces an infectious virus capable of lysing the target cell.

Embodiment 55. A method of killing a cancerous cell comprising exposing the cancerous cell to the particle of any one of Embodiments 50-52, or compositions thereof, under conditions sufficient for the intracellular delivery of the particle to said cancerous cell, wherein the replication-competent virus produced by the encapsulated polynucleotide results in killing of the cancerous cell.

Embodiment 56. The method of Embodiment 55, wherein the method is performed in vivo, in vitro, or ex vivo.

Embodiment 57. A method of treating a cancer in a subject comprising administering to a subject suffering from the cancer an effective amount of the particle of any one of Embodiments 50-52, or compositions thereof.

Embodiment 58. The method of Embodiment 57, wherein the cancer is lung cancer, breast cancer, colon cancer, or pancreatic cancer, and wherein the synthetic RNA viral genome comprises a polynucleotide sequence derived from the KY strain.

Embodiment 59. The method of any of Embodiments 55-58, wherein the cancer is bladder cancer, renal cell carcinoma, ovarian cancer, gastric cancer or liver cancer, and wherein the synthetic RNA viral genome comprises a polynucleotide sequence derived from the EF strain.

Embodiment 60. The method of any one of Embodiments 55-58, wherein the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL).

Embodiment 61. The method of any of Embodiments 55-58, wherein the cancer is selected from the groups consisting of lung cancer, breast cancer, colon cancer, pancreatic cancer, bladder cancer, renal cell carcinoma, ovarian cancer, gastric cancer and liver cancer.

Embodiment 62. The method of any of Embodiments 55-58, wherein the cancer is renal cell carcinoma, lung cancer, or liver cancer.

Embodiment 63. The method of any of Embodiments 55-58, wherein the cancer is small cell lung cancer or non-small cell lung cancer (e.g., squamous cell lung cancer or lung adenocarcinoma).

Embodiment 64. The method of any of Embodiments 55-58, wherein the cancer is hepatocellular carcinoma (HCC) (e.g., Hepatitis B virus associated HCC).

Embodiment 65. The method of any of Embodiments 55-58, wherein the cancer is treatment-emergent neuroendocrine prostate cancer.

Embodiment 66. The method of any of Embodiments 55-58, wherein the cancer is lung cancer, liver cancer, prostate cancer (e.g., CRPC-NE), bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma, or melanoma.

Embodiment 67. The method of any of Embodiments 55-58, wherein the cancer is neuroblastoma.

### EXAMPLES

The following examples are given for the purpose of illustrating various embodiments of the disclosure and are not meant to limit the present disclosure in any fashion. The present examples; along with the methods described herein are presently representative of preferred embodiments; are exemplary; and are not intended as limitations on the scope of the disclosure. Changes therein and other uses which are encompassed within the spirit of the disclosure as defined by the scope of the claims will occur to those skilled in the art.

### Example 1: Production of Infectious Picornavirus Virus from Recombinant RNA Molecules

Experiments were performed to assess the ability to produce infectious CVA21 virus from recombinant RNA molecules. Briefly, RNA polynucleotides comprising CVA21 viral genomes were generated by T7 transcription in vitro and 293T cells were transfected with 1 μg of the CVA21-RNA constructs in Lipofectamine RNAiMax for 4 hours, cells were washed, and complete media was added to each well. Supernatants from transfected 293T were collected after 72 hours, syringe filtered with 0.45 μM filter and serially diluted onto NCI-H1299 cells. After 48 hours, supernatants were removed from the NCI-H1299 cultures and cells were stained with crystal violet to assess viral infectivity. RNA molecules comprising CVA21 viral genomes produced active lytic virus (data not shown).

In addition, supernatants of NCI-H1299 cells treated with 1 μg of CVA21-RNA lipid, CVA21 plasmid DNA, or CVA21-Negative pDNA control were collected after 72 hours and serially diluted onto uninfected NCI-H1299 cells. Cell viability assays were performed according to standard protocols. CVA21-RNA/LNP are capable of producing infectious virus that results in tumor cell lysis in vitro (data not shown).

### Example 2: Formulation of Lipid Nanoparticles for Intravenous Delivery of CVA21-Encoding RNA

Recombinant RNA molecules comprising CVA21 genomes were formulated in lipid nanoparticles for delivery of the RNA in vivo.

### Lipid Nanoparticle Production:

The following lipids were used in formulation of lipid nanoparticles:
(a) D-Lin-MC3-DMA (MC3);
(b)N-(2,3-dioleoyloxy) propyl)-N,N,N-trimethylammonium chloride (DOTAP)
(c) COATSOME® SS-LC (former name: SS-18/4PE-13);
(d) COATSOME® SS-EC (former name: SS-33/4PE-15);
(e) COATSOME® SS—OC;
(f) COATSOME® SS—OP;
(g) Di((Z)-non-2-en-1-yl) 9-((4-dimethylamino) butanoyl)oxy) heptadecanedioate (L-319)
(h) cholesterol;
(i) 1,2-distearoyl-sn-glycero-3-phosphocholine (DSPC);
(j) 1,2-dilauroyl-sn-glycero-3-phosphoethanolamine (DLPE);
(k) 1,2-dioleoyl-sn-glycero-3-phosphocholine (DOPC);
(l) 1,2-dioleoyl-sn-glycero-3-phosphoethanolamine (DOPE);
(m) 1,2-distearoyl-sn-glycero-3-phosphoethanolamine-N-[amino (polyethyleneglycol)-5000] (DSPE-PEG5K);
(n) 1,2-dipalmitoyl-rac-glycerol methoxypolyethylene glycol-2000 (DPG-PEG2K);
(o) 1,2-distearoyl-rac-glycero-3-methylpolyoxyethylene-2000 (DSG-PEG2K);
(p) 1,2-dimyristoyl-rac-glycero-3-methylpolyoxyethylene-2000 (DMG-PEG2K)
(q) polyoxyethylene (100) stearyl ether (BRIJ™ S100; CAS number: 9005-00-9);
(r) polyoxyethylene (20) stearyl ether (BRIJ™ S20; CAS number: 9005-00-9);
(s) polyoxyethylene (20) oleyl ether (BRIJ™ O20; CAS number: 9004-98-2);
(t) polyoxyethylene (20) cetyl ether (BRIJ™ C20, CAS number: 9004-95-9);
(u) Polyoxyethylene (40) stearate (MYRJ™ S40, CAS number: 9004-99-3).

Lipids were prepared in ethanol at various ratios. RNA lipid nanoparticles were then generated using microfluidic micromixture (Precision NanoSystems, Vancouver, BC) at a combined flow rate of 2 mL/min (0.5 mL/min for ethanol, lipid mix and 1.5 mL/min for aqueous buffer, RNA). The resulting particles were washed by tangential flow filtration with PBS containing Ca and Mg.

### Analysis of Physical Characteristics of Lipid Nanoparticles:

Physical characteristics of lipid nanoparticles were evaluated before and after tangential flow filtration. Particle size distribution and zeta potential measurements were determined by light scattering using a Malvern Nano-ZS Zetasizer (Malvern Instruments Ltd, Worcestershire, UK). Size measurements were performed in HBS at pH 7.4 and zeta potential measurements were performed in 0.01 M HBS at pH 7.4. Percentage of RNA entrapment was measured by Ribogreen assay. Lipid nanoparticles that showed greater than 80 percent RNA entrapment were tested in vivo.

### Example 3: In Vivo Efficacy of CVA21-Encoding RNA Lipid Nanoparticles in Melanoma

Experiments were performed to determine the ability of lipid nanoparticles comprising CVA21-encoding RNA molecules to produce infectious virus and inhibit melanoma tumor growth in vivo. CVA21 RNA lipid nanoparticle production, formulation, and analysis are described in Example 2.

The ability of CVA21 RNA lipid nanoparticles to inhibit tumor growth was evaluated using the SK-MEL28 xenograft model. Briefly, SK-MEL28 cells (1×10⁶ cells/0.1 mL in a 1:1 mixture of serum-free PBS and Matrigel®) were subcutaneously inoculated in the right flank of 8-week-old female athymic nude mice (Charles River Laboratories). When median tumor size reached approximately 150 mm³ (120-180 mm³ range), mice were intratumorally administered either PBS or CVA21-encoding RNA formulated with Lipofectamine RNAiMAx (1 μg), or intravenously administered CVA21-encoding RNA lipid nanoparticles (formulation ID: 70032-6C, 5 μg). Mice received intratumoral treatments on days 1 and 5, or intravenous treatment on days 1, 6, 11, and 16. Tumor volume was measured 3 times per week using electronic calipers.

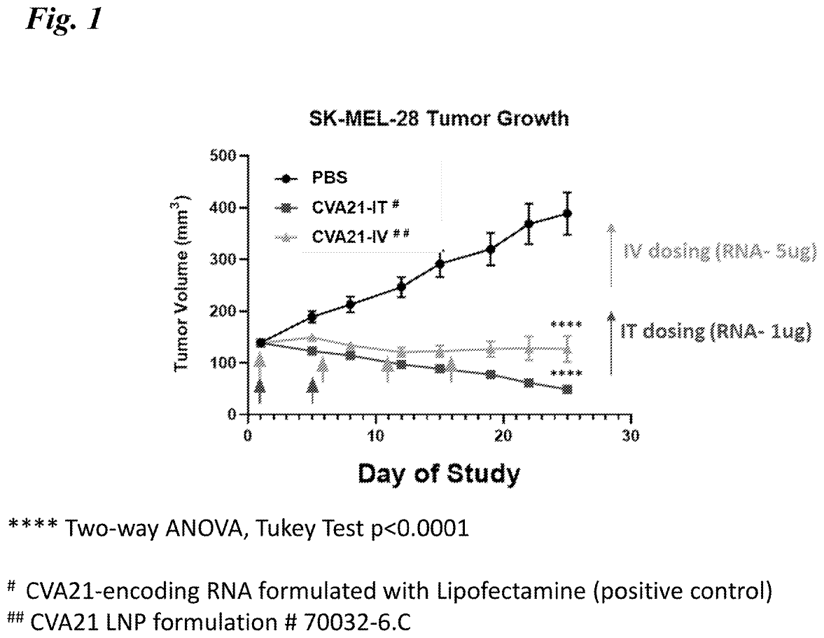

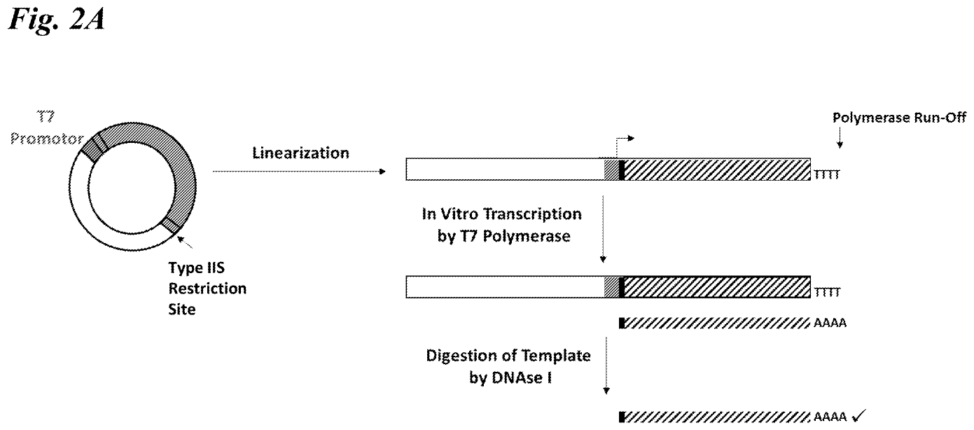

As shown in FIG. 1, intravenous treatment with LNPs comprising CVA21 Kuykendall strain RNA molecules (formulation ID: 70032-6C; MC3:Chol:DSPC:DPG-PEG5K is about 49:39.8:11:0.2 mol %) or intratumoral treatment of CVA21-Kuykendall strain RNA molecules formulated with Lipofectamine prevented tumor growth in tumor-bearing mice compared to mice treated with PBS (two-way ANOVA, p<0.0.001). Collectively, these results suggest that lipid nanoparticles comprising CVA21 RNA molecules are an effective therapeutic strategy for the treatment of melanoma.

### Example 4: Strategies for Generation of Discrete 3′ Termini of CVA21

As described above, the synthetic genomes described herein require discrete 3′ and 5′ ends native to the virus in order to produce a replication-competent and infective virus from the synthetic genome. The RNA transcripts produced by T7 RNA polymerase in vitro mammalian 5′ and 3′ UTRs and therefore do not contain the discrete, native ends required for production of an infectious ssRNA virus.

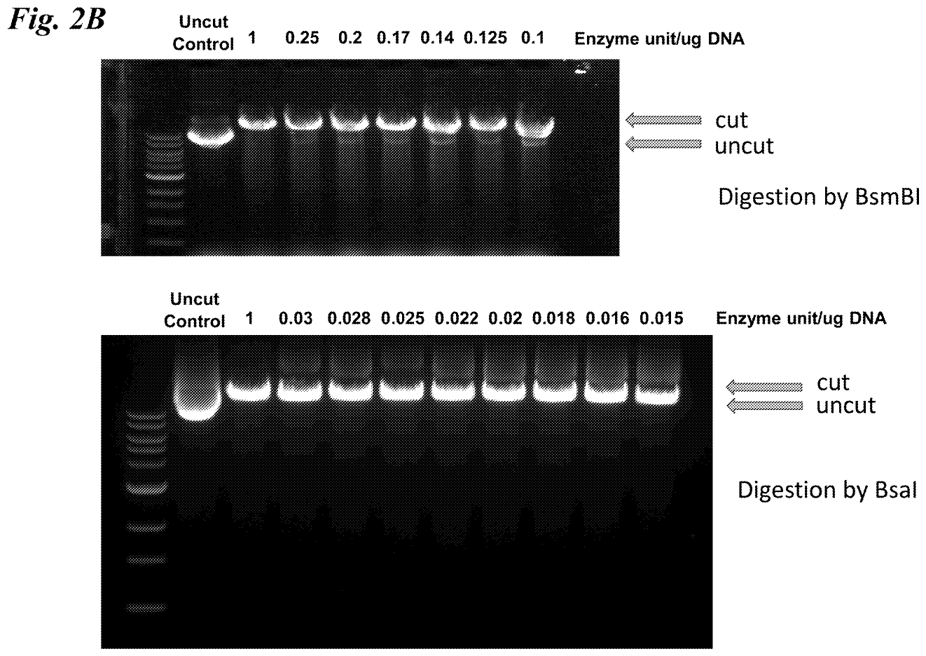

A strategy using 3′ restriction enzyme recognition sequences was employed to generate the discrete 3′ ends required for infectious CVA21. The Type IIS restriction recognition sequence (e.g., BsmBI, BsaI, or SapI recognition sequence) was inserted at the 3′ end of the DNA template. The corresponding restriction enzyme (e.g., BsmBI, BsaI, or SapI) cleaves 5′ of its recognition site to generate a polythymidine run of the appropriate length to generate the discrete virus polyadenylation site native to the virus. This process is illustrated in FIG. 2A.

Both BsaI-HF®v2 and BsmBI-v2 (New England Biolabs) were tested for their efficiency of generating discrete 3′ ends of DNA templates. DNA templates containing either a BsmBI recognition sequence or a BsaI recognition sequence were constructed and subjected to digestion by the corresponding enzyme at the appropriate condition provided in the product manuals. As shown in FIG. 2B (upper gel image), BsmBI achieved complete digestion of the corresponding DNA construct at 1 enzyme unit/μg DNA concentration, but lower concentrations of BsmBI led to incomplete digestion. On the other hand, as shown in FIG. 2B (lower gel image), BsaI achieved completed digestion of the corresponding DNA construct at a lower concentration of 0.015 enzyme unit/μg DNA, suggesting that incorporating a BsaI recognition sequence after the poly-A tail region and the use of corresponding BsaI restriction enzyme can efficiently generate the discrete 3′ end of the in vitro DNA template for the RNA viral genome.

### Example 5: An RNaseH Strategy for Generation of Discrete 5′ Termini of CVA21

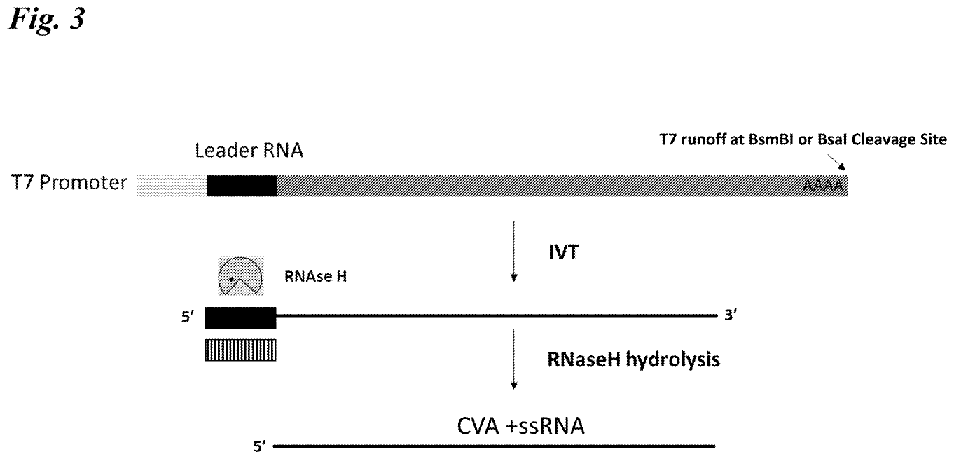

An RNAseH strategy was employed to generate the discrete 5′ termini native to CVA21. The T7 leader must be removed to generate an authentic terminus for the virus. Depicted in FIG. 3 is a diagram of the in vitro transcription (IVT) and 5′ leader processing approach. The IVT template is depicted at the top and the resulting RNA transcript is illustrated in the middle. This CVA21+ssRNA transcript is then annealed to a complementary dsDNA oligo (dashed box) and that portion is hydrolyzed with RNaseH. The final viral ssRNA product, with the correct 5′ terminus, is shown at the bottom.

This strategy, in combination with the 3′ restriction enzyme strategy, produces a final synthetic CVA21 genome with the discrete 5′ and 3′ termini required for production of infectious CVA21.

### Example 6: A Ribozyme Strategy for Generation of Discrete 5′ Termini of CVA21

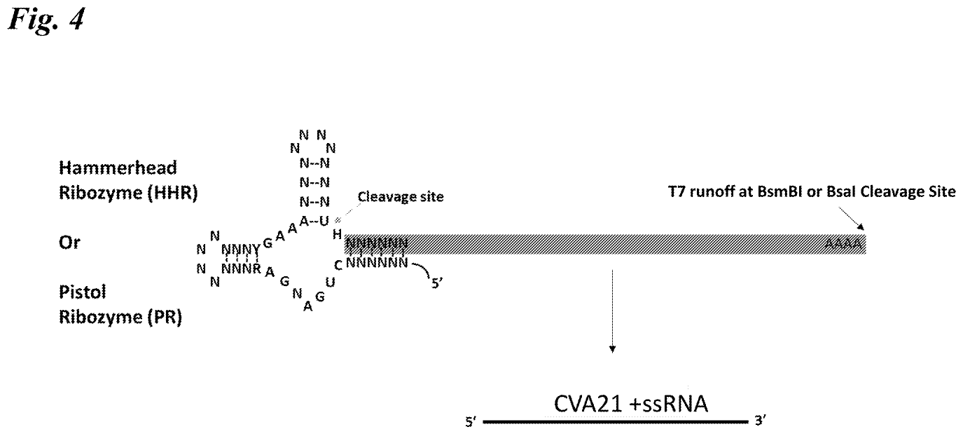

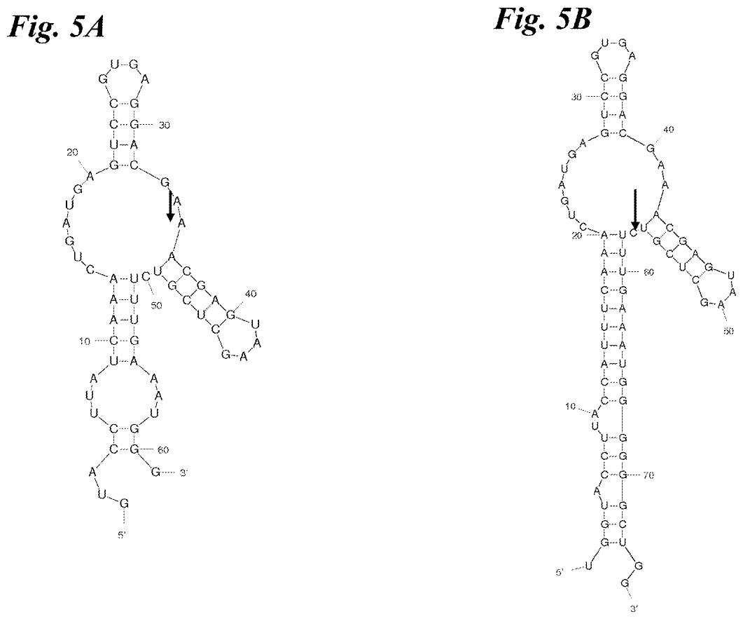

A ribozyme strategy was employed to generate the discrete 5′ termini native to CVA21. A schematic of this approach is illustrated in FIG. 4, showing the design of ribozymes to cleave at the 5′ terminus of a picornavirus. The two ribozymes depicted are hammerhead and pistol ribozymes, however multiple other ribozymes could be adapted to cleave specifically in this context.

Modifications of the hammerhead and pistol ribozymes for implementation in this strategy are shown in FIG. 5 and FIG. 6, respectively. A structural model of a minimal hammerhead ribozyme (HHR) that anneals and cleaves the 5′ end of CVA21 is shown in FIG. 5A (this ribozyme cleaves the 5′ end at the site indicated by the arrow). A structural model of hammerhead ribozyme with a stabilized stem I for cleavage of the CVA21 5′ terminus (STBL) is shown in FIG. 5B (this ribozyme cleaves the 5′ end at the site indicated by the arrow). FIG. 6A shows a schematic of Pistol ribozyme characteristics found in the wild (Pistol WT). FIG. 6B shows a Pistol ribozyme from P. polymyxa modeled by mFOLD with a tetraloop added to fuse the P3 strands.

### Example 7: Optimization of 5′ Ribozyme Sequence

FIG. 10A provides a general schematic of a non-limiting example of the CVA21 expression construct design and corresponding in vitro transcription process to generate synthetic RNA viral genomes with precise 5′ and 3′ end. Picornaviruses such as CVA21 require specific identity of RNA termini for efficient viral replication. Nucleotide “U” at the 5′-end is required for covalent modification with VPg and PolyA tail at the 3′-end is required for (−) strand synthesis priming and RNA stability. On the other hand, eukaryotic promoters typically have a different RNA 5′ end identity requirement for RNA production. For example, T7 RNA polymerase strongly prefers to start with 5′-GGG, which is not optimal for efficient viral replication.

Experiments were performed to optimize the ribozyme sequence at the 5′ junctional cleavage sequence region of the in vitro transcription template for CVA21 viral genome based on the test construct design according to FIG. 10B. Initially, a short, about 60 nt segment(S) of the 5′-end of the viral genome (“virus start”) was used to identify ribozymes that yielded high cleavage efficiency. However, the candidate ribozymes with high cleavage efficiency for the short virus start segment displayed much lower cleavage efficiency when used for production of the much longer, full-length viral genome RNA. Specifically, for an initial candidate ribozyme that yielded >90% cleavage efficiency in the test construct with short ˜60 nt virus start, it yielded only about 10-35% cleavage efficiency upon incorporation of the expression construct for the full-length viral genome. It is hypothesized that the short ˜60 nt virus start did not have secondary structure elements presented in the longer viral genome construct that affected ribozyme folding and cleavage. Thus, the test construct was redesigned to include a longer, about 260 nt segment (L) of the 5′-end of the viral genome sequence. As shown in FIG. 10C, the initial candidate ribozyme, which displayed a high cleavage efficiency for the shorter 60 nt segment virus start, had a much lower cleavage efficiency for the longer ˜260 nt segment virus start. Therefore, the test construct “L” with longer virus start better recapitulates the cleavage efficiency of the candidate ribozyme when incorporated into the full-length viral genome.

Experiments were then performed to screen for ribozymes using the test construct with longer virus start, which identified ENT27-WT (corresponding DNA sequence shown as SEQ ID NO: 130) as a promising candidate of the 5′ junctional cleavage sequence. Further optimization was performed in the DNA template encoding ENT27 ribozyme, including:
Replacing the P2 motif “TTGGTT” of ENT27-WT with “TTTGTT” (ENT27-V1) or “TTTATT” (ENT27-V2, ENT27-V3, ENT27-V4) to complement the 5′ end of the viral start “UUAAA” after ribozyme cleavage.
Inserting additional nucleotides “GAAA” (ENT27-V3) or “ACGAGAAATCGT” (ENT27-V4) into the P3 stem insert of the ENT27 ribozyme.
   These optimizations are illustrated in FIG. 11A and FIG. 11B.

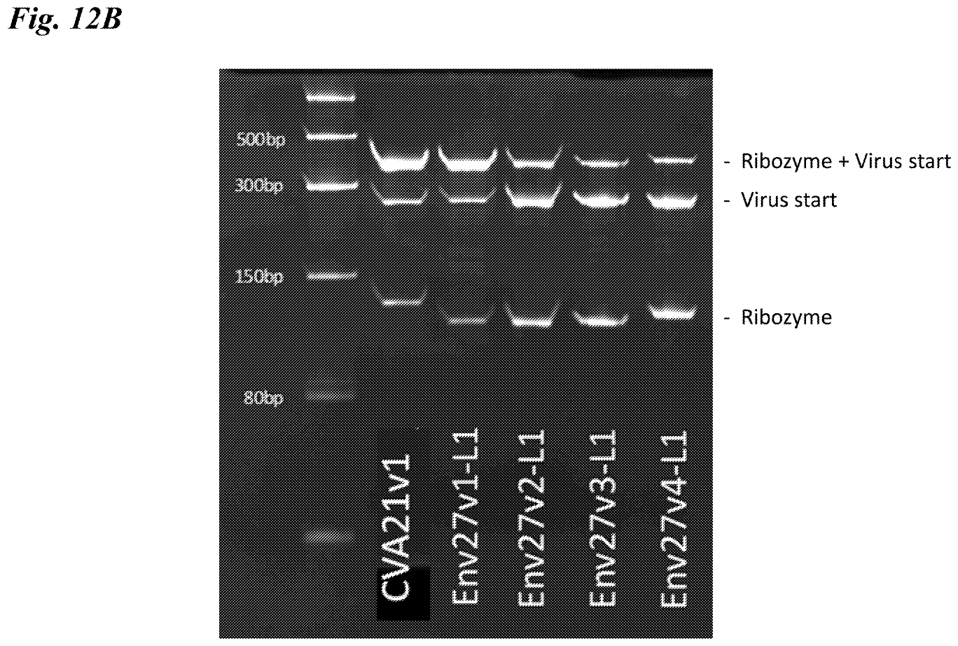

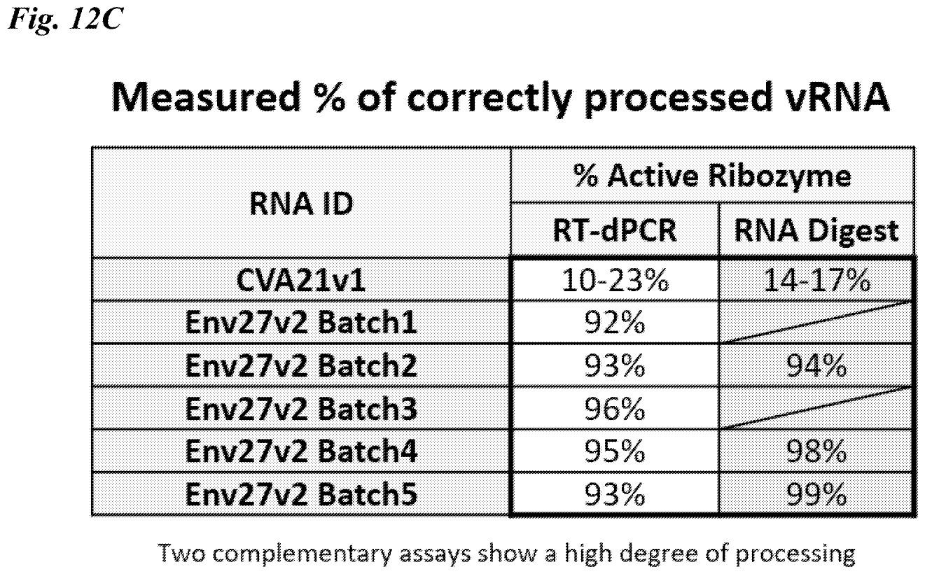

The cleavage efficiencies of these optimized ENT27 ribyzomes were analyzed using the test constructs with longer ˜260 nt virus start. The leader sequence used in these constructs include L1 (SEQ ID NO: 135) and L2 (SEQ ID NO: 136). As shown in FIG. 12A, the test construct with ENV27-V2 ribozyme and L1 leader sequence achieved much better cleavage efficiency than the constructs with either ENV27-V1 ribozyme or L2 leader sequence. And, as shown in FIG. 12B, inserting the additional nucleotides into the P3 stem improved the cleavage efficiency as demonstrated by constructs with ENV27-V3 or ENV27-V4 ribozyme (and L1 leader sequence).

ENV27-V2 ribozyme and L1 leader sequence was then incorporated into the CVA21 viral genome expression construct and tested for cleavage efficiency using both RT-dPCR and RNA digest methods. As shown in FIG. 12C, ENV27-V2 with L1 leader sequence consistently yielded >90% cleavage efficiency, much higher than that of a control vector CVA21v1 that used an alternative ribozyme.

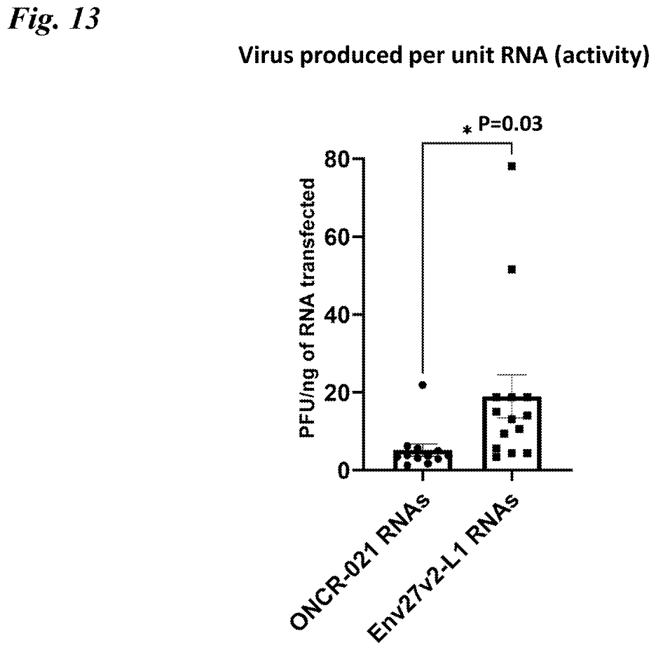

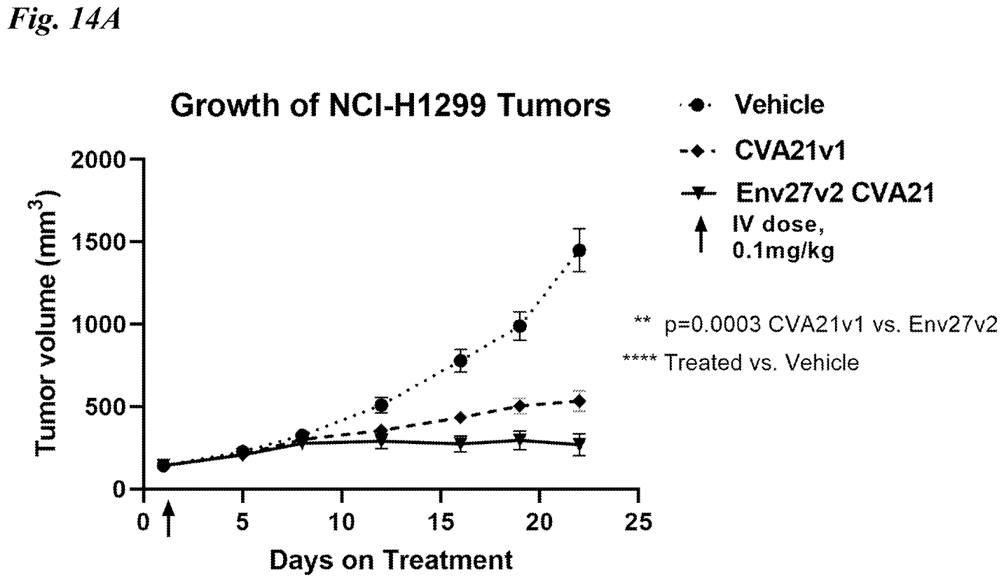

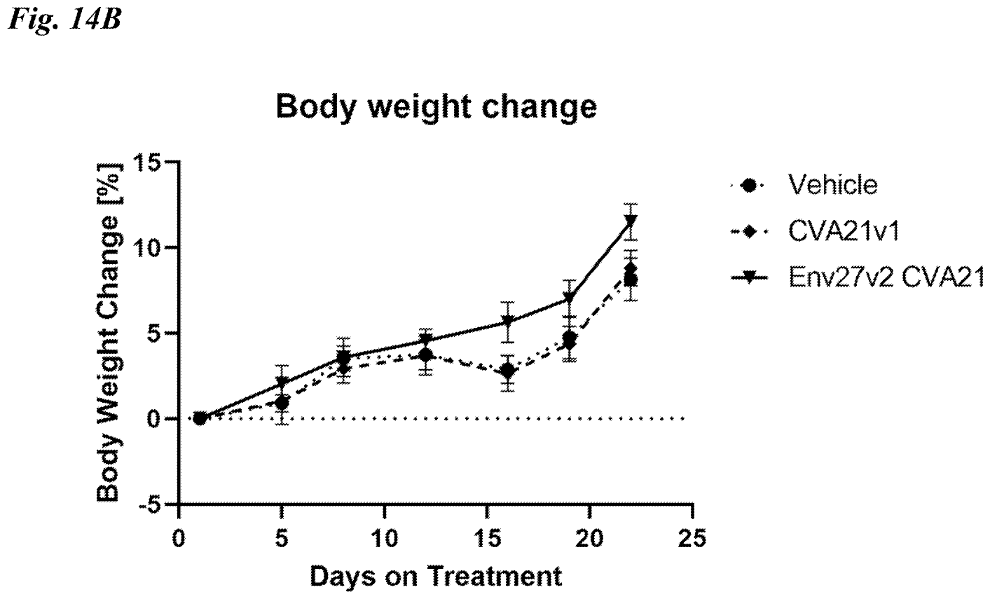

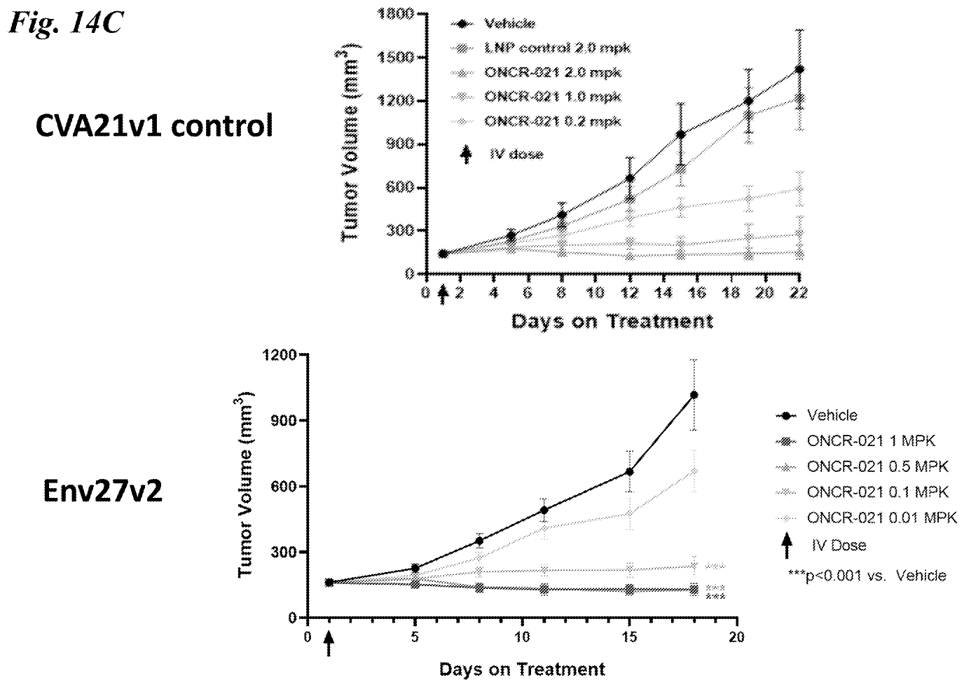

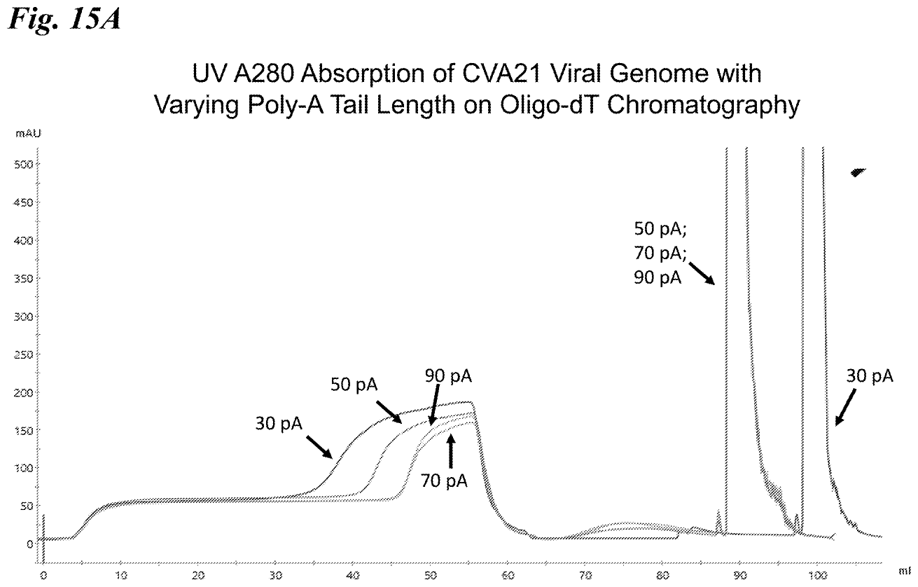

The higher cleavage efficiency of ENV27-V2 ribozyme in the test construct translated to higher potency of the viral genome RNA in cell studies and in vivo animal studies. As shown in FIG. 13, cells transfected with CVA21 viral genome RNAs produced using a DNA template comprising the ENV27-V2 ribozyme and L1 leader sequence (ENV27v2 CVA21) yielded much higher titer of virus as compared to a control DNA template using an alternative ribozyme (CVA21v1). Similarly, as shown in FIG. 14A, in a NSCLC xenograft mouse model, CVA21 viral genome RNAs produced using the ENV27v2 CVA21 template also exhibited much better efficacy of tumor inhibition than those produced using the control CVA21v1 DNA template. This result was reproduced in a second animal model study, in which 0.1 mg/kg dose of the LNP comprising viral genomes derived from the ENV27v2 CVA21 template more effectively inhibited tumor growth than the control LNP comprising viral genomes derived from the control CVA21v1 template (FIG. 14C).

These results demonstrate that a DNA template encoding an optimized ENV27 ribozyme as 5′ junctional cleavage sequence can efficiently induce ribozyme cleavage at the start of the transcribed viral genome RNA and expose the native 5′ end of the viral genome RNA product.

### Example 8: Optimization of Poly-a Tail Sequence

Experiments were performed to optimize the length of the poly-A tail attached to the CVA21 viral genome or SVV viral genome.

Four different lengths of poly-A tails (30 pA, 50 pA, 70 pA, and 90 pA) were assessed by cloning into the corresponding region of the recombinant DNA molecule encoding the CVA21 or SVV viral genome. Purification assays were performed to assess the purification efficiency and recovery rate of the resulting RNA viral genomes on a monolith Oligo-dT chromatography at the following conditions: flow rate: 1 mL/min; loading concentration: 0.1 mg/mL; binding condition: 500 mM NaCl.

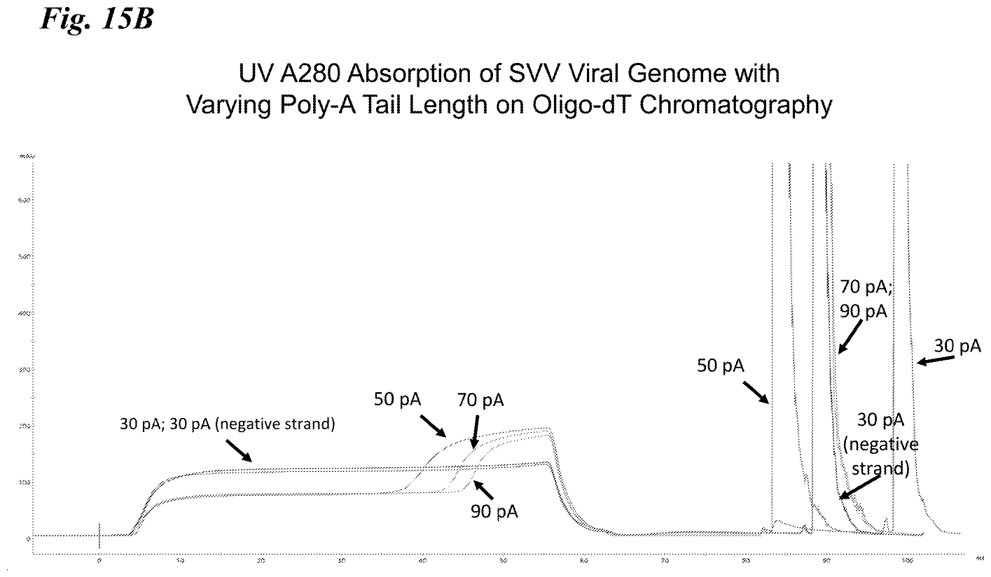

As shown in Table 15A below, a longer poly-A tail (>30 pA) resulted in a higher binding capacity and higher recovery rate of CVA21 RNA viral genome molecules after elution but extending the length of poly-A tail beyond 70 pA provided minimal further improvement of purification efficiency. A representative chromatography profile is shown in FIG. 15A.

TABLE 15A
Oligo-dT Chromatography of CVA21-RNA
with Varying Poly-A Tail Lengths

| Poly-A Tail | Recovery | Binding Capacity |
|---|---|---|
| Length | % after Elution | (mg/mL) |
| 30 pA | 44.5 | 3 |
| 50 pA | 47.3 | 3.77 |
| 70 pA | 49.5 | 4.24 |
| 90 pA | 50.3 | 4.24 |

Similar experiments were performed to assess the purification efficiency and recovery rate of SVV RNA viral genome molecules with varying poly-A tail length, at the following conditions: flow rate: 1 mL/min; loading concentration: 0.1 mg/mL; binding condition: 500 mM NaCl. A negative strand control of SVV-RNA molecule with 30 pA tail was also included as an additional control.

As shown in Table 15B below, a longer poly-A tail resulted in higher binding capacity and higher recovery rate of SVV RNA viral genome molecules after elution but extending the length of poly-A tail beyond 70 pA provided minimal further improvement of purification efficiency. A representative chromatography profile is shown in FIG. 15B.

TABLE 15B
Oligo-dT Chromatography of SVV-RNA
with Varying Poly-A Tail Length

| Poly-A Tail | Recovery % | Binding Capacity |
|---|---|---|
| Length | after Elution | (mg/mL) |
| 30 pA | 33.1 | N/A |
| 50 pA | 52 | 3.41 |
| 70 pA | 56.3 | 3.9 |
| 90 pA | 57.7 | 4.12 |
| 30 pA | 32.4 | N/A |
| (negative strand) | | |

The synthetic RNA viral genomes with the 70 pA length poly-A tails showed increased binding capacity and recovery on oligo-dT chromatography column.

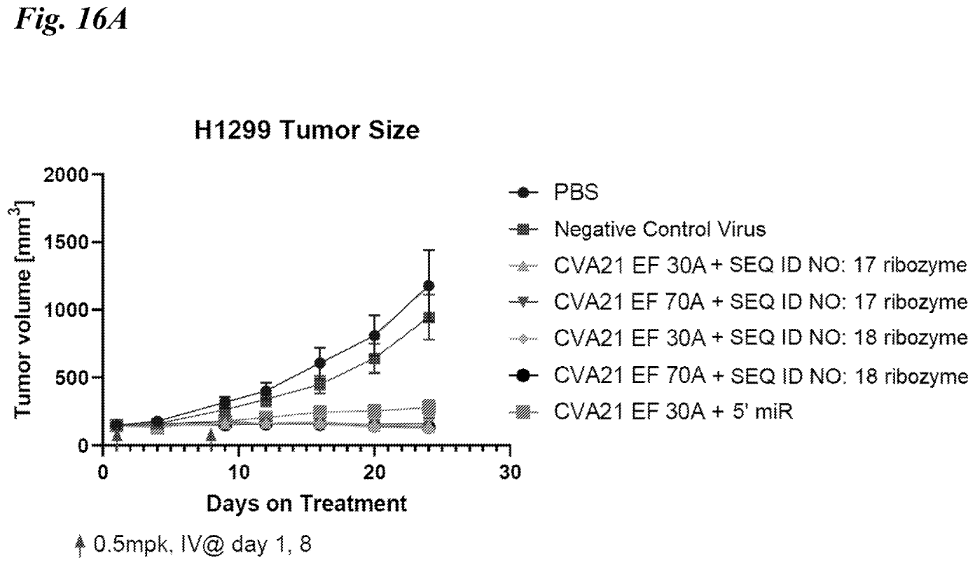

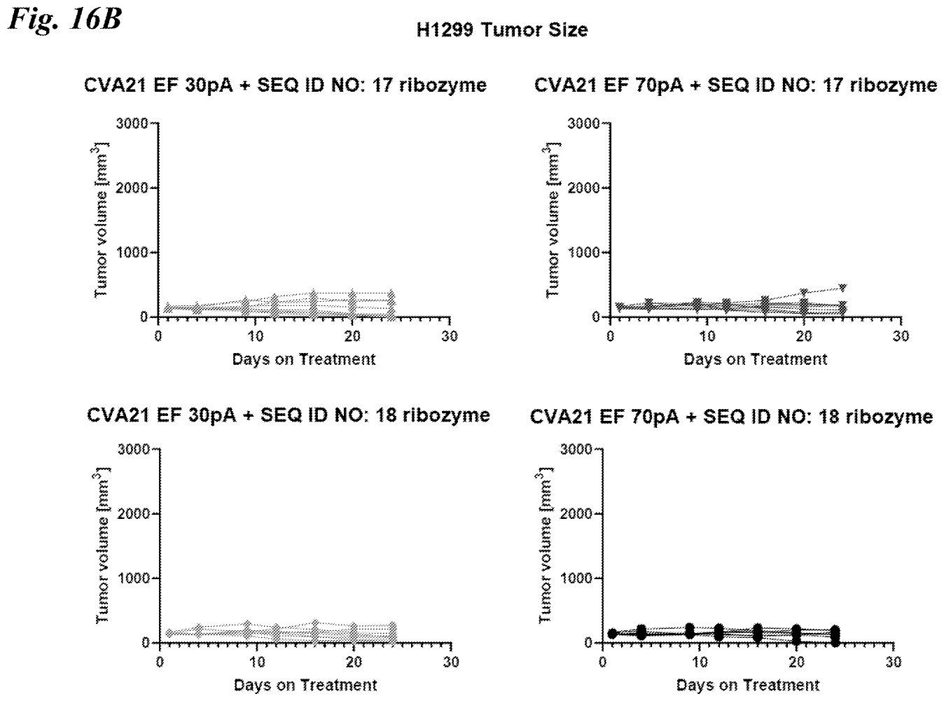

The anti-tumor efficacy of the synthetic CVA21-EF strain viral genomes produced with the 70 pA poly-A tail versus the 30 pA poly-A tail, and the ribozyme sequence SEQ ID NO: 18 versus SEQ ID NO: 17, were then compared. A mouse lung cancer model based on NCI-H1299 cells was used. As shown in FIG. 16A and FIG. 16B, the CVA21-EF strain viral genome produced with the 70 pA poly-A tail and the 5′ ribozyme sequence according to SEQ ID NO: 18 displayed similar or even better anti-tumor efficacy as compared to the other viral genome designs.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

While preferred embodiments of the present disclosure have been shown and described herein; it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A recombinant DNA molecule comprising, from 5′ to 3′, a promoter sequence, a 5′ junctional cleavage sequence, and a polynucleotide sequence encoding an RNA molecule comprising a synthetic RNA viral genome, wherein the 5′ junctional cleavage sequence comprises or consists of a ENV27 ribozyme encoding sequence.

2. The recombinant DNA molecule of claim 1, wherein the ENV27 ribozyme encoding sequence comprises or consists of a polynucleotide sequence (excluding P3 stem insert) having at least 80% identity to SEQ ID NO: 132 (excluding its P3 stem insert corresponding to nucleotides 49-54 of SEQ ID NO: 132).

3. The recombinant DNA molecule of claim 2, wherein the polynucleotide sequence (excluding P3 stem insert) is 100% identical, or has at most 1, at most 2, at most 3, at most 4, at most 5, at most 6, at most 7, at most 8, at most 9, at most 10, or at most 11 mutations (insertions, deletions or substitutions), as compared to SEQ ID NO: 132 (excluding its P3 stem insert corresponding to nucleotides 49-54 of SEQ ID NO: 132).

4. The recombinant DNA molecule of any one of claims 1-3, wherein the polynucleotide sequence is 100% identical, or has at most 1, at most 2, at most 3, at most 4, at most 5, at most 6, at most 7, at most 8, at most 9, at most 10, or at most 11 mutations (insertions, deletions or substitutions), as compared to any one of SEQ ID NO: 130-134.

5. The recombinant DNA molecule of claim 3 or 4, wherein the mutation(s) are substitution(s).

6. The recombinant DNA molecule of any one of claims 1-5, wherein the ENV27 ribozyme encoding sequence comprises the polynucleotides “TTTATT” or “TTTGTT” at the positions corresponding to nucleotides 25-30 of SEQ ID NO: 132.

7. The recombinant DNA molecule of claim 6, wherein the ENV27 ribozyme encoding sequence comprises the polynucleotides “TTTATT” at the positions corresponding to nucleotides 25-30 of SEQ ID NO: 132.

8. The recombinant DNA molecule of any one of claims 2-7, wherein the ENV27 ribozyme encoding sequence comprises the P3 stem insert of about 1-30, about 1-20, about 6-20, or about 6-10 polynucleotides in length.

9. The recombinant DNA molecule of any one of claims 2-8, wherein the P3 stem insert is at least 1, 2, 3, 4, 5, 6, 7, 8, 9, 10, 11, 12, 13, 14, 15, 16, 17, 18, 19, or 20 polynucleotides in length.

10. The recombinant DNA molecule of any one of claims 2-9, wherein the P3 stem insert is 1, 2, 3, 4, 5, 6, 7, 8, 9, 10, 11, 12, 13, 14, 15, 16, 17, 18, 19, or 20 polynucleotides in length.

11. The recombinant DNA molecule of any one of claims 8-10, wherein the P3 stem insert comprises or consists of the polynucleotides “AGATCT” at the region corresponding to nucleotides 49-54 of SEQ ID NO: 132.

12. The recombinant DNA molecule of any one of claims 8-10, wherein the P3 stem insert comprises or consists of the polynucleotides “AGAGAAATCT” (SEQ ID NO: 137) at the region corresponding to nucleotides 49-54 of SEQ ID NO: 132.

13. The recombinant DNA molecule of any one of claims 8-10, wherein the P3 stem insert comprises or consists of the polynucleotides “AGAACGAGAAATCGTTCT” (SEQ ID NO: 138) at the region corresponding to nucleotides 49-54 of SEQ ID NO: 132.

14. The recombinant DNA molecule of any one of claims 1-13, comprising, from 5′ to 3′, the promoter sequence, the 5′ junctional cleavage sequence, the polynucleotide sequence encoding the RNA molecule comprising the synthetic RNA viral genome, and a poly-A tail.

15. The recombinant DNA molecule of claim 14, comprising, from 5′ to 3′, the promoter sequence, the 5′ junctional cleavage sequence, the polynucleotide sequence encoding the RNA molecule comprising the synthetic RNA viral genome, the poly-A tail, and a 3′ junctional cleavage sequence.

16. The recombinant DNA molecule of any one of claims 1-15, wherein the synthetic RNA viral genome encodes a picornavirus.

17. The recombinant DNA molecule of claim 16, wherein the picornavirus is a coxsackievirus virus.

18. The recombinant DNA molecule of any one of claims 1-17, wherein the 5′ end of the RNA viral genome starts with “UUAAA”.

19. The recombinant DNA molecule of any one of claims 17-18, wherein the Coxsackievirus is a CVA21 strain.

20. The recombinant DNA molecule of claim 19, wherein the CVA21 strain is selected from the Kuykendall strain, the EF strain and the KY strain.

21. The recombinant DNA molecule of any one of claims 1-20, wherein the 5′ end of the RNA viral genome comprises a polynucleotide sequence having at least 80%, at least 85%, at least 90%, at least 91%, at least 92%, at least 93%, at least 94%, at least 95%, at least 96%, at least 97%, at least 98%, at least 99%, or 100% identity to nucleotides 1-260 of any one of SEQ ID NO: 1, 5, or 9.

22. The recombinant DNA molecule of any one of claims 1-21, wherein the recombinant DNA molecule does not comprise additional nucleic acid between the 5′ junctional cleavage sequence and the polynucleotide sequence encoding the RNA molecule.

23. The recombinant DNA molecule of any one of claims 1-22, wherein cleavage of the 5′ junctional cleavage sequence and/or the 3′ junctional cleavage sequence produces native 5′ and/or 3′ ends of the synthetic RNA viral genome after transcription.

24. The recombinant DNA molecule of any one of claims 1-23, further comprising a leader sequence between the promoter sequence and the 5′ junctional cleavage sequence.

25. The recombinant DNA molecule of claim 24, wherein the leader sequence is less than 100 bp, less than 90 bp, less than 80 bp, less than 70 bp, less than 60 bp, less than 50 bp, or less than 40 bp in length.

26. The recombinant DNA molecule of any one of claims 24-25, wherein the leader sequence comprises or consists of a polynucleotide sequence having at least 70%, at least 80%, at least 90%, at least 95%, at least 96%, at least 97%, at least 98%, at least 99%, or 100% sequence identity according to SEQ ID NO: 135 or 136.

27. The recombinant DNA molecule of any one of claims 24-25, wherein the leader sequence comprises or consists of a polynucleotide sequence having at least 70%, at least 80%, at least 90%, at least 95%, at least 96%, at least 97%, at least 98%, at least 99%, or 100% sequence identity according to SEQ ID NO: 135.

28. The recombinant DNA molecule of any one of claims 24-27, wherein the recombinant DNA molecule does not comprise additional nucleic acid between the promoter sequence and the leader sequence.

29. The recombinant DNA molecule of any one of claims 24-28, wherein the recombinant DNA molecule does not comprise additional nucleic acid between the leader sequence and the 5′ junctional cleavage sequence.

30. The recombinant DNA molecule of any one of claims 1-27, wherein the promoter sequence is a T7 promoter sequence.

31. The recombinant DNA molecule of claim 30, wherein the T7 promoter sequence comprises or consists of SEQ ID NO: 91.

32. The recombinant DNA molecule of any one of claims 1-31, wherein the poly-A tail is about 50-90 bp in length or about 65-75 bp in length.

33. The recombinant DNA molecule of claim 32, wherein the poly-A tail is about 70 bp in length.

34. The recombinant DNA molecule of any one of claims 1-31, wherein the poly-A tail is about 10-50 bp, or 25-35 bp in length.

35. The recombinant DNA molecule of any one of claims 1-34, wherein the 3′ junctional cleavage sequence comprises or consists of a ribozyme sequence.

36. The recombinant DNA molecule of claim 35, wherein the 3′ ribozyme sequence is a hepatitis delta virus ribozyme sequence.

37. The recombinant DNA molecule of any one of claims 1-34, wherein the 3′ junctional cleavage sequence comprises or consists of a restriction enzyme recognition sequence.

38. The recombinant DNA molecule of any one of claims 1-34, wherein the 3′ junctional cleavage sequence comprises or consists of a Type IIS restriction enzyme recognition sequence.

39. The recombinant DNA molecule of any one of claims 1-38, wherein the 3′ junctional cleavage sequence comprises or consists of a BsmBI recognition sequence.

40. The recombinant DNA molecule of any one of claims 1-38, wherein the 3′ junctional cleavage sequence comprises or consists of a BsaI recognition sequence.

41. The recombinant DNA molecule of any one of claims 1-40, wherein the promoter sequence is a T7 promoter sequence, wherein the leader sequence consists of a polynucleotide sequence according to SEQ ID NO: 135, wherein the 5′ junctional cleavage sequence comprises or consists of a ENV27 ribozyme sequence according to any one of SEQ ID NO: 132-134, wherein the poly-A tail is about 70 bp in length, and wherein the 3′ junctional cleavage sequence comprises or consists of a BsmBI recognition sequence.

42. The recombinant DNA molecule of any one of claims 1-40, wherein the promoter sequence is a T7 promoter sequence, wherein the leader sequence consists of a polynucleotide sequence according to SEQ ID NO: 135, wherein the 5′ junctional cleavage sequence comprises or consists of a ENV27 ribozyme sequence according to any one of SEQ ID NO: 132-134, wherein the poly-A tail is about 70 bp in length, and wherein the 3′ junctional cleavage sequence comprises or consists of a BsaI recognition sequence.

43. The recombinant DNA molecule of any one of claims 1-42, wherein the recombinant DNA molecule does not comprise additional nucleic acid within the region spanning the promoter sequence and the 3′ junctional cleavage sequence.

44. A method of producing a recombinant RNA molecule, comprising in vitro transcription of the recombinant DNA molecule of any one of claims 1-43 and purification of the resulting recombinant RNA molecule.

45. The method of claim 44, wherein the recombinant RNA molecule comprises 5′ and 3′ ends that are native to the viral genome encoded by the recombinant RNA molecule.

46. A recombinant RNA molecule, or a plurality of recombinant RNA molecules, transcribed from the recombinant DNA molecule of any one of claims 1-43.

47. The recombinant RNA molecules of claim 46, wherein at least 70%, at least 75%, at least 80%, at least 85%, at least 90%, at least 95%, at least 96%, at least 97%, at least 98%, at least 99%, at least 99.5%, or at least 99.9%, of the recombinant RNA molecules comprise 5′ and 3′ ends that are native to the viral genome encoded by the recombinant RNA molecule.

48. The recombinant RNA molecules of claim 46 or 47, wherein no more than 30%, no more than 25%, no more than 20%, no more than 15%, no more than 10%, no more than 5%, no more than 4%, no more than 3%, no more than 2%, no more than 1%, no more than 0.5%, or no more than 0.1%, of the recombinant RNA molecules comprise an RNA sequence encoded by the ENV27 ribozyme encoding sequence.

49. A composition comprising an effective amount of the recombinant RNA molecules of any one of claims 46-48, and a carrier suitable for administration to a mammalian subject.

50. A particle comprising the recombinant RNA molecules of any one of claims 46-48.

51. The particle of claim 50, wherein the particle is selected from the group consisting of a nanoparticle, an exosome, a liposome, and a lipoplex.

52. The particle of claim 51, wherein the particle is a lipid nanoparticle.

53. A pharmaceutical composition comprising a plurality of particles according to any one of claims 50-52.

54. The pharmaceutical composition of claim 53, wherein delivery of the composition to a subject delivers the encapsulated recombinant RNA molecule to a target cell, and wherein the encapsulated recombinant RNA molecule produces an infectious virus capable of lysing the target cell.

55. A method of killing a cancerous cell comprising exposing the cancerous cell to the particle of any one of claims 50-52, or compositions thereof, under conditions sufficient for the intracellular delivery of the particle to said cancerous cell, wherein the replication-competent virus produced by the encapsulated polynucleotide results in killing of the cancerous cell.

56. The method of claim 55, wherein the method is performed in vivo, in vitro, or ex vivo.

57. A method of treating a cancer in a subject comprising administering to a subject suffering from the cancer an effective amount of the particle of any one of claims 50-52, or compositions thereof.

58. The method of claim 57, wherein the cancer is lung cancer, breast cancer, colon cancer, or pancreatic cancer, and wherein the synthetic RNA viral genome comprises a polynucleotide sequence derived from the KY strain.

59. The method of any of claims 55-58, wherein the cancer is bladder cancer, renal cell carcinoma, ovarian cancer, gastric cancer or liver cancer, and wherein the synthetic RNA viral genome comprises a polynucleotide sequence derived from the EF strain.

60. The method of any one of claims 55-58, wherein the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL).

61. The method of any of claims 55-58, wherein the cancer is selected from the groups consisting of lung cancer, breast cancer, colon cancer, pancreatic cancer, bladder cancer, renal cell carcinoma, ovarian cancer, gastric cancer and liver cancer.

62. The method of any of claims 55-58, wherein the cancer is renal cell carcinoma, lung cancer, or liver cancer.

63. The method of any of claims 55-58, wherein the cancer is small cell lung cancer or non-small cell lung cancer (e.g., squamous cell lung cancer or lung adenocarcinoma).

64. The method of any of claims 55-58, wherein the cancer is hepatocellular carcinoma (HCC) (e.g., Hepatitis B virus associated HCC).

65. The method of any of claims 55-58, wherein the cancer is treatment-emergent neuroendocrine prostate cancer.

66. The method of any of claims 55-58, wherein the cancer is lung cancer, liver cancer, prostate cancer (e.g., CRPC-NE), bladder cancer, pancreatic cancer, colon cancer, gastric cancer, breast cancer, neuroblastoma, renal cell n cancer, rhabdomyosarcoma, medulloblastoma, neuroendocrine cancer, Merkel cell carcinoma, or melanoma.

67. The method of any of claims 55-58, wherein the cancer is neuroblastoma.


SEQUENCE LISTING (138 sequences)

SEQ ID NO: 1 - RNA, 7405 nt, Enterovirus coxsackievirus A21
  source 1..7405: mol_type = unassigned RNA
ttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaaatgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccacttttgggtttggtcatcagaacaaagcagtgtacgttgccggttacaagatttgcaactaccacctagcaaccccaagtgatcacttgaatgcaattagtatgttatgggacagggatttaatggtggtggaatctagagcccagggaactgataccatcgccagatgtagttgcaggtgtggagtttactattgtgaatctaggaggaagtactaccctgtcacttttactggcccaacgtttcgattcatggaagcaaacgactactatccagcaagataccagtctcacatgctgatagggtgcggatttgcagaacccggggactgcggtgggatactgaggtgcactcatggggtaattggtatcattactgcaggaggtgaaggggtagtagcctttgctgacattagagacctctgggtgtatgaagaggaggccatggaacagggaataacaagctacatcgaatctctcggcacagcctttggcgcagggttcacccacacaatcagtgagaaagtgactgaattgacaacaatggttaccagcactatcacagaaaaactactgaaaaacttggtgaaaatagtgtcggctctagtgattgttgtgagaaattatgaggacactaccacgatccttgcaacactagcactactcgggtgtgatatatctccttggcaatggttgaagaagaaggcatgtgacttactagagattccttatgtgatgcgccaaggtgatgggtggatgaagaaattcacagaggcgtgcaatgcagctaaaggcttagagtggattagcaacaaaatttccaagtttatagattggttgaagtgtaaaattatcccagacgctaaggacaaggtggaatttctcaccaagttgaaacagctagacatgttggaaaatcaaattgcaaccatccaccaatcttgccccagccaagaacaacaagagattcttttcaacaatgtgagatggctagcagtccagtcccgtcggtttgcaccattatacgctgtggaggcacgccgaattaacaaaatggagagcacaataaacaattatatacagttcaagagcaaacaccgtattgaaccagtatgtatgctcattcatgggtcaccagggacgggtaaatctatagctacttcattaataggtagagcaatagcagagaaggaaagcacatcagtctattcaatgccacctgacccatctcactttgatggctataaacaacaaggggtagtgattatggacgacctaaaccaaaaccccgatggtatggacatgaaactgttttgccaaatggtatcaacagtggagtttattcctccaatggcctcattagaggagaagggcattttgtttacatctgattatgtcctggcttctaccaactctcattcaattgtaccacccacagtggctcacagtgatgccttaaccagacgatttgcatttgatgtggaggtttacacgatgtctgaacattcagtcaaaggcaaactgaatatggccacggccactcaattgtgtaaggattgtccaacacctgcaaattttaaaaagtgttgccctctcgtttgtggaaaggccttgcaattaatggacaggtacaccagacaaaggttcactgtagatgagattaccacattaatcatgaatgagaaaaacagaagggccaatatcggcaattgcatggaagccttgtttcaaggaccattaaggtataaagatttgaagatcgatgtgaagacagttcccccccctgagtgcatcagtgatttgttacaagcagtggattctcaagaggttagggattactgtgagaagaaaggctggatcgttaacgttactagccagattcaactagaaaggaacatcaatagggccatgactatactccaagctgttaccacattcgcagcagtcgcaggagtagtgtatgtaatgtacaaactcttcgccggtcaacagggtgcatacactggcttgccaaacaaaaaacccaatgtccctactatcagagtcgctaaagtccaggggccaggatttgactacgcagtggcaatggcaaaaagaaacatagttactgcaaccaccaccaagggtgaatttaccatgctaggggtgcatgataatgtagcaatattgccaacccatgccgctccaggagaaaccattattattgatgggaaagaagtagagatcctagatgccagagccttagaagatcaagcgggaaccaatcttgagatcaccattattactctaaaaagaaatgagaagtttagagacatcagatcacatattcccacccaaattactgaaactaacgatggagtgttgatcgtgaacactagcaagtaccccaatatgtatgtccccgttggtgctgtgaccgaacagggatatcttaatctcagtggacgtcaaactgctcgcactttaatgtacaactttccaacaagggcaggccagtgcggaggaatcatcacttgtactggcaaagtcattgggatgcatgttggcgggaacggttcacatgggtttgcagcagccctcaagcgatcatacttcactcaaaatcagggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaaccctacctcagtcggattggattgggttatactgttgtaggggtaaatttttctttaattcggag

SEQ ID NO: 2 - RNA, 713 nt, Enterovirus coxsackievirus A21
  source 1..713: mol_type = unassigned RNA
ttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaa

SEQ ID NO: 3 - RNA, 2637 nt, Enterovirus coxsackievirus A21
  source 1..2637: mol_type = unassigned RNA
atgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccactttt

SEQ ID NO: 4 - RNA, 1389 nt, Enterovirus coxsackievirus A21
  source 1..1389: mol_type = unassigned RNA
ggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaa

SEQ ID NO: 5 - RNA, 7405 nt, Enterovirus coxsackievirus A21
  source 1..7405: mol_type = unassigned RNA
ttaaaacagctctggggttgttcccaccccagaggcccacgtggcggctagtactctggtatcacggtacctttgtacgcctgttttatatcccttcccccgcaactttagaagcttaacaaaagttcaatagcaggggtgcaagccagtacctctatgaacaagcacttctgtctccccggtgaaattacatagactgtacccacggtcaaaagtaattgatccgttatccgcttgagtacttcgagaagcctagtatcaccttggaatcttcgacgcgttgcgctcaacactctaccccgggtgtagcttaggctgatgagtctgggcaccccccaccggtgacggtggcccaggctgcgttggcggcctacccatggctaacgccatgggacgctagttgtgaacaaggtgtgaagagcctattgagctactcaagagtcctccggcccctgaatgcggctaatcccaaccacggagcaattgctcacgatccagtgagtaggttgtcgtaatgcgcaagttcgtggcggaaccgactactttgggtgtccgtgtttccctttttacttatactggctgcttatggtgacaatttacagattgttaccatatagctattggattggccatccagtattgtgttacacatattaaaatacttttctacaagcatcaacaacattgtgtttggaccacaataatcactacaaatgggagctcaagtatcaacccaaaagaccggtgcacacgaaaatcaaaacgtggctgccaatggatccaccattaattacactactatcaactactacaaagatagtgcaagtaactcagccaccagacaagatctttctcaagatccatcaaaatttacagaaccagttaaagatttaatgttgaaaacagcaccagctttgaattcaccaaatgtggaagcatgtggatacagtgaccgtgtaaggcaaattaccctgggaaactcgactattaccacacaagaagcagctaacgctattgttgcttatggtgagtggcccacttacataaatgactcagaagcaaacccagtagatgcacccactgaaccagatgttagtagcaacaggttttacactctggactcagtgtcttggaagactacctcaaggggttggtggtggaaactaccagactgtctaaaagacatggggatgtttggtcagaatatgtattaccactacctggggcgctccggttacaccattcatgtccagtgcaatgcctcaaagtttcaccaaggggcattaggagttttcttgataccagagtttgttatggcctgtaacaccgagagcaagacatcatatgtttcatacatcaacgcaaatcctggtgagaggggtggtgagtttacaaacatttacaatccatcaagcacagatgctagtcagggcagacaatttgcagcgctagattatttactgggttctggtgttttagctggaaatgcatttgtgtacccacaccagatcattaacctgcgtaccaacaatagtgcaactattgtggtaccatatgtgaactcacttgtcattgattgcatggcaaaacacaacaactggggtattgtcatcctgccactggcgccattggcctttgctgcgacatcatcaccacaagtgcctattacagtgaccattgcacccatgtgcgcagaattcaatggattgagaaacatcactattccagtgcatcaaggattaccaacaatgaacacacctggatccaatcagtttctcacatctgatgacttccagtcaccctgcgctctacccaactttgatgtcactccacctatacacatacccggagaagtgaagaatatgatggaactggccgagattgacacgctgatcccaatgaacgcagtggacggaaaggtaaacactatggagatgtatcaaataccattaaatgatgaactaagcaaagcacccatattttgcctgtccctgtcacctgcttctgacaaacgattaagtcatacgatgttgggtgaaattctaaactattacactcattggacagggtccattaggttcacctttctattctgtggtagcatgatggccactggtaagttgcttctcagttattccccaccaggagctaaaccaccaaccaaccgcaaggatgcaatgttgggcacacatatcatctgggacttgggtttacaatccagttgctccatggttgcaccatggatctctaatacggtatacaggcggtgtgcacgtgatgacttcacagagggtgggtttataacttgcttttatcaaaccagaattgttgtgcctgcctcaacccctaccagcatgttcatgttaggctttgtgagtgcatgtccagatttcagcgtcagactgctgagggacacttcccacattagtcaatcaaaactcatagcacgctcacaaggcattgaagatctcatcgattcagcaataaagaatgctttgagagtgtcccaaccgtccacggctcaatcgactgaagcaaccagtggagcaaacagtcaggaagtaccagcactaactgctgtagaaacaggagcatcaggtcaagcaattcccagtgacgtgatggaaaccagacacgtgataaactacaaaactaggtctgagtcatgtcttgaatcattctttgggagagctgcgtgtgtcacaatcctatctctgaccaactcttcaaaaagtggagaggaaaagaaacatttcaacatttggaacatcacgtataccgacactgtgcagctacgtagaaaattagagttcttcacatattccagatttgaccttgaaatgacttttgtgttcacagagaactaccccagtacagctagtggagaagtgcgcaaccaggtataccagattatgtatattccaccaggggcaccccgaccatcatcctgggatgattatacatggcaatcctcctccaacccttctatcttttacatgtacgggaacgcaccgccacggatgtcaatcccctatgtagggattgctaatgcctactcacacttttatgacggttttgcacgagtgccacttgagggtgagaacactgatgctggtgacacgttttatggattggtatccataaatgattttggagtcttggcagtcagagcagtgaaccgcagcaatccacacacaatacacacatccgtgagagtgtacatgaaaccaaaacacattcggtgctggtgccccagaccccctcgcgcagtattatacagaggagaaggagtggacatgatatccagtgcaattttacccctgaccaaagtagattctattactaccttcggatttggacaccaaagtaaggcagtgtatgttgccggttataagatctgcaactaccacctagcaacccctagtgatcatctaaatgcaattagtgtgttgtgggatagggatttaatggtagtagagtctagagcccaaggaactgacaccatagccagatgtagttgtaggtgtggggtctattactgtgaatcaagaaggaagtactaccctgtcactttcaccggtccaacgttccgattcatggaagcgaatgactactacccagcaagataccagtctcacatgctgataggatgtggatttgcagagcctggagattgcggtggaatactaaggtgcacccatggggtgattggtattattactgcagggggtgagggagtagtagcctttgctgacatcagggatctttgggtgtatgaagaggaggccatggaacagggaataacaagctacattgagtctcttggcacagcctttggtgcagggtttactcacacaattagtgaaaaagtgactgagttaactacaatgattaccagtaccattacagaaaaactactaaaaaatttggtgaaaatagtatcagctttagtaattgttgtgagaaattatgaagacactactacagttcttgcaacactagcattacttgggtgtgacatatctccttggcagtggttgaagaaaaaggcatgtgacctactagaaattcctcatgtactgcgccaaggtgatgggtggatgaagaaattcacagaggcttgcaatgcagccaagggcttagaatggattagcaacaaaatttctaagttcatagactggttgaagtgcaaaattattccagaagccaaagataaggtggaatttctcaccaagctgaaacagttagacatgttagaaaatcaaattgcaactattcaccagtcttgccccagccaggagcaacaagagattctcttcaacaatgtgagatggttagcagtccagtcccgtcgatttgcaccactatatgctgtggaagcacggagaattagcaaaatggagaacacaataaacaactacatacagttcaagagcaaacaccgtattgaaccagtatgtatgctcatccatgggtcaccgggaacaggcaaatcaatagccacctcattgataggcagagcgatagcagaaaaggagaatacatcagtttactcaatgccacctgacccatctcactttgatggctacaaacaacaaggggtcgtgattatggatgatttgaatcagaaccctgatggcatggatatgaaactgttttgccaaatggtgtcaacggtggagttcatcccaccgatggcctcactagaggagaagggtattttgtttacatctgattatgtcctagcctccaccaactcccattcaatagcaccacccacggtagctcacagtgatgccttaaccagacgatttgcattcgatgtggaagtttacacaatgtcagagcactcaatcaaaggcaaattgaacatggccacagccacccagttatgcaaggattgtccaacacccgcaaattttaaaaagtgctgccctcttgtctgtggtaaggctttgcagctaatggatagatataccagacagagattcactgtagatgaaatcaccacattgattatgaatgagaaaaataggagggccaatattggtaactgcatggaagccttgttccaaggaccgctgaggtacagggatctgaaaattgatgtgaagacagtccccccccctgaatgcatcagtgacctactacaagcagtggattctcaagaagttagagattactgtgaaaagaagggctggatagtcaatgttactagccaaatacagttagaaagaaatatcaacagggccatgactatactccaagctgtgactacgtttgcagcagttgcaggagtagtgtatgtgatgtataaactctttgctggtcaacagggtgcatacactggtttgccaagcaaaaagcccaatgtcccaacaatcagagttgccaaagtacagggaccagggtttgattatgcagtggcaatggctaaaagaaacatacttactgcaaccactactaagggtgaattcactatgttaggagtgcatgataatgtagcgatactgccaacccatgccgccccaggagaaactatcattattggtgggaaagaagtggagatcttggacgccagagccctagaagatcaagcgggaaccaatcttgagatcactataatcactctaaaaagaaatgagaagtttagagatatcagaccacatattcccacccaaatcaccgaaaccagtgatggagtattgatcgtgaacactagtaagtaccccaatatgtatgtccctgttggtgctgtgaccgaacaggggtatcttaatctcagtggacgccaaactgctcgtactctgatgtacaactttccaacacgagcaggccagtgcgggggaatcattacctgcactggtaaagtcattgggatgcatgttggtgggaacggttcacatgggtttgcagcagcccttaagcgatcatacttcacccaaagtcaaggtgaaatccaatggatgaggtcatcaaaagaagtgggataccctattataaatgccccatcaaaaacaaagttagaacccagtgcttttcattatgtcttcgaaggtgtcaaagaaccagcagtgctcaccaagagtgaccccagattgaaaacagatttcgaagaagccattttttcaaaatatgtggggaataaaattgttgaagtggacgaatacatgaaagaagcagtggaccactatgcagggcagctaatgtcattagacattaatacagaacaaatgtgcttggaagatgccatgtacggcactgatggtcttgaggctctagatcttagtaccagtgcaggatacccttatgttgcaatgggaaaaaagaaaagagatatcttagacaagcagactagagatactaaagagatgcagaaacttctagacacttatggaatcaacctaccattagttacctatgttaaagatgaactcaggtcaaagactaaagtagagcaaggaaaatcaaggctaattgaagcttccagtctcaatgattcagttgcaatgagaatggcattcggcaatctttacgcagccttccataagaatccaggtgtggtaaccggctcagcagttggttgtgacccagacttattttggagtaagataccagtattaatggaagaaaagctctttgcctttgattacacagggtatgatgcctcgcttagtcctgcttggtttgaggcacttaaaatggtgttagaaaagataggttttggtgatagagcagattacatagattacctgaaccactctcatcatctttacaaaaacaaaacttattgtgttaagggcggtatgccatctggctgttctggcacatcaatttttaactcaatgattaacaatttgatcatcaggacgcttttactgaaaacctacaagggcatggatttagaccatttaaaaatgattgcctatggtgatgatgtgatagcttcctacccccatgaggttgacgctagtctcctagcccaatcaggaaaagactacggactgaccatgactccagcagataaatcagcaacctttgaaacagtcacatgggagaatgtaacgttcctgaaaagatttttcagagcagatgagaaatatccattcctggtgcatccagtaatgccaatgaaagaaattcatgaatcaatcagatggaccaaggaccctagaaatacacaagatcatgtgcgctcgttgtgtttattggcttggcacaacggtgaagaagaatataacaaattcttagctaaaatcagaagtgtgccgatcggtagagctttgttgctcccagagtactctacattgtaccgccggtggcttgactcgttttagtaaccctacctcagtcgaattggattgggttatactgttgtaggggtaaatttttctttaattcggag

SEQ ID NO: 6 - RNA, 713 nt, Enterovirus coxsackievirus A21
  source 1..713: mol_type = unassigned RNA
ttaaaacagctctggggttgttcccaccccagaggcccacgtggcggctagtactctggtatcacggtacctttgtacgcctgttttatatcccttcccccgcaactttagaagcttaacaaaagttcaatagcaggggtgcaagccagtacctctatgaacaagcacttctgtctccccggtgaaattacatagactgtacccacggtcaaaagtaattgatccgttatccgcttgagtacttcgagaagcctagtatcaccttggaatcttcgacgcgttgcgctcaacactctaccccgggtgtagcttaggctgatgagtctgggcaccccccaccggtgacggtggcccaggctgcgttggcggcctacccatggctaacgccatgggacgctagttgtgaacaaggtgtgaagagcctattgagctactcaagagtcctccggcccctgaatgcggctaatcccaaccacggagcaattgctcacgatccagtgagtaggttgtcgtaatgcgcaagttcgtggcggaaccgactactttgggtgtccgtgtttccctttttacttatactggctgcttatggtgacaatttacagattgttaccatatagctattggattggccatccagtattgtgttacacatattaaaatacttttctacaagcatcaacaacattgtgtttggaccacaataatcactacaa

SEQ ID NO: 7 - RNA, 2637 nt, Enterovirus coxsackievirus A21
  source 1..2637: mol_type = unassigned RNA
atgggagctcaagtatcaacccaaaagaccggtgcacacgaaaatcaaaacgtggctgccaatggatccaccattaattacactactatcaactactacaaagatagtgcaagtaactcagccaccagacaagatctttctcaagatccatcaaaatttacagaaccagttaaagatttaatgttgaaaacagcaccagctttgaattcaccaaatgtggaagcatgtggatacagtgaccgtgtaaggcaaattaccctgggaaactcgactattaccacacaagaagcagctaacgctattgttgcttatggtgagtggcccacttacataaatgactcagaagcaaacccagtagatgcacccactgaaccagatgttagtagcaacaggttttacactctggactcagtgtcttggaagactacctcaaggggttggtggtggaaactaccagactgtctaaaagacatggggatgtttggtcagaatatgtattaccactacctggggcgctccggttacaccattcatgtccagtgcaatgcctcaaagtttcaccaaggggcattaggagttttcttgataccagagtttgttatggcctgtaacaccgagagcaagacatcatatgtttcatacatcaacgcaaatcctggtgagaggggtggtgagtttacaaacatttacaatccatcaagcacagatgctagtcagggcagacaatttgcagcgctagattatttactgggttctggtgttttagctggaaatgcatttgtgtacccacaccagatcattaacctgcgtaccaacaatagtgcaactattgtggtaccatatgtgaactcacttgtcattgattgcatggcaaaacacaacaactggggtattgtcatcctgccactggcgccattggcctttgctgcgacatcatcaccacaagtgcctattacagtgaccattgcacccatgtgcgcagaattcaatggattgagaaacatcactattccagtgcatcaaggattaccaacaatgaacacacctggatccaatcagtttctcacatctgatgacttccagtcaccctgcgctctacccaactttgatgtcactccacctatacacatacccggagaagtgaagaatatgatggaactggccgagattgacacgctgatcccaatgaacgcagtggacggaaaggtaaacactatggagatgtatcaaataccattaaatgatgaactaagcaaagcacccatattttgcctgtccctgtcacctgcttctgacaaacgattaagtcatacgatgttgggtgaaattctaaactattacactcattggacagggtccattaggttcacctttctattctgtggtagcatgatggccactggtaagttgcttctcagttattccccaccaggagctaaaccaccaaccaaccgcaaggatgcaatgttgggcacacatatcatctgggacttgggtttacaatccagttgctccatggttgcaccatggatctctaatacggtatacaggcggtgtgcacgtgatgacttcacagagggtgggtttataacttgcttttatcaaaccagaattgttgtgcctgcctcaacccctaccagcatgttcatgttaggctttgtgagtgcatgtccagatttcagcgtcagactgctgagggacacttcccacattagtcaatcaaaactcatagcacgctcacaaggcattgaagatctcatcgattcagcaataaagaatgctttgagagtgtcccaaccgtccacggctcaatcgactgaagcaaccagtggagcaaacagtcaggaagtaccagcactaactgctgtagaaacaggagcatcaggtcaagcaattcccagtgacgtgatggaaaccagacacgtgataaactacaaaactaggtctgagtcatgtcttgaatcattctttgggagagctgcgtgtgtcacaatcctatctctgaccaactcttcaaaaagtggagaggaaaagaaacatttcaacatttggaacatcacgtataccgacactgtgcagctacgtagaaaattagagttcttcacatattccagatttgaccttgaaatgacttttgtgttcacagagaactaccccagtacagctagtggagaagtgcgcaaccaggtataccagattatgtatattccaccaggggcaccccgaccatcatcctgggatgattatacatggcaatcctcctccaacccttctatcttttacatgtacgggaacgcaccgccacggatgtcaatcccctatgtagggattgctaatgcctactcacacttttatgacggttttgcacgagtgccacttgagggtgagaacactgatgctggtgacacgttttatggattggtatccataaatgattttggagtcttggcagtcagagcagtgaaccgcagcaatccacacacaatacacacatccgtgagagtgtacatgaaaccaaaacacattcggtgctggtgccccagaccccctcgcgcagtattatacagaggagaaggagtggacatgatatccagtgcaattttacccctgaccaaagtagattctattactaccttc

SEQ ID NO: 8 - RNA, 1389 nt, Enterovirus coxsackievirus A21
  source 1..1389: mol_type = unassigned RNA
ggtgaaatccaatggatgaggtcatcaaaagaagtgggataccctattataaatgccccatcaaaaacaaagttagaacccagtgcttttcattatgtcttcgaaggtgtcaaagaaccagcagtgctcaccaagagtgaccccagattgaaaacagatttcgaagaagccattttttcaaaatatgtggggaataaaattgttgaagtggacgaatacatgaaagaagcagtggaccactatgcagggcagctaatgtcattagacattaatacagaacaaatgtgcttggaagatgccatgtacggcactgatggtcttgaggctctagatcttagtaccagtgcaggatacccttatgttgcaatgggaaaaaagaaaagagatatcttagacaagcagactagagatactaaagagatgcagaaacttctagacacttatggaatcaacctaccattagttacctatgttaaagatgaactcaggtcaaagactaaagtagagcaaggaaaatcaaggctaattgaagcttccagtctcaatgattcagttgcaatgagaatggcattcggcaatctttacgcagccttccataagaatccaggtgtggtaaccggctcagcagttggttgtgacccagacttattttggagtaagataccagtattaatggaagaaaagctctttgcctttgattacacagggtatgatgcctcgcttagtcctgcttggtttgaggcacttaaaatggtgttagaaaagataggttttggtgatagagcagattacatagattacctgaaccactctcatcatctttacaaaaacaaaacttattgtgttaagggcggtatgccatctggctgttctggcacatcaatttttaactcaatgattaacaatttgatcatcaggacgcttttactgaaaacctacaagggcatggatttagaccatttaaaaatgattgcctatggtgatgatgtgatagcttcctacccccatgaggttgacgctagtctcctagcccaatcaggaaaagactacggactgaccatgactccagcagataaatcagcaacctttgaaacagtcacatgggagaatgtaacgttcctgaaaagatttttcagagcagatgagaaatatccattcctggtgcatccagtaatgccaatgaaagaaattcatgaatcaatcagatggaccaaggaccctagaaatacacaagatcatgtgcgctcgttgtgtttattggcttggcacaacggtgaagaagaatataacaaattcttagctaaaatcagaagtgtgccgatcggtagagctttgttgctcccagagtactctacattgtaccgccggtggcttgactcgttttagtaa

SEQ ID NO: 9 - RNA, 7440 nt, Enterovirus coxsackievirus A21
  source 1..7440: mol_type = unassigned RNA
ttaaaacagctctggggttgttcccaccccagaggcccacgtggcggctagtactctggtatcgcggtacctttgtacgcctgttttatactcccttccccatgtaactttagaagcaattcaccaagttcaatagagggggtacaagccagtaccaccacgaacaagcacttctgttaccccggtgagtttgcataagctgtgcccacggctgaaagtaaatgatccgttatccgcttgagtacttcgagaagcctagtatcaccttggaatcttcgacgcgttgcgctcaacactctaccccgagtgtagcttaggctgatgagtctgggcatcccccaccggcgacggtggcccaggctgcgttggcggcctacccatggccaacgccatgggacgctagttgtgaacaaggtgtgaagagcctattgagctactcaagagtcctccggcccctgaatgcggctaatcctaaccatggagcaagtggccccaacccaggggatggcttgtcgtaacgcgcaagtccgtggcggaaccgactactttgggtgtccgtgtttccttttattttatatatggctgcttatggtgacaatcacagattgttatcataaagcgaattggattggccatccggtgaaatataagcacattatttatatctttgctggattcgctccgttgacacattcttcaccaaagttgatttatacagtcttagtaattagaaatcaccacttcagtcatgggagctcaagtatccactcaaaagactggggcccacgaaaaccagaatgtggccgcgaacgggtccacaatcaattatacaaccatcaactactataaggacagtgcaagcaattcggcgactcggcaggatttctcacaggacccgtccaaattcactgaaccggtcaaggatttaatgttaaaatccgcaccggctttgaattcgccaaacgtcgaagcatgcgggtacagtgatcgcgtgaggcaaatcacactgggaaattccaccataaccacacaggaggctgcaaatgcaattgttgcctatggggagtggccaacctacataaacgataacgaggccaaccctgtcgatgcaccgaccgaaccagatgtgagtagcaaccgcttttacaccctggaatcagtgcagtggaaaacaacatcccgggggtggtggtggaaattgccagactgtttgaaagacatgggtatgttcgggcaaaacatgtattaccactatttaggcaggtcagggtatactatacatgtgcagtgtaatgcctcaaaatttcaccaaggtgcgctgggtgtgtttctaatccctgaatttgtgatggcatgtaacaccgagagcaagacctcatatgtctcatatattaatgccaaccctggtgagaagggtggtgaatttacaaacacctacaacccatctggcacagacgttagcgaagggaggaaatttgccgctctggattatctgctagggtcaggcgtcttggcgggaaacgcttttgtttacccccatcaaattatcaaccttcgcaccaacaatagtgcaacaatagtggttccttacgtcaactcactagtgatcgattgcatggcaaaacacaacaactgggggatagcgatactaccccttgcacccctagcctttgcagcaacgtcatcaccacaagtaccgatcacagtcacaattgcaccaatgtgtacagagttcaatggattgaggaatgtaactgtaccagtacaccagggccttccagtcatgaacaccccaggatcaaaccaattcctcacatcagatgatttccagtcgccatgtgccttacccaatttcgatgtgacccctccaatccacatcccaggggaggtcaaaaacatgatggaactagctgaaattgacacactgattccaatgaatgcagtggatgggaaagtaaataccatggaaatgtaccagatcccattaaacgacaaccttagcactgaacccatcttttgcctatcactatcaccagcgtctgataggcgattgagtcacaccatgctgggagagattctgaactactacacacactggacgggctccatcagatttacttttctattttgtggtagtatgatggccacagggaagttgctccttagctattcacccccaggggcgaagccacccaaaaaccggaaagacgccatgctgggtacgcacataatatgggacctgggtttacagtctagttgttccatggtggcgccctggatttccaatacggtgtacaggcgatgtgttcgcgacgattttaccgaaggtggctacatcacctgtttctatcagactagaatagtggtaccagcctcgaccccaaccagcatgtttatgctaggctttgtcagtgcttgtcccgactttagtgtgagactgctcagggacacgacgcacatcagtcaatcgaaattggtcgccagtaggcagggcatagaggagtttatagacacagcaattaaaaatgcattgaaagtgtcacaacccagcactgcacaatcaacagaagcaaccagcggagtcaacagccaagaggtgcctgcactaacagcagtagagactggcgcatcgggccaagcaatcccaagtgatgtggtagaaactcgccacgtggtgaactataagacaagatcagagtcctgccttgaatcattctttggtagggccgcttgtgtgacaattctctcccttacaaactcttcaggtagcggagacgcaaagaagcactacacaacatggaacatcacatatacagatacggtgcaactgcgaaggaaactggaattttttacgtattccaggtttgacttggaaatgacctttgtattcactgagaactaccctagcacagctagtggagaggttcgaaatcaggtttaccaaatcatgtacattcctccaggggcgcctagacctagttcttgggatgattacacctggcagtcatcatcaaacccttccatattctacatgtatggtaatgcaccaccacggatgtcgattccctatgtcgggattgctaacgcctactcgcacttctacgatggcttcgcgcgagtgccgcttgaaggtgagaacacagatgccggggacaccttctacgggctagtatccatcaatgattttggtgtactggcagttagggcagtgaaccgcagcagccctcacgccatccacacttcagtgagggtgtatatgaaacctaaacacattcgctgctggtgccccaggccaccacgagcagtactttaccgtggtgagggagtggacatgatctcaagtgcaatccaaccactaaccaaggtcgattctataacaacattcggctttggtcaccagaacaaggcagtttatgttgctgggtataaaatttgtaactaccacttagcaacacccagtgaccacctgaacgccattagtgtgatgtgggacagagatttgatagtagtggagtcaagagcacagggaattgacaccatagcccggtgcaactgcaagtgtggcgtatactactgtgaatcaaggaaaaagtattatcccatttcgttcactgggcccacatttagatacatggaggccaacgaatactttccagctagatatcagtcccatttattaattgggtgcgggtttgcagagcctggagactgtggtggcatactcagatgcacacacggtgtgatcggcataattacagcaggtggacctggagtggtggcctttgccgacatcagggacctctgggtgtatgaggaggaagcgatggaacaaggcattacaagctacatcgagactttaggggcagcatttggaactgggttcactcaacaaattggagacaaaattacagagttgactaatatggtgacaagcacttttacggacaaattgtttaaaaacttgattaaaatagtgtcttcgttggttataataactcgaaactacgaagacaccaccacagtattggccactctagcattgttgggttgtgattgctctccgtggcaatggttgaaacacaaagtatgtgacatcctgcaactgccattcgccctcaaacagagtgacagttggctaaagaagttcactgaggcgtgtaatgccgcaaaagggctcgaatggattagcaacaagctctccaaatttgtggaatgggtgaagaacaagataattcctgaagccagagacaaagtggaatttctgcaaaaattgaaacagttagaaatgcttgagaaccagattgctacactacaccaatcctgtccaagtcaagaacagcaagagacactctttaacaatgtccgctggttggcactgcagtcaagacgctttgcgccgttgtacgctgttgaagcgcgcagaatacaaaaattggagaacaccataaacaactacatacagtttaagagcaaacaccgtattgaaccagtatgtatgttgatacatggagctcctggcacgggcaaatctatagcaacctcgttgattgggcgtgccattgctgaaaaagaaagcacctcagtgtattctctaccaccagacccctcacacttcgacggttacaaacaacaaggggttgttatcatggatgacctcaatcagaacccggatggtatggacatgaagttgttttgtcagatggtgtctacagtcgagttcatacccccaatggcatctctcgaggagaaaggaatcctgttcacttcagactatgtgctagcatctacaaattctcactcaattaccccacccactgtcgcacatagtgatgccctaagtagaagatttgcatttgacatggatgtatacaccatgtctgaacactctgtgaagggaaagctcaacatggcgactgcgacgcagttatgcaaagagtgtccaccgcccgctaacttcaaaaagtgctgtccactagtgtgtggtaaagctttacaactgatggacaagcacaccagacagagatttacactagacgaaatcaccactttgatcatcaatgagaagaacagaagggctaacattggtaattgcatggaggctctttttcaaggccctctacaatacaaagatctcaagatcgacatcaagaacactccccctccagagtgcataagtgatctacttcaggctgttgactcgcaagaggttagagattactgtgagaagaaaggatggatcgttaacgtatccagtcaggttcaattagagcgcaacatcaatagagctatgactatcctccaggcagtaaccacgttcgccgcagtggcgggagtggtttatgtcatgtacaagctctttgcagggcatcaaggcgcttatactgggctcccgagcaagaaacccaacgtgcccaccattagaatagctaaagtgcaaggtccagggtttgactatgcagttgccatggcaaagagaaatatcgttactgcaaccaccaataagggggagttcactatgcttggagtgcatgataacgttgccgtactaccaacccatgcatcaccaggtgagaccatagttattgatggtaaggaagttgaagtgctggacgccaaggccctggaggaccaagctggaactaacctagagatcaccattgtcactctgagaaggaatgagaagttcagggacatcagacctcacatccccactcaaatcactgagacaaatgatggagtgttgatcgtgaacactagtaagtaccccaacatgtatgtgcccgttggtgcagtgaccgaacaagggtacttgaacctgggtgggcgtcaaactgctcgcactctaatgtacaatttcccaacccgagccggacaatgtggtggaatcatcacatgcactggcaaggtcattgggatgcatgttggtgggaacggttcacatgggttcgctgcagccctaaaacggtcatacttcactcagagccaaggtgagattcagtggatgagaccatcaaaagaggtcggttacccaataattaatgcaccaagcaagacaaaactagaacccagtgctttccactatgtttttgaaggggtaaaggaacctgcagtactgactaaaaacgatcccagattgaaaacggattttgaggaggccatattctcaaagtatgtagggaacaagatcactgaagtggatgaatatatgaaagaggcagtagaccattatgctgggcaactcatgtctttagacatcaatacagagcaaatgtgcttggaagatgctatgtatggcactgatggtctggaagctttggatctctctactagcgcaggatacccttatgtggctcttgggaaaaagaagagagatatacttaataaacaaacaagagacaccaaagaaatgcaaagactactagatacctatggaataaatttgcctctagttacttacgtaaaagatgagctgagaagcaagagcaaggtggagcagggtaaatccagattaattgaggcctcaagcttgaatgattcagtagcaatgagaatggcatttggtaacctgtatgcagctttccacaagaatccaggagtcatcactggttctgctgtaggatgtgacccagacttgttttggagcaagatcccagtattgatggaagaaaagctctttgcatttgattacaccggttacgatgcctctctcagtcccgcttggtttgaggcactcaaaatggttttggagaaaattggattcggagatagggtagattacattgattatctcaaccattcacaccatctttacaaaaacaagacctattgtgtcaagggcggtatgccatctggctgctcaggcacttcaatatttaattcaatgattaacaatttaataatcaggacactcttactgaagacctacaagggcatagatttggaccacttgaagatgattgcctatggtgatgatgtaattgcctcctacccccacgaagttgacgctagtctcctagcccaatcaggaaaagactatggactaaccatgacaccagctgacaagtcagccacctttgaaactgtcacgtgggagaatgtaacatttttaaaaagattctttagggcagatgaaaagtacccattcttaatacatccagtgatgccaatgaaagaaatacaagaatcgatcagatggactaaagatcctagaaacacgcaggaccatgttcgctcactgtgcttattggcttggcacaatggcgaagaagagtacaataaatttttagctaaaatcagaagtgtgccaatcggaagagctttactgctccccgaatactcaactttgtaccgccgttggctcgactcgttttagtaaccctacctcagtcgaattggattgggtcatactgttgtaggggtaaatttttctttgattcggag

SEQ ID NO: 10 - RNA, 748 nt, Enterovirus coxsackievirus A21
  source 1..748: mol_type = unassigned RNA
ttaaaacagctctggggttgttcccaccccagaggcccacgtggcggctagtactctggtatcgcggtacctttgtacgcctgttttatactcccttccccatgtaactttagaagcaattcaccaagttcaatagagggggtacaagccagtaccaccacgaacaagcacttctgttaccccggtgagtttgcataagctgtgcccacggctgaaagtaaatgatccgttatccgcttgagtacttcgagaagcctagtatcaccttggaatcttcgacgcgttgcgctcaacactctaccccgagtgtagcttaggctgatgagtctgggcatcccccaccggcgacggtggcccaggctgcgttggcggcctacccatggccaacgccatgggacgctagttgtgaacaaggtgtgaagagcctattgagctactcaagagtcctccggcccctgaatgcggctaatcctaaccatggagcaagtggccccaacccaggggatggcttgtcgtaacgcgcaagtccgtggcggaaccgactactttgggtgtccgtgtttccttttattttatatatggctgcttatggtgacaatcacagattgttatcataaagcgaattggattggccatccggtgaaatataagcacattatttatatctttgctggattcgctccgttgacacattcttcaccaaagttgatttatacagtcttagtaattagaaatcaccacttcagtc

SEQ ID NO: 11 - RNA, 2637 nt, Enterovirus coxsackievirus A21
  source 1..2637: mol_type = unassigned RNA
atgggagctcaagtatccactcaaaagactggggcccacgaaaaccagaatgtggccgcgaacgggtccacaatcaattatacaaccatcaactactataaggacagtgcaagcaattcggcgactcggcaggatttctcacaggacccgtccaaattcactgaaccggtcaaggatttaatgttaaaatccgcaccggctttgaattcgccaaacgtcgaagcatgcgggtacagtgatcgcgtgaggcaaatcacactgggaaattccaccataaccacacaggaggctgcaaatgcaattgttgcctatggggagtggccaacctacataaacgataacgaggccaaccctgtcgatgcaccgaccgaaccagatgtgagtagcaaccgcttttacaccctggaatcagtgcagtggaaaacaacatcccgggggtggtggtggaaattgccagactgtttgaaagacatgggtatgttcgggcaaaacatgtattaccactatttaggcaggtcagggtatactatacatgtgcagtgtaatgcctcaaaatttcaccaaggtgcgctgggtgtgtttctaatccctgaatttgtgatggcatgtaacaccgagagcaagacctcatatgtctcatatattaatgccaaccctggtgagaagggtggtgaatttacaaacacctacaacccatctggcacagacgttagcgaagggaggaaatttgccgctctggattatctgctagggtcaggcgtcttggcgggaaacgcttttgtttacccccatcaaattatcaaccttcgcaccaacaatagtgcaacaatagtggttccttacgtcaactcactagtgatcgattgcatggcaaaacacaacaactgggggatagcgatactaccccttgcacccctagcctttgcagcaacgtcatcaccacaagtaccgatcacagtcacaattgcaccaatgtgtacagagttcaatggattgaggaatgtaactgtaccagtacaccagggccttccagtcatgaacaccccaggatcaaaccaattcctcacatcagatgatttccagtcgccatgtgccttacccaatttcgatgtgacccctccaatccacatcccaggggaggtcaaaaacatgatggaactagctgaaattgacacactgattccaatgaatgcagtggatgggaaagtaaataccatggaaatgtaccagatcccattaaacgacaaccttagcactgaacccatcttttgcctatcactatcaccagcgtctgataggcgattgagtcacaccatgctgggagagattctgaactactacacacactggacgggctccatcagatttacttttctattttgtggtagtatgatggccacagggaagttgctccttagctattcacccccaggggcgaagccacccaaaaaccggaaagacgccatgctgggtacgcacataatatgggacctgggtttacagtctagttgttccatggtggcgccctggatttccaatacggtgtacaggcgatgtgttcgcgacgattttaccgaaggtggctacatcacctgtttctatcagactagaatagtggtaccagcctcgaccccaaccagcatgtttatgctaggctttgtcagtgcttgtcccgactttagtgtgagactgctcagggacacgacgcacatcagtcaatcgaaattggtcgccagtaggcagggcatagaggagtttatagacacagcaattaaaaatgcattgaaagtgtcacaacccagcactgcacaatcaacagaagcaaccagcggagtcaacagccaagaggtgcctgcactaacagcagtagagactggcgcatcgggccaagcaatcccaagtgatgtggtagaaactcgccacgtggtgaactataagacaagatcagagtcctgccttgaatcattctttggtagggccgcttgtgtgacaattctctcccttacaaactcttcaggtagcggagacgcaaagaagcactacacaacatggaacatcacatatacagatacggtgcaactgcgaaggaaactggaattttttacgtattccaggtttgacttggaaatgacctttgtattcactgagaactaccctagcacagctagtggagaggttcgaaatcaggtttaccaaatcatgtacattcctccaggggcgcctagacctagttcttgggatgattacacctggcagtcatcatcaaacccttccatattctacatgtatggtaatgcaccaccacggatgtcgattccctatgtcgggattgctaacgcctactcgcacttctacgatggcttcgcgcgagtgccgcttgaaggtgagaacacagatgccggggacaccttctacgggctagtatccatcaatgattttggtgtactggcagttagggcagtgaaccgcagcagccctcacgccatccacacttcagtgagggtgtatatgaaacctaaacacattcgctgctggtgccccaggccaccacgagcagtactttaccgtggtgagggagtggacatgatctcaagtgcaatccaaccactaaccaaggtcgattctataacaacattc

SEQ ID NO: 12 - RNA, 1389 nt, Enterovirus coxsackievirus A21
  source 1..1389: mol_type = unassigned RNA
ggtgagattcagtggatgagaccatcaaaagaggtcggttacccaataattaatgcaccaagcaagacaaaactagaacccagtgctttccactatgtttttgaaggggtaaaggaacctgcagtactgactaaaaacgatcccagattgaaaacggattttgaggaggccatattctcaaagtatgtagggaacaagatcactgaagtggatgaatatatgaaagaggcagtagaccattatgctgggcaactcatgtctttagacatcaatacagagcaaatgtgcttggaagatgctatgtatggcactgatggtctggaagctttggatctctctactagcgcaggatacccttatgtggctcttgggaaaaagaagagagatatacttaataaacaaacaagagacaccaaagaaatgcaaagactactagatacctatggaataaatttgcctctagttacttacgtaaaagatgagctgagaagcaagagcaaggtggagcagggtaaatccagattaattgaggcctcaagcttgaatgattcagtagcaatgagaatggcatttggtaacctgtatgcagctttccacaagaatccaggagtcatcactggttctgctgtaggatgtgacccagacttgttttggagcaagatcccagtattgatggaagaaaagctctttgcatttgattacaccggttacgatgcctctctcagtcccgcttggtttgaggcactcaaaatggttttggagaaaattggattcggagatagggtagattacattgattatctcaaccattcacaccatctttacaaaaacaagacctattgtgtcaagggcggtatgccatctggctgctcaggcacttcaatatttaattcaatgattaacaatttaataatcaggacactcttactgaagacctacaagggcatagatttggaccacttgaagatgattgcctatggtgatgatgtaattgcctcctacccccacgaagttgacgctagtctcctagcccaatcaggaaaagactatggactaaccatgacaccagctgacaagtcagccacctttgaaactgtcacgtgggagaatgtaacatttttaaaaagattctttagggcagatgaaaagtacccattcttaatacatccagtgatgccaatgaaagaaatacaagaatcgatcagatggactaaagatcctagaaacacgcaggaccatgttcgctcactgtgcttattggcttggcacaatggcgaagaagagtacaataaatttttagctaaaatcagaagtgtgccaatcggaagagctttactgctccccgaatactcaactttgtaccgccgttggctcgactcgttttagtaa

SEQ ID NO: 13 - DNA, 57 bp, synthetic construct
  misc_feature 1..57: note = Leader sequence CVA21-L4
  source 1..57: mol_type = other DNA
tcgactgataaagatgcaaaggaaagtacatggacagaaaatgaagatctagctatt

SEQ ID NO: 14 - DNA, 57 bp, synthetic construct
  misc_feature 1..57: note = Leader sequence CVA21-L5
  source 1..57: mol_type = other DNA
tcgactattcctgattagaccctcaatattgatatagaaaacgactacacagcgcat

SEQ ID NO: 15 - DNA, 57 bp, synthetic construct
  misc_feature 1..57: note = Leader sequence CVA21-L6
  source 1..57: mol_type = other DNA
tcgactctacaaataacggataccaatttgagtaacgcggcttcaatgtacaataat

SEQ ID NO: 16 - DNA, 67 bp, Paenibacillus polymyxa
  source 1..67: mol_type = unassigned DNA
ggactcgtctgagcgagtataaacaggtcattaagctcagagcgttcaccggggaaattcggtgagg

SEQ ID NO: 17 - DNA, 67 bp, synthetic construct
  misc_feature 1..67: note = Pistol Polymyxa ribozyme variant PP
  source 1..67: mol_type = other DNA
ggactcgtctgagcgagtataaacagtttattaagctcagagcgttcaccggggaaattcggtgagg

SEQ ID NO: 18 - DNA, 67 bp, synthetic construct
  misc_feature 1..67: note = Pistol Polymyxa ribozyme variant PP-T
  source 1..67: mol_type = other DNA
ggactcgtctgagcgagtataaacagttttttaagctcagagcgttcaccggggaaattcggtgagg

SEQ ID NO: 19 - DNA, 67 bp, synthetic construct
  misc_feature 1..67: note = Pistol Polymyxa ribozyme variant PP-G
  source 1..67: mol_type = other DNA
ggactcgtctgagcgagtataaacagttgtttaagctcagagcgttcaccggggaaattcggtgagg

SEQ ID NO: 20 - DNA, 7626 bp, synthetic construct
  misc_feature 1..7626: note = CVA-PP-TwL6-BsmBI_KY_70pA
  source 1..7626: mol_type = other DNA
taatacgactcactatagggtcgactctacaaataacggataccaatttgagtaacgcggcttcaatgtacaataatggactcgtctgagcgagtataaacagttttttaagctcagagcgttcaccggggaaattcggtgaggttaaaacagctctggggttgttcccaccccagaggcccacgtggcggctagtactctggtatcacggtacctttgtacgcctgttttatatcccttcccccgcaactttagaagcttaacaaaagttcaatagcaggggtgcaagccagtacctctatgaacaagcacttctgtctccccggtgaaattacatagactgtacccacggtcaaaagtaattgatccgttatccgcttgagtacttcgagaagcctagtatcaccttggaatcttcgacgcgttgcgctcaacactctaccccgggtgtagcttaggctgatgagtctgggcaccccccaccggtgacggtggcccaggctgcgttggcggcctacccatggctaacgccatgggacgctagttgtgaacaaggtgtgaagagcctattgagctactcaagagtcctccggcccctgaatgcggctaatcccaaccacggagcaattgctcacgatccagtgagtaggttgtcgtaatgcgcaagttcgtggcggaaccgactactttgggtgtccgtgtttccctttttacttatactggctgcttatggtgacaatttacagattgttaccatatagctattggattggccatccagtattgtgttacacatattaaaatacttttctacaagcatcaacaacattgtgtttggaccacaataatcactacaaatgggagctcaagtatcaacccaaaagaccggtgcacacgaaaatcaaaacgtggctgccaatggatccaccattaattacactactatcaactactacaaagatagtgcaagtaactcagccaccagacaagatctttctcaagatccatcaaaatttacagaaccagttaaagatttaatgttgaaaacagcaccagctttgaattcaccaaatgtggaagcatgtggatacagtgaccgtgtaaggcaaattaccctgggaaactcgactattaccacacaagaagcagctaacgctattgttgcttatggtgagtggcccacttacataaatgactcagaagcaaacccagtagatgcacccactgaaccagatgttagtagcaacaggttttacactctggactcagtgtcttggaagactacctcaaggggttggtggtggaaactaccagactgtctaaaagacatggggatgtttggtcagaatatgtattaccactacctggggcgctccggttacaccattcatgtccagtgcaatgcctcaaagtttcaccaaggggcattaggagttttcttgataccagagtttgttatggcctgtaacaccgagagcaagacatcatatgtttcatacatcaacgcaaatcctggtgagaggggtggtgagtttacaaacatttacaatccatcaagcacagatgctagtcagggcagacaatttgcagcgctagattatttactgggttctggtgttttagctggaaatgcatttgtgtacccacaccagatcattaacctgcgtaccaacaatagtgcaactattgtggtaccatatgtgaactcacttgtcattgattgcatggcaaaacacaacaactggggtattgtcatcctgccactggcgccattggcctttgctgcgacatcatcaccacaagtgcctattacagtgaccattgcacccatgtgcgcagaattcaatggattgagaaacatcactattccagtgcatcaaggattaccaacaatgaacacacctggatccaatcagtttctcacatctgatgacttccagtcaccctgcgctctacccaactttgatgtcactccacctatacacatacccggagaagtgaagaatatgatggaactggccgagattgacacgctgatcccaatgaacgcagtggacggaaaggtaaacactatggagatgtatcaaataccattaaatgatgaactaagcaaagcacccatattttgcctgtccctgtcacctgcttctgacaaacgattaagtcatacgatgttgggtgaaattctaaactattacactcattggacagggtccattaggttcacctttctattctgtggtagcatgatggccactggtaagttgcttctcagttattccccaccaggagctaaaccaccaaccaaccgcaaggatgcaatgttgggcacacatatcatctgggacttgggtttacaatccagttgctccatggttgcaccatggatctctaatacggtatacaggcggtgtgcacgtgatgacttcacagagggtgggtttataacttgcttttatcaaaccagaattgttgtgcctgcctcaacccctaccagcatgttcatgttaggctttgtgagtgcatgtccagatttcagcgtcagactgctgagggacacttcccacattagtcaatcaaaactcatagcacgctcacaaggcattgaagatctcatcgattcagcaataaagaatgctttgagagtgtcccaaccgtccacggctcaatcgactgaagcaaccagtggagcaaacagtcaggaagtaccagcactaactgctgtagaaacaggagcatcaggtcaagcaattcccagtgacgtgatggaaaccagacacgtgataaactacaaaactaggtctgagtcatgtcttgaatcattctttgggagagctgcgtgtgtcacaatcctatctctgaccaactcttcaaaaagtggagaggaaaagaaacatttcaacatttggaacatcacgtataccgacactgtgcagctacgtagaaaattagagttcttcacatattccagatttgaccttgaaatgacttttgtgttcacagagaactaccccagtacagctagtggagaagtgcgcaaccaggtataccagattatgtatattccaccaggggcaccccgaccatcatcctgggatgattatacatggcaatcctcctccaacccttctatcttttacatgtacgggaacgcaccgccacggatgtcaatcccctatgtagggattgctaatgcctactcacacttttatgacggttttgcacgagtgccacttgagggtgagaacactgatgctggtgacacgttttatggattggtatccataaatgattttggagtcttggcagtcagagcagtgaaccgcagcaatccacacacaatacacacatccgtgagagtgtacatgaaaccaaaacacattcggtgctggtgccccagaccccctcgcgcagtattatacagaggagaaggagtggacatgatatccagtgcaattttacccctgaccaaagtagattctattactaccttcggatttggacaccaaagtaaggcagtgtatgttgccggttataagatctgcaactaccacctagcaacccctagtgatcatctaaatgcaattagtgtgttgtgggatagggatttaatggtagtagagtctagagcccaaggaactgacaccatagccagatgtagttgtaggtgtggggtctattactgtgaatcaagaaggaagtactaccctgtcactttcaccggtccaacgttccgattcatggaagcgaatgactactacccagcaagataccagtctcacatgctgataggatgtggatttgcagagcctggagattgcggtggaatactaaggtgcacccatggggtgattggtattattactgcagggggtgagggagtagtagcctttgctgacatcagggatctttgggtgtatgaagaggaggccatggaacagggaataacaagctacattgagtctcttggcacagcctttggtgcagggtttactcacacaattagtgaaaaagtgactgagttaactacaatgattaccagtaccattacagaaaaactactaaaaaatttggtgaaaatagtatcagctttagtaattgttgtgagaaattatgaagacactactacagttcttgcaacactagcattacttgggtgtgacatatctccttggcagtggttgaagaaaaaggcatgtgacctactagaaattcctcatgtactgcgccaaggtgatgggtggatgaagaaattcacagaggcttgcaatgcagccaagggcttagaatggattagcaacaaaatttctaagttcatagactggttgaagtgcaaaattattccagaagccaaagataaggtggaatttctcaccaagctgaaacagttagacatgttagaaaatcaaattgcaactattcaccagtcttgccccagccaggagcaacaagagattctcttcaacaatgtgagatggttagcagtccagtcccgtcgatttgcaccactatatgctgtggaagcacggagaattagcaaaatggagaacacaataaacaactacatacagttcaagagcaaacaccgtattgaaccagtatgtatgctcatccatgggtcaccgggaacaggcaaatcaatagccacctcattgataggcagagcgatagcagaaaaggagaatacatcagtttactcaatgccacctgacccatctcactttgatggctacaaacaacaaggggtcgtgattatggatgatttgaatcagaaccctgatggcatggatatgaaactgttttgccaaatggtgtcaacggtggagttcatcccaccgatggcctcactagaggagaagggtattttgtttacatctgattatgtcctagcctccaccaactcccattcaatagcaccacccacggtagctcacagtgatgccttaaccagacgatttgcattcgatgtggaagtttacacaatgtcagagcactcaatcaaaggcaaattgaacatggccacagccacccagttatgcaaggattgtccaacacccgcaaattttaaaaagtgctgccctcttgtctgtggtaaggctttgcagctaatggatagatataccagacagagattcactgtagatgaaatcaccacattgattatgaatgagaaaaataggagggccaatattggtaactgcatggaagccttgttccaaggaccgctgaggtacagggatctgaaaattgatgtgaagacagtccccccccctgaatgcatcagtgacctactacaagcagtggattctcaagaagttagagattactgtgaaaagaagggctggatagtcaatgttactagccaaatacagttagaaagaaatatcaacagggccatgactatactccaagctgtgactacgtttgcagcagttgcaggagtagtgtatgtgatgtataaactctttgctggtcaacagggtgcatacactggtttgccaagcaaaaagcccaatgtcccaacaatcagagttgccaaagtacagggaccagggtttgattatgcagtggcaatggctaaaagaaacatacttactgcaaccactactaagggtgaattcactatgttaggagtgcatgataatgtagcgatactgccaacccatgccgccccaggagaaactatcattattggtgggaaagaagtggagatcttggacgccagagccctagaagatcaagcgggaaccaatcttgagatcactataatcactctaaaaagaaatgagaagtttagagatatcagaccacatattcccacccaaatcaccgaaaccagtgatggagtattgatcgtgaacactagtaagtaccccaatatgtatgtccctgttggtgctgtgaccgaacaggggtatcttaatctcagtggacgccaaactgctcgtactctgatgtacaactttccaacacgagcaggccagtgcgggggaatcattacctgcactggtaaagtcattgggatgcatgttggtgggaacggttcacatgggtttgcagcagcccttaagcgatcatacttcacccaaagtcaaggtgaaatccaatggatgaggtcatcaaaagaagtgggataccctattataaatgccccatcaaaaacaaagttagaacccagtgcttttcattatgtcttcgaaggtgtcaaagaaccagcagtgctcaccaagagtgaccccagattgaaaacagatttcgaagaagccattttttcaaaatatgtggggaataaaattgttgaagtggacgaatacatgaaagaagcagtggaccactatgcagggcagctaatgtcattagacattaatacagaacaaatgtgcttggaagatgccatgtacggcactgatggtcttgaggctctagatcttagtaccagtgcaggatacccttatgttgcaatgggaaaaaagaaaagagatatcttagacaagcagactagagatactaaagagatgcagaaacttctagacacttatggaatcaacctaccattagttacctatgttaaagatgaactcaggtcaaagactaaagtagagcaaggaaaatcaaggctaattgaagcttccagtctcaatgattcagttgcaatgagaatggcattcggcaatctttacgcagccttccataagaatccaggtgtggtaaccggctcagcagttggttgtgacccagacttattttggagtaagataccagtattaatggaagaaaagctctttgcctttgattacacagggtatgatgcctcgcttagtcctgcttggtttgaggcacttaaaatggtgttagaaaagataggttttggtgatagagcagattacatagattacctgaaccactctcatcatctttacaaaaacaaaacttattgtgttaagggcggtatgccatctggctgttctggcacatcaatttttaactcaatgattaacaatttgatcatcaggacgcttttactgaaaacctacaagggcatggatttagaccatttaaaaatgattgcctatggtgatgatgtgatagcttcctacccccatgaggttgacgctagtctcctagcccaatcaggaaaagactacggactgaccatgactccagcagataaatcagcaacctttgaaacagtcacatgggagaatgtaacgttcctgaaaagatttttcagagcagatgagaaatatccattcctggtgcatccagtaatgccaatgaaagaaattcatgaatcaatcagatggaccaaggaccctagaaatacacaagatcatgtgcgctcgttgtgtttattggcttggcacaacggtgaagaagaatataacaaattcttagctaaaatcagaagtgtgccgatcggtagagctttgttgctcccagagtactctacattgtaccgccggtggcttgactcgttttagtaaccctacctcagtcgaattggattgggttatactgttgtaggggtaaatttttctttaattcggagaaaaaaaaaaaaaaaaaaaaaaaaaaaaaaaaaaaaaaaaaaaaaaaaaaaaaaaaaaaaaaaaaaaaaaagagacg

SEQ ID NO: 21 - DNA, 7661 bp, synthetic construct
  misc_feature 1..7661: note = CVA-PP-TwL6-BsmBI_EF_70pA
  source 1..7661: mol_type = other DNA
taatacgactcactatagggtcgactctacaaataacggataccaatttgagtaacgcggcttcaatgtacaataatggactcgtctgagcgagtataaacagttttttaagctcagagcgttcaccggggaaattcggtgaggttaaaacagctctggggttgttcccaccccagaggcccacgtggcggctagtactctggtatcgcggtacctttgtacgcctgttttatactcccttccccatgtaactttagaagcaattcaccaagttcaatagagggggtacaagccagtaccaccacgaacaagcacttctgttaccccggtgagtttgcataagctgtgcccacggctgaaagtaaatgatccgttatccgcttgagtacttcgagaagcctagtatcaccttggaatcttcgacgcgttgcgctcaacactctaccccgagtgtagcttaggctgatgagtctgggcatcccccaccggcgacggtggcccaggctgcgttggcggcctacccatggccaacgccatgggacgctagttgtgaacaaggtgtgaagagcctattgagctactcaagagtcctccggcccctgaatgcggctaatcctaaccatggagcaagtggccccaacccaggggatggcttgtcgtaacgcgcaagtccgtggcggaaccgactactttgggtgtccgtgtttccttttattttatatatggctgcttatggtgacaatcacagattgttatcataaagcgaattggattggccatccggtgaaatataagcacattatttatatctttgctggattcgctccgttgacacattcttcaccaaagttgatttatacagtcttagtaattagaaatcaccacttcagtcatgggagctcaagtatccactcaaaagactggggcccacgaaaaccagaatgtggccgcgaacgggtccacaatcaattatacaaccatcaactactataaggacagtgcaagcaattcggcgactcggcaggatttctcacaggacccgtccaaattcactgaaccggtcaaggatttaatgttaaaatccgcaccggctttgaattcgccaaacgtcgaagcatgcgggtacagtgatcgcgtgaggcaaatcacactgggaaattccaccataaccacacaggaggctgcaaatgcaattgttgcctatggggagtggccaacctacataaacgataacgaggccaaccctgtcgatgcaccgaccgaaccagatgtgagtagcaaccgcttttacaccctggaatcagtgcagtggaaaacaacatcccgggggtggtggtggaaattgccagactgtttgaaagacatgggtatgttcgggcaaaacatgtattaccactatttaggcaggtcagggtatactatacatgtgcagtgtaatgcctcaaaatttcaccaaggtgcgctgggtgtgtttctaatccctgaatttgtgatggcatgtaacaccgagagcaagacctcatatgtctcatatattaatgccaaccctggtgagaagggtggtgaatttacaaacacctacaacccatctggcacagacgttagcgaagggaggaaatttgccgctctggattatctgctagggtcaggcgtcttggcgggaaacgcttttgtttacccccatcaaattatcaaccttcgcaccaacaatagtgcaacaatagtggttccttacgtcaactcactagtgatcgattgcatggcaaaacacaacaactgggggatagcgatactaccccttgcacccctagcctttgcagcaacgtcatcaccacaagtaccgatcacagtcacaattgcaccaatgtgtacagagttcaatggattgaggaatgtaactgtaccagtacaccagggccttccagtcatgaacaccccaggatcaaaccaattcctcacatcagatgatttccagtcgccatgtgccttacccaatttcgatgtgacccctccaatccacatcccaggggaggtcaaaaacatgatggaactagctgaaattgacacactgattccaatgaatgcagtggatgggaaagtaaataccatggaaatgtaccagatcccattaaacgacaaccttagcactgaacccatcttttgcctatcactatcaccagcgtctgataggcgattgagtcacaccatgctgggagagattctgaactactacacacactggacgggctccatcagatttacttttctattttgtggtagtatgatggccacagggaagttgctccttagctattcacccccaggggcgaagccacccaaaaaccggaaagacgccatgctgggtacgcacataatatgggacctgggtttacagtctagttgttccatggtggcgccctggatttccaatacggtgtacaggcgatgtgttcgcgacgattttaccgaaggtggctacatcacctgtttctatcagactagaatagtggtaccagcctcgaccccaaccagcatgtttatgctaggctttgtcagtgcttgtcccgactttagtgtgagactgctcagggacacgacgcacatcagtcaatcgaaattggtcgccagtaggcagggcatagaggagtttatagacacagcaattaaaaatgcattgaaagtgtcacaacccagcactgcacaatcaacagaagcaaccagcggagtcaacagccaagaggtgcctgcactaacagcagtagagactggcgcatcgggccaagcaatcccaagtgatgtggtagaaactcgccacgtggtgaactataagacaagatcagagtcctgccttgaatcattctttggtagggccgcttgtgtgacaattctctcccttacaaactcttcaggtagcggagatgcaaagaagcactacacaacatggaacatcacatatacagatacggtgcaactgcgaaggaaactggaattttttacgtattccaggtttgacttggaaatgacctttgtattcactgagaactaccctagcacagctagtggagaggttcgaaatcaggtttaccaaatcatgtacattcctccaggggcgcctagacctagttcttgggatgattacacctggcagtcatcatcaaacccttccatattctacatgtatggtaatgcaccaccacggatgtcgattccctatgtcgggattgctaacgcctactcgcacttctacgatggcttcgcgcgagtgccgcttgaaggtgagaacacagatgccggggacaccttctacgggctagtatccatcaatgattttggtgtactggcagttagggcagtgaaccgcagcagccctcacgccatccacacttcagtgagggtgtatatgaaacctaaacacattcgctgctggtgccccaggccaccacgagcagtactttaccgtggtgagggagtggacatgatctcaagtgcaatccaaccactaaccaaggtcgattctataacaacattcggctttggtcaccagaacaaggcagtttatgttgctgggtataaaatttgtaactaccacttagcaacacccagtgaccacctgaacgccattagtgtgatgtgggacagagatttgatagtagtggagtcaagagcacagggaattgacaccatagcccggtgcaactgcaagtgtggcgtatactactgtgaatcaaggaaaaagtattatcccatttcgttcactgggcccacatttagatacatggaggccaacgaatactttccagctagatatcagtcccatttattaattgggtgcgggtttgcagagcctggagactgtggtggcatactcagatgcacacacggtgtgatcggcataattacagcaggtggacctggagtggtggcctttgccgacatcagggacctctgggtgtatgaggaggaagcgatggaacaaggcattacaagctacatcgagactttaggggcagcatttggaactgggttcactcaacaaattggagacaaaattacagagttgactaatatggtgacaagcacttttacggacaaattgtttaaaaacttgattaaaatagtgtcttcgttggttataataactcgaaactacgaagacaccaccacagtattggccactctagcattgttgggttgtgattgctctccgtggcaatggttgaaacacaaagtatgtgacatcctgcaactgccattcgccctcaaacagagtgacagttggctaaagaagttcactgaggcgtgtaatgccgcaaaagggctcgaatggattagcaacaagctctccaaatttgtggaatgggtgaagaacaagataattcctgaagccagagacaaagtggaatttctgcaaaaattgaaacagttagaaatgcttgagaaccagattgctacactacaccaatcctgtccaagtcaagaacagcaagagacactctttaacaatgtccgctggttggcactgcagtcaagacgctttgcgccgttgtacgctgttgaagcgcgcagaatacaaaaattggagaacaccataaacaactacatacagtttaagagcaaacaccgtattgaaccagtatgtatgttgatacatggagctcctggcacgggcaaatctatagcaacctcgttgattgggcgtgccattgctgaaaaagaaagcacctcagtgtattctctaccaccagacccctcacacttcgacggttacaaacaacaaggggttgttatcatggatgacctcaatcagaacccggatggtatggacatgaagttgttttgtcagatggtgtctacagtcgagttcatacccccaatggcatctctcgaggagaaaggaatcctgttcacttcagactatgtgctagcatctacaaattctcactcaattaccccacccactgtcgcacatagtgatgccctaagtagaagatttgcatttgacatggatgtatacaccatgtctgaacactctgtgaagggaaagctcaacatggcgactgcgacgcagttatgcaaagagtgtccaccgcccgctaacttcaaaaagtgctgtccactagtgtgtggtaaagctttacaactgatggacaagcacaccagacagagatttacactagacgaaatcaccactttgatcatcaatgagaagaacagaagggctaacattggtaattgcatggaggctctttttcaaggccctctacaatacaaagatctcaagatcgacatcaagaacactccccctccagagtgcataagtgatctacttcaggctgttgactcgcaagaggttagagattactgtgagaagaaaggatggatcgttaacgtatccagtcaggttcaattagagcgcaacatcaatagagctatgactatcctccaggcagtaaccacgttcgccgcagtggcgggagtggtttatgtcatgtacaagctctttgcagggcatcaaggcgcttatactgggctcccgagcaagaaacccaacgtgcccaccattagaatagctaaagtgcaaggtccagggtttgactatgcagttgccatggcaaagagaaatatcgttactgcaaccaccaataagggggagttcactatgcttggagtgcatgataacgttgccgtactaccaacccatgcatcaccaggtgagaccatagttattgatggtaaggaagttgaagtgctggacgccaaggccctggaggaccaagctggaactaacctagagatcaccattgtcactctgagaaggaatgagaagttcagggacatcagacctcacatccccactcaaatcactgagacaaatgatggagtgttgatcgtgaacactagtaagtaccccaacatgtatgtgcccgttggtgcagtgaccgaacaagggtacttgaacctgggtgggcgtcaaactgctcgcactctaatgtacaatttcccaacccgagccggacaatgtggtggaatcatcacatgcactggcaaggtcattgggatgcatgttggtgggaacggttcacatgggttcgctgcagccctaaaacggtcatacttcactcagagccaaggtgagattcagtggatgagaccatcaaaagaggtcggttacccaataattaatgcaccaagcaagacaaaactagaacccagtgctttccactatgtttttgaaggggtaaaggaacctgcagtactgactaaaaacgatcccagattgaaaacggattttgaggaggccatattctcaaagtatgtagggaacaagatcactgaagtggatgaatatatgaaagaggcagtagaccattatgctgggcaactcatgtctttagacatcaatacagagcaaatgtgcttggaagatgctatgtatggcactgatggtctggaagctttggatctctctactagcgcaggatacccttatgtggctcttgggaaaaagaagagagatatacttaataaacaaacaagagacaccaaagaaatgcaaagactactagatacctatggaataaatttgcctctagttacttacgtaaaagatgagctgagaagcaagagcaaggtggagcagggtaaatccagattaattgaggcctcaagcttgaatgattcagtagcaatgagaatggcatttggtaacctgtatgcagctttccacaagaatccaggagtcatcactggttctgctgtaggatgtgacccagacttgttttggagcaagatcccagtattgatggaagaaaagctctttgcatttgattacaccggttacgatgcctctctcagtcccgcttggtttgaggcactcaaaatggttttggagaaaattggattcggagatagggtagattacattgattatctcaaccattcacaccatctttacaaaaacaagacctattgtgtcaagggcggtatgccatctggctgctcaggcacttcaatatttaattcaatgattaacaatttaataatcaggacactcttactgaagacctacaagggcatagatttggaccacttgaagatgattgcctatggtgatgatgtaattgcctcctacccccacgaagttgacgctagtctcctagcccaatcaggaaaagactatggactaaccatgacaccagctgacaagtcagccacctttgaaactgtcacgtgggagaatgtaacatttttaaaaagattctttagggcagatgaaaagtacccattcttaatacatccagtgatgccaatgaaagaaatacaagaatcgatcagatggactaaagatcctagaaacacgcaggaccatgttcgctcactgtgcttattggcttggcacaatggcgaagaagagtacaataaatttttagctaaaatcagaagtgtgccaatcggaagagctttactgctccccgaatactcaactttgtaccgccgttggctcgactcgttttagtaaccctacctcagtcgaattggattgggtcatactgttgtaggggtaaatttttctttgattcggagaaaaaaaaaaaaaaaaaaaaaaaaaaaaaaaaaaaaaaaaaaaaaaaaaaaaaaaaaaaaaaaaaaaaaaagagacg

SEQ ID NO: 22 - DNA, 7626 bp, synthetic construct
  misc_feature 1..7626: note = CVA-PP-TwL6-BsmBI_Kuykendall_70pA
  source 1..7626: mol_type = other DNA
taatacgactcactatagggtcgactctacaaataacggataccaatttgagtaacgcggcttcaatgtacaataatggactcgtctgagcgagtataaacagttttttaagctcagagcgttcaccggggaaattcggtgaggttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaaatgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccacttttgggtttggtcatcagaacaaagcagtgtacgttgccggttacaagatttgcaactaccacctagcaaccccaagtgatcacttgaatgcaattagtatgttatgggacagggatttaatggtggtggaatctagagcccagggaactgataccatcgccagatgtagttgcaggtgtggagtttactattgtgaatctaggaggaagtactaccctgtcacttttactggcccaacgtttcgattcatggaagcaaacgactactatccagcaagataccagtctcacatgctgatagggtgcggatttgcagaacccggggactgcggtgggatactgaggtgcactcatggggtaattggtatcattactgcaggaggtgaaggggtagtagcctttgctgacattagagacctctgggtgtatgaagaggaggccatggaacagggaataacaagctacatcgaatctctcggcacagcctttggcgcagggttcacccacacaatcagtgagaaagtgactgaattgacaacaatggttaccagcactatcacagaaaaactactgaaaaacttggtgaaaatagtgtcggctctagtgattgttgtgagaaattatgaggacactaccacgatccttgcaacactagcactactcgggtgtgatatatctccttggcaatggttgaagaagaaggcatgtgacttactagagattccttatgtgatgcgccaaggtgatgggtggatgaagaaattcacagaggcgtgcaatgcagctaaaggcttagagtggattagcaacaaaatttccaagtttatagattggttgaagtgtaaaattatcccagacgctaaggacaaggtggaatttctcaccaagttgaaacagctagacatgttggaaaatcaaattgcaaccatccaccaatcttgccccagccaagaacaacaagagattcttttcaacaatgtgagatggctagcagtccagtcccgtcggtttgcaccattatacgctgtggaggcacgccgaattaacaaaatggagagcacaataaacaattatatacagttcaagagcaaacaccgtattgaaccagtatgtatgctcattcatgggtcaccagggacgggtaaatctatagctacttcattaataggtagagcaatagcagagaaggaaagcacatcagtctattcaatgccacctgacccatctcactttgatggctataaacaacaaggggtagtgattatggacgacctaaaccaaaaccccgatggtatggacatgaaactgttttgccaaatggtatcaacagtggagtttattcctccaatggcctcattagaggagaagggcattttgtttacatctgattatgtcctggcttctaccaactctcattcaattgtaccacccacagtggctcacagtgatgccttaaccagacgatttgcatttgatgtggaggtttacacgatgtctgaacattcagtcaaaggcaaactgaatatggccacggccactcaattgtgtaaggattgtccaacacctgcaaattttaaaaagtgttgccctctcgtttgtggaaaggccttgcaattaatggacaggtacaccagacaaaggttcactgtagatgagattaccacattaatcatgaatgagaaaaacagaagggccaatatcggcaattgcatggaagccttgtttcaaggaccattaaggtataaagatttgaagatcgatgtgaagacagttcccccccctgagtgcatcagtgatttgttacaagcagtggattctcaagaggttagggattactgtgagaagaaaggctggatcgttaacgttactagccagattcaactagaaaggaacatcaatagggccatgactatactccaagctgttaccacattcgcagcagtcgcaggagtagtgtatgtaatgtacaaactcttcgccggtcaacagggtgcatacactggcttgccaaacaaaaaacccaatgtccctactatcagagtcgctaaagtccaggggccaggatttgactacgcagtggcaatggcaaaaagaaacatagttactgcaaccaccaccaagggtgaatttaccatgctaggggtgcatgataatgtagcaatattgccaacccatgccgctccaggagaaaccattattattgatgggaaagaagtagagatcctagatgccagagccttagaagatcaagcgggaaccaatcttgagatcaccattattactctaaaaagaaatgagaagtttagagacatcagatcacatattcccacccaaattactgaaactaacgatggagtgttgatcgtgaacactagcaagtaccccaatatgtatgtccccgttggtgctgtgaccgaacagggatatcttaatctcagtggacgtcaaactgctcgcactttaatgtacaactttccaacaagggcaggccagtgcggaggaatcatcacttgtactggcaaagtcattgggatgcatgttggcgggaacggttcacatgggtttgcagcagccctcaagcgatcatacttcactcaaaatcagggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaaccctacctcagtcggattggattgggttatactgttgtaggggtaaatttttctttaattcggagaaaaaaaaaaaaaaaaaaaaaaaaaaaaaaaaaaaaaaaaaaaaaaaaaaaaaaaaaaaaaaaaaaaaaaagagacg

SEQ ID NO: 23 - DNA, 67 bp, Paenibacillus polymyxa
  source 1..67: mol_type = unassigned DNA
aaactcgtctgagcgagtataaacaggtcattaagctcagagcgttcaccgggatcatgcggtgagg

SEQ ID NO: 24 - DNA, 67 bp, Paenibacillus polymyxa
  source 1..67: mol_type = unassigned DNA
aaactcgtctgagcgagtataaacaggtcattaagctcagagcgttcaccgggatcattcggtgagg

SEQ ID NO: 25 - RNA, 7280 nt, synthetic construct
  misc_feature 1..7280: note = SVV-001
  source 1..7280: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 26 - RNA, 7280 nt, synthetic construct
  misc_feature 1..7280: note = SVV-001-S177A
  source 1..7280: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 27 - RNA, 8048 nt, synthetic construct
  misc_feature 1..8048: note = SVV-001-GFP
  source 1..8048: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 28 - RNA, 8050 nt, synthetic construct
  misc_feature 1..8050: note = SVV-IRES-1-GFP
  source 1..8050: mol_type = other RNA
tttgaaatggggggctgggccctcatgcccagtccttcctttccccttccggggggtaaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctatccactcactcacgcacaaaaagtgtgctgtaattacaagatttagccctcgcacgagatgtgcgataaccgcaagattgactcaagcgcggaaagcgctgtaaccacatgctgttagtcccttcatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 29 - RNA, 8050 nt, synthetic construct
  misc_feature 1..8050: note = SVV-IRES-2-GFP
  source 1..8050: mol_type = other RNA
tttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 30 - RNA, 8050 nt, synthetic construct
  misc_feature 1..8050: note = SVV-IRES-3-GFP
  source 1..8050: mol_type = other RNA
tttgaaatggggggctgggccctgatgcccagtccttcctttccccttccggggggttaaccggctgtgtttgctagaggcacagtggagcaacatccaacctgcttttgtggggaacggtgcggctccaattcctgcgtcgccaaaggtgttagcgcacccaaacggcgcgtctaccaatgctattggtgtggtctgcgagttctagcctactcgtttctcccctactcactcattcacgcacaaaaactgtgttgtaactacaaggtttagccctcgcacgggatgtgcgataaccgcaagatcgactcaagcgcggaaagcgctgtaaccacatgctgttagtccctttatggctgcgagatggctacccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 31 - RNA, 8048 nt, synthetic construct
  misc_feature 1..8048: note = SVV-P1-1-GFP
  source 1..8048: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcacttttctttcgatacagcctctggcacctttgaagacgtaaccggaacaaaagtcaagatcgttgaataccctagatcggtgaacaatggtgtttacgattcgtccactcatttagagatactgaacctacagggtgaaattgaaattttaaagtctttcaatgaataccaaattcgcgccgccaaacaacaacttggactggacatcgtatacgaactacagggtaatgttcagacaacctcaaagaatgattttgattcccgcggtaataatggtaacatgaccttcaattactacgcaaacacttaccagaattcagtagacttctcgacctcctcgtcggcgtcaggcgccggacccgggaactcccggggcggattagcgggtctcctcacaaatttcagtggaatcttgaaccctcttggctacctcaaagatcacaataccgaagaaatggaaaactctgctgatcgagtcataacgcaaacggcgggcaacactgccataaacacgcaatcatcactgggtgtgttgtgtgcctacgttgaagacccgaccaaatctgaccctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggctaccctacatagacatttcttaatgaagtgcgggtggcaggtgcaggtccaatgcaatttgacgcaattccaccaaggtgctcttcttgttgccatggtccccgaaaccacccttgatgtcaaacctgacggcaaggcaaagagcttacaggagctgaatgaagaacagtgggtggaaatgtctgacgattaccggaccgggaaaaacatgcctttccagtctcttggcacatactatcggccccctaactggacttggggccccaatttcatcaacccctatcaagtaacagttttcccacaccaaattctgaacgcgagaacctctacctcggtagacataagtgtcccatacatcggggagactcctacacaatcctcagagacacagaactcctggaccctcctcgttatggtgcttgtccccctggactacaaggagggagccacaactgacccagaaattacattttctgtaaggcctacaagtccttacttcaatgggctacgtaaccgtttcacgaccgggacggacgaggaacaggggcccattcccacagcacccagagaaaattcgcttatgtttctctcaaccatccctgacgacactgtccctgcttacgggaatgtgcgtacccctcccgtcaattacctccctggtgaaataaccgacctcttacaactggcccgtatacccactctcatggcgttcgggcgggtgtctgaacccgagcctgcctcagacacatatgtgccctacgttgccgttcctgcccagttcgacgacaagcctctcatctccttcccgatcaccctttcagatcctgtctaccagaacaccctggtaggcgccatcagttcgaatttcgccaactaccgggggtgtatccaaatcactctgacattttgtggacccatgatggcaagagggaaattcctactctcgtattctcctccaaatggagcacaaccacagaccctttctgaagctatgcagtgcacgtactctatttgggatataggcttgaactctagttggacctttgtcatcccctacatctcgcccagtgattaccgtgaaactcgggctattaccaactcagtttattctgctgatggttggtttagcctgcacaagctgactaaaattactctaccacctgactgcccacagagtccctgtattctctttttcgcctctgctggtgaggattacaccctccgtctccctgttgattgtaatccttcctacgtgttccactccaccgacaacgccgagactggggttattgaggcgggtaacactgacaccgacttctctggtgagctggcggctcctggctctaaccacactaatgtcaagttcctgtttgatcgatctcgattactgaatgtaattaaggtactggagaaggacgccgtcttcccccgtcctttccccacagcaacaggtgcacagcaggacgatggttacttttgccttctaacaccccgcccaacagtcgcttcccgacccgccactcgtttcggcctgtacgtcaatccgtctgacagtggcgttctcgctaacacttcactggatttcaatttttacagcttggcctgtttcacttactttagatcagaccttgaagtcacggtggtctcactggagccagatctggaattcgctgtagggtggttcccctctggcagtgagtaccaggcttccagctttgtctacgaccaactgcatgtaccctaccactttactgggcgcactccccgcgctttcaccagcaagggtggaaaggtatctttcgtgcttccttggaactctgtctcatccgtgcttcccgtgcgctgggggggcgcttccaagctttcttccgccacgcggggtctaccggctcatgctgactgggggaccatttacgccttcatcccccgtcctaatgagaagaaaagcaccgctgcaaagcacgtggcggtgtacgttcggtacaagaacgcgcgtgcctggtgccccagcatgctccccttccgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 32 - RNA, 8048 nt, synthetic construct
  misc_feature 1..8048: note = SVV-P1-2-GFP
  source 1..8048: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagacgtaaccggaacaaaagtcaagatcgttgaataccccagatcggtgaacaatggtgtttacgattcgtccactcatttagagatactgaacctacagggtgaaattgaaattttaaggtctttcaatgaataccaaattcgcgccgccaaacaacaacttggactagacattgtatacgaactacagggtaacgttcagacaacctcaaagaatgattttgattcccgcggcaataacggtaacatgaccttcaattactacgcaaacacttatcagaactcagtagacttctcgacctcctcgtcggcgtcaggcgccggacccgggaactcccggggcggattagcgggtctcctcacaaatttcagtggaatcttgaaccctcttggctaccttaaagatcacaataccgaagaaatggaaaactctgctgatcgagtcataacgcaaacggcgggcaacactgccataaacacgcaatcatcactgggtgtgttgtgtgcctatgttgaagacccgaccaaatctgatcctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggctaccctacatagacatttcttaatgaagtgcgggtggcaggtgcaggttcaatgcaatttgacacaattccaccaaggtgctcttcttgtcgccatggtccccgaagctacccttgatgtcaagcccgacggcaaggcaaagagcctgcaggagctgaatgaagagcagtgggtagaaatgtccgacgattaccggaccgggaaaaacatgccttttcagtctcttggcacatactatcggccccctaactggacttggggccctaatttcatcaacccctatcaagtaacagttttcccacaccaaattctgaacgcgagaacctctacctcggtagacataagtgtcccatacatcggggagactcctacacaatcctcagagacacagaactcctggaccctcctcgttatggtgcttgtccccctggactacaaggagggagccacaactgacccagaaattacattttctgtaaggcctacaagtccttacttcaatgggcttcgtaaccgttacacgaccgggacggacgaggaacaggggcccattcccacagcacccagagaaaattcgcttatgtttctctcaaccatccctgacgacactgtccctgcttacgggaatgtgcgtacccctcccgtcaactacctccctggtgaaataactgacctcttacaactggcccgtatacccactctcatggcgtttgggcgggtttctgaacccgaacctgcctcagacgcatatgtgccctacgttgccgttcctgcccagtttgatgacaagcctctcatctccttcccgatcaccctttcagatcctgtctaccagaacaccctggtaggcgccatcagttcgaatttcgccaactaccgggggtgtatccaaatcactctgacattttgtggacccatgatggcaagagggaaattcctactctcgtattctcccccaaatggaacacaaccacagaccctttctgaagctatgcagtgcacgtactccatttgggatataggcttgaactctagttggacctttgtcatcccctacatctcgcccagtgactaccgtgaaactcgggccatcaccaactcggtttattctgctgacggctggtttagcctacacaagctgaccaaaattactctaccacctgactgcccacagagcccctgtattctctttttcgcctctgctggtgaggattacaccctccgtctccccgttgattgcaatccttcctacgtgttccactccaccgataacgccgagactggagttattgaggcgggtaatactgacaccgatttctctggtgaactggcggctcctggctctaaccatactaatgtcaagttcctgtttgatcgatctcgattactgaatgtaattaaggtactggagaaggacgccgtcttcccccgtcctttccccacagcaacaggtgcacagcaggacgatggttacttttgtcttctaacaccccgcccaacagtcgcctcccgacccgccactcgtttcggcctgtatgtcaatccgtctgacagtggcgttctcgccaacacttcactggatttcaatttttacagcttggcctgcttcacttactttagatcagaccttgaagtcacggtggtctcactggagccagatctggaattcgctgtagggtggttcccctctggcagtgagtaccaggcttccagctttgtctacgaccaactgcatgtaccctaccactttactgggcgcactccccgcgctttcaccagcaagggtgggaaggtatctttcgtactcccttggaactctgtctcatccgtgcttcccgtgcgctgggggggtgcttccaagctttcttctgccacgcggggtttgccggctcatgctgactgggggactatttacgcctttatcccccgtcctaatgagaagaaaggcaccgctgtaaagcacgtggccgtgtacgttcggtacaagaacgcgcgtgcctggtgccccagcatgctccccttccgcagttacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 33 - RNA, 8048 nt, synthetic construct
  misc_feature 1..8048: note = SVV-P1-3-GFP
  source 1..8048: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaaaacgtaaccggaacaaaagtcaagatcgttgaataccccagatcggtgaacaatggtgtttacgattcgtccactcatttagagatactgaacctacagggtgaaattgaaattttaaagtctttcaacgaataccaaattcgcgccgccaaacaacaacttggactggacatcgtatacgaactacagggtaatgttcagacaacctcaaagaatgattttgattcccgcggcaataatggtaacatgaccttcaattactacgcaaacacttaccagaattcagtagacttctcgacctcctcgtcggcgtcaggcgccggacccgggaactcccggggcggattagcgggtctcctcacaaatttcagtggaatcttgaaccctcttggctacctcaaagatcacaataccgaagaaatggaaaactctgctgatcgagtcataacgcaaacggcgggcaacactgccataaacacgcaatcatcactgggtgtgttgtgtgcctacgttgaagacccgaccaaatctgaccctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggccaccctacatagacatttcttaatgaagtgcgggtggcaggtgcaggttcaatgcaatttgacgcaattccaccaaggtgctcttcttgttgccatggtccccgaaaccacccttgatgtcaaacctgacggcaaggcaaagagcttacaggagctgaatgaagagcagtgggtggaaatgtctgacgactaccggaccgggaaaaacatgccttttcagtctcttggtacatactatcggccccctaactggacttggggccccaatttcatcaacccctatcaagtaacagttttcccacaccaaattctgaacgcgagaacctctacctcggtagacataagtgtcccgtacatcggggagactcctacacaatcctcagagacacagaactcctggaccctccttgttatggtgcttgtccccctggactacaaggagggagccacaactgacccagaaattacattttctgtaaggcctacaagtccctacttcaatgggcttcgtaaccgtttcacgaccgggacggacgaggaacaggggcccattcccacagcacccagagaaaattcgcttatgtttctctcaaccatccctgacgacactgtccctgcttacgggaatgtgcgcacccctcccgtcaattacctccccggtgaaataaccgacctcttacaactggcccgtatacccactctcatggcgtttgggcgggcgtctgaacccgagcctgcctcagacgcatatgtgccctacgttgccgttcctgcccagttcgacgacaagcctctcatctccttcccgatcaccctttcagaccctgtctaccagaacaccctggtaggcgccatcagttcgaacttcgccaactaccgggggtgtatccaaattactctgacattttgtggacccatgatggcaagagggaaattcctgctctcgtattctcccccaaatggagcacaaccacagaccctttctgaagctatgcagtgcacatactctatttgggatataggcttgaactccagttggacctttgtcatcccctacatctcgcccagtgattaccgtgaaactcgggctattaccaactcagtttattctgctgatggttggtttagcttgcacaagctgaccaaaattactctaccacctgactgcccacagagtccctgtattctctttttcgcctctgctggtgaggattacaccctccgtctccctgttgattgtaatccttcctacgtgttccactccaccgacaacgccgagactggggttattgaggcaggtaacactgacaccgatttctctggtgaactggcggctcctggctctaaccatactaatgtcaaattcctgtttgaccgatctcgactactgaatgtaattaaggtactggagaaggacgccgtcttcccccgtcctttccccacagcaacaggtgcacagcaggacgatggttacttttgtcttctaacaccccgcccaacagtcgcttcccgacccgccactcgtttcggcctgtacgtcaacccgtctgacagtggcgttctcgctaacacttcactggatttcaatttttacagtttggcctgtttcacttactttagatcagaccttgaagtcacggtggtctcactggagccagatttggaattcgccgtggggtggttcccctctggcagtgagtaccaggcttctagctttgtctacgaccaactgcatgtaccctaccactttactgggcgcactccccgcgctttcaccagcaagggtggaaaggtatctttcgtgctcccttggaactctgtctcttccgtgcttcccgtgcgctgggggggtgcctccaagctttcttctgccacgcggggtctgccggctcatgctgactgggggaccatttacgcctttatcccccgtcctaacgagaagaaaagcaccgctgtaaagcacgtggcggtgtacgttcggtacaagaacgcgcgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 34 - RNA, 8048 nt, synthetic construct
  misc_feature 1..8048: note = SVV-P3-1-GFP
  source 1..8048: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaacgacgacacccccgtcgacgaggcgttgggtagagttctcacccccgctgcggtcgacgaggcgcttgtcgacctcgctccagatgccgacccggttggccgtttggctattctcgccaagctaggtcttgccctagctgcggtcacccctggtttgataatcttggcagtgggactctacaggtacttctctggctctgatacagaccaagaagaaacagaaggtgaggagcctgctaaagcgcctaggagcgagaatgcttatgacggcccgaagaaaaactctaagccccctggagcgctctcccttatggaaatgcaacagcccaacgtggacatgggctttgaggctgcggtcgctaagaaagtggtcgtccccattaccttcatggttcccaacagaccttctggacttacacagtccgctcttcttgtggccggccggaccttcctaattaatgagcatacatggtccaacccctcctggaccagcttcacaatccgtggtgaggtgcacacccgtgatgagcctttccaaacggttcattttactcaccatggtgttcccacagatctggtgatggtacgtctcggaccgggcaactccttccctaacaatctagacaagtttggacttgatcagatgccggcacgtaactcccgtgtggttggcgtttcggctagttacggcaacttcttcttctctgggaacttcctcgggtttgttgactccatcacctctgaccaaggaacttatgcgagactttttaggtacagggtgacgacctacaaggggtggtgcggttcggccctggtttgtgaggccggtggtgtccggcgcatcattggcctgcattctgctggtgccgctggtatcggcgccgggacttacatctcaaaattaggactgatcaaagccctcaaacacctcggtgaacctctggctacaatgcaaggactgatgactgagctagagcctggagtcaccgtgcatgtaccccggaaatctaaattgagaaagacgaccgcacacgcggtgtacaaaccggagtttgaacctgctgtgttgtcaaaatttgatcccagactgaacaaggatgttgacctagatgaggtaatttggtctaaacacactgccaacgtcccttatcaacctcctttgttctacacatacatgtcagagtacgctcatcgggttttctcctttttgggaaaagacaatgacattctgaccgtcaaagaagcaatcttgggaatccctggactagaccctatggatccccacacagctccgggtctgccctacgccattagcggccttcgacgtactgatctcgtcgattttgcgaacggcacggtggacccgacactggccgtacaaatccaaaaattcttagacggtgactactctgatcatgtcttccaaacttttctgaaagatgaaatcagaccctcagagaaggtccgagcgggaaaaacccgcattgtcgatgtgccctccctggcgcactgcattgtgggcagaatgttgcttgggcgctttgccgccaagtttcaatcccatcctggctttctccttggctctgctatcgggtctgaccctgatgtcttctggaccgtcataggggctcagctcgagggaagaaagaacacgtatgacgtggactacagtgcctttgactcttcacacggcactggctccttcgaggctctcatctctcactttttcaccgtggacaatggttttagccctgcgctgggatcgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctggcattgacctacaaggaatttgaatatgacatggttgatatcatcgcctacggtgacgaccttctggttggtacggattacgatctggacttcaatgaggtggcgcggcgcgctgccaaactggggtataagatgactcctgccaacaaaggttctgtcttccctccgacttcctctctctccgacgctgtttttctaaaacgcaaattcgtccaaaacaatgacggcttatacaaaccagttatggatttaaagaatttggaagccatgctctcctacttcaaaccaggaacactactcgagaagctgcaatctgtttctatgttggctcaacattctggaaaagaagaatatgatagattgatgcacccctttgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 35 - RNA, 8048 nt, synthetic construct
  misc_feature 1..8048: note = SVV-P3-2-GFP
  source 1..8048: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgagtagagttctcacccccgctgcggtcgacgaggcgcttgtcgacctcgctccagaggccgacccggttggccgcttggctattcttgccaagctaggtcttgccctggctgcggtcacccctggtttgataatcctggcagtgggactctacaggtacttctctggctctgatacagaccaagaagaaacagaaagtgaggaacctgttaaagcgcccaggagcgagaatgcttatgacggcccaaagaaaaactctaagccccctggggcgctctctcttatggaaatgcagcagcccaacgtggacatgggctttgaggctgcggtcgctaagaaagtggtcgtccccattaccttcatggttcccaacagaccttctggacttacacagtccgctcttcttgtggccggccggactttcctaattaatgagcatacatggtccaacccctcctggaccagcttcacaatccgtggtgaggtgcacactcgtgatgagcctttccaaacggttcattttactcaccatggtgttcccacagatctgatgatggtgcgtctcgggccgggcaactctttccctaacaatctagacaagtttggacttgaccagatgccggcacgtaactcccgtgtggttggcgtttcggctagttacggtaacttcttcttctctgggaatttcctcgggtttgttgactccatcacctctgaccaaggaacttatgcgagactttttaggtacagggtgacgacttacaagggatggtgtggttcggccctggtctgtgaggccggtggtgtccggcgcatcattggcttgcactctgctggcgccgctggtatcggcgccgggacttacatctcaaaattgggactgatcaaagcccttaaacacctcggtgaacctctggctacaatgcaaggactgatgactgagctagagcctggagtcaccgtacatgtaccccggaaatctaaattgagaaagacgaccgcacacgcggtgtacaaaccggagtttgaacctgctgtgttgtcaaaatttgatcccagactgaacaaggatgttgacttagatgaggtgatttggtctaaacacactgccaacgtcccttatcaacctcctttgttctacacatatatgtcagagtacgctcaccgggttttctcctttttgggaaaagacaatgacattctgaccgtcaaagaagcaatcttgggcatccctggactagaccctatggatccccacacagctccgggtctgccctacgccattagcggccttcgacgtactgatctcgtcgattttgcgaacggcacggtagacgcggcactggccatgcaaattcagaaattcttagatggtgactactctgatcatgtcttccaaacttttctgaaagatgaaatcagaccctcagagaaggtccgagcgggaaaaacccgcattgtcgatgtgccttccctggcgcactgcattgttggcagaatgctgcttgggcgttttgccgccaagtttcaatcccatcctggctttctccttggctctgctatcgggtctgaccctgatgtcttctggaccgtcataggggctcagctcgagggaagaaagaacacgtatgacgtggactacagtgcctttgactcttcacacggcactggctccttcgaggctctcatctctcactttttcaccgttgacaatggttttagccctgcgctgggaccgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctagcattgacttacaaggaatttgagtatgacatggttgatatcatcgcctacggtgacgaccttctggttggtacggattatgatctggacttcaatgaggtggcgcggcgcgctgccaaactggggtataagatgactcctgccaacaagggttctgtcttccctccgacttcctctctctccgatgctgtttttctaaaacgcaaattcgtccaaaacaatgacggcttatataaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 36 - RNA, 8048 nt, synthetic construct
  misc_feature 1..8048: note = SVV-P3-3-GFP
  source 1..8048: mol_type = other RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccctaacgagaacgacgacacccccgtcgacgaggcgttgggtagagttctcacccccgctgcggtcgacgaggcgcttgtcgacctcgctccagatgccgacccggttggccgcttggctattctcgccaagctaggtcttgccctagctgcggtcacccctggtttgataatcttggcagtgggactctacaagtacttctctggctctgatacagaccaagaagaaacagaaagtgaggagcctgttaaagcgcctaggagcgagaatgcttatgacggcccgaagaaaaactctaagccccctggagcgctctctcttatggaaatgcaacagcccaacgtggacatgggctttgaggctgcggtcgctaagaaagtggtcgtccccattaccttcatggttcccaacagaccttctggacttacacagtccgctcttcttgtgaccggccggaccttcctaatcaatgagcatacatggtccaacccctcctggaccagcttcacaatccgtggtgaggtgcacactcgtgatgagcctttccaaacggttcattttactcaccatggtcttcccacagatctgatgatggtacgtctcggaccgggcaactccttccctaacaatctagacaagtttggacttgaccagatgccggcacgtaactcccgtgtggtcggcgtttcggctagttacggtaacttcttcttctctgggaacttcctcgggtttgttgactccatcacctctgaccaaggaacctatgcgagacttttcaggtacagggtgacgacttacaagggatggtgcggttcggccctggtctgcgaggccggtggtgtccgacgcatcattggcctgcactctgctggtgccgctggtatcggtgccgggacttacatctcaaaattaggactgatcaaagcccttaaacacctcggtgagcctctggctacaatgcaaggactgatgactgagctagagcctggagtcaccgtacatgtaccccgaaaatctaaattgagaaagacgaccgcacacgcggtgtacaaaccggagtttgaacctgctgtgttgtcaaaatttgatcccagactgaacaaggatgttgacctagatgaggtaatttggtccaaacacactgccaacgtcccttatcaacctcctttgttctacacatacatgtcagagtacgctcatcgggttttctcctttttgggaaaagacaatgacattctgaccgtcaaagaagcaatcttgggcatccctggactagaccctatggatccccacacagctccgggtctgccctacgccattaacggccttcgacgtactgatctcgtcgattttgcgaacggcacggtagacccggcactggccatgcaaatccaaaaattcttagacggtgactactctgatcatgtcttccaaacttttctgaaagatgaaatcagaccctcagagaaggtccgggcgggaaaaacccgcattgtcgatgtgccctccctggcgcactgcattgtgggcagaatgctgcttgggcgttttgccgccaagtttcaatcccatcctggctttctccttggctccgctatcgggtctgaccctgatgtcttctggaccgtcataggggctcagctcgagggaagaaagaacacgtatgacgtggactacagtgcctttgactcttcacacggtactggctccttcgaggctctcatctctcactttttcaccgtggacaatggttttagccctgcgctgggaccgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctagcattgacctacaaggaatttgaatatgacatggttgatatcatcgcctacggtgacgaccttctggttggtacggattacgatctggacttcaatgaggtggcgcggcgcgctgccaaactggggtataagatgactcctgccaacaagggttctgtcttccctccgacttcctctctctccgatgctgtttttctaaaacgcaaattcgtccaaaacaatgacggcttatataaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 37 - RNA, 7312 nt, synthetic construct
  misc_feature 1..7312: note = SVV-003 (P30)
  source 1..7312: mol_type = other RNA
tttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaa

SEQ ID NO: 38 - RNA, 7352 nt, synthetic construct
  misc_feature 1..7352: note = SVV-004 (P70)
  source 1..7352: mol_type = other RNA
tttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaaaaaaaaaaaaaaaaaaaaaaaaaaaaaaaaaaaaaaaaa

SEQ ID NO: 39 - DNA, 7491 bp, synthetic construct
  misc_feature 1..7491: note = SVV-001 IVT template
  source 1..7491: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 40 - DNA, 7491 bp, synthetic construct
  misc_feature 1..7491: note = SVV-001-S177A
  source 1..7491: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 41 - DNA, 8259 bp, synthetic construct
  misc_feature 1..8259: note = SVV-001-GFP IVT template
  source 1..8259: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 42 - DNA, 8261 bp, synthetic construct
  misc_feature 1..8261: note = SVV-IRES1-GFP IVT template
  source 1..8261: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctcatgcccagtccttcctttccccttccggggggtaaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctatccactcactcacgcacaaaaagtgtgctgtaattacaagatttagccctcgcacgagatgtgcgataaccgcaagattgactcaagcgcggaaagcgctgtaaccacatgctgttagtcccttcatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 43 - DNA, 8261 bp, synthetic construct
  misc_feature 1..8261: note = SVV-IRES2-GFP IVT template
  source 1..8261: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 44 - DNA, 8261 bp, synthetic construct
  misc_feature 1..8261: note = SVV-IRES3-GFP IVT template
  source 1..8261: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagtggagcaacatccaacctgcttttgtggggaacggtgcggctccaattcctgcgtcgccaaaggtgttagcgcacccaaacggcgcgtctaccaatgctattggtgtggtctgcgagttctagcctactcgtttctcccctactcactcattcacgcacaaaaactgtgttgtaactacaaggtttagccctcgcacgggatgtgcgataaccgcaagatcgactcaagcgcggaaagcgctgtaaccacatgctgttagtccctttatggctgcgagatggctacccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 45 - DNA, 8259 bp, synthetic construct
  misc_feature 1..8259: note = SVV-P1-1-GFP IVT template
  source 1..8259: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcacttttctttcgatacagcctctggcacctttgaagacgtaaccggaacaaaagtcaagatcgttgaataccctagatcggtgaacaatggtgtttacgattcgtccactcatttagagatactgaacctacagggtgaaattgaaattttaaagtctttcaatgaataccaaattcgcgccgccaaacaacaacttggactggacatcgtatacgaactacagggtaatgttcagacaacctcaaagaatgattttgattcccgcggtaataatggtaacatgaccttcaattactacgcaaacacttaccagaattcagtagacttctcgacctcctcgtcggcgtcaggcgccggacccgggaactcccggggcggattagcgggtctcctcacaaatttcagtggaatcttgaaccctcttggctacctcaaagatcacaataccgaagaaatggaaaactctgctgatcgagtcataacgcaaacggcgggcaacactgccataaacacgcaatcatcactgggtgtgttgtgtgcctacgttgaagacccgaccaaatctgaccctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggctaccctacatagacatttcttaatgaagtgcgggtggcaggtgcaggtccaatgcaatttgacgcaattccaccaaggtgctcttcttgttgccatggtccccgaaaccacccttgatgtcaaacctgacggcaaggcaaagagcttacaggagctgaatgaagaacagtgggtggaaatgtctgacgattaccggaccgggaaaaacatgcctttccagtctcttggcacatactatcggccccctaactggacttggggccccaatttcatcaacccctatcaagtaacagttttcccacaccaaattctgaacgcgagaacctctacctcggtagacataagtgtcccatacatcggggagactcctacacaatcctcagagacacagaactcctggaccctcctcgttatggtgcttgtccccctggactacaaggagggagccacaactgacccagaaattacattttctgtaaggcctacaagtccttacttcaatgggctacgtaaccgtttcacgaccgggacggacgaggaacaggggcccattcccacagcacccagagaaaattcgcttatgtttctctcaaccatccctgacgacactgtccctgcttacgggaatgtgcgtacccctcccgtcaattacctccctggtgaaataaccgacctcttacaactggcccgtatacccactctcatggcgttcgggcgggtgtctgaacccgagcctgcctcagacacatatgtgccctacgttgccgttcctgcccagttcgacgacaagcctctcatctccttcccgatcaccctttcagatcctgtctaccagaacaccctggtaggcgccatcagttcgaatttcgccaactaccgggggtgtatccaaatcactctgacattttgtggacccatgatggcaagagggaaattcctactctcgtattctcctccaaatggagcacaaccacagaccctttctgaagctatgcagtgcacgtactctatttgggatataggcttgaactctagttggacctttgtcatcccctacatctcgcccagtgattaccgtgaaactcgggctattaccaactcagtttattctgctgatggttggtttagcctgcacaagctgactaaaattactctaccacctgactgcccacagagtccctgtattctctttttcgcctctgctggtgaggattacaccctccgtctccctgttgattgtaatccttcctacgtgttccactccaccgacaacgccgagactggggttattgaggcgggtaacactgacaccgacttctctggtgagctggcggctcctggctctaaccacactaatgtcaagttcctgtttgatcgatctcgattactgaatgtaattaaggtactggagaaggacgccgtcttcccccgtcctttccccacagcaacaggtgcacagcaggacgatggttacttttgccttctaacaccccgcccaacagtcgcttcccgacccgccactcgtttcggcctgtacgtcaatccgtctgacagtggcgttctcgctaacacttcactggatttcaatttttacagcttggcctgtttcacttactttagatcagaccttgaagtcacggtggtctcactggagccagatctggaattcgctgtagggtggttcccctctggcagtgagtaccaggcttccagctttgtctacgaccaactgcatgtaccctaccactttactgggcgcactccccgcgctttcaccagcaagggtggaaaggtatctttcgtgcttccttggaactctgtctcatccgtgcttcccgtgcgctgggggggcgcttccaagctttcttccgccacgcggggtctaccggctcatgctgactgggggaccatttacgccttcatcccccgtcctaatgagaagaaaagcaccgctgcaaagcacgtggcggtgtacgttcggtacaagaacgcgcgtgcctggtgccccagcatgctccccttccgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 46 - DNA, 8259 bp, synthetic construct
  misc_feature 1..8259: note = SVV-P1-2-GFP IVT template
  source 1..8259: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagacgtaaccggaacaaaagtcaagatcgttgaataccccagatcggtgaacaatggtgtttacgattcgtccactcatttagagatactgaacctacagggtgaaattgaaattttaaggtctttcaatgaataccaaattcgcgccgccaaacaacaacttggactagacattgtatacgaactacagggtaacgttcagacaacctcaaagaatgattttgattcccgcggcaataacggtaacatgaccttcaattactacgcaaacacttatcagaactcagtagacttctcgacctcctcgtcggcgtcaggcgccggacccgggaactcccggggcggattagcgggtctcctcacaaatttcagtggaatcttgaaccctcttggctaccttaaagatcacaataccgaagaaatggaaaactctgctgatcgagtcataacgcaaacggcgggcaacactgccataaacacgcaatcatcactgggtgtgttgtgtgcctatgttgaagacccgaccaaatctgatcctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggctaccctacatagacatttcttaatgaagtgcgggtggcaggtgcaggttcaatgcaatttgacacaattccaccaaggtgctcttcttgtcgccatggtccccgaagctacccttgatgtcaagcccgacggcaaggcaaagagcctgcaggagctgaatgaagagcagtgggtagaaatgtccgacgattaccggaccgggaaaaacatgccttttcagtctcttggcacatactatcggccccctaactggacttggggccctaatttcatcaacccctatcaagtaacagttttcccacaccaaattctgaacgcgagaacctctacctcggtagacataagtgtcccatacatcggggagactcctacacaatcctcagagacacagaactcctggaccctcctcgttatggtgcttgtccccctggactacaaggagggagccacaactgacccagaaattacattttctgtaaggcctacaagtccttacttcaatgggcttcgtaaccgttacacgaccgggacggacgaggaacaggggcccattcccacagcacccagagaaaattcgcttatgtttctctcaaccatccctgacgacactgtccctgcttacgggaatgtgcgtacccctcccgtcaactacctccctggtgaaataactgacctcttacaactggcccgtatacccactctcatggcgtttgggcgggtttctgaacccgaacctgcctcagacgcatatgtgccctacgttgccgttcctgcccagtttgatgacaagcctctcatctccttcccgatcaccctttcagatcctgtctaccagaacaccctggtaggcgccatcagttcgaatttcgccaactaccgggggtgtatccaaatcactctgacattttgtggacccatgatggcaagagggaaattcctactctcgtattctcccccaaatggaacacaaccacagaccctttctgaagctatgcagtgcacgtactccatttgggatataggcttgaactctagttggacctttgtcatcccctacatctcgcccagtgactaccgtgaaactcgggccatcaccaactcggtttattctgctgacggctggtttagcctacacaagctgaccaaaattactctaccacctgactgcccacagagcccctgtattctctttttcgcctctgctggtgaggattacaccctccgtctccccgttgattgcaatccttcctacgtgttccactccaccgataacgccgagactggagttattgaggcgggtaatactgacaccgatttctctggtgaactggcggctcctggctctaaccatactaatgtcaagttcctgtttgatcgatctcgattactgaatgtaattaaggtactggagaaggacgccgtcttcccccgtcctttccccacagcaacaggtgcacagcaggacgatggttacttttgtcttctaacaccccgcccaacagtcgcctcccgacccgccactcgtttcggcctgtatgtcaatccgtctgacagtggcgttctcgccaacacttcactggatttcaatttttacagcttggcctgcttcacttactttagatcagaccttgaagtcacggtggtctcactggagccagatctggaattcgctgtagggtggttcccctctggcagtgagtaccaggcttccagctttgtctacgaccaactgcatgtaccctaccactttactgggcgcactccccgcgctttcaccagcaagggtgggaaggtatctttcgtactcccttggaactctgtctcatccgtgcttcccgtgcgctgggggggtgcttccaagctttcttctgccacgcggggtttgccggctcatgctgactgggggactatttacgcctttatcccccgtcctaatgagaagaaaggcaccgctgtaaagcacgtggccgtgtacgttcggtacaagaacgcgcgtgcctggtgccccagcatgctccccttccgcagttacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 47 - DNA, 8259 bp, synthetic construct
  misc_feature 1..8259: note = SVV-P1-3-GFP IVT template
  source 1..8259: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaaaacgtaaccggaacaaaagtcaagatcgttgaataccccagatcggtgaacaatggtgtttacgattcgtccactcatttagagatactgaacctacagggtgaaattgaaattttaaagtctttcaacgaataccaaattcgcgccgccaaacaacaacttggactggacatcgtatacgaactacagggtaatgttcagacaacctcaaagaatgattttgattcccgcggcaataatggtaacatgaccttcaattactacgcaaacacttaccagaattcagtagacttctcgacctcctcgtcggcgtcaggcgccggacccgggaactcccggggcggattagcgggtctcctcacaaatttcagtggaatcttgaaccctcttggctacctcaaagatcacaataccgaagaaatggaaaactctgctgatcgagtcataacgcaaacggcgggcaacactgccataaacacgcaatcatcactgggtgtgttgtgtgcctacgttgaagacccgaccaaatctgaccctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggccaccctacatagacatttcttaatgaagtgcgggtggcaggtgcaggttcaatgcaatttgacgcaattccaccaaggtgctcttcttgttgccatggtccccgaaaccacccttgatgtcaaacctgacggcaaggcaaagagcttacaggagctgaatgaagagcagtgggtggaaatgtctgacgactaccggaccgggaaaaacatgccttttcagtctcttggtacatactatcggccccctaactggacttggggccccaatttcatcaacccctatcaagtaacagttttcccacaccaaattctgaacgcgagaacctctacctcggtagacataagtgtcccgtacatcggggagactcctacacaatcctcagagacacagaactcctggaccctccttgttatggtgcttgtccccctggactacaaggagggagccacaactgacccagaaattacattttctgtaaggcctacaagtccctacttcaatgggcttcgtaaccgtttcacgaccgggacggacgaggaacaggggcccattcccacagcacccagagaaaattcgcttatgtttctctcaaccatccctgacgacactgtccctgcttacgggaatgtgcgcacccctcccgtcaattacctccccggtgaaataaccgacctcttacaactggcccgtatacccactctcatggcgtttgggcgggcgtctgaacccgagcctgcctcagacgcatatgtgccctacgttgccgttcctgcccagttcgacgacaagcctctcatctccttcccgatcaccctttcagaccctgtctaccagaacaccctggtaggcgccatcagttcgaacttcgccaactaccgggggtgtatccaaattactctgacattttgtggacccatgatggcaagagggaaattcctgctctcgtattctcccccaaatggagcacaaccacagaccctttctgaagctatgcagtgcacatactctatttgggatataggcttgaactccagttggacctttgtcatcccctacatctcgcccagtgattaccgtgaaactcgggctattaccaactcagtttattctgctgatggttggtttagcttgcacaagctgaccaaaattactctaccacctgactgcccacagagtccctgtattctctttttcgcctctgctggtgaggattacaccctccgtctccctgttgattgtaatccttcctacgtgttccactccaccgacaacgccgagactggggttattgaggcaggtaacactgacaccgatttctctggtgaactggcggctcctggctctaaccatactaatgtcaaattcctgtttgaccgatctcgactactgaatgtaattaaggtactggagaaggacgccgtcttcccccgtcctttccccacagcaacaggtgcacagcaggacgatggttacttttgtcttctaacaccccgcccaacagtcgcttcccgacccgccactcgtttcggcctgtacgtcaacccgtctgacagtggcgttctcgctaacacttcactggatttcaatttttacagtttggcctgtttcacttactttagatcagaccttgaagtcacggtggtctcactggagccagatttggaattcgccgtggggtggttcccctctggcagtgagtaccaggcttctagctttgtctacgaccaactgcatgtaccctaccactttactgggcgcactccccgcgctttcaccagcaagggtggaaaggtatctttcgtgctcccttggaactctgtctcttccgtgcttcccgtgcgctgggggggtgcctccaagctttcttctgccacgcggggtctgccggctcatgctgactgggggaccatttacgcctttatcccccgtcctaacgagaagaaaagcaccgctgtaaagcacgtggcggtgtacgttcggtacaagaacgcgcgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 48 - DNA, 8259 bp, synthetic construct
  misc_feature 1..8259: note = SVV-P3-1-GFP IVT template
  source 1..8259: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaacgacgacacccccgtcgacgaggcgttgggtagagttctcacccccgctgcggtcgacgaggcgcttgtcgacctcgctccagatgccgacccggttggccgtttggctattctcgccaagctaggtcttgccctagctgcggtcacccctggtttgataatcttggcagtgggactctacaggtacttctctggctctgatacagaccaagaagaaacagaaggtgaggagcctgctaaagcgcctaggagcgagaatgcttatgacggcccgaagaaaaactctaagccccctggagcgctctcccttatggaaatgcaacagcccaacgtggacatgggctttgaggctgcggtcgctaagaaagtggtcgtccccattaccttcatggttcccaacagaccttctggacttacacagtccgctcttcttgtggccggccggaccttcctaattaatgagcatacatggtccaacccctcctggaccagcttcacaatccgtggtgaggtgcacacccgtgatgagcctttccaaacggttcattttactcaccatggtgttcccacagatctggtgatggtacgtctcggaccgggcaactccttccctaacaatctagacaagtttggacttgatcagatgccggcacgtaactcccgtgtggttggcgtttcggctagttacggcaacttcttcttctctgggaacttcctcgggtttgttgactccatcacctctgaccaaggaacttatgcgagactttttaggtacagggtgacgacctacaaggggtggtgcggttcggccctggtttgtgaggccggtggtgtccggcgcatcattggcctgcattctgctggtgccgctggtatcggcgccgggacttacatctcaaaattaggactgatcaaagccctcaaacacctcggtgaacctctggctacaatgcaaggactgatgactgagctagagcctggagtcaccgtgcatgtaccccggaaatctaaattgagaaagacgaccgcacacgcggtgtacaaaccggagtttgaacctgctgtgttgtcaaaatttgatcccagactgaacaaggatgttgacctagatgaggtaatttggtctaaacacactgccaacgtcccttatcaacctcctttgttctacacatacatgtcagagtacgctcatcgggttttctcctttttgggaaaagacaatgacattctgaccgtcaaagaagcaatcttgggaatccctggactagaccctatggatccccacacagctccgggtctgccctacgccattagcggccttcgacgtactgatctcgtcgattttgcgaacggcacggtggacccgacactggccgtacaaatccaaaaattcttagacggtgactactctgatcatgtcttccaaacttttctgaaagatgaaatcagaccctcagagaaggtccgagcgggaaaaacccgcattgtcgatgtgccctccctggcgcactgcattgtgggcagaatgttgcttgggcgctttgccgccaagtttcaatcccatcctggctttctccttggctctgctatcgggtctgaccctgatgtcttctggaccgtcataggggctcagctcgagggaagaaagaacacgtatgacgtggactacagtgcctttgactcttcacacggcactggctccttcgaggctctcatctctcactttttcaccgtggacaatggttttagccctgcgctgggatcgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctggcattgacctacaaggaatttgaatatgacatggttgatatcatcgcctacggtgacgaccttctggttggtacggattacgatctggacttcaatgaggtggcgcggcgcgctgccaaactggggtataagatgactcctgccaacaaaggttctgtcttccctccgacttcctctctctccgacgctgtttttctaaaacgcaaattcgtccaaaacaatgacggcttatacaaaccagttatggatttaaagaatttggaagccatgctctcctacttcaaaccaggaacactactcgagaagctgcaatctgtttctatgttggctcaacattctggaaaagaagaatatgatagattgatgcacccctttgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 49 - DNA, 8259 bp, synthetic construct
  misc_feature 1..8259: note = SVV-P3-2-GFP IVT template
  source 1..8259: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgagtagagttctcacccccgctgcggtcgacgaggcgcttgtcgacctcgctccagaggccgacccggttggccgcttggctattcttgccaagctaggtcttgccctggctgcggtcacccctggtttgataatcctggcagtgggactctacaggtacttctctggctctgatacagaccaagaagaaacagaaagtgaggaacctgttaaagcgcccaggagcgagaatgcttatgacggcccaaagaaaaactctaagccccctggggcgctctctcttatggaaatgcagcagcccaacgtggacatgggctttgaggctgcggtcgctaagaaagtggtcgtccccattaccttcatggttcccaacagaccttctggacttacacagtccgctcttcttgtggccggccggactttcctaattaatgagcatacatggtccaacccctcctggaccagcttcacaatccgtggtgaggtgcacactcgtgatgagcctttccaaacggttcattttactcaccatggtgttcccacagatctgatgatggtgcgtctcgggccgggcaactctttccctaacaatctagacaagtttggacttgaccagatgccggcacgtaactcccgtgtggttggcgtttcggctagttacggtaacttcttcttctctgggaatttcctcgggtttgttgactccatcacctctgaccaaggaacttatgcgagactttttaggtacagggtgacgacttacaagggatggtgtggttcggccctggtctgtgaggccggtggtgtccggcgcatcattggcttgcactctgctggcgccgctggtatcggcgccgggacttacatctcaaaattgggactgatcaaagcccttaaacacctcggtgaacctctggctacaatgcaaggactgatgactgagctagagcctggagtcaccgtacatgtaccccggaaatctaaattgagaaagacgaccgcacacgcggtgtacaaaccggagtttgaacctgctgtgttgtcaaaatttgatcccagactgaacaaggatgttgacttagatgaggtgatttggtctaaacacactgccaacgtcccttatcaacctcctttgttctacacatatatgtcagagtacgctcaccgggttttctcctttttgggaaaagacaatgacattctgaccgtcaaagaagcaatcttgggcatccctggactagaccctatggatccccacacagctccgggtctgccctacgccattagcggccttcgacgtactgatctcgtcgattttgcgaacggcacggtagacgcggcactggccatgcaaattcagaaattcttagatggtgactactctgatcatgtcttccaaacttttctgaaagatgaaatcagaccctcagagaaggtccgagcgggaaaaacccgcattgtcgatgtgccttccctggcgcactgcattgttggcagaatgctgcttgggcgttttgccgccaagtttcaatcccatcctggctttctccttggctctgctatcgggtctgaccctgatgtcttctggaccgtcataggggctcagctcgagggaagaaagaacacgtatgacgtggactacagtgcctttgactcttcacacggcactggctccttcgaggctctcatctctcactttttcaccgttgacaatggttttagccctgcgctgggaccgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctagcattgacttacaaggaatttgagtatgacatggttgatatcatcgcctacggtgacgaccttctggttggtacggattatgatctggacttcaatgaggtggcgcggcgcgctgccaaactggggtataagatgactcctgccaacaagggttctgtcttccctccgacttcctctctctccgatgctgtttttctaaaacgcaaattcgtccaaaacaatgacggcttatataaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 50 - DNA, 8259 bp, synthetic construct
  misc_feature 1..8259: note = SVV-P3-3-GFP IVT template
  source 1..8259: mol_type = other DNA
taatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccctaacgagaacgacgacacccccgtcgacgaggcgttgggtagagttctcacccccgctgcggtcgacgaggcgcttgtcgacctcgctccagatgccgacccggttggccgcttggctattctcgccaagctaggtcttgccctagctgcggtcacccctggtttgataatcttggcagtgggactctacaagtacttctctggctctgatacagaccaagaagaaacagaaagtgaggagcctgttaaagcgcctaggagcgagaatgcttatgacggcccgaagaaaaactctaagccccctggagcgctctctcttatggaaatgcaacagcccaacgtggacatgggctttgaggctgcggtcgctaagaaagtggtcgtccccattaccttcatggttcccaacagaccttctggacttacacagtccgctcttcttgtgaccggccggaccttcctaatcaatgagcatacatggtccaacccctcctggaccagcttcacaatccgtggtgaggtgcacactcgtgatgagcctttccaaacggttcattttactcaccatggtcttcccacagatctgatgatggtacgtctcggaccgggcaactccttccctaacaatctagacaagtttggacttgaccagatgccggcacgtaactcccgtgtggtcggcgtttcggctagttacggtaacttcttcttctctgggaacttcctcgggtttgttgactccatcacctctgaccaaggaacctatgcgagacttttcaggtacagggtgacgacttacaagggatggtgcggttcggccctggtctgcgaggccggtggtgtccgacgcatcattggcctgcactctgctggtgccgctggtatcggtgccgggacttacatctcaaaattaggactgatcaaagcccttaaacacctcggtgagcctctggctacaatgcaaggactgatgactgagctagagcctggagtcaccgtacatgtaccccgaaaatctaaattgagaaagacgaccgcacacgcggtgtacaaaccggagtttgaacctgctgtgttgtcaaaatttgatcccagactgaacaaggatgttgacctagatgaggtaatttggtccaaacacactgccaacgtcccttatcaacctcctttgttctacacatacatgtcagagtacgctcatcgggttttctcctttttgggaaaagacaatgacattctgaccgtcaaagaagcaatcttgggcatccctggactagaccctatggatccccacacagctccgggtctgccctacgccattaacggccttcgacgtactgatctcgtcgattttgcgaacggcacggtagacccggcactggccatgcaaatccaaaaattcttagacggtgactactctgatcatgtcttccaaacttttctgaaagatgaaatcagaccctcagagaaggtccgggcgggaaaaacccgcattgtcgatgtgccctccctggcgcactgcattgtgggcagaatgctgcttgggcgttttgccgccaagtttcaatcccatcctggctttctccttggctccgctatcgggtctgaccctgatgtcttctggaccgtcataggggctcagctcgagggaagaaagaacacgtatgacgtggactacagtgcctttgactcttcacacggtactggctccttcgaggctctcatctctcactttttcaccgtggacaatggttttagccctgcgctgggaccgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctagcattgacctacaaggaatttgaatatgacatggttgatatcatcgcctacggtgacgaccttctggttggtacggattacgatctggacttcaatgaggtggcgcggcgcgctgccaaactggggtataagatgactcctgccaacaagggttctgtcttccctccgacttcctctctctccgatgctgtttttctaaaacgcaaattcgtccaaaacaatgacggcttatataaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgc

SEQ ID NO: 51 - DNA, 7447 bp, synthetic construct
  misc_feature 1..7447: note = SVV-003 (P30) IVT template
  source 1..7447: mol_type = other DNA
taatacgactcactatagggcgtcatagtaggaagtattgggttgggaagaacttatcgtggactcgtctgagcgagtataaacagtcaattaagctcagagcgttcaccggggaaattcggtgaggtttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaagaagagc

SEQ ID NO: 52 - DNA, 7487 bp, synthetic construct
  misc_feature 1..7487: note = SVV-004 (P70) IVT template
  source 1..7487: mol_type = other DNA
taatacgactcactatagggcgtcatagtaggaagtattgggttgggaagaacttatcgtggactcgtctgagcgagtataaacagtcaattaagctcagagcgttcaccggggaaattcggtgaggtttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaaaaaaaaaaaaaaaaaaaaaaaaaaaaaaaaaaaaaaaaaagaagagc

SEQ ID NO: 53 - DNA, 43 bp, synthetic construct
  misc_feature 1..43: note = Leader sequence SVV-L0
  source 1..43: mol_type = other DNA
gggcgtcatagtaggaagtattgggttgggaagaacttatcgt

SEQ ID NO: 54 - DNA, 54 bp, synthetic construct
  misc_feature 1..54: note = Leader sequence SVV-L1
  source 1..54: mol_type = other DNA
gggaagtagggggttgagcacattttaatacagcaaggtaaagataagaacatt

SEQ ID NO: 55 - DNA, 54 bp, synthetic construct
  misc_feature 1..54: note = Leader sequence SVV-L2
  source 1..54: mol_type = other DNA
gggatataaacgaatagcactttcaagacactattttaaactataaaggtaact

SEQ ID NO: 56 - DNA, 54 bp, synthetic construct
  misc_feature 1..54: note = Leader sequence SVV-L3
  source 1..54: mol_type = other DNA
gggatttaagcaaccatctcttttcctttcactcaccatacagcaatcaattct

SEQ ID NO: 57 - DNA, 54 bp, synthetic construct
  misc_feature 1..54: note = Leader sequence SVV-L4
  source 1..54: mol_type = other DNA
gggctcgataccgttctattcttaaatcctgacaaaatcactcattcattactt

SEQ ID NO: 58 - DNA, 54 bp, synthetic construct
  misc_feature 1..54: note = Leader sequence SVV-L5
  source 1..54: mol_type = other DNA
gggacgcgtcgaaacgttactttaaatggcagctaagaacttaaactacaacat

SEQ ID NO: 59 - DNA, 54 bp, synthetic construct
  misc_feature 1..54: note = Leader sequence SVV-L6
  source 1..54: mol_type = other DNA
gggactaattgtgaataaaagactaacgccctaggagagacgcagatcattaat

SEQ ID NO: 60 - DNA, 54 bp, synthetic construct
  misc_feature 1..54: note = Leader sequence SVV-L7
  source 1..54: mol_type = other DNA
gggagacatgaaatacagcgagatggatcgactaccaatgaattacttacgaat

SEQ ID NO: 61 - DNA, 54 bp, synthetic construct
  misc_feature 1..54: note = Leader sequence SVV-L8
  source 1..54: mol_type = other DNA
gggacgcactagtcgtagaacttcacacgcttcactattaaactttacttctct

SEQ ID NO: 62 - DNA, 54 bp, synthetic construct
  misc_feature 1..54: note = Leader sequence SVV-L9
  source 1..54: mol_type = other DNA
gggttgctaaacgtactttcagaacctgcataaacacacgaaagattgattaat

SEQ ID NO: 63 - DNA, 54 bp, synthetic construct
  misc_feature 1..54: note = Leader sequence SVV-L10
  source 1..54: mol_type = other DNA
gggaaagcaaccttcctgaaagataagagcgtacatggatacaacaaggggact

SEQ ID NO: 64 - DNA, 67 bp, synthetic construct
  misc_feature 1..67: note = Pistol Polymyxa ribozyme 1
  source 1..67: mol_type = other DNA
ggactcgtctgagcgagtataaacagtcaattaagctcagagcgttcaccggggaaattcggtgagg

SEQ ID NO: 65 - DNA, 67 bp, synthetic construct
  misc_feature 1..67: note = Pistol Polymyxa ribozyme 2
  source 1..67: mol_type = other DNA
ggactcgtctgagcgagtataaacagttaattaagctcagagcgttcaccggggaaattcggtgagg

SEQ ID NO: 66 - DNA, 42 bp, synthetic construct
  misc_feature 1..42: note = Hammerhead ribozyme
  source 1..42: mol_type = other DNA
tcaaactgatgagtccgtgaggacgaaacgagtaagctcgtc

SEQ ID NO: 67 - DNA, 47 bp, synthetic construct
  misc_feature 1..47: note = ribozyme with a stabilized stem I (STBL)
  source 1..47: mol_type = other DNA
ccatttcaaactgatgagtccgtgaggacgaaacgagtaagctcgtc

SEQ ID NO: 68 - RNA, 7282 nt, synthetic construct
  misc_feature 1..7282: note = SVV-IRES-2-S177A
  source 1..7282: mol_type = other RNA
tttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 69 - RNA, 7270 nt, Senecavirus Seneca Valley virus A
  source 1..7270: mol_type = unassigned RNA
ctgggccctcatgcccagtccttcctttccccttccggggggtaaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctatccactcactcacgcacaaaaagtgtgctgtaattacaagatttagccctcgcacgagatgtgcgataaccgcaagattgactcaagcgcggaaagcgctgtaaccacatgctgttagtcccttcatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcacttttctttcgatacagcctctggcacctttgaagacgtaaccggaacaaaagtcaagatcgttgaataccctagatcggtgaacaatggtgtttacgattcgtccactcatttagagatactgaacctacagggtgaaattgaaattttaaagtctttcaatgaataccaaattcgcgccgccaaacaacaacttggactggacatcgtatacgaactacagggtaatgttcagacaacctcaaagaatgattttgattcccgcggtaataatggtaacatgaccttcaattactacgcaaacacttaccagaattcagtagacttctcgacctcctcgtcggcgtcaggcgccggacccgggaactcccggggcggattagcgggtctcctcacaaatttcagtggaatcttgaaccctcttggctacctcaaagatcacaataccgaagaaatggaaaactctgctgatcgagtcataacgcaaacggcgggcaacactgccataaacacgcaatcatcactgggtgtgttgtgtgcctacgttgaagacccgaccaaatctgaccctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggctaccctacatagacatttcttaatgaagtgcgggtggcaggtgcaggtccaatgcaatttgacgcaattccaccaaggtgctcttcttgttgccatggtccccgaaaccacccttgatgtcaaacctgacggcaaggcaaagagcttacaggagctgaatgaagaacagtgggtggaaatgtctgacgattaccggaccgggaaaaacatgcctttccagtctcttggcacatactatcggccccctaactggacttggggccccaatttcatcaacccctatcaagtaacagttttcccacaccaaattctgaacgcgagaacctctacctcggtagacataagtgtcccatacatcggggagactcctacacaatcctcagagacacagaactcctggaccctcctcgttatggtgcttgtccccctggactacaaggagggagccacaactgacccagaaattacattttctgtaaggcctacaagtccttacttcaatgggctacgtaaccgtttcacgaccgggacggacgaggaacaggggcccattcccacagcacccagagaaaattcgcttatgtttctctcaaccatccctgacgacactgtccctgcttacgggaatgtgcgtacccctcccgtcaattacctccctggtgaaataaccgacctcttacaactggcccgtatacccactctcatggcgttcgggcgggtgtctgaacccgagcctgcctcagacacatatgtgccctacgttgccgttcctgcccagttcgacgacaagcctctcatctccttcccgatcaccctttcagatcctgtctaccagaacaccctggtaggcgccatcagttcgaatttcgccaactaccgggggtgtatccaaatcactctgacattttgtggacccatgatggcaagagggaaattcctactctcgtattctcctccaaatggagcacaaccacagaccctttctgaagctatgcagtgcacgtactctatttgggatataggcttgaactctagttggacctttgtcatcccctacatctcgcccagtgattaccgtgaaactcgggctattaccaactcagtttattctgctgatggttggtttagcctgcacaagctgactaaaattactctaccacctgactgcccacagagtccctgtattctctttttcgcctctgctggtgaggattacaccctccgtctccctgttgattgtaatccttcctacgtgttccactccaccgacaacgccgagactggggttattgaggcgggtaacactgacaccgacttctctggtgagctggcggctcctggctctaaccacactaatgtcaagttcctgtttgatcgatctcgattactgaatgtaattaaggtactggagaaggacgccgtcttcccccgtcctttccccacagcaacaggtgcacagcaggacgatggttacttttgccttctaacaccccgcccaacagtcgcttcccgacccgccactcgtttcggcctgtacgtcaatccgtctgacagtggcgttctcgctaacacttcactggatttcaatttttacagcttggcctgtttcacttactttagatcagaccttgaagtcacggtggtctcactggagccagatctggaattcgctgtagggtggttcccctctggcagtgagtaccaggcttccagctttgtctacgaccaactgcatgtaccctaccactttactgggcgcactccccgcgctttcaccagcaagggtggaaaggtatctttcgtgcttccttggaactctgtctcatccgtgcttcccgtgcgctgggggggcgcttccaagctttcttccgccacgcggggtctaccggctcatgctgactgggggaccatttacgccttcatcccccgtcctaatgagaagaaaagcaccgctgcaaagcacgtggcggtgtacgttcggtacaagaacgcgcgtgcctggtgccccagcatgctccccttccgcagctacaagcagaagatgctgatgcaatcaggcgacgtcgagaccaaccctggccctgcttctgacaacccaatcttggagtttcttgaagctgaaaacgatctagtcactctggcctctctctggaagatggtacactctgttcaacagacctggagaaagtatgtgaagaatgacaatttttggcccaatttgctcagtgagctagtgggggaaggctccatcgccttggccgccacgctatctaaccaagcttcagtaaaagctctcttaggcctgcattttctctcccgagggctcaattatacagatttttactctttactgatagagaaatgctctagtttctttactgtagaaccgcctcctccaccagctgaaaatctgatgaccaagccctccgtgaggtcgaaattccgaaagctgtttaagatgcaaggacccatggacacagtcaaagactggaaccaaatagccgccggcctgaagaatttccaatttgttcgtgacctggtcaaagaggtggtcgattggctccaggcctggattaataaagagaaagccagccctgtcctccagtaccagctggagatgaagaaactcggacccgtggctttggctcatgatgccttcatggccggttccgggccccctcttagtgacgaccagattgaatatctccagaacctcaaatctcttgccctaacactggggaagactaatttggcccaaagtctcaccactatgatcaatgccaagcaaagctccgctcaacgagtcgaacccgttgtggtggtccttagaggcaagccgggatgtggcaagagcttggcctccacgttgattgcccaggctgtgtccaagcgtctctacggctcacaaagtgtgtattctcttcctccggatccagacttcttcgacggatacaaaggacagtttgtaaccttgatggacgatctgggacaaaacccggatgggcaagatttctccaccttttgtcagatggtgtcgaccgcccaatttcttcccaacatggcggaccttacagagaaggggcgtcccttcacctccaatcttatcattgcaactacaaacctccctcacttcagccctgtcaccattgctgatccttctgcagtctctcgtcgtatcaactacgacctgactctagaagtatctgaggcctataagaagcacacacggctgaattttgacctggctttcagacgcacagacgccccccccatttatccttttgctgcccatgtgcccttcgtggacgtggctgtgcgcttcaaaaatggtcatcagagcttcaatctcctagagttggtcgactctatttgtgcagacattcgagccaagcaacaaggtgcccgaaatatgcagactctggttctacagagccccaacgagaacgacgacacccccgtcgacgaggcgttgggtagagttctcacccccgctgcggtcgacgaggcgcttgtcgacctcgctccagatgccgacccggttggccgtttggctattctcgccaagctaggtcttgccctagctgcggtcacccctggtttgataatcttggcagtgggactctacaggtacttctctggctctgatacagaccaagaagaaacagaaggtgaggagcctgctaaagcgcctaggagcgagaatgcttatgacggcccgaagaaaaactctaagccccctggagcgctctcccttatggaaatgcaacagcccaacgtggacatgggctttgaggctgcggtcgctaagaaagtggtcgtccccattaccttcatggttcccaacagaccttctggacttacacagtccgctcttcttgtggccggccggaccttcctaattaatgagcatacatggtccaacccctcctggaccagcttcacaatccgtggtgaggtgcacacccgtgatgagcctttccaaacggttcattttactcaccatggtgttcccacagatctggtgatggtacgtctcggaccgggcaactccttccctaacaatctagacaagtttggacttgatcagatgccggcacgtaactcccgtgtggttggcgtttcggctagttacggcaacttcttcttctctgggaacttcctcgggtttgttgactccatcacctctgaccaaggaacttatgcgagactttttaggtacagggtgacgacctacaaggggtggtgcggttcggccctggtttgtgaggccggtggtgtccggcgcatcattggcctgcattctgctggtgccgctggtatcggcgccgggacttacatctcaaaattaggactgatcaaagccctcaaacacctcggtgaacctctggctacaatgcaaggactgatgactgagctagagcctggagtcaccgtgcatgtaccccggaaatctaaattgagaaagacgaccgcacacgcggtgtacaaaccggagtttgaacctgctgtgttgtcaaaatttgatcccagactgaacaaggatgttgacctagatgaggtaatttggtctaaacacactgccaacgtcccttatcaacctcctttgttctacacatacatgtcagagtacgctcatcgggttttctcctttttgggaaaagacaatgacattctgaccgtcaaagaagcaatcttgggaatccctggactagaccctatggatccccacacagctccgggtctgccctacgccattagcggccttcgacgtactgatctcgtcgattttgcgaacggcacggtggacccgacactggccgtacaaatccaaaaattcttagacggtgactactctgatcatgtcttccaaacttttctgaaagatgaaatcagaccctcagagaaggtccgagcgggaaaaacccgcattgtcgatgtgccctccctggcgcactgcattgtgggcagaatgttgcttgggcgctttgccgccaagtttcaatcccatcctggctttctccttggctctgctatcgggtctgaccctgatgtcttctggaccgtcataggggctcagctcgagggaagaaagaacacgtatgacgtggactacagtgcctttgactcttcacacggcactggctccttcgaggctctcatctctcactttttcaccgtggacaatggttttagccctgcgctgggatcgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctggcattgacctacaaggaatttgaatatgacatggttgatatcatcgcctacggtgacgaccttctggttggtacggattacgatctggacttcaatgaggtggcgcggcgcgctgccaaactggggtataagatgactcctgccaacaaaggttctgtcttccctccgacttcctctctctccgacgctgtttttctaaaacgcaaattcgtccaaaacaatgacggcttatacaaaccagttatggatttaaagaatttggaagccatgctctcctacttcaaaccaggaacactactcgagaagctgcaatctgtttctatgttggctcaacattctggaaaagaagaatatgatagattgatgcacccctttgctgactacggtgccgtaccgagtcacgagtacctgcaggcaagatggagggccttgttcgactgacctagatagcccaacgcgctccggtgctgccggcgattctgggagaactcagtcggaacagaaaagggaa

SEQ ID NO: 70 - RNA, 7212 nt, Senecavirus Seneca Valley virus A
  source 1..7212: mol_type = unassigned RNA
aaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagacgtaaccggaacaaaagtcaagatcgttgaataccccagatcggtgaacaatggtgtttacgattcgtccactcatttagagatactgaacctacagggtgaaattgaaattttaaggtctttcaatgaataccaaattcgcgccgccaaacaacaacttggactagacattgtatacgaactacagggtaacgttcagacaacctcaaagaatgattttgattcccgcggcaataacggtaacatgaccttcaattactacgcaaacacttatcagaactcagtagacttctcgacctcctcgtcggcgtcaggcgccggacccgggaactcccggggcggattagcgggtctcctcacaaatttcagtggaatcttgaaccctcttggctaccttaaagatcacaataccgaagaaatggaaaactctgctgatcgagtcataacgcaaacggcgggcaacactgccataaacacgcaatcatcactgggtgtgttgtgtgcctatgttgaagacccgaccaaatctgatcctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggctaccctacatagacatttcttaatgaagtgcgggtggcaggtgcaggttcaatgcaatttgacacaattccaccaaggtgctcttcttgtcgccatggtccccgaagctacccttgatgtcaagcccgacggcaaggcaaagagcctgcaggagctgaatgaagagcagtgggtagaaatgtccgacgattaccggaccgggaaaaacatgccttttcagtctcttggcacatactatcggccccctaactggacttggggccctaatttcatcaacccctatcaagtaacagttttcccacaccaaattctgaacgcgagaacctctacctcggtagacataagtgtcccatacatcggggagactcctacacaatcctcagagacacagaactcctggaccctcctcgttatggtgcttgtccccctggactacaaggagggagccacaactgacccagaaattacattttctgtaaggcctacaagtccttacttcaatgggcttcgtaaccgttacacgaccgggacggacgaggaacaggggcccattcccacagcacccagagaaaattcgcttatgtttctctcaaccatccctgacgacactgtccctgcttacgggaatgtgcgtacccctcccgtcaactacctccctggtgaaataactgacctcttacaactggcccgtatacccactctcatggcgtttgggcgggtttctgaacccgaacctgcctcagacgcatatgtgccctacgttgccgttcctgcccagtttgatgacaagcctctcatctccttcccgatcaccctttcagatcctgtctaccagaacaccctggtaggcgccatcagttcgaatttcgccaactaccgggggtgtatccaaatcactctgacattttgtggacccatgatggcaagagggaaattcctactctcgtattctcccccaaatggaacacaaccacagaccctttctgaagctatgcagtgcacgtactccatttgggatataggcttgaactctagttggacctttgtcatcccctacatctcgcccagtgactaccgtgaaactcgggccatcaccaactcggtttattctgctgacggctggtttagcctacacaagctgaccaaaattactctaccacctgactgcccacagagcccctgtattctctttttcgcctctgctggtgaggattacaccctccgtctccccgttgattgcaatccttcctacgtgttccactccaccgataacgccgagactggagttattgaggcgggtaatactgacaccgatttctctggtgaactggcggctcctggctctaaccatactaatgtcaagttcctgtttgatcgatctcgattactgaatgtaattaaggtactggagaaggacgccgtcttcccccgtcctttccccacagcaacaggtgcacagcaggacgatggttacttttgtcttctaacaccccgcccaacagtcgcctcccgacccgccactcgtttcggcctgtatgtcaatccgtctgacagtggcgttctcgccaacacttcactggatttcaatttttacagcttggcctgcttcacttactttagatcagaccttgaagtcacggtggtctcactggagccagatctggaattcgctgtagggtggttcccctctggcagtgagtaccaggcttccagctttgtctacgaccaactgcatgtaccctaccactttactgggcgcactccccgcgctttcaccagcaagggtgggaaggtatctttcgtactcccttggaactctgtctcatccgtgcttcccgtgcgctgggggggtgcttccaagctttcttctgccacgcggggtttgccggctcatgctgactgggggactatttacgcctttatcccccgtcctaatgagaagaaaggcaccgctgtaaagcacgtggccgtgtacgttcggtacaagaacgcgcgtgcctggtgccccagcatgctccccttccgcagttacaagcagaagatgctgatgcaatcaggcgacgtcgagaccaaccctggccctgcttctgacaacccaatcttggagtttcttgaagcagaaaacgatctagtcactctggcctctctctggaagatggtacactctgttcaacagacctggagaaagtatgtgaagaacgacaatttttggcccaatttactcagtgagctagtgggggaaggctccatcgccttggccgccacgctatctaatcaagcttcagtaaaagctctcttgggcctgcattttctctctcgcgggctcaattacacagacttttactctttactgatagagaaatgctctagtttctttactgtagaaccgcctcctccaccagctgaaaatctgatgaccaagccctccgtgaagtcgaaattccgaaagctgtttaagatgcaaggacccatggacaaagtcaaagactggaaccaaatagccgccggcttgaagaatttccaatttgttcgtgacctagtcagagaggtggtcgactggctccaggcctggatcaacaaagagaaagccagccctgtcctccagtaccagttggagatgaagaagctcgggcccgtggctttggctcatgatgccttcatggccggttccgggccccctcttggtgacgaccagattgaatacctccaaaacctcaaatctcttgccctaacactggggaagactaatttggctcaaagtctcaccactatgatcaatgccaagcaaagttccgcccaacgagtcgaacccgttgtggtggtccttagaggcaagccgggatgcggcaagagcttggcctccacgttgattgcccaggctgtgtctaagcgtctctacggctcacaaagtgtgtattctcttcctccggatccagacttcttcgacggatacaaaggacagtttgtaaccttgatggatgatttgggacaaaacccggatgggcaagatttctccaccttttgccagatggtgtcgaccgcccaatttcttcccaacatggcggaccttgctgagaaggggcgtcccttcacctccaatcttatcattgcaactacaaacctccctcacttcagccctgtcaccattgctgatccttctgcagtctcccgtcgtatcaactacgacctgactctagaagtatctgaggcctacaagaagcacacacggctgaattttgacctggctttcaggcgcacagacgccccccccatttatccttttgctgcccacgtgcctttcgtggacgtggctgtgcgcttcaaaaatggtcaccagagcttcaatctcctagagttggttgactctatttgtgcagacattcgagccaagcaacaaggtgcccgaaatatgcagactctggttctgcagagccccaacgagaatgatgacacccccgtcgacgaggcgttgagtagagttctcacccccgctgcggtcgacgaggcgcttgtcgacctcgctccagaggccgacccggttggccgcttggctattcttgccaagctaggtcttgccctggctgcggtcacccctggtttgataatcctggcagtgggactctacaggtacttctctggctctgatacagaccaagaagaaacagaaagtgaggaacctgttaaagcgcccaggagcgagaatgcttatgacggcccaaagaaaaactctaagccccctggggcgctctctcttatggaaatgcagcagcccaacgtggacatgggctttgaggctgcggtcgctaagaaagtggtcgtccccattaccttcatggttcccaacagaccttctggacttacacagtccgctcttcttgtggccggccggactttcctaattaatgagcatacatggtccaacccctcctggaccagcttcacaatccgtggtgaggtgcacactcgtgatgagcctttccaaacggttcattttactcaccatggtgttcccacagatctgatgatggtgcgtctcgggccgggcaactctttccctaacaatctagacaagtttggacttgaccagatgccggcacgtaactcccgtgtggttggcgtttcggctagttacggtaacttcttcttctctgggaatttcctcgggtttgttgactccatcacctctgaccaaggaacttatgcgagactttttaggtacagggtgacgacttacaagggatggtgtggttcggccctggtctgtgaggccggtggtgtccggcgcatcattggcttgcactctgctggcgccgctggtatcggcgccgggacttacatctcaaaattgggactgatcaaagcccttaaacacctcggtgaacctctggctacaatgcaaggactgatgactgagctagagcctggagtcaccgtacatgtaccccggaaatctaaattgagaaagacgaccgcacacgcggtgtacaaaccggagtttgaacctgctgtgttgtcaaaatttgatcccagactgaacaaggatgttgacttagatgaggtgatttggtctaaacacactgccaacgtcccttatcaacctcctttgttctacacatatatgtcagagtacgctcaccgggttttctcctttttgggaaaagacaatgacattctgaccgtcaaagaagcaatcttgggcatccctggactagaccctatggatccccacacagctccgggtctgccctacgccattagcggccttcgacgtactgatctcgtcgattttgcgaacggcacggtagacgcggcactggccatgcaaattcagaaattcttagatggtgactactctgatcatgtcttccaaacttttctgaaagatgaaatcagaccctcagagaaggtccgagcgggaaaaacccgcattgtcgatgtgccttccctggcgcactgcattgttggcagaatgctgcttgggcgttttgccgccaagtttcaatcccatcctggctttctccttggctctgctatcgggtctgaccctgatgtcttctggaccgtcataggggctcagctcgagggaagaaagaacacgtatgacgtggactacagtgcctttgactcttcacacggcactggctccttcgaggctctcatctctcactttttcaccgttgacaatggttttagccctgcgctgggaccgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctagcattgacttacaaggaatttgagtatgacatggttgatatcatcgcctacggtgacgaccttctggttggtacggattatgatctggacttcaatgaggtggcgcggcgcgctgccaaactggggtataagatgactcctgccaacaagggttctgtcttccctccgacttcctctctctccgatgctgtttttctaaaacgcaaattcgtccaaaacaatgacggcttatataaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacctagatagcccaacgcgcctcggtgctgccggcgattctgggagaactcagtcgg

SEQ ID NO: 71 - RNA, 7174 nt, Senecavirus Seneca Valley virus A
  source 1..7174: mol_type = unassigned RNA
ccggctgtgtttgctagaggcacagtggagcaacatccaacctgcttttgtggggaacggtgcggctccaattcctgcgtcgccaaaggtgttagcgcacccaaacggcgcgtctaccaatgctattggtgtggtctgcgagttctagcctactcgtttctcccctactcactcattcacgcacaaaaactgtgttgtaactacaaggtttagccctcgcacgggatgtgcgataaccgcaagatcgactcaagcgcggaaagcgctgtaaccacatgctgttagtccctttatggctgcgagatggctacccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaaaacgtaaccggaacaaaagtcaagatcgttgaataccccagatcggtgaacaatggtgtttacgattcgtccactcatttagagatactgaacctacagggtgaaattgaaattttaaagtctttcaacgaataccaaattcgcgccgccaaacaacaacttggactggacatcgtatacgaactacagggtaatgttcagacaacctcaaagaatgattttgattcccgcggcaataatggtaacatgaccttcaattactacgcaaacacttaccagaattcagtagacttctcgacctcctcgtcggcgtcaggcgccggacccgggaactcccggggcggattagcgggtctcctcacaaatttcagtggaatcttgaaccctcttggctacctcaaagatcacaataccgaagaaatggaaaactctgctgatcgagtcataacgcaaacggcgggcaacactgccataaacacgcaatcatcactgggtgtgttgtgtgcctacgttgaagacccgaccaaatctgaccctccgtccagcagcacagatcaacccaccaccacttttactgccatcgacaggtggtacactggacgcctcaattcttggacaaaagctgtaaaaaccttctcttttcaggccgtcccgctccctggagccttcctgtctagacagggaggcctcaacggaggggccttcacggccaccctacatagacatttcttaatgaagtgcgggtggcaggtgcaggttcaatgcaatttgacgcaattccaccaaggtgctcttcttgttgccatggtccccgaaaccacccttgatgtcaaacctgacggcaaggcaaagagcttacaggagctgaatgaagagcagtgggtggaaatgtctgacgactaccggaccgggaaaaacatgccttttcagtctcttggtacatactatcggccccctaactggacttggggccccaatttcatcaacccctatcaagtaacagttttcccacaccaaattctgaacgcgagaacctctacctcggtagacataagtgtcccgtacatcggggagactcctacacaatcctcagagacacagaactcctggaccctccttgttatggtgcttgtccccctggactacaaggagggagccacaactgacccagaaattacattttctgtaaggcctacaagtccctacttcaatgggcttcgtaaccgtttcacgaccgggacggacgaggaacaggggcccattcccacagcacccagagaaaattcgcttatgtttctctcaaccatccctgacgacactgtccctgcttacgggaatgtgcgcacccctcccgtcaattacctccccggtgaaataaccgacctcttacaactggcccgtatacccactctcatggcgtttgggcgggcgtctgaacccgagcctgcctcagacgcatatgtgccctacgttgccgttcctgcccagttcgacgacaagcctctcatctccttcccgatcaccctttcagaccctgtctaccagaacaccctggtaggcgccatcagttcgaacttcgccaactaccgggggtgtatccaaattactctgacattttgtggacccatgatggcaagagggaaattcctgctctcgtattctcccccaaatggagcacaaccacagaccctttctgaagctatgcagtgcacatactctatttgggatataggcttgaactccagttggacctttgtcatcccctacatctcgcccagtgattaccgtgaaactcgggctattaccaactcagtttattctgctgatggttggtttagcttgcacaagctgaccaaaattactctaccacctgactgcccacagagtccctgtattctctttttcgcctctgctggtgaggattacaccctccgtctccctgttgattgtaatccttcctacgtgttccactccaccgacaacgccgagactggggttattgaggcaggtaacactgacaccgatttctctggtgaactggcggctcctggctctaaccatactaatgtcaaattcctgtttgaccgatctcgactactgaatgtaattaaggtactggagaaggacgccgtcttcccccgtcctttccccacagcaacaggtgcacagcaggacgatggttacttttgtcttctaacaccccgcccaacagtcgcttcccgacccgccactcgtttcggcctgtacgtcaacccgtctgacagtggcgttctcgctaacacttcactggatttcaatttttacagtttggcctgtttcacttactttagatcagaccttgaagtcacggtggtctcactggagccagatttggaattcgccgtggggtggttcccctctggcagtgagtaccaggcttctagctttgtctacgaccaactgcatgtaccctaccactttactgggcgcactccccgcgctttcaccagcaagggtggaaaggtatctttcgtgctcccttggaactctgtctcttccgtgcttcccgtgcgctgggggggtgcctccaagctttcttctgccacgcggggtctgccggctcatgctgactgggggaccatttacgcctttatcccccgtcctaacgagaagaaaagcaccgctgtaaagcacgtggcggtgtacgttcggtacaagaacgcgcgtgcctggtgccccagcatgcttccctttcgcagctacaagcagaagatgctgatgcaatcaggcgacgtcgagaccaaccctggccctgcttctgacaacccgatcttggagtttcttgaagcggaaaacgatctagtcactctggcctctctctggaagatggtacactctgttcaacagacctggagaaagtatgtgaagaacgacaatttttggcccaacttgctcagtgagctagtgggggaaggctccatcgccttggccgccacgctatctaaccaagcttcagtgaaagctctcttgggcctgcattttctctctcgggggctcaattacacagatttttactctttactgatagagaaatgctctagtttctttactgtggaaccgcctcctccaccagctgaaaatctgatgaccaagccctccgtgaagtcgaaattccgaaagctgtttaagatgcaaggacccatggatacagtcaaagactggaaccaaatagccgccggcttgaagaatttccaatttgttcgtgacctagtcaaagaggtggtcgactggctccaggcctggatcaataaagagaaagccagccctgtcctccagtaccagctggagatgaagaagctcgggcctgtagctttggctcatgattccttcatggccggttctgggccccctcttggtgacgaccagattgaatacctccagaacctcaaatctcttgccctaacactggggaagaccaatttggcccaaagtctcaccactatgatcaatgccaagcagagctccgcccaacgagtcgaacccgttgtggtggtcctcagaggcaagccgggatgcggcaaaagcttggcctccacgttgattgcccaggctgtgtccaagcgcctctatggctcacaaagtgtgtattctcttcctccggacccagacttcttcgacggatacaaaggacagtttgtaaccttgatggacgatctgggacaaaacccggatgggcaagatttctccaccttttgccagatggtgtcgaccgcccaatttcttcccaacatggcggaccttgcagagaaggggcgccccttcacctccaatcttatcattgcaactacaaacctccctcactttagccctgtcaccattgctgatccttctgcagtctctcggcgtatcaactacgacctgactctggaagtatctgaggcctacaagaagcacacacggctgaattttgacttggctttcagacgcactgacgccccccccatttatccttttgctgcccacgtgcccttcgtggacgtggctgtgcgctttaaaaatggtcaccaaagcttcaatctcctagagttggtcgactctatttgtgcagacattcgggccaagcaacaaggtgcccgaaatatgcagactctggttctacagagccctaacgagaacgacgacacccccgtcgacgaggcgttgggtagagttctcacccccgctgcggtcgacgaggcgcttgtcgacctcgctccagatgccgacccggttggccgcttggctattctcgccaagctaggtcttgccctagctgcggtcacccctggtttgataatcttggcagtgggactctacaagtacttctctggctctgatacagaccaagaagaaacagaaagtgaggagcctgttaaagcgcctaggagcgagaatgcttatgacggcccgaagaaaaactctaagccccctggagcgctctctcttatggaaatgcaacagcccaacgtggacatgggctttgaggctgcggtcgctaagaaagtggtcgtccccattaccttcatggttcccaacagaccttctggacttacacagtccgctcttcttgtgaccggccggaccttcctaatcaatgagcatacatggtccaacccctcctggaccagcttcacaatccgtggtgaggtgcacactcgtgatgagcctttccaaacggttcattttactcaccatggtcttcccacagatctgatgatggtacgtctcggaccgggcaactccttccctaacaatctagacaagtttggacttgaccagatgccggcacgtaactcccgtgtggtcggcgtttcggctagttacggtaacttcttcttctctgggaacttcctcgggtttgttgactccatcacctctgaccaaggaacctatgcgagacttttcaggtacagggtgacgacttacaagggatggtgcggttcggccctggtctgcgaggccggtggtgtccgacgcatcattggcctgcactctgctggtgccgctggtatcggtgccgggacttacatctcaaaattaggactgatcaaagcccttaaacacctcggtgagcctctggctacaatgcaaggactgatgactgagctagagcctggagtcaccgtacatgtaccccgaaaatctaaattgagaaagacgaccgcacacgcggtgtacaaaccggagtttgaacctgctgtgttgtcaaaatttgatcccagactgaacaaggatgttgacctagatgaggtaatttggtccaaacacactgccaacgtcccttatcaacctcctttgttctacacatacatgtcagagtacgctcatcgggttttctcctttttgggaaaagacaatgacattctgaccgtcaaagaagcaatcttgggcatccctggactagaccctatggatccccacacagctccgggtctgccctacgccattaacggccttcgacgtactgatctcgtcgattttgcgaacggcacggtagacccggcactggccatgcaaatccaaaaattcttagacggtgactactctgatcatgtcttccaaacttttctgaaagatgaaatcagaccctcagagaaggtccgggcgggaaaaacccgcattgtcgatgtgccctccctggcgcactgcattgtgggcagaatgctgcttgggcgttttgccgccaagtttcaatcccatcctggctttctccttggctccgctatcgggtctgaccctgatgtcttctggaccgtcataggggctcagctcgagggaagaaagaacacgtatgacgtggactacagtgcctttgactcttcacacggtactggctccttcgaggctctcatctctcactttttcaccgtggacaatggttttagccctgcgctgggaccgtatctcagatccctggctgtctcggtgcacgcttacggcgagcgtcgcatcaagattaccggaggcctcccctctggttgtgccgcgaccagcctgctgaacacagtgctcaacaatgtgatcatcaggactgctctagcattgacctacaaggaatttgaatatgacatggttgatatcatcgcctacggtgacgaccttctggttggtacggattacgatctggacttcaatgaggtggcgcggcgcgctgccaaactggggtataagatgactcctgccaacaagggttctgtcttccctccgacttcctctctctccgatgctgtttttctaaaacgcaaattcgtccaaaacaatgacggcttatataaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacctggatagcccaacgcgct

SEQ ID NO: 72 - RNA, 7280 nt, Senecavirus Seneca Valley virus A
  source 1..7280: mol_type = unassigned RNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 73 - RNA, 7282 nt, Senecavirus Seneca Valley virus A
  source 1..7282: mol_type = unassigned RNA
tttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 74 - RNA, 46 nt, synthetic construct
  misc_feature 1..46: note = ribozyme
  misc_feature 1..6: note = n is a, c, g, or u
  misc_feature 11: note = n is a, c, g, or u
  misc_feature 15..24: note = n is a, c, g, or u
  misc_feature 29..38: note = n is a, c, g, or u
  misc_feature 41..46: note = n is a, c, g, or u
  source 1..46: mol_type = other RNA
nnnnnnctgangarnnnnnnnnnngaaannnnnnnnnnthnnnnnn

SEQ ID NO: 75 - RNA, 61 nt, synthetic construct
  misc_feature 1..61: note = hammerhead ribozyme
  source 1..61: mol_type = other RNA
gtaccttatcaaactgatgagtccgtgaggacgaaacgagtaagctcgtctttgaaatggg

SEQ ID NO: 76 - RNA, 75 nt, synthetic construct
  misc_feature 1..75: note = hammerhead ribozyme
  source 1..75: mol_type = other RNA
tggtaccttaccatttcaaactgatgagtccgtgaggacgaaacgagtaagctcgtctttgaaatggggggctgg

SEQ ID NO: 77 - RNA, 59 nt, synthetic construct
  misc_feature 1..59: note = Pistol ribozyme
  misc_feature 2: note = n is a, c, g, or u
  misc_feature 6: note = n is a, c, g, or u
  misc_feature 8..9: note = n is a, c, g, or u
  misc_feature 15: note = n is a, c, g, or u
  misc_feature 17: note = n is a, c, g, or u
  misc_feature 25: note = n is a, c, g, or u
  misc_feature 27: note = n is a, c, g, or u
  misc_feature 35..36: note = n is a, c, g, or u
  misc_feature 44..51: note = n is a, c, g, or u
  misc_feature 56: note = n is a, c, g, or u
  misc_feature 58..59: note = n is a, c, g, or u
  source 1..59: mol_type = other RNA
rntcgnynnrgcganyntaaayarnynttargcynnrrgcgtynnnnnnnnrggtnrnn

SEQ ID NO: 78 - RNA, 72 nt, Paenibacillus polymyxa
  source 1..72: mol_type = unassigned RNA
ggactcgtctgagcgagtataaacaggtcattaagctcagagcgttcaccggggaaattcggtgaggttggc

SEQ ID NO: 79-90 - skipped (12 entries)

SEQ ID NO: 91 - DNA, 17 bp, synthetic construct
  misc_feature 1..17: note = T7 promoter
  source 1..17: mol_type = other DNA
taatacgactcactata

SEQ ID NO: 92 - DNA, 7498 bp, synthetic construct
  misc_feature 1..7498: note = SVV-S177A-SapI_3p-Pistol1_5p IRES2_L5 -70pA-SVV-5.0
  source 1..7498: mol_type = other DNA
taatacgactcactatagggacgcgtcgaaacgttactttaaatggcagctaagaacttaaactacaacatggactcgtctgagcgagtataaacagtcaattaagctcagagcgttcaccggggaaattcggtgaggtttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaaaaaaaaaaaaaaaaaaaaaaaaaaaaaaaaaaaaaaaaaagaagagc

SEQ ID NO: 93 - DNA, 7626 bp, synthetic construct
  misc_feature 1..7626: note = CVA-PP-TwL6-BsaI_KY_70pA
  source 1..7626: mol_type = other DNA
taatacgactcactatagggtcgactctacaaataacggataccaatttgagtaacgcggcttcaatgtacaataatggactcgtctgagcgagtataaacagttttttaagctcagagcgttcaccggggaaattcggtgaggttaaaacagctctggggttgttcccaccccagaggcccacgtggcggctagtactctggtatcacggtacctttgtacgcctgttttatatcccttcccccgcaactttagaagcttaacaaaagttcaatagcaggggtgcaagccagtacctctatgaacaagcacttctgtctccccggtgaaattacatagactgtacccacggtcaaaagtaattgatccgttatccgcttgagtacttcgagaagcctagtatcaccttggaatcttcgacgcgttgcgctcaacactctaccccgggtgtagcttaggctgatgagtctgggcaccccccaccggtgacggtggcccaggctgcgttggcggcctacccatggctaacgccatgggacgctagttgtgaacaaggtgtgaagagcctattgagctactcaagagtcctccggcccctgaatgcggctaatcccaaccacggagcaattgctcacgatccagtgagtaggttgtcgtaatgcgcaagttcgtggcggaaccgactactttgggtgtccgtgtttccctttttacttatactggctgcttatggtgacaatttacagattgttaccatatagctattggattggccatccagtattgtgttacacatattaaaatacttttctacaagcatcaacaacattgtgtttggaccacaataatcactacaaatgggagctcaagtatcaacccaaaagaccggtgcacacgaaaatcaaaacgtggctgccaatggatccaccattaattacactactatcaactactacaaagatagtgcaagtaactcagccaccagacaagatctttctcaagatccatcaaaatttacagaaccagttaaagatttaatgttgaaaacagcaccagctttgaattcaccaaatgtggaagcatgtggatacagtgaccgtgtaaggcaaattaccctgggaaactcgactattaccacacaagaagcagctaacgctattgttgcttatggtgagtggcccacttacataaatgactcagaagcaaacccagtagatgcacccactgaaccagatgttagtagcaacaggttttacactctggactcagtgtcttggaagactacctcaaggggttggtggtggaaactaccagactgtctaaaagacatggggatgtttggtcagaatatgtattaccactacctggggcgctccggttacaccattcatgtccagtgcaatgcctcaaagtttcaccaaggggcattaggagttttcttgataccagagtttgttatggcctgtaacaccgagagcaagacatcatatgtttcatacatcaacgcaaatcctggtgagaggggtggtgagtttacaaacatttacaatccatcaagcacagatgctagtcagggcagacaatttgcagcgctagattatttactgggttctggtgttttagctggaaatgcatttgtgtacccacaccagatcattaacctgcgtaccaacaatagtgcaactattgtggtaccatatgtgaactcacttgtcattgattgcatggcaaaacacaacaactggggtattgtcatcctgccactggcgccattggcctttgctgcgacatcatcaccacaagtgcctattacagtgaccattgcacccatgtgcgcagaattcaatggattgagaaacatcactattccagtgcatcaaggattaccaacaatgaacacacctggatccaatcagtttctcacatctgatgacttccagtcaccctgcgctctacccaactttgatgtcactccacctatacacatacccggagaagtgaagaatatgatggaactggccgagattgacacgctgatcccaatgaacgcagtggacggaaaggtaaacactatggagatgtatcaaataccattaaatgatgaactaagcaaagcacccatattttgcctgtccctgtcacctgcttctgacaaacgattaagtcatacgatgttgggtgaaattctaaactattacactcattggacagggtccattaggttcacctttctattctgtggtagcatgatggccactggtaagttgcttctcagttattccccaccaggagctaaaccaccaaccaaccgcaaggatgcaatgttgggcacacatatcatctgggacttgggtttacaatccagttgctccatggttgcaccatggatctctaatacggtatacaggcggtgtgcacgtgatgacttcacagagggtgggtttataacttgcttttatcaaaccagaattgttgtgcctgcctcaacccctaccagcatgttcatgttaggctttgtgagtgcatgtccagatttcagcgtcagactgctgagggacacttcccacattagtcaatcaaaactcatagcacgctcacaaggcattgaagatctcatcgattcagcaataaagaatgctttgagagtgtcccaaccgtccacggctcaatcgactgaagcaaccagtggagcaaacagtcaggaagtaccagcactaactgctgtagaaacaggagcatcaggtcaagcaattcccagtgacgtgatggaaaccagacacgtgataaactacaaaactaggtctgagtcatgtcttgaatcattctttgggagagctgcgtgtgtcacaatcctatctctgaccaactcttcaaaaagtggagaggaaaagaaacatttcaacatttggaacatcacgtataccgacactgtgcagctacgtagaaaattagagttcttcacatattccagatttgaccttgaaatgacttttgtgttcacagagaactaccccagtacagctagtggagaagtgcgcaaccaggtataccagattatgtatattccaccaggggcaccccgaccatcatcctgggatgattatacatggcaatcctcctccaacccttctatcttttacatgtacgggaacgcaccgccacggatgtcaatcccctatgtagggattgctaatgcctactcacacttttatgacggttttgcacgagtgccacttgagggtgagaacactgatgctggtgacacgttttatggattggtatccataaatgattttggagtcttggcagtcagagcagtgaaccgcagcaatccacacacaatacacacatccgtgagagtgtacatgaaaccaaaacacattcggtgctggtgccccagaccccctcgcgcagtattatacagaggagaaggagtggacatgatatccagtgcaattttacccctgaccaaagtagattctattactaccttcggatttggacaccaaagtaaggcagtgtatgttgccggttataagatctgcaactaccacctagcaacccctagtgatcatctaaatgcaattagtgtgttgtgggatagggatttaatggtagtagagtctagagcccaaggaactgacaccatagccagatgtagttgtaggtgtggggtctattactgtgaatcaagaaggaagtactaccctgtcactttcaccggtccaacgttccgattcatggaagcgaatgactactacccagcaagataccagtctcacatgctgataggatgtggatttgcagagcctggagattgcggtggaatactaaggtgcacccatggggtgattggtattattactgcagggggtgagggagtagtagcctttgctgacatcagggatctttgggtgtatgaagaggaggccatggaacagggaataacaagctacattgagtctcttggcacagcctttggtgcagggtttactcacacaattagtgaaaaagtgactgagttaactacaatgattaccagtaccattacagaaaaactactaaaaaatttggtgaaaatagtatcagctttagtaattgttgtgagaaattatgaagacactactacagttcttgcaacactagcattacttgggtgtgacatatctccttggcagtggttgaagaaaaaggcatgtgacctactagaaattcctcatgtactgcgccaaggtgatgggtggatgaagaaattcacagaggcttgcaatgcagccaagggcttagaatggattagcaacaaaatttctaagttcatagactggttgaagtgcaaaattattccagaagccaaagataaggtggaatttctcaccaagctgaaacagttagacatgttagaaaatcaaattgcaactattcaccagtcttgccccagccaggagcaacaagagattctcttcaacaatgtgagatggttagcagtccagtcccgtcgatttgcaccactatatgctgtggaagcacggagaattagcaaaatggagaacacaataaacaactacatacagttcaagagcaaacaccgtattgaaccagtatgtatgctcatccatgggtcaccgggaacaggcaaatcaatagccacctcattgataggcagagcgatagcagaaaaggagaatacatcagtttactcaatgccacctgacccatctcactttgatggctacaaacaacaaggggtcgtgattatggatgatttgaatcagaaccctgatggcatggatatgaaactgttttgccaaatggtgtcaacggtggagttcatcccaccgatggcctcactagaggagaagggtattttgtttacatctgattatgtcctagcctccaccaactcccattcaatagcaccacccacggtagctcacagtgatgccttaaccagacgatttgcattcgatgtggaagtttacacaatgtcagagcactcaatcaaaggcaaattgaacatggccacagccacccagttatgcaaggattgtccaacacccgcaaattttaaaaagtgctgccctcttgtctgtggtaaggctttgcagctaatggatagatataccagacagagattcactgtagatgaaatcaccacattgattatgaatgagaaaaataggagggccaatattggtaactgcatggaagccttgttccaaggaccgctgaggtacagggatctgaaaattgatgtgaagacagtccccccccctgaatgcatcagtgacctactacaagcagtggattctcaagaagttagagattactgtgaaaagaagggctggatagtcaatgttactagccaaatacagttagaaagaaatatcaacagggccatgactatactccaagctgtgactacgtttgcagcagttgcaggagtagtgtatgtgatgtataaactctttgctggtcaacagggtgcatacactggtttgccaagcaaaaagcccaatgtcccaacaatcagagttgccaaagtacagggaccagggtttgattatgcagtggcaatggctaaaagaaacatacttactgcaaccactactaagggtgaattcactatgttaggagtgcatgataatgtagcgatactgccaacccatgccgccccaggagaaactatcattattggtgggaaagaagtggagatcttggacgccagagccctagaagatcaagcgggaaccaatcttgagatcactataatcactctaaaaagaaatgagaagtttagagatatcagaccacatattcccacccaaatcaccgaaaccagtgatggagtattgatcgtgaacactagtaagtaccccaatatgtatgtccctgttggtgctgtgaccgaacaggggtatcttaatctcagtggacgccaaactgctcgtactctgatgtacaactttccaacacgagcaggccagtgcgggggaatcattacctgcactggtaaagtcattgggatgcatgttggtgggaacggttcacatgggtttgcagcagcccttaagcgatcatacttcacccaaagtcaaggtgaaatccaatggatgaggtcatcaaaagaagtgggataccctattataaatgccccatcaaaaacaaagttagaacccagtgcttttcattatgtcttcgaaggtgtcaaagaaccagcagtgctcaccaagagtgaccccagattgaaaacagatttcgaagaagccattttttcaaaatatgtggggaataaaattgttgaagtggacgaatacatgaaagaagcagtggaccactatgcagggcagctaatgtcattagacattaatacagaacaaatgtgcttggaagatgccatgtacggcactgatggtcttgaggctctagatcttagtaccagtgcaggatacccttatgttgcaatgggaaaaaagaaaagagatatcttagacaagcagactagagatactaaagagatgcagaaacttctagacacttatggaatcaacctaccattagttacctatgttaaagatgaactcaggtcaaagactaaagtagagcaaggaaaatcaaggctaattgaagcttccagtctcaatgattcagttgcaatgagaatggcattcggcaatctttacgcagccttccataagaatccaggtgtggtaaccggctcagcagttggttgtgacccagacttattttggagtaagataccagtattaatggaagaaaagctctttgcctttgattacacagggtatgatgcctcgcttagtcctgcttggtttgaggcacttaaaatggtgttagaaaagataggttttggtgatagagcagattacatagattacctgaaccactctcatcatctttacaaaaacaaaacttattgtgttaagggcggtatgccatctggctgttctggcacatcaatttttaactcaatgattaacaatttgatcatcaggacgcttttactgaaaacctacaagggcatggatttagaccatttaaaaatgattgcctatggtgatgatgtgatagcttcctacccccatgaggttgacgctagtctcctagcccaatcaggaaaagactacggactgaccatgactccagcagataaatcagcaacctttgaaacagtcacatgggagaatgtaacgttcctgaaaagatttttcagagcagatgagaaatatccattcctggtgcatccagtaatgccaatgaaagaaattcatgaatcaatcagatggaccaaggaccctagaaatacacaagatcatgtgcgctcgttgtgtttattggcttggcacaacggtgaagaagaatataacaaattcttagctaaaatcagaagtgtgccgatcggtagagctttgttgctcccagagtactctacattgtaccgccggtggcttgactcgttttagtaaccctacctcagtcgaattggattgggttatactgttgtaggggtaaatttttctttaattcggagaaaaaaaaaaaaaaaaaaaaaaaaaaaaaaaaaaaaaaaaaaaaaaaaaaaaaaaaaaaaaaaaaaaaaaagagacc

SEQ ID NO: 94 - DNA, 7626 bp, synthetic construct
  misc_feature 1..7626: note = CVA-PP-TwL6-BsaI_Kuykendall_70pA
  source 1..7626: mol_type = other DNA
taatacgactcactatagggtcgactctacaaataacggataccaatttgagtaacgcggcttcaatgtacaataatggactcgtctgagcgagtataaacagttttttaagctcagagcgttcaccggggaaattcggtgaggttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaaatgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccacttttgggtttggtcatcagaacaaagcagtgtacgttgccggttacaagatttgcaactaccacctagcaaccccaagtgatcacttgaatgcaattagtatgttatgggacagggatttaatggtggtggaatctagagcccagggaactgataccatcgccagatgtagttgcaggtgtggagtttactattgtgaatctaggaggaagtactaccctgtcacttttactggcccaacgtttcgattcatggaagcaaacgactactatccagcaagataccagtctcacatgctgatagggtgcggatttgcagaacccggggactgcggtgggatactgaggtgcactcatggggtaattggtatcattactgcaggaggtgaaggggtagtagcctttgctgacattagagatctctgggtgtatgaagaggaggccatggaacagggaataacaagctacatcgaatctctcggcacagcctttggcgcagggttcacccacacaatcagtgagaaagtgactgaattgacaacaatggttaccagcactatcacagaaaaactactgaaaaacttggtgaaaatagtgtcggctctagtgattgttgtgagaaattatgaggacactaccacgatccttgcaacactagcactactcgggtgtgatatatctccttggcaatggttgaagaagaaggcatgtgacttactagagattccttatgtgatgcgccaaggtgatgggtggatgaagaaattcacagaggcgtgcaatgcagctaaaggcttagagtggattagcaacaaaatttccaagtttatagattggttgaagtgtaaaattatcccagacgctaaggacaaggtggaatttctcaccaagttgaaacagctagacatgttggaaaatcaaattgcaaccatccaccaatcttgccccagccaagaacaacaagagattcttttcaacaatgtgagatggctagcagtccagtcccgtcggtttgcaccattatacgctgtggaggcacgccgaattaacaaaatggagagcacaataaacaattatatacagttcaagagcaaacaccgtattgaaccagtatgtatgctcattcatgggtcaccagggacgggtaaatctatagctacttcattaataggtagagcaatagcagagaaggaaagcacatcagtctattcaatgccacctgacccatctcactttgatggctataaacaacaaggggtagtgattatggacgacctaaaccaaaaccccgatggtatggacatgaaactgttttgccaaatggtatcaacagtggagtttattcctccaatggcctcattagaggagaagggcattttgtttacatctgattatgtcctggcttctaccaactctcattcaattgtaccacccacagtggctcacagtgatgccttaaccagacgatttgcatttgatgtggaggtttacacgatgtctgaacattcagtcaaaggcaaactgaatatggccacggccactcaattgtgtaaggattgtccaacacctgcaaattttaaaaagtgttgccctctcgtttgtggaaaggccttgcaattaatggacaggtacaccagacaaaggttcactgtagatgagattaccacattaatcatgaatgagaaaaacagaagggccaatatcggcaattgcatggaagccttgtttcaaggaccattaaggtataaagatttgaagatcgatgtgaagacagttcccccccctgagtgcatcagtgatttgttacaagcagtggattctcaagaggttagggattactgtgagaagaaaggctggatcgttaacgttactagccagattcaactagaaaggaacatcaatagggccatgactatactccaagctgttaccacattcgcagcagtcgcaggagtagtgtatgtaatgtacaaactcttcgccggtcaacagggtgcatacactggcttgccaaacaaaaaacccaatgtccctactatcagagtcgctaaagtccaggggccaggatttgactacgcagtggcaatggcaaaaagaaacatagttactgcaaccaccaccaagggtgaatttaccatgctaggggtgcatgataatgtagcaatattgccaacccatgccgctccaggagaaaccattattattgatgggaaagaagtagagatcctagatgccagagccttagaagatcaagcgggaaccaatcttgagatcaccattattactctaaaaagaaatgagaagtttagagacatcagatcacatattcccacccaaattactgaaactaacgatggagtgttgatcgtgaacactagcaagtaccccaatatgtatgtccccgttggtgctgtgaccgaacagggatatcttaatctcagtggacgtcaaactgctcgcactttaatgtacaactttccaacaagggcaggccagtgcggaggaatcatcacttgtactggcaaagtcattgggatgcatgttggcgggaacggttcacatgggtttgcagcagccctcaagcgatcatacttcactcaaaatcagggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaaccctacctcagtcggattggattgggttatactgttgtaggggtaaatttttctttaattcggagaaaaaaaaaaaaaaaaaaaaaaaaaaaaaaaaaaaaaaaaaaaaaaaaaaaaaaaaaaaaaaaaaaaaaaagagacc

SEQ ID NO: 95 - DNA, 7661 bp, synthetic construct
  misc_feature 1..7661: note = CVA-PP-TwL6-BsaI_EF_70pA
  source 1..7661: mol_type = other DNA
taatacgactcactatagggtcgactctacaaataacggataccaatttgagtaacgcggcttcaatgtacaataatggactcgtctgagcgagtataaacagttttttaagctcagagcgttcaccggggaaattcggtgaggttaaaacagctctggggttgttcccaccccagaggcccacgtggcggctagtactctggtatcgcggtacctttgtacgcctgttttatactcccttccccatgtaactttagaagcaattcaccaagttcaatagagggggtacaagccagtaccaccacgaacaagcacttctgttaccccggtgagtttgcataagctgtgcccacggctgaaagtaaatgatccgttatccgcttgagtacttcgagaagcctagtatcaccttggaatcttcgacgcgttgcgctcaacactctaccccgagtgtagcttaggctgatgagtctgggcatcccccaccggcgacggtggcccaggctgcgttggcggcctacccatggccaacgccatgggacgctagttgtgaacaaggtgtgaagagcctattgagctactcaagagtcctccggcccctgaatgcggctaatcctaaccatggagcaagtggccccaacccaggggatggcttgtcgtaacgcgcaagtccgtggcggaaccgactactttgggtgtccgtgtttccttttattttatatatggctgcttatggtgacaatcacagattgttatcataaagcgaattggattggccatccggtgaaatataagcacattatttatatctttgctggattcgctccgttgacacattcttcaccaaagttgatttatacagtcttagtaattagaaatcaccacttcagtcatgggagctcaagtatccactcaaaagactggggcccacgaaaaccagaatgtggccgcgaacgggtccacaatcaattatacaaccatcaactactataaggacagtgcaagcaattcggcgactcggcaggatttctcacaggacccgtccaaattcactgaaccggtcaaggatttaatgttaaaatccgcaccggctttgaattcgccaaacgtcgaagcatgcgggtacagtgatcgcgtgaggcaaatcacactgggaaattccaccataaccacacaggaggctgcaaatgcaattgttgcctatggggagtggccaacctacataaacgataacgaggccaaccctgtcgatgcaccgaccgaaccagatgtgagtagcaaccgcttttacaccctggaatcagtgcagtggaaaacaacatcccgggggtggtggtggaaattgccagactgtttgaaagacatgggtatgttcgggcaaaacatgtattaccactatttaggcaggtcagggtatactatacatgtgcagtgtaatgcctcaaaatttcaccaaggtgcgctgggtgtgtttctaatccctgaatttgtgatggcatgtaacaccgagagcaagacctcatatgtctcatatattaatgccaaccctggtgagaagggtggtgaatttacaaacacctacaacccatctggcacagacgttagcgaagggaggaaatttgccgctctggattatctgctagggtcaggcgtcttggcgggaaacgcttttgtttacccccatcaaattatcaaccttcgcaccaacaatagtgcaacaatagtggttccttacgtcaactcactagtgatcgattgcatggcaaaacacaacaactgggggatagcgatactaccccttgcacccctagcctttgcagcaacgtcatcaccacaagtaccgatcacagtcacaattgcaccaatgtgtacagagttcaatggattgaggaatgtaactgtaccagtacaccagggccttccagtcatgaacaccccaggatcaaaccaattcctcacatcagatgatttccagtcgccatgtgccttacccaatttcgatgtgacccctccaatccacatcccaggggaggtcaaaaacatgatggaactagctgaaattgacacactgattccaatgaatgcagtggatgggaaagtaaataccatggaaatgtaccagatcccattaaacgacaaccttagcactgaacccatcttttgcctatcactatcaccagcgtctgataggcgattgagtcacaccatgctgggagagattctgaactactacacacactggacgggctccatcagatttacttttctattttgtggtagtatgatggccacagggaagttgctccttagctattcacccccaggggcgaagccacccaaaaaccggaaagacgccatgctgggtacgcacataatatgggacctgggtttacagtctagttgttccatggtggcgccctggatttccaatacggtgtacaggcgatgtgttcgcgacgattttaccgaaggtggctacatcacctgtttctatcagactagaatagtggtaccagcctcgaccccaaccagcatgtttatgctaggctttgtcagtgcttgtcccgactttagtgtgagactgctcagggacacgacgcacatcagtcaatcgaaattggtcgccagtaggcagggcatagaggagtttatagacacagcaattaaaaatgcattgaaagtgtcacaacccagcactgcacaatcaacagaagcaaccagcggagtcaacagccaagaggtgcctgcactaacagcagtagagactggcgcatcgggccaagcaatcccaagtgatgtggtagaaactcgccacgtggtgaactataagacaagatcagagtcctgccttgaatcattctttggtagggccgcttgtgtgacaattctctcccttacaaactcttcaggtagcggagacgcaaagaagcactacacaacatggaacatcacatatacagatacggtgcaactgcgaaggaaactggaattttttacgtattccaggtttgacttggaaatgacctttgtattcactgagaactaccctagcacagctagtggagaggttcgaaatcaggtttaccaaatcatgtacattcctccaggggcgcctagacctagttcttgggatgattacacctggcagtcatcatcaaacccttccatattctacatgtatggtaatgcaccaccacggatgtcgattccctatgtcgggattgctaacgcctactcgcacttctacgatggcttcgcgcgagtgccgcttgaaggtgagaacacagatgccggggacaccttctacgggctagtatccatcaatgattttggtgtactggcagttagggcagtgaaccgcagcagccctcacgccatccacacttcagtgagggtgtatatgaaacctaaacacattcgctgctggtgccccaggccaccacgagcagtactttaccgtggtgagggagtggacatgatctcaagtgcaatccaaccactaaccaaggtcgattctataacaacattcggctttggtcaccagaacaaggcagtttatgttgctgggtataaaatttgtaactaccacttagcaacacccagtgaccacctgaacgccattagtgtgatgtgggacagagatttgatagtagtggagtcaagagcacagggaattgacaccatagcccggtgcaactgcaagtgtggcgtatactactgtgaatcaaggaaaaagtattatcccatttcgttcactgggcccacatttagatacatggaggccaacgaatactttccagctagatatcagtcccatttattaattgggtgcgggtttgcagagcctggagactgtggtggcatactcagatgcacacacggtgtgatcggcataattacagcaggtggacctggagtggtggcctttgccgacatcagggacctctgggtgtatgaggaggaagcgatggaacaaggcattacaagctacatcgagactttaggggcagcatttggaactgggttcactcaacaaattggagacaaaattacagagttgactaatatggtgacaagcacttttacggacaaattgtttaaaaacttgattaaaatagtgtcttcgttggttataataactcgaaactacgaagacaccaccacagtattggccactctagcattgttgggttgtgattgctctccgtggcaatggttgaaacacaaagtatgtgacatcctgcaactgccattcgccctcaaacagagtgacagttggctaaagaagttcactgaggcgtgtaatgccgcaaaagggctcgaatggattagcaacaagctctccaaatttgtggaatgggtgaagaacaagataattcctgaagccagagacaaagtggaatttctgcaaaaattgaaacagttagaaatgcttgagaaccagattgctacactacaccaatcctgtccaagtcaagaacagcaagagacactctttaacaatgtccgctggttggcactgcagtcaagacgctttgcgccgttgtacgctgttgaagcgcgcagaatacaaaaattggagaacaccataaacaactacatacagtttaagagcaaacaccgtattgaaccagtatgtatgttgatacatggagctcctggcacgggcaaatctatagcaacctcgttgattgggcgtgccattgctgaaaaagaaagcacctcagtgtattctctaccaccagacccctcacacttcgacggttacaaacaacaaggggttgttatcatggatgacctcaatcagaacccggatggtatggacatgaagttgttttgtcagatggtgtctacagtcgagttcatacccccaatggcatctctcgaggagaaaggaatcctgttcacttcagactatgtgctagcatctacaaattctcactcaattaccccacccactgtcgcacatagtgatgccctaagtagaagatttgcatttgacatggatgtatacaccatgtctgaacactctgtgaagggaaagctcaacatggcgactgcgacgcagttatgcaaagagtgtccaccgcccgctaacttcaaaaagtgctgtccactagtgtgtggtaaagctttacaactgatggacaagcacaccagacagagatttacactagacgaaatcaccactttgatcatcaatgagaagaacagaagggctaacattggtaattgcatggaggctctttttcaaggccctctacaatacaaagatctcaagatcgacatcaagaacactccccctccagagtgcataagtgatctacttcaggctgttgactcgcaagaggttagagattactgtgagaagaaaggatggatcgttaacgtatccagtcaggttcaattagagcgcaacatcaatagagctatgactatcctccaggcagtaaccacgttcgccgcagtggcgggagtggtttatgtcatgtacaagctctttgcagggcatcaaggcgcttatactgggctcccgagcaagaaacccaacgtgcccaccattagaatagctaaagtgcaaggtccagggtttgactatgcagttgccatggcaaagagaaatatcgttactgcaaccaccaataagggggagttcactatgcttggagtgcatgataacgttgccgtactaccaacccatgcatcaccaggtgagactatagttattgatggtaaggaagttgaagtgctggacgccaaggccctggaggaccaagctggaactaacctagagatcaccattgtcactctgagaaggaatgagaagttcagggacatcagacctcacatccccactcaaatcactgagacaaatgatggagtgttgatcgtgaacactagtaagtaccccaacatgtatgtgcccgttggtgcagtgaccgaacaagggtacttgaacctgggtgggcgtcaaactgctcgcactctaatgtacaatttcccaacccgagccggacaatgtggtggaatcatcacatgcactggcaaggtcattgggatgcatgttggtgggaacggttcacatgggttcgctgcagccctaaaacggtcatacttcactcagagccaaggtgagattcagtggatgaggccatcaaaagaggtcggttacccaataattaatgcaccaagcaagacaaaactagaacccagtgctttccactatgtttttgaaggggtaaaggaacctgcagtactgactaaaaacgatcccagattgaaaacggattttgaggaggccatattctcaaagtatgtagggaacaagatcactgaagtggatgaatatatgaaagaggcagtagaccattatgctgggcaactcatgtctttagacatcaatacagagcaaatgtgcttggaagatgctatgtatggcactgatggtctggaagctttggatctctctactagcgcaggatacccttatgtggctcttgggaaaaagaagagagatatacttaataaacaaacaagagacaccaaagaaatgcaaagactactagatacctatggaataaatttgcctctagttacttacgtaaaagatgagctgagaagcaagagcaaggtggagcagggtaaatccagattaattgaggcctcaagcttgaatgattcagtagcaatgagaatggcatttggtaacctgtatgcagctttccacaagaatccaggagtcatcactggttctgctgtaggatgtgacccagacttgttttggagcaagatcccagtattgatggaagaaaagctctttgcatttgattacaccggttacgatgcctctctcagtcccgcttggtttgaggcactcaaaatggttttggagaaaattggattcggagatagggtagattacattgattatctcaaccattcacaccatctttacaaaaacaagacctattgtgtcaagggcggtatgccatctggctgctcaggcacttcaatatttaattcaatgattaacaatttaataatcaggacactcttactgaagacctacaagggcatagatttggaccacttgaagatgattgcctatggtgatgatgtaattgcctcctacccccacgaagttgacgctagtctcctagcccaatcaggaaaagactatggactaaccatgacaccagctgacaagtcagccacctttgaaactgtcacgtgggagaatgtaacatttttaaaaagattctttagggcagatgaaaagtacccattcttaatacatccagtgatgccaatgaaagaaatacaagaatcgatcagatggactaaagatcctagaaacacgcaggaccatgttcgctcactgtgcttattggcttggcacaatggcgaagaagagtacaataaatttttagctaaaatcagaagtgtgccaatcggaagagctttactgctccccgaatactcaactttgtaccgccgttggctcgactcgttttagtaaccctacctcagtcgaattggattgggtcatactgttgtaggggtaaatttttctttgattcggagaaaaaaaaaaaaaaaaaaaaaaaaaaaaaaaaaaaaaaaaaaaaaaaaaaaaaaaaaaaaaaaaaaaaaaagagacc

SEQ ID NO: 96 - DNA, 57 bp, synthetic construct
  misc_feature 1..57: note = Env25 Pistol Ribozyme
  source 1..57: mol_type = other DNA
cgtggttagggccacgttaaatagtttattaagccctaagcgttgatgaaaatcagg

SEQ ID NO: 97 - DNA, 62 bp, synthetic construct
  misc_feature 1..62: note = Alistipes Putredinis Pistol Ribozyme
  source 1..62: mol_type = other DNA
ggagccgttcgggcggctataaacatttatcaggcccgaagcgtggcggcgaaagccgccgg

SEQ ID NO: 98 - DNA, 70 bp, synthetic construct
  misc_feature 1..70: note = Twister Sister1 Ribozyme
  source 1..70: mol_type = other DNA
ggacccgcaaggccgacggcatccgccgccgctggtgcaagtccagccgccccgaaaggggcgggcgctc

SEQ ID NO: 99 - DNA, 70 bp, synthetic construct
  misc_feature 1..70: note = Twister Sister2 Ribozyme
  source 1..70: mol_type = other DNA
ggacccggaaggccgacggcatccgccgccgctggtgcaagtccagccgccccgaaaggggcgggcgctc

SEQ ID NO: 100 - RNA, 57 nt, synthetic construct
  misc_feature 1..57: note = Env25 Pistol Ribozyme
  source 1..57: mol_type = other RNA
cgtggttagggccacgttaaatagtttattaagccctaagcgttgatgaaaatcagg

SEQ ID NO: 101 - RNA, 62 nt, synthetic construct
  misc_feature 1..62: note = Alistipes Putredinis Pistol Ribozyme
  source 1..62: mol_type = other RNA
ggagccgttcgggcggctataaacatttatcaggcccgaagcgtggcggcgaaagccgccgg

SEQ ID NO: 102 - RNA, 70 nt, synthetic construct
  misc_feature 1..70: note = Twister Sister1 Ribozyme
  source 1..70: mol_type = other RNA
ggacccgcaaggccgacggcatccgccgccgctggtgcaagtccagccgccccgaaaggggcgggcgctc

SEQ ID NO: 103 - DNA, 70 bp, synthetic construct; identical to SEQ ID NO: 99
  misc_feature 1..70: note = Twister Sister2 Ribozyme
  source 1..70: mol_type = other DNA

SEQ ID NO: 104 - protein, 471 aa, Homo sapiens
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK

SEQ ID NO: 105 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 106 - protein, 496 aa, Homo sapiens
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPDAAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHS

SEQ ID NO: 107 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 108 - protein, 239 aa, Homo sapiens
MAALKSWLSRSVTSFFRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 109 - protein, 26 aa, Apis mellifera
GIGAVLKVLTTGLPALISWIKRKRQQ

SEQ ID NO: 110 - protein, 485 aa, Bothrops pauloensis
ADDGNPLEECFRETDYEEFLEIAKNGLSATSNPKHVVIVGAGMSGLSAAYVLANAGHQVTVLEASKRAGGRVRTYRNDKEGWYANLGPMRLPEKHRIVREYIRKFGLQLNEFSQENENAWYFIKNIRKRVGEVNKDPGVLEYPVKPSEVGKSAGQLYEESLQKAVEELRRTNCSYMLNKYDTYSTKEYLLKEGNLSPGAVDMIGDLLNEDSGYYVSFIESLKHDDIFAYEKRFDEIVGGMDKLPTSMYQAIQEKVRLNVRVIKIQQDVKEVTVTYQTSAKETLSVTADYVIVCTTSRAARRIKFEPPLPPKKAHALRSVHYRSGTKIFLTCTKKFWEDDGIHGGKSTTDLPSRFIYYPNHNFPSGVGVIIAYGIGDDANFFQALDFKDCGDIVINDLSLIHQLPKEEIQAFCRPSMIQRWSLDKYAMGGITTFTPYQFQHFSEALTAPVDRIYFAGEYTAQAHGWIDSTIKSGLTAARDVNRASE

SEQ ID NO: 111 - protein, 82 aa, Crotalus atrox
TPVSGNELLEAGIECDCGSLENPCCYATTCKMRPGSQCAEGLCCDQCRFMKKGTVCRVSMVDRNDDTCTGQSADCPRNGLYG

SEQ ID NO: 112 - protein, 281 aa, Homo sapiens [sequence omitted]

SEQ ID NO: 113 - protein, 217 aa, Escherichia coli
MDIISVALKRHSTKAFDASKKLTPEQAEQIKTLLQYSPSSTNSQPWHFIVASTEEGKARVAKSAAGNYVFNERKMLDASHVVVFCAKTAMDDVWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV

SEQ ID NO: 114 - protein, 240 aa, Escherichia coli [sequence omitted]

SEQ ID NO: 115 - protein, 125 aa, synthetic construct
  REGION 1..125: note = ARV p14
MGSGPSNFVNHAPGEAIVTGLEKGADKVAGTISHTIWEVIAGLVALLTFLAFGFWLFKYLQKRRERRRQLTEFQKRYLRNSYRLSEIQRPISQHEYEDPYEPPSRRKPPPPPYSTYVNIDNVSAI

SEQ ID NO: 116 - protein, 140 aa, synthetic construct
  REGION 1..140: note = BRV p15
MGQRHSIVQPPAPPPNAFVEIVSSSTGIIIAVGIFAFIFSFLYKLLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 117 - protein, 154 aa, synthetic construct
  REGION 1..154: note = p14-p15 hybrid
MGSGPSNFVNHAPGEAIVTGLEKGADKVAGTISHTIWEVIAGLVALLTFLAFGFWLFKLLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 118 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 119 - protein, 482 aa, Homo sapiens
IDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 120 - protein, 340 aa, Homo sapiens
MALKEGLRAWKRIFWRQILLTLGLLGLFLYGLPKFRHLEALIPMGVCPSATMSQLRDNFTGALRPWARPEVLTCTPWGAPIIWDGSFDPDVAKQEARQQNLTIGLTIFAVGRYLEKYLERFLETAEQHFMAGQSVMYYVFTELPGAVPRVALGPGRRLPVERVARERRWQDVSMARMRTLHAALGGLPGREAHFMFCMDVDQHFSGTFGPEALAESVAQLHSWHYHWPSWLLPFERDAHSAAAMAWGQGDFYNHAAVFGGSVAALRGLTAHCAGGLDWDRARGLEARWHDESHLNKFFWLHKPAKVLSPEFCWSPDIGPRAEIRRPRLLWAPKGYRLLRN

SEQ ID NO: 121 - protein, 133 aa, Homo sapiens [sequence omitted]

SEQ ID NO: 122 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 123 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 124 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 125 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 126 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 127 - protein, 152 aa, Homo sapiens
SMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 128 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 129 - protein, 111 aa, Homo sapiens
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 130 - DNA, 66 bp, synthetic construct
  source 1..66: mol_type = other DNA
actcgtttgagcgagtataaacagttggttaggctcaaagcggagagcagatctgctctcgtccaa

SEQ ID NO: 131 - DNA, 61 bp, synthetic construct
  source 1..61: mol_type = other DNA
actcgtttgagcgagtataaacagtttgttaggctcaaagcggagagcagatctgctctcg

SEQ ID NO: 132 - DNA, 61 bp, synthetic construct
  source 1..61: mol_type = other DNA
actcgtttgagcgagtataaacagtttattaggctcaaagcggagagcagatctgctctcg

SEQ ID NO: 133 - DNA, 65 bp, synthetic construct
  source 1..65: mol_type = other DNA
actcgtttgagcgagtataaacagtttattaggctcaaagcggagagcagagaaatctgctctcg

SEQ ID NO: 134 - DNA, 73 bp, synthetic construct
  source 1..73: mol_type = other DNA
actcgtttgagcgagtataaacagtttattaggctcaaagcggagagcagaacgagaaatcgttctgctctcg

SEQ ID NO: 135 - DNA, 50 bp, synthetic construct
  source 1..50: mol_type = other DNA
ggatgttaatcaggatatacatacagaaggaaaagtgaactagggcatgg

SEQ ID NO: 136 - DNA, 50 bp, synthetic construct
  source 1..50: mol_type = other DNA
acataattctggtgtaaacgacttaacaatacgaaagcctgcgatcacaa

SEQ ID NO: 137 - DNA, 10 bp, synthetic construct
  source 1..10: mol_type = other DNA
agagaaatct

SEQ ID NO: 138 - DNA, 18 bp, synthetic construct
  source 1..18: mol_type = other DNA
agaacgagaaatcgttct